SEQUENCE LISTING (245 sequences)

SEQ ID NO: 1 - protein, 5 aa, Artificial Sequence
  Description of Artificial Sequence: Synthetic peptide
SYVMH

SEQ ID NO: 2 - protein, 5 aa, Artificial Sequence
  Description of Artificial Sequence: Synthetic peptide
DTYMH

SEQ ID NO: 3 - protein, 5 aa, Artificial Sequence
  Description of Artificial Sequence: Synthetic peptide
EYTIH

SEQ ID NO: 4 - protein, 5 aa, Artificial Sequence
  Description of Artificial Sequence: Synthetic peptide
SYWMN

SEQ ID NO: 5 - protein, 6 aa, Artificial Sequence
  Description of Artificial Sequence: Synthetic peptide
SGYYMC

SEQ ID NO: 6 - protein, 5 aa, Artificial Sequence
  Description of Artificial Sequence: Synthetic peptide
SYWMA

SEQ ID NO: 7 - protein, 6 aa, Artificial Sequence
  Description of Artificial Sequence: Synthetic peptide
SYYYVC

SEQ ID NO: 8 - protein, 5 aa, Artificial Sequence
  Description of Artificial Sequence: Synthetic peptide
SYHMC

SEQ ID NO: 9 - protein, 5 aa, Artificial Sequence
  Description of Artificial Sequence: Synthetic peptide
SYHMN

SEQ ID NO: 10 - protein, 5 aa, Artificial Sequence
  Description of Artificial Sequence: Synthetic peptide
SGYMC

SEQ ID NO: 11 - protein, 6 aa, Artificial Sequence
  Description of Artificial Sequence: Synthetic peptide
SGYYMC

SEQ ID NO: 12 - protein, 5 aa, Artificial Sequence
  Description of Artificial Sequence: Synthetic peptide
SYHMS

SEQ ID NO: 13 - protein, 5 aa, Artificial Sequence
  Description of Artificial Sequence: Synthetic peptide
SGYMS

SEQ ID NO: 14 - protein, 17 aa, Artificial Sequence
  Description of Artificial Sequence: Synthetic peptide
YIDPYNDGTKYNEKFKG

SEQ ID NO: 15 - protein, 17 aa, Artificial Sequence
  Description of Artificial Sequence: Synthetic peptide
RIDPANGKTKSDPKFQG

SEQ ID NO: 16 - protein, 17 aa, Artificial Sequence
  Description of Artificial Sequence: Synthetic peptide
SINPNNGLFRYNQKFKG

SEQ ID NO: 17 - protein, 17 aa, Artificial Sequence
  Description of Artificial Sequence: Synthetic peptide
RIDPANGNTKYDPKFQG

SEQ ID NO: 18 - protein, 17 aa, Artificial Sequence
  Description of Artificial Sequence: Synthetic peptide
SIDPYDSETHYNQKFKD

SEQ ID NO: 19 - protein, 17 aa, Artificial Sequence
  Description of Artificial Sequence: Synthetic peptide
CIYMRSGDTYLASWAKG

SEQ ID NO: 20 - protein, 18 aa, Artificial Sequence
  Description of Artificial Sequence: Synthetic peptide
CIYIGSGSGTTYYASWAK

SEQ ID NO: 21 - protein, 18 aa, Artificial Sequence
  Description of Artificial Sequence: Synthetic peptide
CIYAGSSGDTYYASWAKG

SEQ ID NO: 22 - protein, 17 aa, Artificial Sequence
  Description of Artificial Sequence: Synthetic peptide
CIYPNNGFTWYASWAKG

SEQ ID NO: 23 - protein, 17 aa, Artificial Sequence
  Description of Artificial Sequence: Synthetic peptide
IIYVGSGNADYASWAKG

SEQ ID NO: 24 - protein, 18 aa, Artificial Sequence
  Description of Artificial Sequence: Synthetic peptide
CIYAGSSGKTYYASWATG

SEQ ID NO: 25 - protein, 17 aa, Artificial Sequence
  Description of Artificial Sequence: Synthetic peptide
CIYPNNDFTWYATWAKG

SEQ ID NO: 26 - protein, 17 aa, Artificial Sequence
  Description of Artificial Sequence: Synthetic peptide
CIYVRSDSTYYASWAKG

SEQ ID NO: 27 - protein, 17 aa, Artificial Sequence
  Description of Artificial Sequence: Synthetic peptide
IIYADTGNADYASWTKG

SEQ ID NO: 28 - protein, 17 aa, Artificial Sequence
  Description of Artificial Sequence: Synthetic peptide
YIDPYNEGTKYNEKFKG

SEQ ID NO: 29 - protein, 18 aa, Artificial Sequence
  Description of Artificial Sequence: Synthetic peptide
SIYAGSSGKTYYADWAKG

SEQ ID NO: 30 - protein, 10 aa, Artificial Sequence
  Description of Artificial Sequence: Synthetic peptide
GGMARRSFDY

SEQ ID NO: 31 - protein, 11 aa, Artificial Sequence
  Description of Artificial Sequence: Synthetic peptide
GDGYYVGGLDY

SEQ ID NO: 32 - protein, 9 aa, Artificial Sequence
  Description of Artificial Sequence: Synthetic peptide
DYGFGYSDY

SEQ ID NO: 33 - protein, 11 aa, Artificial Sequence
  Description of Artificial Sequence: Synthetic peptide
GDGHYVGGLNY

SEQ ID NO: 34 - protein, 8 aa, Artificial Sequence
  Description of Artificial Sequence: Synthetic peptide
GRGYAMDY

SEQ ID NO: 35 - protein, 9 aa, Artificial Sequence
  Description of Artificial Sequence: Synthetic peptide
GGTSYALNL

SEQ ID NO: 36 - protein, 11 aa, Artificial Sequence
  Description of Artificial Sequence: Synthetic peptide
GYTGYSYQFNF

SEQ ID NO: 37 - protein, 11 aa, Artificial Sequence
  Description of Artificial Sequence: Synthetic peptide
GSGNSYNQLDL

SEQ ID NO: 38 - protein, 12 aa, Artificial Sequence
  Description of Artificial Sequence: Synthetic peptide
GPTNTYPTYFNL

SEQ ID NO: 39 - protein, 13 aa, Artificial Sequence
  Description of Artificial Sequence: Synthetic peptide
DDGGTYYTDYLYL

SEQ ID NO: 40 - protein, 11 aa, Artificial Sequence
  Description of Artificial Sequence: Synthetic peptide
ASGNAYNLLDL

SEQ ID NO: 41 - protein, 11 aa, Artificial Sequence
  Description of Artificial Sequence: Synthetic peptide
GVTGDSYRFNF

SEQ ID NO: 42 - protein, 12 aa, Artificial Sequence
  Description of Artificial Sequence: Synthetic peptide
GPTNTYPTYFAL

SEQ ID NO: 43 - protein, 13 aa, Artificial Sequence
  Description of Artificial Sequence: Synthetic peptide
DSGGTYYTDYLYA

SEQ ID NO: 44 - protein, 13 aa, Artificial Sequence
  Description of Artificial Sequence: Synthetic peptide
DSGGTYYTDYLYL

SEQ ID NO: 45 - protein, 11 aa, Artificial Sequence
  Description of Artificial Sequence: Synthetic peptide
LASQTIGTWLT

SEQ ID NO: 46 - protein, 11 aa, Artificial Sequence
  Description of Artificial Sequence: Synthetic peptide
HASQNIHVWLS

SEQ ID NO: 47 - protein, 11 aa, Artificial Sequence
  Description of Artificial Sequence: Synthetic peptide
RASQGIRGNLD

SEQ ID NO: 48 - protein, 11 aa, Artificial Sequence
  Description of Artificial Sequence: Synthetic peptide
HASQNINVWLS

SEQ ID NO: 49 - protein, 11 aa, Artificial Sequence
  Description of Artificial Sequence: Synthetic peptide
RASGNIHNYLA

SEQ ID NO: 50 - protein, 11 aa, Artificial Sequence
  Description of Artificial Sequence: Synthetic peptide
QASQSISNECS

SEQ ID NO: 51 - protein, 11 aa, Artificial Sequence
  Description of Artificial Sequence: Synthetic peptide
QASQSINNLLA

SEQ ID NO: 52 - protein, 11 aa, Artificial Sequence
  Description of Artificial Sequence: Synthetic peptide
QASQSISNELS

SEQ ID NO: 53 - protein, 13 aa, Artificial Sequence
  Description of Artificial Sequence: Synthetic peptide
QSSESVYRDNCLG

SEQ ID NO: 54 - protein, 11 aa, Artificial Sequence
  Description of Artificial Sequence: Synthetic peptide
QASRSITSYLA

SEQ ID NO: 55 - protein, 11 aa, Artificial Sequence
  Description of Artificial Sequence: Synthetic peptide
QASEGISSYLS

SEQ ID NO: 56 - protein, 11 aa, Artificial Sequence
  Description of Artificial Sequence: Synthetic peptide
QASQSITTYLA

SEQ ID NO: 57 - protein, 11 aa, Artificial Sequence
  Description of Artificial Sequence: Synthetic peptide
RASEGISSYLS

SEQ ID NO: 58 - protein, 7 aa, Artificial Sequence
  Description of Artificial Sequence: Synthetic peptide
AATTLAD

SEQ ID NO: 59 - protein, 7 aa, Artificial Sequence
  Description of Artificial Sequence: Synthetic peptide
KASNLHT

SEQ ID NO: 60 - protein, 7 aa, Artificial Sequence
  Description of Artificial Sequence: Synthetic peptide
STSNLNS

SEQ ID NO: 61 - protein, 7 aa, Artificial Sequence
  Description of Artificial Sequence: Synthetic peptide
NAKTLAD

SEQ ID NO: 62 - protein, 7 aa, Artificial Sequence
  Description of Artificial Sequence: Synthetic peptide
RASTLES

SEQ ID NO: 63 - protein, 7 aa, Artificial Sequence
  Description of Artificial Sequence: Synthetic peptide
DASDLAS

SEQ ID NO: 64 - protein, 7 aa, Artificial Sequence
  Description of Artificial Sequence: Synthetic peptide
KASTLAS

SEQ ID NO: 65 - protein, 7 aa, Artificial Sequence
  Description of Artificial Sequence: Synthetic peptide
AASNVAS

SEQ ID NO: 66 - protein, 7 aa, Artificial Sequence
  Description of Artificial Sequence: Synthetic peptide
KASTLTS

SEQ ID NO: 67 - protein, 7 aa, Artificial Sequence
  Description of Artificial Sequence: Synthetic peptide
RASTLAS

SEQ ID NO: 68 - protein, 7 aa, Artificial Sequence
  Description of Artificial Sequence: Synthetic peptide
GASNLES

SEQ ID NO: 69 - protein, 7 aa, Artificial Sequence
  Description of Artificial Sequence: Synthetic peptide
AATTLAE

SEQ ID NO: 70 - protein, 9 aa, Artificial Sequence
  Description of Artificial Sequence: Synthetic peptide
QQLYSTPWT

SEQ ID NO: 71 - protein, 9 aa, Artificial Sequence
  Description of Artificial Sequence: Synthetic peptide
QQGQSYPYT

SEQ ID NO: 72 - protein, 9 aa, Artificial Sequence
  Description of Artificial Sequence: Synthetic peptide
LQRNAFPWT

SEQ ID NO: 73 - protein, 9 aa, Artificial Sequence
  Description of Artificial Sequence: Synthetic peptide
QQGQNYPYT

SEQ ID NO: 74 - protein, 9 aa, Artificial Sequence
  Description of Artificial Sequence: Synthetic peptide
QHFWSTPPT

SEQ ID NO: 75 - protein, 11 aa, Artificial Sequence
  Description of Artificial Sequence: Synthetic peptide
QSTYYGTTGNT

SEQ ID NO: 76 - protein, 12 aa, Artificial Sequence
  Description of Artificial Sequence: Synthetic peptide
QGYFYGSSVDLV

SEQ ID NO: 77 - protein, 10 aa, Artificial Sequence
  Description of Artificial Sequence: Synthetic peptide
QCTYITYGNA

SEQ ID NO: 78 - protein, 12 aa, Artificial Sequence
  Description of Artificial Sequence: Synthetic peptide
AGYKSTITDGTA

SEQ ID NO: 79 - protein, 10 aa, Artificial Sequence
  Description of Artificial Sequence: Synthetic peptide
QSYYAISTNT

SEQ ID NO: 80 - protein, 15 aa, Artificial Sequence
  Description of Artificial Sequence: Synthetic peptide
QCTYGSSGSSGYGAA

SEQ ID NO: 81 - protein, 12 aa, Artificial Sequence
  Description of Artificial Sequence: Synthetic peptide
QSTYYGTSYGNA

SEQ ID NO: 82 - protein, 10 aa, Artificial Sequence
  Description of Artificial Sequence: Synthetic peptide
QSYYAISKSA

SEQ ID NO: 83 - protein, 15 aa, Artificial Sequence
  Description of Artificial Sequence: Synthetic peptide
QSTYGSSGSSGYGAA

SEQ ID NO: 84 - protein, 15 aa, Artificial Sequence
  Description of Artificial Sequence: Synthetic peptide
QSAYGSSGSSGYGAA

SEQ ID NO: 85 - protein, 15 aa, Artificial Sequence
  Description of Artificial Sequence: Synthetic peptide
QSTYGSSGSSGYAAA

SEQ ID NO: 86 - protein, 121 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSP

SEQ ID NO: 87 - protein, 122 aa, Macaca fascicularis
QLLFNKTKSVEFTFCNDTVVIPCFVTNMEAQNTTEVYVKWKFKGRDIYTFDGALNKSTAPANFSSAKIEVSQLLKGDASLKMDKSDAVSHTGNYTCEVTELTREGETIIELKYRVVSWFSPN

SEQ ID NO: 88 - protein, 449 aa, Artificial Sequence
  Description of Artificial Sequence: Synthetic polypeptide
EVQLQQSGPELVKPGAPVKMSCKASGYTFTSYVMHWVKQKPGQGLEWIGYIDPYNDGTKYNEKFKGKATLTSDKSSSTAYMELSSLTSEDSAVYYCSRGGMARRSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - DNA, 1347 bp, Artificial Sequence
  Description of Artificial Sequence: Synthetic polynucleotide
gaggtgcagctgcagcagagcgggccagagctggtgaagccaggagccccagtgaagatgagctgtaaggccagcgggtacacatttacaagctacgtgatgcactgggtgaagcagaagcccggacagggcctggaatggatcggatatattgacccctataatgacggcacaaagtacaacgagaaattcaagggcaaagccacactgaccagcgacaagagcagcagcaccgcctacatggagctgagcagcctgaccagcgaagacagcgccgtgtactactgcagcagaggcggaatggccaggagatcctttgactactggggccagggcacaacactgacagtgtctagcgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 90 - protein, 214 aa, Artificial Sequence
  Description of Artificial Sequence: Synthetic polypeptide
DIQMTQSPASQSASLGESVTITCLASQTIGTWLTWYQQKPGKSPQLLIYAATTLADGVPSRFSGSGSGTKFSFKISSLQAEDFVSYYCQQLYSTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - DNA, 645 bp, Artificial Sequence
  Description of Artificial Sequence: Synthetic polynucleotide
gacattcagatgacccagagccccgccagccagagcgcctcactgggagagagcgtgaccatcacctgcctggccagccagaccatcggaacctggctgacctggtaccagcagaaacccggcaaaagcccccagctgctgatttacgccgccaccaccctggccgacggcgtgccttcaagattttccggcagcggcagcggcaccaaattctccttcaagatcagcagcctgcaggccgaagacttcgtgtcctactactgccagcagctgtactccaccccctggaccttcggcggcggcacaaaactggagatcaagcgtacggtggcagctccatcagtttttatcttcccaccaagcgacgagcaattgaagtccggcactgcctctgtggtgtgccttctgaacaacttctatccaagggaggccaaggtccagtggaaggtcgataatgcgctgcagagcgggaacagccaagagtcagtgaccgagcaggactcaaaagatagcacatactctctgagttccaccctgaccctgtcaaaggctgactacgaaaagcataaggtatacgcatgcgaagtgacccatcagggtctctcatctcccgtaaccaaatcttttaatagaggagaatgctag

SEQ ID NO: 92 - protein, 450 aa, Artificial Sequence
  Description of Artificial Sequence: Synthetic polypeptide
EVQLQQSGAELVRPGASVKLSCTASGFNIEDTYMHWVKQRPEQGLEWIGRIDPANGKTKSDPKFQGKATITTDTSSNTAYLQLSSLTSEDTAVYYCAKGDGYYVGGLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - DNA, 1350 bp, Artificial Sequence
  Description of Artificial Sequence: Synthetic polynucleotide
gaggtgcagctgcagcagagcggggccgagctggtgagaccaggagccagcgtgaagctgagctgtacagccagcggcttcaacatcgaggacacatatatgcactgggtgaaacagaggcccgagcagggcctggagtggatcggcagaatcgaccccgctaacggaaaaacaaaatcagaccccaaattccagggcaaagccaccatcaccaccgacaccagcagcaacaccgcctatctgcagctgagcagcctgaccagcgaggacaccgccgtgtactactgcgccaaaggcgacggctactacgtgggcggactggactactggggccagggaaccaccctgacagtgagcagcgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 94 - protein, 214 aa, Artificial Sequence
  Description of Artificial Sequence: Synthetic polypeptide
DIQMNQSPSSLSASLGDTITITCHASQNIHVWLSWYQQKPGNIPKLLIYKASNLHTGVPSRFSGSGSGTGFTLTISSLQPEDIATYYCQQGQSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - DNA, 645 bp, Artificial Sequence
  Description of Artificial Sequence: Synthetic polynucleotide
gacattcagatgaaccagagccccagcagcctgagcgccagcctgggagacaccatcaccatcacctgccacgccagccagaacatccacgtgtggctgagctggtatcagcagaaacccggcaacatccccaaactgctgatctacaaagcctccaacctgcacaccggcgtgcccagcagattcagcggaagcggcagcggcaccggattcaccctgaccatcagcagcctgcagcccgaagacatcgccacctactactgccagcagggccagagctacccctacacattcggcggcggcacaaagctggaaatcaagcgtacggtggcagctccatcagtttttatcttcccaccaagcgacgagcaattgaagtccggcactgcctctgtggtgtgccttctgaacaacttctatccaagggaggccaaggtccagtggaaggtcgataatgcgctgcagagcgggaacagccaagagtcagtgaccgagcaggactcaaaagatagcacatactctctgagttccaccctgaccctgtcaaaggctgactacgaaaagcataaggtatacgcatgcgaagtgacccatcagggtctctcatctcccgtaaccaaatcttttaatagaggagaatgctag

SEQ ID NO: 96 - protein, 448 aa, Artificial Sequence
  Description of Artificial Sequence: Synthetic polypeptide
EVQLQQSGPELVKPGASVKMSCKTSGYTFTEYTIHWVKQSHEKSLEWIGSINPNNGLFRYNQKFKGKATLTVDKSSSTAYMELRSLTSEDSAVYYCARDYGFGYSDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 214 aa, Artificial Sequence
  Description of Artificial Sequence: Synthetic polypeptide
DIQMIQSPSSMFASLGDRVSLSCRASQGIRGNLDWYQQKPGGTIKLLIYSTSNLNSGVPSRFSGSGSGSDYSLTISSLESEDFADYYCLQRNAFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - DNA, 645 bp, Artificial Sequence
  Description of Artificial Sequence: Synthetic polynucleotide
gacattcagatgattcagagccccagcagcatgtttgccagcctgggcgatagagtgagcctgagctgtagggccagccaggggatcaggggcaatctggattggtatcagcagaagcctggcggaaccatcaagctgctgatctacagcaccagcaacctgaacagcggagtgccaagcagattcagcggaagcggcagcggcagcgattacagcctgaccatcagctcactggaaagcgaggacttcgccgactactactgcctgcagagaaacgccttcccctggaccttcggcggcggcacaaaactggagatcaagcgtacggtggcagctccatcagtttttatcttcccaccaagcgacgagcaattgaagtccggcactgcctctgtggtgtgccttctgaacaacttctatccaagggaggccaaggtccagtggaaggtcgataatgcgctgcagagcgggaacagccaagagtcagtgaccgagcaggactcaaaagatagcacatactctctgagttccaccctgaccctgtcaaaggctgactacgaaaagcataaggtatacgcatgcgaagtgacccatcagggtctctcatctcccgtaaccaaatcttttaatagaggagaatgctag

SEQ ID NO: 99 - protein, 450 aa, Artificial Sequence
  Description of Artificial Sequence: Synthetic polypeptide
EVQLQQSGAELVKPGASVKLSCTASGFNIEDTYMHWVKQRPEQGLEWIGRIDPANGNTKYDPKFQGKATVTADTSSNTAYLQLSSLTSEDTAVYCCARGDGHYVGGLNYWGQGTSLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - DNA, 1350 bp, Artificial Sequence
  Description of Artificial Sequence: Synthetic polynucleotide
gaggtgcagctgcagcagagcggggccgagctggtgaagccaggagccagcgtgaagctgagctgtacagccagcggcttcaacatcgaggacacatatatgcactgggtgaaacagaggcccgagcagggcctggagtggatcggcagaatcgaccccgctaacggaaacaccaaatacgaccccaagttccagggcaaagccaccgtgaccgccgacacctccagcaacaccgcctatctgcagctgagcagcctgaccagcgaggacaccgccgtgtactgctgcgccagaggcgacggccactacgtgggaggactgaactactggggacagggcaccagcctgacagtgtccagcgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 101 - protein, 214 aa, Artificial Sequence
  Description of Artificial Sequence: Synthetic polypeptide
DIQMNQSPSSLSASLGDTITITCHASQNINVWLSWYQQKPGNIPKLLIYKASNLHTGVPSRFSGSGSGTGFTLTISSLQPEDIATYYCQQGQNYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - DNA, 645 bp, Artificial Sequence
  Description of Artificial Sequence: Synthetic polynucleotide
gacattcagatgaaccagagccccagcagcctgagcgccagcctgggagacaccatcaccatcacctgccacgccagccagaacatcaacgtgtggctgagctggtatcagcagaaacccggcaacatccccaaactgctgatctacaaagcctccaacctgcacaccggcgtgcccagcagattcagcggaagcggcagcggcaccggattcaccctgaccatcagcagcctgcagcccgaagacatcgccacctactactgccagcagggccagaactacccctacaccttcggcggcggcaccaagctggagatcaagcgtacggtggcagctccatcagtttttatcttcccaccaagcgacgagcaattgaagtccggcactgcctctgtggtgtgccttctgaacaacttctatccaagggaggccaaggtccagtggaaggtcgataatgcgctgcagagcgggaacagccaagagtcagtgaccgagcaggactcaaaagatagcacatactctctgagttccaccctgaccctgtcaaaggctgactacgaaaagcataaggtatacgcatgcgaagtgacccatcagggtctctcatctcccgtaaccaaatcttttaatagaggagaatgctag

SEQ ID NO: 103 - DNA, 1341 bp, Artificial Sequence
  Description of Artificial Sequence: Synthetic polynucleotide
caggtgcagctgcagcagccaggcgccgagctggtgagacccggagctagcgtgaaactgagctgcaaagccagcggctacaccttcacctcctactggatgaactgggtgaagcagagacccgaacagggactggagtggattggaagcattgacccctacgatagcgagacccactacaaccagaaattcaaggacaaagccatcctgaccgtggacaaaagcagcagcacagcctacatgcagctgagcagcctgaccagcgaggacagcgccgtgtactactgcgccagaggcagaggctacgccatggactactggggccagggcaccagcgtgaccgtgagcagcgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 104 - protein, 214 aa, Artificial Sequence
  Description of Artificial Sequence: Synthetic polypeptide
DIQMTQSPASLSASVGETVTITCRASGNIHNYLAWYQQKQGKSPQLLVYNAKTLADGVPSRFSGSGSGTQYSLKINSLQPEDFGSYYCQHFWSTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - DNA, 645 bp, Artificial Sequence
  Description of Artificial Sequence: Synthetic polynucleotide
gacattcagatgacccagagccccgccagcctgagcgcctctgtgggagagaccgtgaccatcacctgcagagccagcggcaacatccacaactacctggcctggtaccagcagaaacagggcaagagcccccagctgctggtgtacaacgccaagaccctggccgacggcgtgccctcaagattcagcggaagcggaagcggcacacagtacagcctgaagatcaacagcctgcagcccgaggacttcggctcctactattgccagcacttctggtctaccccccccaccttcggcggcggaacaaaactggagatcaagcgtacggtggcagctccatcagtttttatcttcccaccaagcgacgagcaattgaagtccggcactgcctctgtggtgtgccttctgaacaacttctatccaagggaggccaaggtccagtggaaggtcgataatgcgctgcagagcgggaacagccaagagtcagtgaccgagcaggactcaaaagatagcacatactctctgagttccaccctgaccctgtcaaaggctgactacgaaaagcataaggtatacgcatgcgaagtgacccatcagggtctctcatctcccgtaaccaaatcttttaatagaggagaatgctag

SEQ ID NO: 106 - protein, 447 aa, Artificial Sequence
  Description of Artificial Sequence: Synthetic polypeptide
QSLEESGGDLVKPGASLTLTCTASGFSFSSGYYMCWVRQAPGKGLEWIACIYMRSGDTYLASWAKGQFTISKTSSTTVTLQMTSLTAADTATYFCGRGGTSYALNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - DNA, 1341 bp, Artificial Sequence
  Description of Artificial Sequence: Synthetic polynucleotide
cagtctctggaagaatccggcggagacctggtgaagcctggagcttctctgaccctgacctgcacagcatccggattttccttttcctccggctactacatgtgctgggtgaggcaggcacccggaaaaggactggaatggattgcctgtatctacatgcgttccggcgacacctacctggcttcttgggctaagggccagttcaccatttccaagacctcctccaccaccgtcaccctgcagatgacatccctgaccgctgctgacaccgctacatacttctgtggccgcggaggtacctcctacgctctgaacctgtggggacagggaaccctggtgacagtgtcttccgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 108 - protein, 216 aa, Artificial Sequence
  Description of Artificial Sequence: Synthetic polypeptide
DVVMTQTPASVSEPVGGTVTINCQASQSISNECSWYQQKPGQPPKLLIYRASTLESGVPSRFSGSGSGTEFTLTISDLESADAATYYCQSTYYGTTGNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - DNA, 648 bp, Artificial Sequence
  Description of Artificial Sequence: Synthetic polynucleotide
gatgtggtgatgacccagaccccagcaagcgtgagtgagcctgtgggaggaacagtgaccattaattgccaggcctcccagtccattagtaacgagtgtagctggtaccagcagaagcccggtcagcctcctaagctgctgatttacagggcctccaccctggagagcggagtgccttctagattctccggatccggctccggaaccgaatttaccctgaccatctccgacctggaatctgccgacgctgcaacttactactgccagtccacctactacgggacaaccggaaacaccttcggcggaggaaccgaagtggtggtgaagcgtacggtggcagctccatcagtttttatcttcccaccaagcgacgagcaattgaagtccggcactgcctctgtggtgtgccttctgaacaacttctatccaagggaggccaaggtccagtggaaggtcgataatgcgctgcagagcgggaacagccaagagtcagtgaccgagcaggactcaaaagatagcacatactctctgagttccaccctgaccctgtcaaaggctgactacgaaaagcataaggtatacgcatgcgaagtgacccatcagggtctctcatctcccgtaaccaaatcttttaatagaggagaatgc

SEQ ID NO: 110 - protein, 450 aa, Artificial Sequence
  Description of Artificial Sequence: Synthetic polypeptide
QEQLEESGGDLVKPEGSLTLTCTASGFSFSSYWMAWVRQAPGKGLEWIACIYIGSGSGTTYYASWAKGRFTISKTSSTTVTLQMTSLTAADTAIYFCARWAGSDGHFTLWGPGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - DNA, 1350 bp, Artificial Sequence
  Description of Artificial Sequence: Synthetic polynucleotide
caggaacagctggaggaaagcggaggagacctggtgaagccagagggaagtctgacactgacctgcaccgctagtggcttctcctttagcagctactggatggcctgggtgaggcaggctcctggaaaaggactggagtggatcgcatgcatttacatcggcagcggctctggcaccacatactacgcttcctgggccaagggcaggttcactatctccaaaaccagttcaacaaccgtcaccctgcagatgacaagcctgaccgctgctgacaccgctatctatttttgtgcccggtgggccggttccgacggacatttcacactgtggggccctggaaccctggtgacagtgtctctggctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 112 - protein, 217 aa, Artificial Sequence
  Description of Artificial Sequence: Synthetic polypeptide
ALVMTQTPASVEAAVGGTVTINCQASQSINNLLAWYQQKPGQRPKLLIYDASDLASGVSSRFKGSGSGTEYTLTISGVQSADAATYYCQGYFYGSSVDLVFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - DNA, 651 bp, Artificial Sequence
  Description of Artificial Sequence: Synthetic polynucleotide
gctctggtgatgacccagacacctgcttccgtggaggctgctgtgggaggaacagtgacaatcaactgccaggccagccagtccattaacaacctgctggcctggtaccagcagaaaccaggacagaggcccaagctgctgatttacgatgcatccgatctggcaagcggcgtgtcttctaggttcaagggctccggatccggcacagaatacaccctgaccatctccggcgtgcagtctgctgatgctgcaacatactactgccagggatacttttacggctcctccgtggacctggtgttcggaggaggaacagaggtggtggtgaagcgtacggtggcagctccatcagtttttatcttcccaccaagcgacgagcaattgaagtccggcactgcctctgtggtgtgccttctgaacaacttctatccaagggaggccaaggtccagtggaaggtcgataatgcgctgcagagcgggaacagccaagagtcagtgaccgagcaggactcaaaagatagcacatactctctgagttccaccctgaccctgtcaaaggctgactacgaaaagcataaggtatacgcatgcgaagtgacccatcagggtctctcatctcccgtaaccaaatcttttaatagaggagaatgc

SEQ ID NO: 114 - protein, 450 aa, Artificial Sequence
  Description of Artificial Sequence: Synthetic polypeptide
QSLEESGGDLVKPGASLTVTCTASGFSFSSYYYVCWVRQAPGKGLEWIACIYAGSSGDTYYASWAKGRFTISKTSSTTVTLQMTSLTAADTATYFCARGYTGYSYQFNFWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - DNA, 1350 bp, Artificial Sequence
  Description of Artificial Sequence: Synthetic polynucleotide
cagtctctggaagaatccggcggagacctggtgaagcctggagcttctctgaccgtcacctgcacagcatccggatttagcttttcctcctactactacgtctgctgggtcaggcaggcccctggaaagggactggaatggattgcctgcatctacgccggttcctccggagatacctactacgcttcttgggccaagggccgtttcaccatctccaagacctcttccaccaccgtgaccctgcagatgacctctctgaccgctgctgacaccgctacatacttttgcgcccgcggatacaccggctactcttatcagttcaacttctggggccccggcaccctggtgacagtgtcttctgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 116 - protein, 215 aa, Artificial Sequence
  Description of Artificial Sequence: Synthetic polypeptide
DVVMTQTPASVEAAVGGTVTIKCQASQSISNELSWYQQKPGQPPKLLIYRASTLESGVPSRFKGSGSGTEFTLTISDLESADAATYYCQCTYITYGNAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - DNA, 645 bp, Artificial Sequence
  Description of Artificial Sequence: Synthetic polynucleotide
gatgtggtgatgacccagaccccagcaagcgtggaagcagctgtgggaggaacagtgaccattaagtgccaggcctcccagtccatttccaacgagctgagctggtaccagcagaagcccggacagcctcctaaactgctgatctatagggcaagtaccctggagagcggagtgccttctaggttcaaaggatccggcagcggaactgaatttaccctgactatctccgacctggagagcgctgacgctgctacatactactgccagtgtacctacatcacatacgggaatgcctttgggggcggcacagaagtggtggtgaagcgtacggtggcagctccatcagtttttatcttcccaccaagcgacgagcaattgaagtccggcactgcctctgtggtgtgccttctgaacaacttctatccaagggaggccaaggtccagtggaaggtcgataatgcgctgcagagcgggaacagccaagagtcagtgaccgagcaggactcaaaagatagcacatactctctgagttccaccctgaccctgtcaaaggctgactacgaaaagcataaggtatacgcatgcgaagtgacccatcagggtctctcatctcccgtaaccaaatcttttaatagaggagaatgc

SEQ ID NO: 118 - protein, 449 aa, Artificial Sequence
  Description of Artificial Sequence: Synthetic polypeptide
QEQLKESGGGLVTPGGTLTLTCTASGFTITSYHMCWVRQAPGKGLEWIGCIYPNNGFTWYASWAKGRFTISKTSSTTVDLKMTSPTTEEMATYFCARGSGNSYNQLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - DNA, 1347 bp, Artificial Sequence
  Description of Artificial Sequence: Synthetic polynucleotide
caggaacagctgaaggagagcggaggaggactggtgacaccaggaggaacactgacactgacctgcaccgcaagcggattcaccatcaccagctatcacatgtgctgggtgagacaggccccaggaaagggactggaatggatcggatgcatttacccaaataacggctttacctggtacgcctcctgggcaaagggcagattcaccatctccaaaacctcctccaccacagtcgatctgaagatgacatcccccaccaccgaagagatggccacatacttctgcgcccgcggatctggaaacagctacaaccagctggacctgtggggccagggaacactggtgacagtgagtagcgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 120 - protein, 219 aa, Artificial Sequence
  Description of Artificial Sequence: Synthetic polypeptide
AIVMTQTPASVSATLGGSVTINCQSSESVYRDNCLGWFQQKPGQPPKLLIYKASTLASGVPSRFKGSGSGTQFTLTISDVVSDDAATYYCAGYKSTITDGTAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - DNA, 657 bp, Artificial Sequence
  Description of Artificial Sequence: Synthetic polynucleotide
gctattgtgatgacccagacccccgcatccgtgtctgctacactgggaggaagtgtgaccattaattgtcagtcctccgagtccgtgtaccgcgataattgtctgggatggttccagcagaagcctgggcagcctcctaaactgctgatttacaaagcctctacactggcctccggcgtcccttctagattcaagggatccgggtccggcacccagtttactctgactatctccgacgtggtgtctgacgacgccgctacatactactgcgcaggttacaagtccaccatcaccgatggcaccgccttcggaggaggaacagaagtggtggtgaagcgtacggtggcagctccatcagtttttatcttcccaccaagcgacgagcaattgaagtccggcactgcctctgtggtgtgccttctgaacaacttctatccaagggaggccaaggtccagtggaaggtcgataatgcgctgcagagcgggaacagccaagagtcagtgaccgagcaggactcaaaagatagcacatactctctgagttccaccctgaccctgtcaaaggctgactacgaaaagcataaggtatacgcatgcgaagtgacccatcagggtctctcatctcccgtaaccaaatcttttaatagaggagaatgc

SEQ ID NO: 122 - skipped

SEQ ID NO: 123 - protein, 448 aa, Artificial Sequence
  Description of Artificial Sequence: Synthetic polypeptide
QSLEESGGRLVTPGTPLTLTCTASGFTISSYHMNWVRQAPGEGLEWIGIIYVGSGNADYASWAKGRFTISKASTTVDLKITSPTTEDTATYFCARGPTNTYPTYFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - DNA, 1344 bp, Artificial Sequence
  Description of Artificial Sequence: Synthetic polynucleotide
cagtctctggaagaatccggcggaaggctggtgacaccaggaacacctctgaccctgacctgcacagcatccggattcaccatctcctcctaccacatgaactgggtccggcaggcacctggagaaggactggaatggatcggaatcatctacgtcggctccggcaacgcagactacgcttcttgggctaaaggccgcttcactatctccaaggcctccactaccgtcgacctgaaaatcacaagccccaccaccgaagacaccgcaacatatttttgcgctaggggaccaaccaacacctaccctacatacttcaacctgtggggccccggaaccctggtgacagtgtcttctgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 125 - protein, 215 aa, Artificial Sequence
  Description of Artificial Sequence: Synthetic polypeptide
AFELTQTPSSVEAAVGGTVTIKCQASRSITSYLAWYQHKPGQPPTLLIYAASNVASGVSSRFKGSRSGTEYTLTISDLESADAATYYCQSYYAISTNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - DNA, 645 bp, Artificial Sequence
  Description of Artificial Sequence: Synthetic polynucleotide
gcttttgaactgacccagaccccctcctccgtggaagctgctgtgggaggaacagtgaccattaagtgtcaggcctctaggtctatcacctcctacctggcttggtatcagcacaagcccggtcagcctccaaccctgctgatttacgctgcctctaatgtggcttctggcgtgtcctcccgcttcaaaggatccagatccggcaccgagtacaccctgactatctccgacctggaatctgccgacgccgctacatactactgccagtcttactacgccatttccaccaataccttcggcggcggaaccgaagtggtcgtgaaacgtacggtggcagctccatcagtttttatcttcccaccaagcgacgagcaattgaagtccggcactgcctctgtggtgtgccttctgaacaacttctatccaagggaggccaaggtccagtggaaggtcgataatgcgctgcagagcgggaacagccaagagtcagtgaccgagcaggactcaaaagatagcacatactctctgagttccaccctgaccctgtcaaaggctgactacgaaaagcataaggtatacgcatgcgaagtgacccatcagggtctctcatctcccgtaaccaaatcttttaatagaggagaatgc

SEQ ID NO: 127 - protein, 451 aa, Artificial Sequence
  Description of Artificial Sequence: Synthetic polypeptide
QSLEESGGDLVKPGASLTLTCKASGFSFNSGYMCWVRQAPGKGLEWIACIYAGSSGKTYYASWATGRFTISKTSSTTVTLQMTSLTVADTATYFCARDDGGTYYTDYLYLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - DNA, 1353 bp, Artificial Sequence
  Description of Artificial Sequence: Synthetic polynucleotide
cagtctctggaagaatccggcggagacctggtgaagcctggagcttctctgaccctgacctgcaaagcctccggattttcctttaactccggctacatgtgctgggtgcggcaggctcctggaaaaggactggaatggatcgcctgcatttacgccgggtcttccggaaagacctactacgcatcctgggctaccggcagattcaccatttccaagactagcagtacaaccgtgacactgcagatgacctccctgaccgtggctgataccgctacatacttctgtgcccgggacgacggaggtacatattacaccgactacctgtacctgtggggcccaggaacactggtgacagtgtcttccgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 129 - protein, 220 aa, Artificial Sequence
  Description of Artificial Sequence: Synthetic polypeptide
DVVMTQTPASVEAAVGGTVTIKCQASEGISSYLSWYQQKPGQPPKLLIYKASTLTSGVPSRFKGSGSGTDFTLTISDLESADAATYYCQCTYGSSGSSGYGAAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - DNA, 660 bp, Artificial Sequence
  Description of Artificial Sequence: Synthetic polynucleotide
gatgtggtgatgacccagaccccagcaagcgtggaagcagctgtgggaggaacagtgaccattaagtgccaggcctccgaaggaatcagcagctatctgagctggtatcagcagaagcccgggcagcctcctaaactgctgatttacaaggcaagtaccctgacatctggcgtgccctctaggtttaaaggaagcggaagcggcacagacttcaccctgacaatctccgacctggaaagcgccgacgcagctacatactactgccagtgcacctacggctcctccggatcttctggttacggcgctgcttttggcgggggaacagaagtggtggtgaagcgtacggtggcagctccatcagtttttatcttcccaccaagcgacgagcaattgaagtccggcactgcctctgtggtgtgccttctgaacaacttctatccaagggaggccaaggtccagtggaaggtcgataatgcgctgcagagcgggaacagccaagagtcagtgaccgagcaggactcaaaagatagcacatactctctgagttccaccctgaccctgtcaaaggctgactacgaaaagcataaggtatacgcatgcgaagtgacccatcagggtctctcatctcccgtaaccaaatcttttaatagaggagaatgc

SEQ ID NO: 131 - protein, 449 aa, Artificial Sequence
  Description of Artificial Sequence: Synthetic polypeptide
QEQLKESGGGLVTPGGTLTLTCTASGFTISSYHMCWVRQAPGKGLEWIGCIYPNNDFTWYATWAKGRFTISKTSSTTVDLRMTTPTTEETATYFCARASGNAYNLLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - DNA, 1347 bp, Artificial Sequence
  Description of Artificial Sequence: Synthetic polynucleotide
caggaacagctgaaggagagcggaggaggactggtgacaccaggaggaacactgacactgacctgcaccgcaagcggattcaccatcagcagctaccacatgtgctgggtgaggcaggcaccaggaaagggactggaatggattggctgcatttacccaaataacgacttcacctggtacgctacatgggccaagggcagattcaccatctccaaaacctcctccaccaccgtcgatctgaggatgaccacccctactacagaggagaccgccacatacttctgcgccagagcttccggtaacgcctacaacctgctggacctgtggggacagggaacactggtgacagtctcctccgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 133 - protein, 219 aa, Artificial Sequence
  Description of Artificial Sequence: Synthetic polypeptide
AIVMTQTPASVSVAVGGSVTINCQSSESVYRDNCLGWFQQKPGQPPKLLIYRASTLASGVPSRFKGSGSGTQFTLTISDVVSDDAATYYCAGYKSTITDGTAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - DNA, 657 bp, Artificial Sequence
  Description of Artificial Sequence: Synthetic polynucleotide
gctattgtgatgacccagacccccgcatccgtgtctgtggctgtgggaggatctgtgaccattaattgtcagtccagtgaatccgtgtatcgtgacaactgtctgggatggttccagcagaaacccgggcagcctcctaaactgctgatttacagggcctctacactggcttccggcgtgccttctaggttcaagggatccggatccggcacacagttcactctgactatctccgacgtggtgtctgacgacgccgctacatactactgcgcaggatacaagtccaccatcaccgacggcaccgcattcggaggaggaacagaagtggtggtgaagcgtacggtggcagctccatcagtttttatcttcccaccaagcgacgagcaattgaagtccggcactgcctctgtggtgtgccttctgaacaacttctatccaagggaggccaaggtccagtggaaggtcgataatgcgctgcagagcgggaacagccaagagtcagtgaccgagcaggactcaaaagatagcacatactctctgagttccaccctgaccctgtcaaaggctgactacgaaaagcataaggtatacgcatgcgaagtgacccatcagggtctctcatctcccgtaaccaaatcttttaatagaggagaatgc

SEQ ID NO: 135 - protein, 450 aa, Artificial Sequence
  Description of Artificial Sequence: Synthetic polypeptide
QEQLVESGGGLVKPGASLTLTCKASGFSFSSGYYMCWVRQAPGKGLEWIGCIYVRSDSTYYASWAKGRFTISKASSTTVTLQMTSLTAADTATYFCARGVTGDSYRFNFWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - DNA, 1350 bp, Artificial Sequence
  Description of Artificial Sequence: Synthetic polynucleotide
caggaacagctggtggaaagtggcggaggactggtgaagcctggagcttctctgaccctgacatgcaaggcatccggcttcagcttcagtagcggctactatatgtgctgggtgagacaggccccaggcaaaggactggaatggatcggatgtatctacgtgcgatccgactccacctactacgcctcttgggctaagggaaggttcaccatttccaaggcctcctccaccaccgtcaccctgcagatgacatccctgactgccgctgacaccgctacatatttctgcgccaggggcgtgaccggagattcttataggttcaacttctggggccccggaaccctggtgacagtgtcttctgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 137 - protein, 217 aa, Artificial Sequence
  Description of Artificial Sequence: Synthetic polypeptide
DVVMTQTPASVEAAVGGTVTIKCQASQSISNELSWYQQKSGQPPKLLIYRASTLESGVPSRFKGSGSGTEFTLTISDLESADAATYYCQSTYYGTSYGNAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - DNA, 651 bp, Artificial Sequence
  Description of Artificial Sequence: Synthetic polynucleotide
gatgtggtgatgacccagaccccagcaagcgtggaagcagctgtgggaggaacagtgaccattaagtgccaggcctcccagtccatttccaacgagctgagctggtaccagcagaagagcggacagcctccaaaactgctgatttacagggcaagtacactggagagcggagtgccatccaggttcaagggatccggaagcggaaccgagttcaccctgactatctccgacctggagtctgccgacgctgctacatactactgccagtccacctactacggcaccagttacggcaatgcattcggcggaggcacagaggtggtggtgaaacgtacggtggcagctccatcagtttttatcttcccaccaagcgacgagcaattgaagtccggcactgcctctgtggtgtgccttctgaacaacttctatccaagggaggccaaggtccagtggaaggtcgataatgcgctgcagagcgggaacagccaagagtcagtgaccgagcaggactcaaaagatagcacatactctctgagttccaccctgaccctgtcaaaggctgactacgaaaagcataaggtatacgcatgcgaagtgacccatcagggtctctcatctcccgtaaccaaatcttttaatagaggagaatgc

SEQ ID NO: 139 - protein, 448 aa, Artificial Sequence
  Description of Artificial Sequence: Synthetic polypeptide
QSLEESGGRLVTPGTPLTLTCTASGFSLNSYHMSWVRQAPGEGLEWIGIIYADTGNADYASWTKGRFTISKASTTVELRITSPTSEDTATYFCARGPTNTYPTYFALWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - DNA, 1347 bp, Artificial Sequence
  Description of Artificial Sequence: Synthetic polynucleotide
cagtctctggaagaatccggcggaaggctggtgacaccaggaacacctctgaccctgacctgcacagcatccggattcagcctgaactcctaccacatgtcctgggtccgccaggctcctggagaaggactggaatggatcggaatcatttacgccgacaccggcaatgccgactacgcttcttggaccaagggcagatttaccatctccaaagcctccaccaccgtggaactgaggattacctctcccacctccgaagacaccgccacatacttctgcgctaggggacctaccaacacctaccctacctacttcgccctgtggggaccaggaacactggtgacagtgagcagcgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaagtag

SEQ ID NO: 141 - protein, 215 aa, Artificial Sequence
  Description of Artificial Sequence: Synthetic polypeptide
AFELTQTPSSVEAAVGGTVTIKCQASQSITTYLAWYQQKPGQPPKLLIYGASNLESGVPSRFKGSRSGTEYTLTISDLESADAATYYCQSYYAISKSAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - DNA, 648 bp, Artificial Sequence
  Description of Artificial Sequence: Synthetic polynucleotide
gcttttgaactgacccagaccccctcctccgtggaagctgctgtgggaggaacagtgaccattaagtgtcaggcctctcagtccattaccacctatctggcctggtaccagcagaagcccggtcagcctcctaagctgctgatctacggcgcaagtaacctggagagcggagtgccttccaggttcaaaggatccaggtccggcacagagtacaccctgacaattagcgacctggaaagcgccgacgccgctacatattactgccagtcctactacgccatttccaagtccgccttcggcggtggaacagaagtggtggtgaaacgtacggtggcagctccatcagtttttatcttcccaccaagcgacgagcaattgaagtccggcactgcctctgtggtgtgccttctgaacaacttctatccaagggaggccaaggtccagtggaaggtcgataatgcgctgcagagcgggaacagccaagagtcagtgaccgagcaggactcaaaagatagcacatactctctgagttccaccctgaccctgtcaaaggctgactacgaaaagcataaggtatacgcatgcgaagtgacccatcagggtctctcatctcccgtaaccaaatcttttaatagaggagaatgctga

SEQ ID NO: 143 - protein, 119 aa, Artificial Sequence
  Description of Artificial Sequence: Synthetic polypeptide
EVQLVQSGAEVKKPGASVKVSCKASGYTFTSYVMHWVRQAPGQRLEWMGYIDPYNEGTKYNEKFKGRVTITRDTSASTAYMELSSLRSEDTAVYYCSRGGMARRSFDYWGQGTLVTVSS

SEQ ID NO: 144 - protein, 107 aa, Artificial Sequence
  Description of Artificial Sequence: Synthetic polypeptide
DIQMTQSPSSLSASVGDRVTITCLASQTIGTWLTWYQQKPGKAPKLLIYAATTLAEGVPSRFSGSGSGTDFTLTISSLQPEDFATYYCQQLYSTPWTFGGGTKVEIK

SEQ ID NO: 145 - protein, 449 aa, Artificial Sequence
  Description of Artificial Sequence: Synthetic polypeptide
EVQLVQSGAEVKKPGASVKVSCKASGYTFTSYVMHWVRQAPGQRLEWMGYIDPYNEGTKYNEKFKGRVTITRDTSASTAYMELSSLRSEDTAVYYCSRGGMARRSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - DNA, 1347 bp, Artificial Sequence
  Description of Artificial Sequence: Synthetic polynucleotide
gaggtgcagctggtgcagagcggagccgaggtgaagaagccaggagccagcgtgaaggtgagctgtaaggctagtggatacacattcacaagctacgtgatgcactgggtgagacaggcccctggacagagactggagtggatgggatatatcgacccctataatgagggcaccaagtacaacgaaaaattcaagggcagagtgaccatcaccagagacaccagcgccagcaccgcctacatggagctgagcagcctgagaagcgaggacaccgccgtgtactactgcagcagaggcggcatggccaggagatcctttgactactggggccagggaacactggtgaccgtgagcagcgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 147 - protein, 214 aa, Artificial Sequence
  Description of Artificial Sequence: Synthetic polypeptide
DIQMTQSPSSLSASVGDRVTITCLASQTIGTWLTWYQQKPGKAPKLLIYAATTLAEGVPSRFSGSGSGTDFTLTISSLQPEDFATYYCQQLYSTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - DNA, 642 bp, Artificial Sequence
  Description of Artificial Sequence: Synthetic polynucleotide
gacattcagatgacccagagccccagcagcctgagcgccagcgtgggagacagagtgaccatcacctgcctggccagccagaccatcggaacctggctgacctggtaccagcagaaacccggcaaagcccccaaactgctgatctacgccgccaccacactggccgagggcgtgccttccagattcagcgggagcggcagcggcacagacttcacactgactatcagcagcctgcagcctgaggatttcgccacctactactgccagcagctgtactccaccccctggacattcggcggcggcaccaaggtggagatcaagcgtacggtggcagctccatcagtttttatcttcccaccaagcgacgagcaattgaagtccggcactgcctctgtggtgtgccttctgaacaacttctatccaagggaggccaaggtccagtggaaggtcgataatgcgctgcagagcgggaacagccaagagtcagtgaccgagcaggactcaaaagatagcacatactctctgagttccaccctgaccctgtcaaaggctgactacgaaaagcataaggtatacgcatgcgaagtgacccatcagggtctctcatctcccgtaaccaaatcttttaatagaggagaatgc

SEQ ID NO: 149 - protein, 119 aa, Artificial Sequence
  Description of Artificial Sequence: Synthetic polypeptide
EVQLVQSGAEVKKPGASVKVSCKASGYTFTSYVMHWVRQAPGQRLEWMGYIDPYNEGTKYNEKFKGRVTITSDTSASTAYMELSSLRSEDTAVYYCSRGGMARRSFDYWGQGTLVTVSS

SEQ ID NO: 150 - protein, 107 aa, Artificial Sequence; identical to SEQ ID NO: 144
  Description of Artificial Sequence: Synthetic polypeptide

SEQ ID NO: 151 - protein, 449 aa, Artificial Sequence
  Description of Artificial Sequence: Synthetic polypeptide
EVQLVQSGAEVKKPGASVKVSCKASGYTFTSYVMHWVRQAPGQRLEWMGYIDPYNEGTKYNEKFKGRVTITSDTSASTAYMELSSLRSEDTAVYYCSRGGMARRSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - DNA, 1347 bp, Artificial Sequence
  Description of Artificial Sequence: Synthetic polynucleotide
gaggtgcagctggtgcagagcggagccgaggtgaagaagccaggagccagcgtgaaggtgagctgtaaggctagtggatacacattcacaagctacgtgatgcactgggtgagacaggcccctggacagagactggagtggatgggatatatcgacccctataatgagggcaccaagtacaacgaaaaattcaagggcagagtgaccatcaccagcgacacctctgccagcaccgcctacatggagctgagctcactgagaagcgaggacaccgccgtgtactactgcagcagaggcggcatggccaggagatcctttgactactggggccagggaacactggtgaccgtgagcagcgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 153 - protein, 214 aa, Artificial Sequence; identical to SEQ ID NO: 147
  Description of Artificial Sequence: Synthetic polypeptide

SEQ ID NO: 154 - DNA, 642 bp, Artificial Sequence; identical to SEQ ID NO: 148
  Description of Artificial Sequence: Synthetic polynucleotide

SEQ ID NO: 155 - protein, 123 aa, Artificial Sequence
  Description of Artificial Sequence: Synthetic polypeptide
EVQLVESGGGLVQPGGSLRLSCAASGFSFNSGYMSWVRQAPGKGLEWIASIYAGSSGKTYYADWAKGRFTISKTSSKTTVYLQMNSLRAEDTAVYYCARDSGGTYYTDYLYAWGQGTLVTVSS

SEQ ID NO: 156 - protein, 113 aa, Artificial Sequence
  Description of Artificial Sequence: Synthetic polypeptide
DIQMTQSPSSLSASVGDRVTITCRASEGISSYLSWYQQKPGKAPKLLIYKASTLTSGVPSRFSGSGSGTDFTLTISSLQPEDFATYYCQSTYGSSGSSGYGAAFGGGTKVEIK

SEQ ID NO: 157 - protein, 453 aa, Artificial Sequence
  Description of Artificial Sequence: Synthetic polypeptide
EVQLVESGGGLVQPGGSLRLSCAASGFSFNSGYMSWVRQAPGKGLEWIASIYAGSSGKTYYADWAKGRFTISKTSSKTTVYLQMNSLRAEDTAVYYCARDSGGTYYTDYLYAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - DNA, 1359 bp, Artificial Sequence
  Description of Artificial Sequence: Synthetic polynucleotide
gaggtgcagctggtggagagcggggggggactggtgcagccaggaggaagcctgagactgagctgtgccgccagcgggtttagctttaatagcggctatatgagctgggtgagacaggcccccggaaaaggactggagtggatcgcctccatttacgccggaagtagcggcaagacctactacgccgattgggctaagggaagattcaccattagcaagaccagcagtaagacaaccgtgtacctgcagatgaacagcctgagagccgaggacaccgccgtgtactattgcgcccgcgacagcggaggaacctactacaccgactacctgtacgcctggggacagggaaccctggtgaccgtgagcagcgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 159 - protein, 220 aa, Artificial Sequence
  Description of Artificial Sequence: Synthetic polypeptide
DIQMTQSPSSLSASVGDRVTITCRASEGISSYLSWYQQKPGKAPKLLIYKASTLTSGVPSRFSGSGSGTDFTLTISSLQPEDFATYYCQSTYGSSGSSGYGAAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - DNA, 660 bp, Artificial Sequence
  Description of Artificial Sequence: Synthetic polynucleotide
gacattcagatgacccagagccccagcagcctgagcgccagcgtgggagacagagtgaccatcacctgcagagccagcgagggaatcagcagctacctgagctggtatcagcagaaacccggcaaagcccccaaactgctgatctacaaagcctccacactgacaagcggagtgcccagcagattcagcggaagcggcagcggcacagacttcaccctgaccatcagcagcctgcagcccgaagatttcgccacctactactgccagagcacctacggcagcagcggcagtagcggatacggcgccgccttcggaggcggaaccaaggtggagatcaaacgtacggtggcagctccatcagtttttatcttcccaccaagcgacgagcaattgaagtccggcactgcctctgtggtgtgccttctgaacaacttctatccaagggaggccaaggtccagtggaaggtcgataatgcgctgcagagcgggaacagccaagagtcagtgaccgagcaggactcaaaagatagcacatactctctgagttccaccctgaccctgtcaaaggctgactacgaaaagcataaggtatacgcatgcgaagtgacccatcagggtctctcatctcccgtaaccaaatcttttaatagaggagaatgc

SEQ ID NO: 161 - protein, 123 aa, Artificial Sequence
  Description of Artificial Sequence: Synthetic polypeptide
EVQLVESGGGLVQPGGSLRLSCAASGFSFNSGYMSWVRQAPGKGLEWIASIYAGSSGKTYYADWAKGRFTISKTSSKTTVYLQMNSLRAEDTAVYYCARDSGGTYYTDYLYLWGQGTLVTVSS

SEQ ID NO: 162 - protein, 113 aa, Artificial Sequence
  Description of Artificial Sequence: Synthetic polypeptide
DIQMTQSPSSLSASVGDRVTITCRASEGISSYLSWYQQKPGKAPKLLIYKASTLTSGVPSRFSGSGSGTDFTLTISSLQPEDFATYYCQSAYGSSGSSGYGAAFGGGTKVEIK

SEQ ID NO: 163 - protein, 453 aa, Artificial Sequence
  Description of Artificial Sequence: Synthetic polypeptide
EVQLVESGGGLVQPGGSLRLSCAASGFSFNSGYMSWVRQAPGKGLEWIASIYAGSSGKTYYADWAKGRFTISKTSSKTTVYLQMNSLRAEDTAVYYCARDSGGTYYTDYLYLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - DNA, 1359 bp, Artificial Sequence
  Description of Artificial Sequence: Synthetic polynucleotide
gaggtgcagctggtggagagcggggggggactggtgcagccaggaggaagcctgagactgagctgtgccgccagcgggtttagctttaatagcggctatatgagctgggtgagacaggcccccggaaaaggactggagtggatcgcctccatttacgccggaagtagcggcaagacctactacgccgattgggctaagggaagattcaccattagcaagaccagcagtaagacaaccgtgtacctgcagatgaacagcctgagagccgaggacaccgccgtgtactattgcgcccgcgacagcggaggaacctactacaccgactacctgtacctgtggggacagggcaccctggtgaccgtgagcagcgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 165 - protein, 220 aa, Artificial Sequence
  Description of Artificial Sequence: Synthetic polypeptide
DIQMTQSPSSLSASVGDRVTITCRASEGISSYLSWYQQKPGKAPKLLIYKASTLTSGVPSRFSGSGSGTDFTLTISSLQPEDFATYYCQSAYGSSGSSGYGAAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - DNA, 660 bp, Artificial Sequence
  Description of Artificial Sequence: Synthetic polynucleotide
gacattcagatgacccagagccccagcagcctgagcgccagcgtgggagacagagtgaccatcacctgcagagccagcgagggaatcagcagctacctgagctggtatcagcagaaacccggcaaagcccccaaactgctgatctacaaagcctccacactgacaagcggagtgcccagcagattcagcggaagcggcagcggcacagacttcaccctgaccatcagcagcctgcagcccgaagatttcgccacctactactgccagagcgcttacggcagcagcggaagcagcggatacggcgccgccttcgggggaggaaccaaagtggaaatcaagcgtacggtggcagctccatcagtttttatcttcccaccaagcgacgagcaattgaagtccggcactgcctctgtggtgtgccttctgaacaacttctatccaagggaggccaaggtccagtggaaggtcgataatgcgctgcagagcgggaacagccaagagtcagtgaccgagcaggactcaaaagatagcacatactctctgagttccaccctgaccctgtcaaaggctgactacgaaaagcataaggtatacgcatgcgaagtgacccatcagggtctctcatctcccgtaaccaaatcttttaatagaggagaatgc

SEQ ID NO: 167 - protein, 123 aa, Artificial Sequence; identical to SEQ ID NO: 161
  Description of Artificial Sequence: Synthetic polypeptide

SEQ ID NO: 168 - protein, 113 aa, Artificial Sequence
  Description of Artificial Sequence: Synthetic polypeptide
DIQMTQSPSSLSASVGDRVTITCRASEGISSYLSWYQQKPGKAPKLLIYKASTLTSGVPSRFSGSGSGTDFTLTISSLQPEDFATYYCQSTYGSSGSSGYAAAFGGGTKVEIK

SEQ ID NO: 169 - protein, 453 aa, Artificial Sequence; identical to SEQ ID NO: 163
  Description of Artificial Sequence: Synthetic polypeptide

SEQ ID NO: 170 - DNA, 1359 bp, Artificial Sequence; identical to SEQ ID NO: 164
  Description of Artificial Sequence: Synthetic polynucleotide

SEQ ID NO: 171 - protein, 220 aa, Artificial Sequence
  Description of Artificial Sequence: Synthetic polypeptide
DIQMTQSPSSLSASVGDRVTITCRASEGISSYLSWYQQKPGKAPKLLIYKASTLTSGVPSRFSGSGSGTDFTLTISSLQPEDFATYYCQSTYGSSGSSGYAAAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - DNA, 660 bp, Artificial Sequence
  Description of Artificial Sequence: Synthetic polynucleotide
gacattcagatgacccagagccccagcagcctgagcgccagcgtgggagacagagtgaccatcacctgcagagccagcgagggaatcagcagctacctgagctggtatcagcagaaacccggcaaagcccccaaactgctgatctacaaagcctccacactgacaagcggagtgcccagcagattcagcggaagcggcagcggcacagacttcaccctgaccatcagcagcctgcagcccgaagatttcgccacctactactgccagagcacctacggcagcagcggcagtagcggatacgccgccgccttcggaggcggaaccaaggtggaaatcaagcgtacggtggcagctccatcagtttttatcttcccaccaagcgacgagcaattgaagtccggcactgcctctgtggtgtgccttctgaacaacttctatccaagggaggccaaggtccagtggaaggtcgataatgcgctgcagagcgggaacagccaagagtcagtgaccgagcaggactcaaaagatagcacatactctctgagttccaccctgaccctgtcaaaggctgactacgaaaagcataaggtatacgcatgcgaagtgacccatcagggtctctcatctcccgtaaccaaatcttttaatagaggagaatgc

SEQ ID NO: 173 - protein, 444 aa, Artificial Sequence
  Description of Artificial Sequence: Synthetic polypeptide
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 174 - protein, 219 aa, Artificial Sequence
  Description of Artificial Sequence: Synthetic polypeptide
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 345 aa, Artificial Sequence
  Description of Artificial Sequence: Synthetic polypeptide
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 10 aa, Artificial Sequence
  Description of Artificial Sequence: Synthetic peptide
WAGSDGHFTL

SEQ ID NO: 177 - protein, 10 aa, Artificial Sequence
  Description of Artificial Sequence: Synthetic peptide
GGIARRSFDY

SEQ ID NO: 178 - protein, 10 aa, Artificial Sequence
  Description of Artificial Sequence: Synthetic peptide
GGVARRSFDY

SEQ ID NO: 179 - DNA, 1344 bp, Artificial Sequence
  Description of Artificial Sequence: Synthetic polynucleotide
gaggtgcagctgcagcagagcgggccagagctggtgaagccaggagccagcgtgaagatgagctgtaagacaagcgggtacacatttacagagtacacaattcactgggtgaagcagagccatgagaaaagcctggaatggatcgggagcatcaaccccaacaatggactgttcaggtacaaccagaaattcaagggcaaagccaccctgaccgtggacaaaagcagtagcacagcctacatggaactgagaagcctgaccagcgaggacagcgccgtgtactattgtgccagagactacggcttcgggtacagcgactactggggccagggaaccaccctgaccgtgagctccgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 180 - protein, 447 aa, Artificial Sequence
  Description of Artificial Sequence: Synthetic polypeptide
QVQLQQPGAELVRPGASVKLSCKASGYTFTSYWMNWVKQRPEQGLEWIGSIDPYDSETHYNQKFKDKAILTVDKSSSTAYMQLSSLTSEDSAVYYCARGRGYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 119 aa, Artificial Sequence
  Description of Artificial Sequence: Synthetic polypeptide
EVQLVQSGAEVKKPGASVKVSCKASGYTFTSYVMHWVRQAPGQRLEWMGYIDPYNEGTKYNEKFKGRVTITRDTSASTAYMELSSLRSEDTAVYYCSRGGIARRSFDYWGQGTLVTVSS

SEQ ID NO: 182 - protein, 449 aa, Artificial Sequence
  Description of Artificial Sequence: Synthetic polypeptide
EVQLVQSGAEVKKPGASVKVSCKASGYTFTSYVMHWVRQAPGQRLEWMGYIDPYNEGTKYNEKFKGRVTITRDTSASTAYMELSSLRSEDTAVYYCSRGGIARRSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - DNA, 1347 bp, Artificial Sequence
  Description of Artificial Sequence: Synthetic polynucleotide
gaggtgcagctggtgcagagcggagccgaggtgaagaagccaggagccagcgtgaaggtgagctgtaaggctagtggatacacattcacaagctacgtgatgcactgggtgagacaggcccctggacagagactggagtggatgggatatatcgacccctataatgagggcaccaagtacaacgaaaaattcaagggcagagtgaccatcaccagagacaccagcgccagcaccgcctacatggagctgagcagcctgagaagcgaggacaccgccgtgtactactgcagcagaggcggcatcgccaggagatcctttgactactggggccagggaacactggtgaccgtgagcagcgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 184 - protein, 119 aa, Artificial Sequence
  Description of Artificial Sequence: Synthetic polypeptide
EVQLVQSGAEVKKPGASVKVSCKASGYTFTSYVMHWVRQAPGQRLEWMGYIDPYNEGTKYNEKFKGRVTITRDTSASTAYMELSSLRSEDTAVYYCSRGGVARRSFDYWGQGTLVTVSS

SEQ ID NO: 185 - protein, 449 aa, Artificial Sequence
  Description of Artificial Sequence: Synthetic polypeptide
EVQLVQSGAEVKKPGASVKVSCKASGYTFTSYVMHWVRQAPGQRLEWMGYIDPYNEGTKYNEKFKGRVTITRDTSASTAYMELSSLRSEDTAVYYCSRGGVARRSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - DNA, 1347 bp, Artificial Sequence
  Description of Artificial Sequence: Synthetic polynucleotide
gaggtgcagctggtgcagagcggagccgaggtgaagaagccaggagccagcgtgaaggtgagctgtaaggctagtggatacacattcacaagctacgtgatgcactgggtgagacaggcccctggacagagactggagtggatgggatatatcgacccctataatgagggcaccaagtacaacgaaaaattcaagggcagagtgaccatcaccagagacaccagcgccagcaccgcctacatggagctgagcagcctgagaagcgaggacaccgccgtgtactactgcagcagaggcggcgtggccaggagatcctttgactactggggccagggaacactggtgaccgtgagcagcgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 187 - protein, 6 aa, Artificial Sequence
  Description of Artificial Sequence: Synthetic 6xHis tag
HHHHHH

SEQ ID NO: 188 - protein, 107 aa, Artificial Sequence
  Description of Artificial Sequence: Synthetic polypeptide
DIQMTQSPASQSASLGESVTITCLASQTIGTWLTWYQQKPGKSPQLLIYAATTLADGVPSRFSGSGSGTKFSFKISSLQAEDFVSYYCQQLYSTPWTFGGGTKLEIK

SEQ ID NO: 189 - protein, 107 aa, Artificial Sequence
  Description of Artificial Sequence: Synthetic polypeptide
DIQMTQSPSSLSASVGDRVTITCLASQTIGTWLTWYQQKPGKAPKLLIYAATTLADGVPSRFSGSGSGTDFTLTISSLQPEDFATYYCQQLYSTPWTFGGGTKVEIK

SEQ ID NO: 190 - protein, 107 aa, Artificial Sequence
  Description of Artificial Sequence: Synthetic polypeptide
DIQMTQSPSSLSASVGDRVTITCLASQTIGTWLTWYQQKPGKAPKLLIYAATTLASGVPSRFSGSGSGTDFTLTISSLQPEDFATYYCQQLYSTPWTFGGGTKVEIK

SEQ ID NO: 191 - protein, 107 aa, Artificial Sequence
  Description of Artificial Sequence: Synthetic polypeptide
DIQMTQSPSSLSASVGDRVTITCLASQTIGTWLTWYQQKPGKAPKLLIYAATTLATGVPSRFSGSGSGTDFTLTISSLQPEDFATYYCQQLYSTPWTFGGGTKVEIK

SEQ ID NO: 192 - protein, 119 aa, Artificial Sequence
  Description of Artificial Sequence: Synthetic polypeptide
EVQLQQSGPELVKPGAPVKMSCKASGYTFTSYVMHWVKQKPGQGLEWIGYIDPYNDGTKYNEKFKGKATLTSDKSSSTAYMELSSLTSEDSAVYYCSRGGMARRSFDYWGQGTTLTVSS

SEQ ID NO: 193 - protein, 119 aa, Artificial Sequence
  Description of Artificial Sequence: Synthetic polypeptide
EVQLVQSGAEVKKPGASVKVSCKASGYTFTSYVMHWVRQAPGQRLEWMGYIDPYNDGTKYNEKFKGRVTITRDTSASTAYMELSSLRSEDTAVYYCSRGGMARRSFDYWGQGTLVTVSS

SEQ ID NO: 194 - protein, 119 aa, Artificial Sequence
  Description of Artificial Sequence: Synthetic polypeptide
EVQLVQSGAEVKKPGASVKVSCKASGYTFTSYVMHWVRQAPGQRLEWIGYIDPYNDGTKYNEKFKGRVTITRDTSASTAYMELSSLRSEDTAVYYCSRGGMARRSFDYWGQGTLVTVSS

SEQ ID NO: 195 - protein, 119 aa, Artificial Sequence
  Description of Artificial Sequence: Synthetic polypeptide
EVQLVQSGAEVKKPGASVKVSCKASGYTFTSYVMHWVRQAPGQRLEWMGYIDPYNDGTKYNEKFKGRATITRDTSASTAYMELSSLRSEDTAVYYCSRGGMARRSFDYWGQGTLVTVSS

SEQ ID NO: 196 - protein, 119 aa, Artificial Sequence
  Description of Artificial Sequence: Synthetic polypeptide
EVQLVQSGAEVKKPGASVKVSCKASGYTFTSYVMHWVRQAPGQRLEWMGYIDPYNDGTKYNEKFKGRVTLTRDTSASTAYMELSSLRSEDTAVYYCSRGGMARRSFDYWGQGTLVTVSS

SEQ ID NO: 197 - protein, 119 aa, Artificial Sequence
  Description of Artificial Sequence: Synthetic polypeptide
EVQLVQSGAEVKKPGASVKVSCKASGYTFTSYVMHWVRQAPGQRLEWMGYIDPYNDGTKYNEKFKGRVTITSDTSASTAYMELSSLRSEDTAVYYCSRGGMARRSFDYWGQGTLVTVSS

SEQ ID NO: 198 - protein, 119 aa, Artificial Sequence
  Description of Artificial Sequence: Synthetic polypeptide
EVQLVQSGAEVKKPGASVKVSCKASGYTFTSYVMHWVRQAPGQRLEWMGYIDPYNDGTKYNEKFKGRVTITRDKSASTAYMELSSLRSEDTAVYYCSRGGMARRSFDYWGQGTLVTVSS

SEQ ID NO: 199 - protein, 119 aa, Artificial Sequence
  Description of Artificial Sequence: Synthetic polypeptide
EVQLVQSGAEVKKPGASVKVSCKASGYTFTSYVMHWVRQAPGQRLEWMGYIDPYNDATKYNEKFKGRVTITRDTSASTAYMELSSLRSEDTAVYYCSRGGMARRSFDYWGQGTLVTVSS

SEQ ID NO: 200 - protein, 119 aa, Artificial Sequence
  Description of Artificial Sequence: Synthetic polypeptide
EVQLVQSGAEVKKPGASVKVSCKASGYTFTSYVMHWVRQAPGQRLEWMGYIDPYNDSTKYNEKFKGRVTITRDTSASTAYMELSSLRSEDTAVYYCSRGGMARRSFDYWGQGTLVTVSS

SEQ ID NO: 201 - protein, 119 aa, Artificial Sequence
  Description of Artificial Sequence: Synthetic polypeptide
EVQLVQSGAEVKKPGASVKVSCKASGYTFTSYVMHWVRQAPGQRLEWMGYIDPYNSGTKYNEKFKGRVTITRDTSASTAYMELSSLRSEDTAVYYCSRGGMARRSFDYWGQGTLVTVSS

SEQ ID NO: 202 - protein, 119 aa, Artificial Sequence
  Description of Artificial Sequence: Synthetic polypeptide
EVQLVQSGAEVKKPGASVKVSCKASGYTFTSYVMHWVRQAPGQRLEWMGYIDPYNTGTKYNEKFKGRVTITRDTSASTAYMELSSLRSEDTAVYYCSRGGMARRSFDYWGQGTLVTVSS

SEQ ID NO: 203 - protein, 113 aa, Artificial Sequence
  Description of Artificial Sequence: Synthetic polypeptide
DVVMTQTPASVEAAVGGTVTIKCQASEGISSYLSWYQQKPGQPPKLLIYKASTLTSGVPSRFKGSGSGTDFTLTISDLESADAATYYCQCTYGSSGSSGYGAAFGGGTEVVVK

SEQ ID NO: 204 - protein, 113 aa, Artificial Sequence
  Description of Artificial Sequence: Synthetic polypeptide
DIQMTQSPSSLSASVGDRVTITCRASEGISSYLSWYQQKPGKAPKLLIYKASTLTSGVPSRFSGSGSGTDFTLTISSLQPEDFATYYCQCTYGSSGSSGYGAAFGGGTKVEIK

SEQ ID NO: 205 - protein, 113 aa, Artificial Sequence
  Description of Artificial Sequence: Synthetic polypeptide
DVQMTQSPSSLSASVGDRVTITCRASEGISSYLSWYQQKPGKAPKLLIYKASTLTSGVPSRFSGSGSGTDFTLTISSLQPEDAATYYCQCTYGSSGSSGYGAAFGGGTKVEVK

SEQ ID NO: 206 - protein, 113 aa, Artificial Sequence
  Description of Artificial Sequence: Synthetic polypeptide
DIQMTQSPSSVSASVGDRVTITCRASEGISSYLSWYQQKPGKAPKLLIYKASTLTSGVPSRFSGSGSGTDFTLTISSLQPEDFATYYCQCTYGSSGSSGYGAAFGGGTKVEIK

SEQ ID NO: 207 - protein, 113 aa, Artificial Sequence
  Description of Artificial Sequence: Synthetic polypeptide
DIQMTQSPSSVSASVGDRVTITCQASEGISSYLSWYQQKPGKAPKLLIYKASTLTSGVPSRFSGSGSGTDFTLTISSLQPEDFATYYCQCTYGSSGSSGYGAAFGGGTKVEIK

SEQ ID NO: 208 - protein, 121 aa, Artificial Sequence
  Description of Artificial Sequence: Synthetic polypeptide
QSLEESGGDLVKPGASLTLTCKASGFSFNSGYMCWVRQAPGKGLEWIACIYAGSSGKTYYASWATGRFTISKTSSTTVTLQMTSLTVADTATYFCARDDGGTYYTDYLYLWGPGTLVTVSS

SEQ ID NO: 209 - protein, 123 aa, Artificial Sequence
  Description of Artificial Sequence: Synthetic polypeptide
EVQLVESGGGLVQPGGSLRLSCAASGFSFNSGYMCWVRQAPGKGLEWVSCIYAGSSGKTYYADSVKGRFTISRDNSKNTLYLQMNSLRAEDTAVYYCARDDGGTYYTDYLYLWGQGTLVTVSS

SEQ ID NO: 210 - protein, 123 aa, Artificial Sequence
  Description of Artificial Sequence: Synthetic polypeptide
EVQLVESGGGLVQPGGSLRLSCAASGFSFNSGYMCWVRQAPGKGLEWIACIYAGSSGKTYYADSVKGRFTISKDSSKNTVYLQMNSLRAEDTAVYYCARDDGGTYYTDYLYLWGQGTLVTVSS

SEQ ID NO: 211 - protein, 123 aa, Artificial Sequence
  Description of Artificial Sequence: Synthetic polypeptide
EVQLVESGGGLVQPGGSLRLSCAASGFSFNSGYMCWVRQAPGKGLEWIACIYAGSSGKTYYADWAKGRFTISKDSSKNTVYLQMNSLRAEDTAVYYCARDDGGTYYTDYLYLWGQGTLVTVSS

SEQ ID NO: 212 - protein, 123 aa, Artificial Sequence
  Description of Artificial Sequence: Synthetic polypeptide
EVQLVESGGGLVQPGGSLRLSCAASGFSFNSGYMCWVRQAPGKGLEWIACIYAGSSGKTYYADSVKGRFTISKDSSKTTVYLQMNSLRAEDTAVYYCARDDGGTYYTDYLYLWGQGTLVTVSS

SEQ ID NO: 213 - protein, 123 aa, Artificial Sequence
  Description of Artificial Sequence: Synthetic polypeptide
EVQLVESGGGLVQPGGSLRLSCAASGFSFNSGYMCWVRQAPGKGLEWIACIYAGSSGKTYYADWAKGRFTISKDSSKTTVYLQMNSLRAEDTAVYYCARDDGGTYYTDYLYLWGQGTLVTVSS

SEQ ID NO: 214 - protein, 122 aa, Artificial Sequence
  Description of Artificial Sequence: Synthetic polypeptide
EVQLVESGGGLVQPGGSLRLSCAASGFSFNSGYMCWVRQAPGKGLEWIACIYAGSSGKTYYADSVKGRFTISKDSSTTVYLQMNSLRAEDTAVYYCARDDGGTYYTDYLYLWGQGTLVTVSS

SEQ ID NO: 215 - protein, 122 aa, Artificial Sequence
  Description of Artificial Sequence: Synthetic polypeptide
EVQLVESGGGLVQPGGSLRLSCAASGFSFNSGYMCWVRQAPGKGLEWIACIYAGSSGKTYYADWAKGRFTISKDSSTTVYLQMNSLRAEDTAVYYCARDDGGTYYTDYLYLWGQGTLVTVSS

SEQ ID NO: 216 - protein, 122 aa, Artificial Sequence
  Description of Artificial Sequence: Synthetic polypeptide
QSLVESGGGLVQPGGSLRLSCAASGFSFNSGYMCWVRQAPGKGLEWIACIYAGSSGKTYYADWAKGRFTISKDSSKTTVYLQMNSLRAEDTAVYYCARDDGGTYYTDYLYLWGQGTLVTVSS

SEQ ID NO: 217 - protein, 121 aa, Artificial Sequence
  Description of Artificial Sequence: Synthetic polypeptide
QSLVESGGGLVQPGGSLRLSCAASGFSFNSGYMCWVRQAPGKGLEWIACIYAGSSGKTYYADWAKGRFTISKTSSTTVYLQMNSLRAEDTAVYYCARDDGGTYYTDYLYLWGQGTLVTVSS

SEQ ID NO: 218 - protein, 122 aa, Artificial Sequence
  Description of Artificial Sequence: Synthetic polypeptide
EVQLVESGGGLVQPGGSLRLSCAASGFSFNSGYMCWVRQAPGKGLEWIACIYAGSSGKTYYADWAKGRFTISKTSSTTVYLQMNSLRAEDTAVYYCARDDGGTYYTDYLYLWGQGTLVTVSS

SEQ ID NO: 219 - protein, 123 aa, Artificial Sequence
  Description of Artificial Sequence: Synthetic polypeptide
EVQLVESGGGLVQPGGSLRLSCAASGFSFNSGYMCWVRQAPGKGLEWIACIYAGSSGKTYYADWAKGRFTISKTSSKTTVYLQMNSLRAEDTAVYYCARDDGGTYYTDYLYLWGQGTLVTVSS

SEQ ID NO: 220 - protein, 122 aa, Artificial Sequence
  Description of Artificial Sequence: Synthetic polypeptide
EVQLVESGGGLVQPGGSLRLSCAASGFSFNSGYMSWVRQAPGKGLEWIASIYAGSSGKTYYADWAKGRFTISKTSSTTVYLQMNSLRAEDTAVYYCARDSGGTYYTDYLYLWGQGTLVTVSS

SEQ ID NO: 221 - protein, 113 aa, Artificial Sequence
  Description of Artificial Sequence: Synthetic polypeptide
DIQMTQSPSSLSASVGDRVTITCRASEGISSYLSWYQQKPGKAPKLLIYKASTLTSGVPSRFSGSGSGTDFTLTISSLQPEDFATYYCASTYGSSGSSGYGAAFGGGTKVEIK

SEQ ID NO: 222 - protein, 113 aa, Artificial Sequence
  Description of Artificial Sequence: Synthetic polypeptide
DIQMTQSPSSLSASVGDRVTITCRASEGISSYLSWYQQKPGKAPKLLIYKASTLTSGVPSRFSGSGSGTDFTLTISSLQPEDFATYYCQATYGSSGSSGYGAAFGGGTKVEIK

SEQ ID NO: 223 - protein, 113 aa, Artificial Sequence
  Description of Artificial Sequence: Synthetic polypeptide
DIQMTQSPSSLSASVGDRVTITCRASEGISSYLSWYQQKPGKAPKLLIYKASTLTSGVPSRFSGSGSGTDFTLTISSLQPEDFATYYCQSTAGSSGSSGYGAAFGGGTKVEIK

SEQ ID NO: 224 - protein, 113 aa, Artificial Sequence
  Description of Artificial Sequence: Synthetic polypeptide
DIQMTQSPSSLSASVGDRVTITCRASEGISSYLSWYQQKPGKAPKLLIYKASTLTSGVPSRFSGSGSGTDFTLTISSLQPEDFATYYCQSTYASSGSSGYGAAFGGGTKVEIK

SEQ ID NO: 225 - protein, 113 aa, Artificial Sequence
  Description of Artificial Sequence: Synthetic polypeptide
DIQMTQSPSSLSASVGDRVTITCRASEGISSYLSWYQQKPGKAPKLLIYKASTLTSGVPSRFSGSGSGTDFTLTISSLQPEDFATYYCQSTYGASGSSGYGAAFGGGTKVEIK

SEQ ID NO: 226 - protein, 113 aa, Artificial Sequence
  Description of Artificial Sequence: Synthetic polypeptide
DIQMTQSPSSLSASVGDRVTITCRASEGISSYLSWYQQKPGKAPKLLIYKASTLTSGVPSRFSGSGSGTDFTLTISSLQPEDFATYYCQSTYGSAGSSGYGAAFGGGTKVEIK

SEQ ID NO: 227 - protein, 113 aa, Artificial Sequence
  Description of Artificial Sequence: Synthetic polypeptide
DIQMTQSPSSLSASVGDRVTITCRASEGISSYLSWYQQKPGKAPKLLIYKASTLTSGVPSRFSGSGSGTDFTLTISSLQPEDFATYYCQSTYGSSASSGYGAAFGGGTKVEIK

SEQ ID NO: 228 - protein, 113 aa, Artificial Sequence
  Description of Artificial Sequence: Synthetic polypeptide
DIQMTQSPSSLSASVGDRVTITCRASEGISSYLSWYQQKPGKAPKLLIYKASTLTSGVPSRFSGSGSGTDFTLTISSLQPEDFATYYCQSTYGSSGASGYGAAFGGGTKVEIK

SEQ ID NO: 229 - protein, 113 aa, Artificial Sequence
  Description of Artificial Sequence: Synthetic polypeptide
DIQMTQSPSSLSASVGDRVTITCRASEGISSYLSWYQQKPGKAPKLLIYKASTLTSGVPSRFSGSGSGTDFTLTISSLQPEDFATYYCQSTYGSSGSAGYGAAFGGGTKVEIK

SEQ ID NO: 230 - protein, 113 aa, Artificial Sequence
  Description of Artificial Sequence: Synthetic polypeptide
DIQMTQSPSSLSASVGDRVTITCRASEGISSYLSWYQQKPGKAPKLLIYKASTLTSGVPSRFSGSGSGTDFTLTISSLQPEDFATYYCQSTYGSSGSSAYGAAFGGGTKVEIK

SEQ ID NO: 231 - protein, 113 aa, Artificial Sequence
  Description of Artificial Sequence: Synthetic polypeptide
DIQMTQSPSSLSASVGDRVTITCRASEGISSYLSWYQQKPGKAPKLLIYKASTLTSGVPSRFSGSGSGTDFTLTISSLQPEDFATYYCQSTYGSSGSSGAGAAFGGGTKVEIK

SEQ ID NO: 232 - protein, 123 aa, Artificial Sequence
  Description of Artificial Sequence: Synthetic polypeptide
EVQLVESGGGLVQPGGSLRLSCAASGFSFNSGYMSWVRQAPGKGLEWIASIYAGSSGKTYYADWAKGRFTISKTSSKTTVYLQMNSLRAEDTAVYYCARASGGTYYTDYLYLWGQGTLVTVSS

SEQ ID NO: 233 - protein, 123 aa, Artificial Sequence
  Description of Artificial Sequence: Synthetic polypeptide
EVQLVESGGGLVQPGGSLRLSCAASGFSFNSGYMSWVRQAPGKGLEWIASIYAGSSGKTYYADWAKGRFTISKTSSKTTVYLQMNSLRAEDTAVYYCARDAGGTYYTDYLYLWGQGTLVTVSS

SEQ ID NO: 234 - protein, 123 aa, Artificial Sequence
  Description of Artificial Sequence: Synthetic polypeptide
EVQLVESGGGLVQPGGSLRLSCAASGFSFNSGYMSWVRQAPGKGLEWIASIYAGSSGKTYYADWAKGRFTISKTSSKTTVYLQMNSLRAEDTAVYYCARDSAGTYYTDYLYLWGQGTLVTVSS

SEQ ID NO: 235 - protein, 123 aa, Artificial Sequence
  Description of Artificial Sequence: Synthetic polypeptide
EVQLVESGGGLVQPGGSLRLSCAASGFSFNSGYMSWVRQAPGKGLEWIASIYAGSSGKTYYADWAKGRFTISKTSSKTTVYLQMNSLRAEDTAVYYCARDSGATYYTDYLYLWGQGTLVTVSS

SEQ ID NO: 236 - protein, 123 aa, Artificial Sequence
  Description of Artificial Sequence: Synthetic polypeptide
EVQLVESGGGLVQPGGSLRLSCAASGFSFNSGYMSWVRQAPGKGLEWIASIYAGSSGKTYYADWAKGRFTISKTSSKTTVYLQMNSLRAEDTAVYYCARDSGGAYYTDYLYLWGQGTLVTVSS

SEQ ID NO: 237 - protein, 123 aa, Artificial Sequence
  Description of Artificial Sequence: Synthetic polypeptide
EVQLVESGGGLVQPGGSLRLSCAASGFSFNSGYMSWVRQAPGKGLEWIASIYAGSSGKTYYADWAKGRFTISKTSSKTTVYLQMNSLRAEDTAVYYCARDSGGTAYTDYLYLWGQGTLVTVSS

SEQ ID NO: 238 - protein, 123 aa, Artificial Sequence
  Description of Artificial Sequence: Synthetic polypeptide
EVQLVESGGGLVQPGGSLRLSCAASGFSFNSGYMSWVRQAPGKGLEWIASIYAGSSGKTYYADWAKGRFTISKTSSKTTVYLQMNSLRAEDTAVYYCARDSGGTYATDYLYLWGQGTLVTVSS

SEQ ID NO: 239 - protein, 123 aa, Artificial Sequence
  Description of Artificial Sequence: Synthetic polypeptide
EVQLVESGGGLVQPGGSLRLSCAASGFSFNSGYMSWVRQAPGKGLEWIASIYAGSSGKTYYADWAKGRFTISKTSSKTTVYLQMNSLRAEDTAVYYCARDSGGTYYADYLYLWGQGTLVTVSS

SEQ ID NO: 240 - protein, 123 aa, Artificial Sequence
  Description of Artificial Sequence: Synthetic polypeptide
EVQLVESGGGLVQPGGSLRLSCAASGFSFNSGYMSWVRQAPGKGLEWIASIYAGSSGKTYYADWAKGRFTISKTSSKTTVYLQMNSLRAEDTAVYYCARDSGGTYYTAYLYLWGQGTLVTVSS

SEQ ID NO: 241 - protein, 123 aa, Artificial Sequence
  Description of Artificial Sequence: Synthetic polypeptide
EVQLVESGGGLVQPGGSLRLSCAASGFSFNSGYMSWVRQAPGKGLEWIASIYAGSSGKTYYADWAKGRFTISKTSSKTTVYLQMNSLRAEDTAVYYCARDSGGTYYTDALYLWGQGTLVTVSS

SEQ ID NO: 242 - protein, 123 aa, Artificial Sequence
  Description of Artificial Sequence: Synthetic polypeptide
EVQLVESGGGLVQPGGSLRLSCAASGFSFNSGYMSWVRQAPGKGLEWIASIYAGSSGKTYYADWAKGRFTISKTSSKTTVYLQMNSLRAEDTAVYYCARDSGGTYYTDYAYLWGQGTLVTVSS

SEQ ID NO: 243 - protein, 123 aa, Artificial Sequence
  Description of Artificial Sequence: Synthetic polypeptide
EVQLVESGGGLVQPGGSLRLSCAASGFSFNSGYMSWVRQAPGKGLEWIASIYAGSSGKTYYADWAKGRFTISKTSSKTTVYLQMNSLRAEDTAVYYCARDSGGTYYTDYLALWGQGTLVTVSS

SEQ ID NO: 244 - protein, 119 aa, Artificial Sequence
  Description of Artificial Sequence: Synthetic polypeptide
EVQLVQSGAEVKKPGASVKVSCKASGYTFTSYVMHWVRQAPGQRLEWMGYIDPYNDGTKYNEKFKGRVTITRDTSASTAYMELSSLRSEDTAVYYCSRGGIARRSFDYWGQGTLVTVSS

SEQ ID NO: 245 - protein, 119 aa, Artificial Sequence
  Description of Artificial Sequence: Synthetic polypeptide
EVQLVQSGAEVKKPGASVKVSCKASGYTFTSYVMHWVRQAPGQRLEWMGYIDPYNDGTKYNEKFKGRVTITRDTSASTAYMELSSLRSEDTAVYYCSRGGVARRSFDYWGQGTLVTVSS

(19) United States
(12) Patent Application Publication
(10) Pub. No.: US 2024/0174750 A1
(43) Pub. Date: May 30, 2024

(54) ANTI-CD47 ANTIBODIES AND USES THEREOF

(71) Applicant: Virtuoso BINco, Inc., San Mateo, CA (US)

(72) Inventors: Xinhua WANG, San Mateo, CA (US); Xiaocheng CHEN, San Mateo, CA (US); Oi Kwan WONG, San Mateo, CA (US); Leonard POST, San Mateo, CA (US)

(21) Appl. No.: 18/551,729

(22) PCT Filed: Mar. 22, 2022
(86) PCT No.: PCT/US2022/021364

§ 371 Date: Sep. 21, 2023

Related U.S. Application Data
(60) Provisional application No. 63/164,658, filed on Mar. 23, 2021.

(51) Int. Cl. C07K 16/28; A61K 45/06; A61P 35/00; A61K 39/00

(52) U.S. Cl. CPC C07K 16/2803; A61K 45/06; A61P 35/00; A61K 2039/505; C07K 2317/565; C07K 2317/92

(57) ABSTRACT

Disclosed herein are antibodies that bind specifically to CD47 and have optimized functional and safety properties.

### CROSS-REFERENCE

This application claims the benefit of U.S. Provisional Application No. 63/164,658, filed Mar. 23, 2021, which is entirely incorporated herein by reference for all purposes.

### SEQUENCE LISTING

The instant application contains a Sequence Listing which has been submitted electronically in ASCII format and is hereby incorporated by reference in its entirety. Said ASCII copy, created on Mar. 21, 2022, is named 55429-731_601_SL.txt and is 296,116 bytes in size.

### SUMMARY

Disclosed herein are antibodies that bind specifically to CD47 comprising at least one complementarity determining region (CDR) according to SEQ ID NOs: 1-85, 176-178 or an amino acid sequence that has 0-1 amino acid mutations, substitutions, or deletions relative to SEQ ID NOs: 1-13, 58-69, or an amino acid sequence that has 0-3 amino acid mutations, substitutions, or deletions to relative to SEQ ID NOs: 14-57, 70-85, 176-178. In some embodiments, the antibody comprises a heavy chain variable domain that comprises CDRs: HC-CDR1, HC-CDR2, HC-CDR3, wherein the HC-CDR1, HC-CDR2, HC-CDR3 of the heavy chain variable domain comprises a set of sequences selected from the group consisting of the following set of sequences: HC-CDR1: SEQ ID NO: 1, HC-CDR2: SEQ ID NO: 14, HC-CDR3: SEQ ID NO: 30; HC-CDR1: SEQ ID NO: 2, HC-CDR2: SEQ ID NO: 15, HC-CDR3: SEQ ID NO: 31; HC-CDR1: SEQ ID NO: 3, HC-CDR2: SEQ ID NO: 16, HC-CDR3: SEQ ID NO: 32; HC-CDR1: SEQ ID NO: 2, HC-CDR2: SEQ ID NO: 17, HC-CDR3: SEQ ID NO: 33; HC-CDR1: SEQ ID NO: 4, HC-CDR2: SEQ ID NO: 18, HC-CDR3: SEQ ID NO: 34; HC-CDR1: SEQ ID NO: 5, HC-CDR2: SEQ ID NO: 19, HC-CDR3: SEQ ID NO: 35; HC-CDR1: SEQ ID NO: 6, HC-CDR2: SEQ ID NO: 20, HC-CDR3: SEQ ID NO: 176; HC-CDR1: SEQ ID NO: 7, HC-CDR2: SEQ ID NO: 21, HC-CDR3: SEQ ID NO: 36; HC-CDR1: SEQ ID NO: 8, HC-CDR2: SEQ ID NO: 22, HC-CDR3: SEQ ID NO: 37; HC-CDR1: SEQ ID NO: 9, HC-CDR2: SEQ ID NO: 23, HC-CDR3: SEQ ID NO: 38; HC-CDR1: SEQ ID NO: 10, HC-CDR2: SEQ ID NO: 24, HC-CDR3: SEQ ID NO: 39; HC-CDR1: SEQ ID NO: 8, HC-CDR2: SEQ ID NO: 25, HC-CDR3: SEQ ID NO: 40; HC-CDR1: SEQ ID NO: 11, HC-CDR2: SEQ ID NO: 26, HC-CDR3: SEQ ID NO: 41; HC-CDR1: SEQ ID NO: 12, HC-CDR2: SEQ ID NO: 27, HC-CDR3: SEQ ID NO: 42; HC-CDR1: SEQ ID NO: 1, HC-CDR2: SEQ ID NO: 28, HC-CDR3: SEQ ID NO: 30; HC-CDR1: SEQ ID NO: 1, HC-CDR2: SEQ ID NO: 28, HC-CDR3: SEQ ID NO: 177; HC-CDR1: SEQ ID NO: 1, HC-CDR2: SEQ ID NO: 28, HC-CDR3: SEQ ID NO: 178; HC-CDR1: SEQ ID NO: 13, HC-CDR2: SEQ ID NO: 29, HC-CDR3: SEQ ID NO: 43; and HC-CDR1: SEQ ID NO: 13, HC-CDR2: SEQ ID NO: 29, HC-CDR3: SEQ ID NO: 44. In some embodiments, the antibody comprises a light chain variable domain that comprises CDRs: LC-CDR1, LC-CDR2, LC-CDR3, wherein the LC-CDR1, LC-CDR2, LC-CDR3 of the light chain variable domain comprises a set of sequences selected from the group consisting of the following set of sequences: LC-CDR1: SEQ ID NO: 45, LC-CDR2: SEQ ID NO: 58, LC-CDR3: SEQ ID NO: 70; LC-CDR1: SEQ ID NO: 46, LC-CDR2: SEQ ID NO: 59, LC-CDR3: SEQ ID NO: 71; LC-CDR1: SEQ ID NO: 47, LC-CDR2: SEQ ID NO: 60, LC-CDR3: SEQ ID NO: 72; LC-CDR1: SEQ ID NO: 48, LC-CDR2: SEQ ID NO: 59, LC-CDR3: SEQ ID NO: 73; LC-CDR1: SEQ ID NO: 49, LC-CDR2: SEQ ID NO: 61, LC-CDR3: SEQ ID NO: 74; LC-CDR1: SEQ ID NO: 50, LC-CDR2: SEQ ID NO: 62, LC-CDR3: SEQ ID NO: 75; LC-CDR1: SEQ ID NO: 51, LC-CDR2: SEQ ID NO: 63, LC-CDR3: SEQ ID NO: 76; LC-CDR1: SEQ ID NO: 52, LC-CDR2: SEQ ID NO: 62, LC-CDR3: SEQ ID NO: 77; LC-CDR1: SEQ ID NO: 53, LC-CDR2: SEQ ID NO: 64, LC-CDR3: SEQ ID NO: 78; LC-CDR1: SEQ ID NO: 54, LC-CDR2: SEQ ID NO: 65, LC-CDR3: SEQ ID NO: 79; LC-CDR1: SEQ ID NO: 55, LC-CDR2: SEQ ID NO: 66, LC-CDR3: SEQ ID NO: 80; LC-CDR1: SEQ ID NO: 53, LC-CDR2: SEQ ID NO: 67, LC-CDR3: SEQ ID NO: 78; LC-CDR1: SEQ ID NO: 52, LC-CDR2: SEQ ID NO: 62, LC-CDR3: SEQ ID NO: 81; LC-CDR1: SEQ ID NO: 56, LC-CDR2: SEQ ID NO: 68, LC-CDR3: SEQ ID NO: 82; LC-CDR1: SEQ ID NO: 45, LC-CDR2: SEQ ID NO: 69, LC-CDR3: SEQ ID NO: 70; LC-CDR1: SEQ ID NO: 57, LC-CDR2: SEQ ID NO: 66, LC-CDR3: SEQ ID NO: 83; LC-CDR1: SEQ ID NO: 57, LC-CDR2: SEQ ID NO: 66, LC-CDR3: SEQ ID NO: 84; LC-CDR1: SEQ ID NO: 57, LC-CDR2: SEQ ID NO: 66, LC-CDR3: SEQ ID NO: 85. In some embodiments, the antibody comprises at least one complementarity determining region (CDR) according to SEQ ID NOs: 1, 13, 66, 69, 28-30, 30, 43, 44, 45, 57, 70, 83-85, 177-178 or an amino acid sequence that has 0-1 amino acid mutations, substitutions, or deletions relative to SEQ ID NOs: 1, 13, 66, 69, or an amino acid sequence that has 0-3 amino acid mutations, substitutions, or deletions to relative to SEQ ID NOs:28-30, 30, 43, 44, 45, 57, 70, 83-85, 177-178. In some embodiments, the antibody comprises a heavy chain variable domain that comprises CDRs: HC-CDR1, HC-CDR2, HC-CDR3, wherein the HC-CDR1, HC-CDR2, HC-CDR3 of the heavy chain variable domain comprises a set of sequences selected from the group consisting of the following set of sequences: HC-CDR1: SEQ ID NO: 1, HC-CDR2: SEQ ID NO: 28, HC-CDR3: SEQ ID NO: 30; HC-CDR1: SEQ ID NO: 1, HC-CDR2: SEQ ID NO: 28, HC-CDR3: SEQ ID NO: 177; HC-CDR1: SEQ ID NO: 1, HC-CDR2: SEQ ID NO: 28, HC-CDR3: SEQ ID NO: 178; HC-CDR1: SEQ ID NO: 13, HC-CDR2: SEQ ID NO: 29, HC-CDR3: SEQ ID NO: 43; and HC-CDR1: SEQ ID NO: 13, HC-CDR2: SEQ ID NO: 29, HC-CDR3: SEQ ID NO: 44; and wherein the CDRs comprise 0-2 amino acid mutations, substitutions, or deletions in at least one of the HC-CDR1, HC-CDR2, HC-CDR3. In some embodiments, the antibody comprises a heavy chain variable domain that comprises CDRs: HC-CDR1, HC-CDR2, HC-CDR3, wherein the HC-CDR1, HC-CDR2, HC-CDR3 of the heavy chain variable domain comprises a set of sequences selected from the group consisting of the following set of sequences: HC-CDR1: SEQ ID NO: 1, HC-CDR2: SEQ ID NO: 28, HC-CDR3: SEQ ID NO: 30; HC-CDR1: SEQ ID NO: 1, HC-CDR2: SEQ ID NO: 28, HC-CDR3: SEQ ID NO: 177; HC-CDR1: SEQ ID NO: 1, HC-CDR2: SEQ ID NO: 28, HC-CDR3: SEQ ID NO: 178; HC-CDR1: SEQ ID NO: 13, HC-CDR2: SEQ ID NO: 29, HC-CDR3: SEQ ID NO: 43; and HC-CDR1: SEQ ID NO: 13, HC-CDR2: SEQ ID NO: 29, HC-CDR3: SEQ ID NO: 44. In some embodiments, the antibody comprises a light chain variable domain that comprises CDRs: LC-CDR1, LC-CDR2, LC-CDR3, wherein the LC-CDR1, LC-CDR2, LC-CDR3 of the light chain variable domain comprises a set of sequences selected from the group consisting of the following set of sequences: LC-CDR1: SEQ ID NO: 45, LC-CDR2: SEQ ID NO: 69, LC-CDR3: SEQ ID NO: 70; LC-CDR1: SEQ ID NO: 57, LC-CDR2: SEQ ID NO: 66, LC-CDR3: SEQ ID NO: 83; LC-CDR1: SEQ ID NO: 57, LC-CDR2: SEQ ID NO: 66, LC-CDR3: SEQ ID NO: 84; LC-CDR1: SEQ ID NO: 57, LC-CDR2: SEQ ID NO: 66, LC-CDR3: SEQ ID NO: 85; and wherein the CDRs comprise 0-2 amino acid mutations, substitutions, or deletions in at least one of the LC-CDR1, LC-CDR2, LC-CDR3. In some embodiments, the antibody comprises a light chain variable domain that comprises CDRs: LC-CDR1, LC-CDR2, LC-CDR3, wherein the LC-CDR1, LC-CDR2, LC-CDR3 of the light chain variable domain comprises a set of sequences selected from the group consisting of the following set of sequences: LC-CDR1: SEQ ID NO: 45, LC-CDR2: SEQ ID NO: 69, LC-CDR3: SEQ ID NO: 70; LC-CDR1: SEQ ID NO: 57, LC-CDR2: SEQ ID NO: 66, LC-CDR3: SEQ ID NO: 83; LC-CDR1: SEQ ID NO: 57, LC-CDR2: SEQ ID NO: 66, LC-CDR3: SEQ ID NO: 84; and LC-CDR1: SEQ ID NO: 57, LC-CDR2: SEQ ID NO: 66, LC-CDR3: SEQ ID NO: 85. In some embodiments, the antibody comprises a heavy chain variable domain that comprises CDRs: HC-CDR1, HC-CDR2, HC-CDR3, wherein the HC-CDR1, HC-CDR2, HC-CDR3 of the heavy chain variable domain comprises a set of sequences: HC-CDR1: SEQ ID NO: 13, HC-CDR2: SEQ ID NO: 29, HC-CDR3: SEQ ID NO: 44; and wherein the CDRs comprise 0-2 amino acid mutations, substitutions, or deletions in at least one of the HC-CDR1, HC-CDR2, HC-CDR3. In some embodiments, the antibody comprises a heavy chain variable domain that comprises CDRs: HC-CDR1, HC-CDR2, HC-CDR3, wherein the HC-CDR1, HC-CDR2, HC-CDR3 of the heavy chain variable domain comprises a set of sequences: HC-CDR1: SEQ ID NO: 13, HC-CDR2: SEQ ID NO: 29, HC-CDR3: SEQ ID NO: 44. In some embodiments, the antibody comprises a light chain variable domain that comprises CDRs: LC-CDR1, LC-CDR2, LC-CDR3, wherein the LC-CDR1, LC-CDR2, LC-CDR3 of the light chain variable domain comprises: LC-CDR1; SEQ ID NO: 57, LC-CDR2: SEQ ID NO: 66, LC-CDR3: SEQ ID NO: 84; or LC-CDR1: SEQ ID NO: 57, LC-CDR2: SEQ ID NO: 66, LC-CDR3: SEQ ID NO: 85; and wherein the CDRs comprise 0-2 amino acid mutations, substitutions, or deletions in at least one of the LC-CDR1, LC-CDR2, LC-CDR3. In some embodiments, the antibody comprises a light chain variable domain that comprises CDRs: LC-CDR1, LC-CDR2, LC-CDR3, wherein the LC-CDR1, LC-CDR2, LC-CDR3 of the light chain variable domain comprises LC-CDR1, SEQ ID NO: 57, LC-CDR2: SEQ ID NO: 66, LC-CDR3: SEQ ID NO: 84; or LC-CDR1, SEQ ID NO: 57, LC-CDR2: SEQ ID NO: 66, LC-CDR3: SEQ ID NO: 85. In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise a set of sequences selected from the group consisting of the following set of sequences: HC-CDR1: SEQ ID NO: 1, HC-CDR2: SEQ ID NO: 28, HC-CDR3: SEQ ID NO: 30, LC-CDR1: SEQ ID NO: 45, LC-CDR2: SEQ ID NO: 69, LC-CDR3: SEQ ID NO: 70; HC-CDR1: SEQ ID NO: 1, HC-CDR2: SEQ ID NO: 28, HC-CDR3: SEQ ID NO: 177, LC-CDR1: SEQ ID NO: 45, LC-CDR2: SEQ ID NO: 69, LC-CDR3: SEQ ID NO: 70; HC-CDR1: SEQ ID NO: 1, HC-CDR2: SEQ ID NO: 28, HC-CDR3: SEQ ID NO: 178, LC-CDR1: SEQ ID NO: 45, LC-CDR2: SEQ ID NO: 69, LC-CDR3: SEQ ID NO: 70; HC-CDR1: SEQ ID NO: 13, HC-CDR2: SEQ ID NO: 29, HC-CDR3: SEQ ID NO: 43, LC-CDR1: SEQ ID NO: 57, LC-CDR2: SEQ ID NO: 66, LC-CDR3: SEQ ID NO: 83; HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 84; and HC-CDR1: SEQ ID NO: 13, HC-CDR2: SEQ ID NO: 29, HC-CDR3: SEQ ID NO: 44; LC-CDR1: SEQ ID NO: 57, LC-CDR2: SEQ ID NO: 66, LC-CDR3: SEQ ID NO: 85; wherein the CDRs comprise 0-2 amino acid mutations, substitutions, or deletions in at least one of the HC-CDR1, HC-CDR2, HC-CDR3, LC-CDR1, LC-CDR2, LC-CDR3. In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise a set of sequences selected from the group consisting of the following set of sequences: HC-CDR1: SEQ ID NO: 1, HC-CDR2: SEQ ID NO: 28, HC-CDR3: SEQ ID NO: 30, LC-CDR1: SEQ ID NO: 45, LC-CDR2: SEQ ID NO: 69, LC-CDR3: SEQ ID NO: 70; HC-CDR1: SEQ ID NO: 1, HC-CDR2: SEQ ID NO: 28, HC-CDR3: SEQ ID NO: 177, LC-CDR1: SEQ ID NO: 45, LC-CDR2: SEQ ID NO: 69, LC-CDR3: SEQ ID NO: 70; HC-CDR1: SEQ ID NO: 1, HC-CDR2: SEQ ID NO: 28, HC-CDR3: SEQ ID NO: 178, LC-CDR1: SEQ ID NO: 45, LC-CDR2: SEQ ID NO: 69, LC-CDR3: SEQ ID NO: 70; HC-CDR1: SEQ ID NO: 13, HC-CDR2: SEQ ID NO: 29, HC-CDR3: SEQ ID NO: 43, LC-CDR1: SEQ ID NO: 57, LC-CDR2: SEQ ID NO: 66, LC-CDR3: SEQ ID NO: 83; HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 84; and HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 85. In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 30; LC-CDR1: SEQ ID NO: 45, LC-CDR2: SEQ ID NO: 69, LC-CDR3: SEQ ID NO: 70; wherein the CDRs comprise 0-2 amino acid mutations, substitutions, or deletions in at least one of the HC-CDR1, HC-CDR2, HC-CDR3, LC-CDR1, LC-CDR2, LC-CDR3. In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 30; LC-CDR1: SEQ ID NO: 45, LC-CDR2: SEQ ID NO: 69, LC-CDR3: SEQ ID NO: 70. In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 177; LC-CDR1: SEQ ID NO: 45, LC-CDR2: SEQ ID NO: 69, LC-CDR3: SEQ ID NO: 70; wherein the CDRs comprise 0-2 amino acid mutations, substitutions, or deletions in at least one of the HC-CDR1, HC-CDR2, HC-CDR3, LC-CDR1, LC-CDR2, LC-CDR3. In some embodiments, the antibody comprises a heavy chain variable domain that comprises CDRs: HC-CDR1, HC-CDR2, HC-CDR3, and alight chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 177; LC-CDR1: SEQ ID NO: 45, LC-CDR2: SEQ ID NO: 69, LC-CDR3: SEQ ID NO: 70. In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 178; LC-CDR1: SEQ ID NO: 45, LC-CDR2: SEQ ID NO: 69, LC-CDR3: SEQ ID NO: 70; wherein the CDRs comprise 0-2 amino acid mutations, substitutions, or deletions in at least one of the HC-CDR1, HC-CDR2, HC-CDR3, LC-CDR1, LC-CDR2, LC-CDR3. In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 178; LC-CDR1: SEQ ID NO: 45, LC-CDR2: SEQ ID NO: 69, LC-CDR3: SEQ ID NO: 70. In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 43; LC-CDR1: SEQ ID NO: 57, LC-CDR2: SEQ ID NO: 66, LC-CDR3: SEQ ID NO: 83 wherein the CDRs comprise 0-2 amino acid mutations, substitutions, or deletions in at least one of the HC-CDR1, HC-CDR2, HC-CDR3, LC-CDR1, LC-CDR2, LC-CDR3. In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 43; LC-CDR1: SEQ ID NO: 57, LC-CDR2: SEQ ID NO: 66, LC-CDR3: SEQ ID NO: 83. In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 44; LC-CDR1: SEQ ID NO: 57, LC-CDR2: SEQ ID NO: 66, LC-CDR3: SEQ ID NO: 84; wherein the CDRs comprise 0-2 amino acid mutations, substitutions, or deletions in at least one of the HC-CDR1, HC-CDR2, HC-CDR3, LC-CDR1, LC-CDR2, LC-CDR3. In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 44; LC-CDR1: SEQ ID NO: 57, LC-CDR2: SEQ ID NO: 66, LC-CDR3: SEQ ID NO: 84. In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 44; LC-CDR1: SEQ ID NO: 57, LC-CDR2: SEQ ID NO: 66, LC-CDR3: SEQ ID NO: 85; wherein the CDRs comprise 0-2 amino acid mutations, substitutions, or deletions in at least one of the HC-CDR1, HC-CDR2, HC-CDR3, LC-CDR1, LC-CDR2, LC-CDR3. In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 44; LC-CDR1: SEQ ID NO: 57, LC-CDR2: SEQ ID NO: 66, LC-CDR3: SEQ ID NO: 85. In some embodiments, the antibody comprises an antibody format selected from Fab, Fab′, scFv, and (Fab′)₂. In some embodiments, the heavy chain variable domain is fused to a human IgG1 constant region. In some embodiments, the heavy chain variable domain is fused to a human IgG4 constant region. In some embodiments, the light chain variable domain is fused to a human Kappa constant region. In some embodiments, the heavy chain variable domain comprises a variable domain of an IgG1, IgG2, IgG3, or IgG4 heavy chain. In some embodiments, the light chain variable domain comprises a variable domain of a Kappa light chain. In some embodiments, the heavy chain variable domain comprises an amino acid sequence with at least 90%, 95%, 96%, 97%, 98%, 99%, or 100% identity to any one of SEQ ID NOs: 143, 149, 181, 184, 155, 161, or 167. In some embodiments, the light chain variable domain comprises an amino acid sequence with at least 90%, 95%, 96%, 97%, 98%, 99%, or 100% identity to any one of SEQ ID NOs: 144, 150, 156, 162, or 168. In some embodiments, the heavy chain variable domain comprises an amino acid sequence with at least 90% sequence identity to SEQ ID NO: 143, and the light chain variable domain comprises an amino acid sequence with at least 90% sequence identity to SEQ ID NO: 144. In some embodiments, the heavy chain variable domain comprises an amino acid sequence with at least 90% sequence identity to SEQ ID NO: 149, and the light chain variable domain comprises an amino acid sequence with at least 90% sequence identity to SEQ ID NO: 150. In some embodiments, the heavy chain variable domain comprises an amino acid sequence with at least 90% sequence identity to SEQ ID NO: 181, and the light chain variable domain comprises an amino acid sequence with at least 90% sequence identity to SEQ ID NO: 144. In some embodiments, the heavy chain variable domain comprises an amino acid sequence with at least 90% sequence identity to SEQ ID NO: 184, and the light chain variable domain comprises an amino acid sequence with at least 90% sequence identity to SEQ ID NO: 144. In some embodiments, the heavy chain variable domain comprises an amino acid sequence with at least 90% sequence identity to SEQ ID NO: 155, and the light chain variable domain comprises an amino acid sequence with at least 90% sequence identity to SEQ ID NO: 156. In some embodiments, the heavy chain variable domain comprises an amino acid sequence with at least 90% sequence identity to SEQ ID NO: 161, and the light chain variable domain comprises an amino acid sequence with at least 90% sequence identity to SEQ ID NO: 162. In some embodiments, the heavy chain variable domain comprises an amino acid sequence with at least 90% sequence identity to SEQ ID NO: 167, and the light chain variable domain comprises an amino acid sequence with at least 90% sequence identity to SEQ ID NO: 168. In some embodiments, the antibody comprises amino acid sequences with at least 90% sequence identity to SEQ ID NO: 145, and at least 90% sequence identity to SEQ ID NO: 147. In some embodiments, the antibody comprises amino acid sequences with at least 90% sequence identity to SEQ ID NO: 151, and at least 90% sequence identity to SEQ ID NO: 153. In some embodiments, the antibody comprises amino acid sequences with at least 90% sequence identity to SEQ ID NO: 182, and at least 90% sequence identity to SEQ ID NO: 147. In some embodiments, the antibody comprises amino acid sequences with at least 90% sequence identity to SEQ ID NO: 185, and at least 90% sequence identity to SEQ ID NO: 147. In some embodiments, the antibody comprises amino acid sequences with at least 90% sequence identity to SEQ ID NO: 157, and at least 90% sequence identity to SEQ ID NO: 159. In some embodiments, the antibody comprises amino acid sequences with at least 90% sequence identity to SEQ ID NO: 163, and at least 90% sequence identity to SEQ ID NO: 165. In some embodiments, the antibody comprises amino acid sequences with at least 90% sequence identity to SEQ ID NO: 169, and at least 90% sequence identity to SEQ ID NO: 171. In some embodiments, the antibody binds to extracellular domain of human CD47 with an EC50 of between 0.01 nM-0.5 nM as determined by an ELISA binding assay. In some embodiments, the ELISA binding assay comprises the following steps: coating a 96-well plate for at least 12 hours with 1 μg/ml of recombinant CD47; washing the plate three times; blocking the plate with 300 μL of 1% bovine serum albumin in phosphate-buffered saline solution with tween (PBST) at 37° C. for 1 hour; washing the plate four times with PBST; incubating a serial dilution of the antibody that binds specifically to CD47 at 37° C. for 1 hour; washing the plate 3 times with PBST; incubating a 1:5000 dilution of anti-human IgG-peroxidase antibody at 37° C. for 1 hour within the plate; washing the plate 4 times with PBST; incubating 3,3′,5,5′ tetramethylbenzidine substrate for 15 minutes at room temperature within the plate; terminating the reaction within the plate with 1N HCl; and reading the plate at 450 nM to determine the EC50 of the antibody for binding to the extracellular domain of human CD47. In some embodiments, the extracellular domain of human CD47 comprises an amino acid sequence according to SEQ ID NO: 86. In some embodiments, the antibody binds to CD47+ cell lines with an EC50 of between 0.01 nM-5 nM as determined by flow cytometry. In some embodiments, the EC50 as determined by flow cytometry comprises the following steps and assay conditions: centrifuging CD47+ cells at 2000 rpm for 5 minutes to obtain centrifuged cells; resuspending the centrifuged cells in 10-15 mL of culture medium; resuspending cells in blocking buffer comprising phosphate buffer saline with 2% fetal bovine serum (PBS plus 2% FBS) at a concentration of 3×10⁶ cells/mL to obtain a cell suspension; dispensing the cell suspension into wells of a 96-well plate; diluting the antibody that binds specifically to CD47 in the blocking buffer to a desired concentration followed by addition of 100 μL of the antibody per well and incubating at 1 hour for 4° C.; washing the cells three times with PBS plus 2% FBS; resuspending the cells in 100 μL 1:500 diluted Alexa Fluor 488 labeled Mouse anti-Human IgG1 Fe secondary antibody and incubating at 4° C. in the dark for one hour; washing the cells three times with 200 μL PBS and centrifuging at 2000 rpm for 5 minutes; resuspending cells in 300 μL cold PBS; and analyzing with a flow cytometer to determine the EC50 of the antibody for the CD47+ cell line. In some embodiments, the antibody blocks SIRPa activity with a IC50 of between 0.1 nM-5 nM as determined by flow cytometry. In some embodiments, the IC50 as determined by flow cytometry comprises the following steps and assay conditions: harvesting, centrifuging, and resuspending Raji tumor cells or HCT-15 tumor cells in FACS buffer comprising phosphate buffer saline with 2% fetal bovine serum (PBS plus 2% FBS) at a concentration of 2×10⁶ cells/mL; dispensing 100 μL of the cell suspension into wells of a 96-well plate; centrifuging the plate for 5 minutes at 300×g followed by discarding of supernatants; incubating cells from the plate with 50 μL per well of serially diluted antibody and a constant amount of SIRPA-mIgG2a fusion protein (0.2 μg/mL for Raji cells, 1 μg/mL for HCT-15 cells) in FACS buffer for 1 hour at 4° C.; washing the plate with FACS buffer followed by incubating for one hour at 4° C. in the dark with 100 μL of Alexa Fluor 488 donkey anti-Mouse IgG(H+L) secondary antibody; washing twice with FACS buffer, resuspending cells in the plate with 300 μL FACS buffer; and analyzing with a flow cytometer to determine the IC50 of the antibody for blocking SIRPa activity. In some embodiments, the antibody induces increased antibody-dependent cellular phagocytosis (ADCP) as compared to a control antibody that comprises amino acid sequences according to SEQ ID NOs: 173 and 174 when tested under substantially equivalent assay conditions in an assay for ADCP. In some embodiments, the antibody has less binding on human red blood cells (RBC) as compared to a control antibody that comprises amino acid sequences according to SEQ ID NOs: 173 and 174 when tested under substantially equivalent assay conditions for assessing RBC binding. In some embodiments, a concentration of 600 nM of the antibody does not induce hemolysis of red blood cells in a hemagglutination assay. In some embodiments, the antibody induces increased reduction in tumor volume as compared to a control antibody that comprises amino acid sequences according to SEQ ID NOs: 173 and 174 when tested under substantially equivalent assay conditions in a tumor growth animal model.

Disclosed herein are nucleic acid molecules encoding the antibody of any one of the above embodiments.

Disclosed herein are vectors comprising the nucleic acid molecule of the above embodiment.

Disclosed herein are pharmaceutical compositions comprising the antibody of any one of the above embodiments. In some embodiments, the pharmaceutical composition further comprises a pharmaceutically acceptable carrier, an excipient, or any combinations thereof.

Disclosed herein are methods of treating a subject having cancer, the method comprising: administering to the subject the antibody of any one of the above embodiments or the pharmaceutical composition of any one of the above embodiments. In some embodiments, the cancer comprises cancer cells that express CD47. In some embodiments, the antibody induces antibody-dependent cellular phagocytosis (ADCP) of the cancer cells that express CD47. In some embodiments, the cancer is a hematological malignancy. In some embodiments, the cancer is B cell cancer or T cell cancer. In some embodiments, the cancer is leukemia or lymphoma. In some embodiments, the cancer is lymphoma, and wherein the lymphoma is B-cell lymphoma. In some embodiments, the cancer is lymphoma, and wherein the lymphoma is T-cell lymphoma. In some embodiments, the cancer is a solid tumor. In some embodiments, the solid tumor is sarcoma, breast cancer, lung cancer, carcinoma, ovarian cancer, pancreatic cancer, gastric cancer, colorectal cancer, endometrial cancer, esophageal cancer, prostate cancer, cervical cancer, kidney cancer, urothelial cancer, or head and neck cancer. In some embodiments, the solid tumor is lung cancer, and wherein the lung cancer is non-small cell lung cancer. In some embodiments, the solid tumor is lung cancer, and wherein the lung cancer is small cell lung cancer. In some embodiments, further comprising administering to the subject an anti-cancer agent. In some embodiments, the anti-cancer agent is a chemotherapeutic agent or a biologic agent. In some embodiments, the administering is sufficient to reduce or eliminate the cancer as compared to a comparable method lacking the administering. In some embodiments, the reduction is at least about 1 fold, 5 fold, 10 fold, 20 fold, 40 fold, 60 fold, 80 fold, or up to about 100 fold. In some embodiments, the cancer is metastatic.

Disclosed herein are kits that comprise the antibody of any one of the above embodiments, the vector of any one of the above embodiments, the nucleic acid molecule of any one of the above embodiments, or the pharmaceutical composition of any one of the above embodiments.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1B show amino acid sequence alignments of the VL (FIG. 1A) and VH (FIG. 1B) domains of chimeric and humanized and engineered variants of ID15E11. The CDRs are highlighted in bold. Vertical lines denote conserved residues. FIG. 1A discloses SEQ ID NOS 188-190, 144, and 191, respectively, in order of appearance. FIG. 1B discloses SEQ ID NOS 192-200, 143, 201-202, and 244-245, respectively, in order of appearance.

FIGS. 2A-2B show amino acid sequences of the VL (FIG. 2A) and VH (FIG. 2B) domains of chimeric and humanized variants of 18B9. FIG. 2A discloses SEQ ID NOS 203-207, and 156, respectively, in order of appearance. FIG. 2B discloses SEQ ID NOS 208-220, and 161, respectively, in order of appearance.

FIGS. 3A-3B show amino acid sequence alignments of alanine scan variants of the hu.18B9.3i light chain (FIG. 3A) and heavy chain (FIG. 3B). FIG. 3A discloses SEQ ID NOS 156, 221-222, 162, 223-231, and 168, respectively, in order of appearance. FIG. 3B discloses SEQ ID NOS 161, 232-243, and 155, respectively, in order of appearance.

FIGS. 4A-4B show ELISA binding of hu.1D15E11 variants on human CD47 and cynomolgus monkey CD47

FIG. 5A-5B show a SIRPA blocking assay utilizing HCT-15 cells cultured with anti-CD47 antibodies as compared to various controls.

FIG. 6 shows Antibody Dependent Cellular Phagocytosis (ADCP) of HCT-15 cells cultured with anti-CD47 antibodies as compared to various controls.

FIGS. 7A-7B show binding of human red blood cells (RBC) cultured with anti-CD47 antibodies as compared to various controls.

FIGS. 8A-8B show binding of human platelet cells cultured with anti-CD47 antibodies as compared to various controls.

FIG. 9 shows hemagglutination of human red blood cells by anti-CD47 antibodies as compared to various controls.

FIG. 10 shows an in vivo Burkitts lymphoma Raji model of mice administered 0.3 mg/kg of anti-CD47 antibodies as compared to various controls.

FIG. 11 shows an in vivo renal cell carcinoma 786-O model of mice administered 10 mg/kg of anti-CD47 antibody as compared to BMK-1.

### DETAILED DESCRIPTION

Cluster of differentiation 47 (CD47), also known as integrin-associated protein (IAP), is a ˜50 kDa immunoglobulin superfamily membrane glycoprotein that is overexpressed in numerous blood cancers and solid tumors. High CD47 expression often correlates with more aggressive diseases and poorer clinical outcomes.

CD47 on the surface of CD47+ cells interacts with signal regulatory protein alpha (SIRPA) expressed mainly on myeloid cells, such as macrophages. This interaction sends a “don't eat me” signal that inhibits phagocytosis, thereby allowing CD47+ cells to evade immune surveillance. These data suggest that CD47 may serve as an immune checkpoint and that blocking the CD47-SIRPA interaction could have therapeutic value by switching off the “don't eat me” signal. Thus, blocking CD47 has emerged as a promising therapeutic strategy with numerous studies showing that interrupting the CD47-SIRPA signaling pathway promotes anti-tumor activity against human cancers in vitro and in vivo.

Several anti-CD47 monoclonal antibodies (mAbs) have been shown to increase phagocytosis of acute myeloid leukemia cells, non-Hodgkin's lymphoma cells, breast cancer cells, and ovarian cancer cells. In clinical studies, CD47 mAbs enhanced the anti-tumor activity of other therapeutic antibodies. At least six anti-CD47 mAbs and three SIRPA fusion proteins are in active phase I or II clinical trials for the treatment of human hematological malignancies and solid tumors.

The efficacy of anti-CD47 mAbs is limited by their interactions with red blood cells (RBCs), which also express CD47. RBCs act as a sink to sequester anti-CD47 antibodies, thereby preventing them from binding to malignant CD47-expressing (CD47+) cells. Furthermore, anti-CD47 mAb binding to RBCs leads to hemagglutination and lysis of the RBCs, resulting in anemia. Thus, there is a need for improved methods of treating malignant diseases mediated by CD47+ cells with reduced off-tumor effects.

### Compositions

Antibodies that Bind Specifically to CD47

Disclosed herein are antibodies that bind specifically to CD47 that have optimized functional properties or optimized safety profiles relative to control antibodies that bind specifically to CD47. In some embodiments, are antibodies that bind specifically to CD47 that have optimized functional properties and optimized safety profiles relative to control antibodies that bind specifically to CD47. In some embodiments, the optimized functional properties include improved binding to CD47, improved SIRPa blocking activity, or increased tumor growth inhibition. In some embodiments, the optimized safety profile includes reduced hemagglutination and lysis of RBCs.

Disclosed herein are antibodies that binds specifically to CD47 comprising at least one complementarity determining region (CDR) according to SEQ ID NOs: 1-85, 176-178 or an amino acid sequence that has 0-1 amino acid mutations, substitutions, or deletions relative to SEQ ID NOs: 1-13, 58-69, or an amino acid sequence that has 0-3 amino acid mutations, substitutions, or deletions to relative to SEQ ID NOs: 14-57, 70-85, and 176-178. In some embodiments, the antibody that binds specifically to CD47 comprises at least one CDR according to SEQ ID NOs: 1-85, or 176-178.

In some embodiments, the antibody comprises a heavy chain variable domain that comprises CDRs: HC-CDR1, HC-CDR2, HC-CDR3, wherein the HC-CDR3 comprises an amino acid sequence selected from SEQ ID NOs: 30-44, and 176-178. In some embodiments, the antibody comprises a heavy chain variable domain that comprises CDRs: HC-CDR1, HC-CDR2, HC-CDR3, wherein the HC-CDR3 comprises an amino acid sequence selected from SEQ ID NOs: 30-44, and 176-178 wherein the HC-CDR3 comprises 0-2 amino acid mutations, substitutions, or deletions.

In some embodiments, the antibody comprises a heavy chain variable domain that comprises CDRs: HC-CDR1, HC-CDR2, HC-CDR3, wherein the HC-CDR3 comprises an amino acid sequence selected from SEQ ID NOs: 30, 177-178, 43-44. In some embodiments, the antibody comprises a heavy chain variable domain that comprises CDRs: HC-CDR1, HC-CDR2, HC-CDR3, wherein the HC-CDR3 comprises an amino acid sequence selected from SEQ ID NOs: 30, 177-178, 43-44, wherein the HC-CDR3 comprises 0-2 amino acid mutations, substitutions, or deletions.

In some embodiments, the antibody comprises a heavy chain variable domain that comprises CDRs: HC-CDR1, HC-CDR2, HC-CDR3, wherein the HC-CDR1, HC-CDR2, HC-CDR3 of the heavy chain variable domain comprises a set of sequences selected from the group consisting of the following set of sequences: HC-CDR1: SEQ ID NO: 1, HC-CDR2: SEQ ID NO: 14, HC-CDR3: SEQ ID NO: 30; HC-CDR1: SEQ ID NO: 2, HC-CDR2: SEQ ID NO: 15, HC-CDR3: SEQ ID NO: 31; HC-CDR1: SEQ ID NO: 3, HC-CDR2: SEQ ID NO: 16, HC-CDR3: SEQ ID NO: 32; HC-CDR1: SEQ ID NO: 2, HC-CDR2: SEQ ID NO: 17, HC-CDR3: SEQ ID NO: 33; HC-CDR1: SEQ ID NO: 4, HC-CDR2: SEQ ID NO: 18, HC-CDR3: SEQ ID NO: 34; HC-CDR1: SEQ ID NO: 5, HC-CDR2: SEQ ID NO: 19, HC-CDR3: SEQ ID NO: 35; HC-CDR1: SEQ ID NO: 6, HC-CDR2: SEQ ID NO: 20, HC-CDR3: SEQ ID NO: 176; HC-CDR1: SEQ ID NO: 7, HC-CDR2: SEQ ID NO: 21, HC-CDR3: SEQ ID NO: 36; HC-CDR1: SEQ ID NO: 8, HC-CDR2: SEQ ID NO: 22, HC-CDR3: SEQ ID NO: 37; HC-CDR1: SEQ ID NO: 9, HC-CDR2: SEQ ID NO: 23, HC-CDR3: SEQ ID NO: 38; HC-CDR1: SEQ ID NO: 10, HC-CDR2: SEQ ID NO: 24, HC-CDR3: SEQ ID NO: 39; HC-CDR1: SEQ ID NO: 8, HC-CDR2: SEQ ID NO: 25, HC-CDR3: SEQ ID NO: 40; HC-CDR1: SEQ ID NO: 11, HC-CDR2: SEQ ID NO: 26, HC-CDR3: SEQ ID NO: 41; HC-CDR1: SEQ ID NO: 12, HC-CDR2: SEQ ID NO: 27, HC-CDR3: SEQ ID NO: 42; HC-CDR1: SEQ ID NO: 1, HC-CDR2: SEQ ID NO: 28, HC-CDR3: SEQ ID NO: 30; HC-CDR1: SEQ ID NO: 1, HC-CDR2: SEQ ID NO: 28, HC-CDR3: SEQ ID NO: 177; HC-CDR1: SEQ ID NO: 1, HC-CDR2: SEQ ID NO: 28, HC-CDR3: SEQ ID NO: 178; HC-CDR1: SEQ ID NO: 13, HC-CDR2: SEQ ID NO: 29, HC-CDR3: SEQ ID NO: 43; and HC-CDR1: SEQ ID NO: 13, HC-CDR2: SEQ ID NO: 29, HC-CDR3: SEQ ID NO: 44; and wherein the CDRs comprise 0-2 amino acid mutations, substitutions, or deletions in at least one of the HC-CDR1, HC-CDR2, HC-CDR3. In some embodiments, the antibody comprises a heavy chain variable domain that comprises CDRs: HC-CDR1, HC-CDR2, HC-CDR3, wherein the HC-CDR1, HC-CDR2, HC-CDR3 of the heavy chain variable domain comprises a set of sequences selected from the group consisting of the following set of sequences: HC-CDR1: SEQ ID NO: 1, HC-CDR2: SEQ ID NO: 14, HC-CDR3: SEQ ID NO: 30; HC-CDR1: SEQ ID NO: 2, HC-CDR2: SEQ ID NO: 15, HC-CDR3: SEQ ID NO: 31; HC-CDR1: SEQ ID NO: 3, HC-CDR2: SEQ ID NO: 16, HC-CDR3: SEQ ID NO: 32; HC-CDR1: SEQ ID NO: 2, HC-CDR2: SEQ ID NO: 17, HC-CDR3: SEQ ID NO: 33; HC-CDR1: SEQ ID NO: 4, HC-CDR2: SEQ ID NO: 18, HC-CDR3: SEQ ID NO: 34; HC-CDR1: SEQ ID NO: 5, HC-CDR2: SEQ ID NO: 19, HC-CDR3: SEQ ID NO: 35; HC-CDR1: SEQ ID NO: 6, HC-CDR2: SEQ ID NO: 20, HC-CDR3: SEQ ID NO: 176; HC-CDR1: SEQ ID NO: 7, HC-CDR2: SEQ ID NO: 21, HC-CDR3: SEQ ID NO: 36; HC-CDR1: SEQ ID NO: 8, HC-CDR2: SEQ ID NO: 22, HC-CDR3: SEQ ID NO: 37; HC-CDR1: SEQ ID NO: 9, HC-CDR2: SEQ ID NO: 23, HC-CDR3: SEQ ID NO: 38; HC-CDR1: SEQ ID NO: 10, HC-CDR2: SEQ ID NO: 24, HC-CDR3: SEQ ID NO: 39; HC-CDR1: SEQ ID NO: 8, HC-CDR2: SEQ ID NO: 25, HC-CDR3: SEQ ID NO: 40; HC-CDR1: SEQ ID NO: 11, HC-CDR2: SEQ ID NO: 26, HC-CDR3: SEQ ID NO: 41; HC-CDR1: SEQ ID NO: 12, HC-CDR2: SEQ ID NO: 27, HC-CDR3: SEQ ID NO: 42; HC-CDR1: SEQ ID NO: 1, HC-CDR2: SEQ ID NO: 28, HC-CDR3: SEQ ID NO: 30; HC-CDR1: SEQ ID NO: 1, HC-CDR2: SEQ ID NO: 28, HC-CDR3: SEQ ID NO: 177; HC-CDR1: SEQ ID NO: 1, HC-CDR2: SEQ ID NO: 28, HC-CDR3: SEQ ID NO: 178; HC-CDR1: SEQ ID NO: 13, HC-CDR2: SEQ ID NO: 29, HC-CDR3: SEQ ID NO: 43; and HC-CDR1: SEQ ID NO: 13, HC-CDR2: SEQ ID NO: 29, HC-CDR3: SEQ ID NO: 44.

In some embodiments, the antibody comprises a light chain variable domain that comprises CDRs: LC-CDR1, LC-CDR2, LC-CDR3, wherein the LC-CDR3 comprises an amino acid sequence according to SEQ ID NOs: 70-85. In some embodiments, the antibody comprises a light chain variable domain that comprises CDRs: LC-CDR1, LC-CDR2, LC-CDR3, wherein the LC-CDR3 comprises an amino acid sequence according to SEQ ID NOs: 70-85 wherein the LC-CDR3 comprises 0-2 amino acid mutations, substitutions, or deletions. In some embodiments, the antibody comprises a light chain variable domain that comprises CDRs: LC-CDR1, LC-CDR2, LC-CDR3, wherein the LC-CDR3 comprises an amino acid sequence according to SEQ ID NOs: 70, 83-85. In some embodiments, the antibody comprises a light chain variable domain that comprises CDRs: LC-CDR1, LC-CDR2, LC-CDR3, wherein the LC-CDR3 comprises an amino acid sequence according to SEQ ID NOs: 70, 83-85 wherein the LC-CDR3 comprises 0-2 amino acid mutations, substitutions, or deletions.

In some embodiments, the antibody comprises a light chain variable domain that comprises CDRs: LC-CDR1, LC-CDR2, LC-CDR3, wherein the LC-CDR1, LC-CDR2, LC-CDR3 of the light chain variable domain comprises a set of sequences selected from the group consisting of the following set of sequences: LC-CDR1: SEQ ID NO: 45, LC-CDR2: SEQ ID NO: 58, LC-CDR3: SEQ ID NO: 70; LC-CDR1: SEQ ID NO: 46, LC-CDR2: SEQ ID NO: 59, LC-CDR3: SEQ ID NO: 71; LC-CDR1: SEQ ID NO: 47, LC-CDR2: SEQ ID NO: 60, LC-CDR3: SEQ ID NO: 72; LC-CDR1: SEQ ID NO: 48, LC-CDR2: SEQ ID NO: 59, LC-CDR3: SEQ ID NO: 73; LC-CDR1: SEQ ID NO: 49, LC-CDR2: SEQ ID NO: 61, LC-CDR3: SEQ ID NO: 74; LC-CDR1: SEQ ID NO: 50, LC-CDR2: SEQ ID NO: 62, LC-CDR3: SEQ ID NO: 75; LC-CDR1: SEQ ID NO: 51, LC-CDR2: SEQ ID NO: 63, LC-CDR3: SEQ ID NO: 76; LC-CDR1: SEQ ID NO: 52, LC-CDR2: SEQ ID NO: 62, LC-CDR3: SEQ ID NO: 77; LC-CDR1: SEQ ID NO: 53, LC-CDR2: SEQ ID NO: 64, LC-CDR3: SEQ ID NO: 78; LC-CDR1: SEQ ID NO: 54, LC-CDR2: SEQ ID NO: 65, LC-CDR3: SEQ ID NO: 79; LC-CDR1: SEQ ID NO: 55, LC-CDR2: SEQ ID NO: 66, LC-CDR3: SEQ ID NO: 80; LC-CDR1: SEQ ID NO: 53, LC-CDR2: SEQ ID NO: 67, LC-CDR3: SEQ ID NO: 78; LC-CDR1: SEQ ID NO: 52, LC-CDR2: SEQ ID NO: 62, LC-CDR3: SEQ ID NO: 81; LC-CDR1: SEQ ID NO: 56, LC-CDR2: SEQ ID NO: 68, LC-CDR3: SEQ ID NO: 82; LC-CDR1: SEQ ID NO: 45, LC-CDR2: SEQ ID NO: 69, LC-CDR3: SEQ ID NO: 70; LC-CDR1: SEQ ID NO: 57, LC-CDR2: SEQ ID NO: 66, LC-CDR3: SEQ ID NO: 83 LC-CDR1: SEQ ID NO: 57, LC-CDR2: SEQ ID NO: 66, LC-CDR3: SEQ ID NO: 84; and LC-CDR1: SEQ ID NO: 57, LC-CDR2: SEQ ID NO: 66, LC-CDR3: SEQ ID NO: 85, wherein the CDRs comprise 0-2 amino acid mutations, substitutions, or deletions in at least one of the LC-CDR1, LC-CDR2, LC-CDR3. In some embodiments, the antibody comprises a light chain variable domain that comprises CDRs: LC-CDR1, LC-CDR2, LC-CDR3, wherein the LC-CDR1, LC-CDR2, LC-CDR3 of the light chain variable domain comprises a set of sequences selected from the group consisting of the following set of sequences: LC-CDR1: SEQ ID NO: 45, LC-CDR2: SEQ ID NO: 58, LC-CDR3: SEQ ID NO: 70; LC-CDR1: SEQ ID NO: 46, LC-CDR2: SEQ ID NO: 59, LC-CDR3: SEQ ID NO: 71; LC-CDR1: SEQ ID NO: 47, LC-CDR2: SEQ ID NO: 60, LC-CDR3: SEQ ID NO: 72; LC-CDR1: SEQ ID NO: 48, LC-CDR2: SEQ ID NO: 59, LC-CDR3: SEQ ID NO: 73; LC-CDR1: SEQ ID NO: 49, LC-CDR2: SEQ ID NO: 61, LC-CDR3: SEQ ID NO: 74; LC-CDR1: SEQ ID NO: 50, LC-CDR2: SEQ ID NO: 62, LC-CDR3: SEQ ID NO: 75; LC-CDR1: SEQ ID NO: 51, LC-CDR2: SEQ ID NO: 63, LC-CDR3: SEQ ID NO: 76; LC-CDR1: SEQ ID NO: 52, LC-CDR2: SEQ ID NO: 62, LC-CDR3: SEQ ID NO: 77; LC-CDR1: SEQ ID NO: 53, LC-CDR2: SEQ ID NO: 64, LC-CDR3: SEQ ID NO: 78; LC-CDR1: SEQ ID NO: 54, LC-CDR2: SEQ ID NO: 65, LC-CDR3: SEQ ID NO: 79; LC-CDR1: SEQ ID NO: 55, LC-CDR2: SEQ ID NO: 66, LC-CDR3: SEQ ID NO: 80; LC-CDR1: SEQ ID NO: 53, LC-CDR2: SEQ ID NO: 67, LC-CDR3: SEQ ID NO: 78; LC-CDR1: SEQ ID NO: 52, LC-CDR2: SEQ ID NO: 62, LC-CDR3: SEQ ID NO: 81; LC-CDR1: SEQ ID NO: 56, LC-CDR2: SEQ ID NO: 68, LC-CDR3: SEQ ID NO: 82; LC-CDR1: SEQ ID NO: 45, LC-CDR2: SEQ ID NO: 69, LC-CDR3: SEQ ID NO: 70; LC-CDR1: SEQ ID NO: 57, LC-CDR2: SEQ ID NO: 66, LC-CDR3: SEQ ID NO: 83; LC-CDR1: SEQ ID NO: 57, LC-CDR2: SEQ ID NO: 66, LC-CDR3: SEQ ID NO: 84; LC-CDR1: SEQ ID NO: 57, LC-CDR2: SEQ ID NO: 66, LC-CDR3: SEQ ID NO: 85.

In some embodiments, the antibody comprises at least one complementarity determining region (CDR) according to SEQ ID NOs: 1, 13, 66, 69, 28-30, 30, 43, 44, 45, 57, 70, 83-85, 177-178 or an amino acid sequence that has 0-1 amino acid mutations, substitutions, or deletions relative to SEQ ID NOs: 1, 13, 66, 69, or an amino acid sequence that has 0-3 amino acid mutations, substitutions, or deletions to relative to SEQ ID NOs:28-30, 30, 43, 44, 45, 57, 70, 83-85, 177-178.

In some embodiments, the antibody comprises a heavy chain variable domain that comprises CDRs: HC-CDR1, HC-CDR2, HC-CDR3, wherein the HC-CDR1, HC-CDR2, HC-CDR3 of the heavy chain variable domain comprises a set of sequences selected from the group consisting of the following set of sequences: HC-CDR1: SEQ ID NO: 1, HC-CDR2: SEQ ID NO: 28, HC-CDR3: SEQ ID NO: 30; HC-CDR1: SEQ ID NO: 1, HC-CDR2: SEQ ID NO: 28, HC-CDR3: SEQ ID NO: 177; HC-CDR1: SEQ ID NO: 1, HC-CDR2: SEQ ID NO: 28, HC-CDR3: SEQ ID NO: 178; HC-CDR1: SEQ ID NO: 13, HC-CDR2: SEQ ID NO: 29, HC-CDR3: SEQ ID NO: 43; and HC-CDR1: SEQ ID NO: 13, HC-CDR2: SEQ ID NO: 29, HC-CDR3: SEQ ID NO: 44; and wherein the CDRs comprise 0-2 amino acid mutations, substitutions, or deletions in at least one of the HC-CDR1, HC-CDR2, HC-CDR3.

In some embodiments, the antibody comprises a heavy chain variable domain that comprises CDRs: HC-CDR1, HC-CDR2, HC-CDR3, wherein the HC-CDR1, HC-CDR2, HC-CDR3 of the heavy chain variable domain comprises a set of sequences selected from the group consisting of the following set of sequences: HC-CDR1: SEQ ID NO: 1, HC-CDR2: SEQ ID NO: 28, HC-CDR3: SEQ ID NO: 30; HC-CDR1: SEQ ID NO: 1, HC-CDR2: SEQ ID NO: 28, HC-CDR3: SEQ ID NO: 177; HC-CDR1: SEQ ID NO: 1, HC-CDR2: SEQ ID NO: 28, HC-CDR3: SEQ ID NO: 178; HC-CDR1: SEQ ID NO: 13, HC-CDR2: SEQ ID NO: 29, HC-CDR3: SEQ ID NO: 43; and HC-CDR1: SEQ ID NO: 13, HC-CDR2: SEQ ID NO: 29, HC-CDR3: SEQ ID NO: 44.

In some embodiments, the antibody comprises a light chain variable domain that comprises CDRs: LC-CDR1, LC-CDR2, LC-CDR3, wherein the LC-CDR1, LC-CDR2, LC-CDR3 of the light chain variable domain comprises a set of sequences selected from the group consisting of the following set of sequences: LC-CDR1: SEQ ID NO: 45, LC-CDR2: SEQ ID NO: 69, LC-CDR3: SEQ ID NO: 70; LC-CDR1: SEQ ID NO: 57, LC-CDR2: SEQ ID NO: 66, LC-CDR3: SEQ ID NO: 83; LC-CDR1: SEQ ID NO: 57, LC-CDR2: SEQ ID NO: 66, LC-CDR3: SEQ ID NO: 84; LC-CDR1: SEQ ID NO: 57, LC-CDR2: SEQ ID NO: 66, LC-CDR3: SEQ ID NO: 85; and wherein the CDRs comprise 0-2 amino acid mutations, substitutions, or deletions in at least one of the LC-CDR1, LC-CDR2, LC-CDR3.

In some embodiments, the antibody comprises a light chain variable domain that comprises CDRs: LC-CDR1, LC-CDR2, LC-CDR3, wherein the LC-CDR1, LC-CDR2, LC-CDR3 of the light chain variable domain comprises a set of sequences selected from the group consisting of the following set of sequences: LC-CDR1: SEQ ID NO: 45, LC-CDR2: SEQ ID NO: 69, LC-CDR3: SEQ ID NO: 70; LC-CDR1: SEQ ID NO: 57, LC-CDR2: SEQ ID NO: 66, LC-CDR3: SEQ ID NO: 83; LC-CDR1: SEQ ID NO: 57, LC-CDR2: SEQ ID NO: 66, LC-CDR3: SEQ ID NO: 84; and LC-CDR1: SEQ ID NO: 57, LC-CDR2: SEQ ID NO: 66, LC-CDR3: SEQ ID NO: 85.

In some embodiments, the antibody comprises a heavy chain variable domain that comprises CDRs: HC-CDR1, HC-CDR2, HC-CDR3, wherein the HC-CDR1, HC-CDR2, HC-CDR3 of the heavy chain variable domain comprises a set of sequences HC-CDR1: SEQ ID NO: 13, HC-CDR2: SEQ ID NO: 29, HC-CDR3: SEQ ID NO: 44; and wherein the CDRs comprise 0-2 amino acid mutations, substitutions, or deletions in at least one of the HC-CDR1, HC-CDR2, HC-CDR3.

In some embodiments, the antibody comprises a heavy chain variable domain that comprises CDRs: HC-CDR1, HC-CDR2, HC-CDR3, wherein the HC-CDR1, HC-CDR2, HC-CDR3 of the heavy chain variable domain comprises a set of sequences: HC-CDR1: SEQ ID NO: 13, HC-CDR2: SEQ ID NO: 29, HC-CDR3: SEQ ID NO: 44.

In some embodiments, the antibody comprises a light chain variable domain that comprises CDRs: LC-CDR1, LC-CDR2, LC-CDR3, wherein the LC-CDR1, LC-CDR2, LC-CDR3 of the light chain variable domain comprises LC-CDR1: SEQ ID NO: 57, LC-CDR2: SEQ ID NO: 66, LC-CDR3: SEQ ID NO: 84; or SEQ ID NO: 57, LC-CDR2: SEQ ID NO: 66, LC-CDR3: SEQ ID NO: 85; and wherein the CDRs comprise 0-2 amino acid mutations, substitutions, or deletions in at least one of the LC-CDR1, LC-CDR2, LC-CDR3.

In some embodiments, the antibody comprises a light chain variable domain that comprises CDRs: LC-CDR1, LC-CDR2, LC-CDR3, wherein the LC-CDR1, LC-CDR2, LC-CDR3 of the light chain variable domain comprises SEQ ID NO: 57, LC-CDR2: SEQ ID NO: 66, LC-CDR3: SEQ ID NO: 84; or SEQ ID NO: 57, LC-CDR2: SEQ ID NO: 66, LC-CDR3: SEQ ID NO: 85.

In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise a set of sequences selected from the group consisting of the following set of sequences: HC-CDR1: SEQ ID NO: 1, HC-CDR2: SEQ ID NO: 28, HC-CDR3: SEQ ID NO: 30, LC-CDR1: SEQ ID NO: 45, LC-CDR2: SEQ ID NO: 69, LC-CDR3: SEQ ID NO: 70; HC-CDR1: SEQ ID NO: 1, HC-CDR2: SEQ ID NO: 28, HC-CDR3: SEQ ID NO: 177, LC-CDR1: SEQ ID NO: 45, LC-CDR2: SEQ ID NO: 69, LC-CDR3: SEQ ID NO: 70; HC-CDR1: SEQ ID NO: 1, HC-CDR2: SEQ ID NO: 28, HC-CDR3: SEQ ID NO: 178, LC-CDR1: SEQ ID NO: 45, LC-CDR2: SEQ ID NO: 69, LC-CDR3: SEQ ID NO: 70; HC-CDR1: SEQ ID NO: 13, HC-CDR2: SEQ ID NO: 29, HC-CDR3: SEQ ID NO: 43, LC-CDR1: SEQ ID NO: 57, LC-CDR2: SEQ ID NO: 66, LC-CDR3: SEQ ID NO: 83; HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 84; and HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 85; wherein the CDRs comprise 0-2 amino acid mutations, substitutions, or deletions in at least one of the HC-CDR1, HC-CDR2, HC-CDR3, LC-CDR1, LC-CDR2, LC-CDR3.

In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise a set of sequences selected from the group consisting of the following set of sequences: HC-CDR1: SEQ ID NO: 1, HC-CDR2: SEQ ID NO: 28, HC-CDR3: SEQ ID NO: 30, LC-CDR1: SEQ ID NO: 45, LC-CDR2: SEQ ID NO: 69, LC-CDR3: SEQ ID NO: 70; HC-CDR1: SEQ ID NO: 1, HC-CDR2: SEQ ID NO: 28, HC-CDR3: SEQ ID NO: 177, LC-CDR1: SEQ ID NO: 45, LC-CDR2: SEQ ID NO: 69, LC-CDR3: SEQ ID NO: 70; HC-CDR1: SEQ ID NO: 1, HC-CDR2: SEQ ID NO: 28, HC-CDR3: SEQ ID NO: 178, LC-CDR1: SEQ ID NO: 45, LC-CDR2: SEQ ID NO: 69, LC-CDR3: SEQ ID NO: 70; HC-CDR1: SEQ ID NO: 13, HC-CDR2: SEQ ID NO: 29, HC-CDR3: SEQ ID NO: 43, LC-CDR1: SEQ ID NO: 57, LC-CDR2: SEQ ID NO: 66, LC-CDR3: SEQ ID NO: 83; HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 84; and HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 85.

In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 30; LC-CDR1: SEQ ID NO: 45, LC-CDR2: SEQ ID NO: 69, LC-CDR3: SEQ ID NO: 70; wherein the CDRs comprise 0-2 amino acid mutations, substitutions, or deletions in at least one of the HC-CDR1, HC-CDR2, HC-CDR3, LC-CDR1, LC-CDR2, LC-CDR3. In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 30; LC-CDR1: SEQ ID NO: 45, LC-CDR2: SEQ ID NO: 69, LC-CDR3: SEQ ID NO: 70.

In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 177; LC-CDR1: SEQ ID NO: 45, LC-CDR2: SEQ ID NO: 69, LC-CDR3: SEQ ID NO: 70; wherein the CDRs comprise 0-2 amino acid mutations, substitutions, or deletions in at least one of the HC-CDR1, HC-CDR2, HC-CDR3, LC-CDR1, LC-CDR2, LC-CDR3. In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 177; LC-CDR1: SEQ ID NO: 45, LC-CDR2: SEQ ID NO: 69, LC-CDR3: SEQ ID NO: 70.

In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 178; LC-CDR1: SEQ ID NO: 45, LC-CDR2: SEQ ID NO: 69, LC-CDR3: SEQ ID NO: 70; wherein the CDRs comprise 0-2 amino acid mutations, substitutions, or deletions in at least one of the HC-CDR1, HC-CDR2, HC-CDR3, LC-CDR1, LC-CDR2, LC-CDR3. In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 178; LC-CDR1: SEQ ID NO: 45, LC-CDR2: SEQ ID NO: 69, LC-CDR3: SEQ ID NO: 70.

In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 43; LC-CDR1: SEQ ID NO: 57, LC-CDR2: SEQ ID NO: 66, LC-CDR3: SEQ ID NO: 83; wherein the CDRs comprise 0-2 amino acid mutations, substitutions, or deletions in at least one of the HC-CDR1, HC-CDR2, HC-CDR3, LC-CDR1, LC-CDR2, LC-CDR3. In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 43; LC-CDR1: SEQ ID NO: 57, LC-CDR2: SEQ ID NO: 66, LC-CDR3: SEQ ID NO: 83.

In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 44; LC-CDR1: SEQ ID NO: 57, LC-CDR2: SEQ ID NO: 66, LC-CDR3: SEQ ID NO: 84; wherein the CDRs comprise 0-2 amino acid mutations, substitutions, or deletions in at least one of the HC-CDR1, HC-CDR2, HC-CDR3, LC-CDR1, LC-CDR2, LC-CDR3. In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 44; LC-CDR1: SEQ ID NO: 57, LC-CDR2: SEQ ID NO: 66, LC-CDR3: SEQ ID NO: 84.

In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 44; LC-CDR1: SEQ ID NO: 57, LC-CDR2: SEQ ID NO: 66, LC-CDR3: SEQ ID NO: 85; wherein the CDRs comprise 0-2 amino acid mutations, substitutions, or deletions in at least one of the HC-CDR1, HC-CDR2, HC-CDR3, LC-CDR1, LC-CDR2, LC-CDR3. In some embodiments, the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 44; LC-CDR1: SEQ ID NO: 57, LC-CDR2: SEQ ID NO: 66, LC-CDR3: SEQ ID NO: 85.

Table 1 and Table 2 provide the amino acid sequences for the CDRs referenced above. CDRs were determined by Kabat numbering system.

TABLE 1
Anti-CD47 Antibody Heavy Chain CDR sequences

| | | SEQ | | SEQ | | SEQ |
|---|---|---|---|---|---|---|
| | HC- | ID | | ID | | ID |
| Antibody Name | CDR-1 | NO: | HC-CDR-2 | NO: | HC-CDR-3 | NO: |
| M.1D15E11 | SYVMH | 1 | YIDPYNDGTKYNEKFKG | 14 | GGMARRSFDY | 30 |
| M.20B10E9 | DTYMH | 2 | RIDPANGKTKSDPKFQG | 15 | GDGYYVGGLDY | 31 |
| M.69H1H9 | EYTIH | 3 | SINPNNGLFRYNQKFKG | 16 | DYGFGYSDY | 32 |
| M.30A5C6 | DTYMH | 2 | RIDPANGNTKYDPKFQG | 17 | GDGHYVGGLNY | 33 |
| M.95D4A11 | SYWMN | 4 | SIDPYDSETHYNQKFKD | 18 | GRGYAMDY | 34 |
| R.1H3 | SGYYMC | 5 | CIYMRSGDTYLASWAKG | 19 | GGTSYALNL | 35 |
| R.9B11 | SYWMA | 6 | CIYIGSGSGTTYYASWAK | 20 | WAGSDGHFTL | 176 |
| R.13A2 | SYYYVC | 7 | CIYAGSSGDTYYASWAKG | 21 | GYTGYSYQFNF | 36 |
| R.14G4 | SYHMC | 8 | CIYPNNGFTWYASWAKG | 22 | GSGNSYNQLDL | 37 |
| R.15G10 | SYHMN | 9 | IIYVGSGNADYASWAKG | 23 | GPTNTYPTYFNL | 38 |
| R.18B9 | SGYMC | 10 | CIYAGSSGKTYYASWATG | 24 | DDGGTYYTDYLYL | 39 |
| R.19H7 | SYHMC | 8 | CIYPNNDFTWYATWAKG | 25 | ASGNAYNLLDL | 40 |
| R.20H1 | SGYYMC | 11 | CIYVRSDSTYYASWAKG | 26 | GVTGDSYRFNF | 41 |
| R.1B11 | SYHMS | 12 | IIYADTGNADYASWTKG | 27 | GPTNTYPTYFAL | 42 |
| hu.1D15E11-V1 | SYVMH | 1 | YIDPYNEGTKYNEKFKG | 28 | GGMARRSFDY | 30 |
| hu.1D15E11-V2 | SYVMH | 1 | YIDPYNEGTKYNEKFKG | 28 | GGMARRSFDY | 30 |
| hu.1D15E11-V3 | SYVMH | 1 | YIDPYNEGTKYNEKFKG | 28 | GGIARRSFDY | 177 |
| hu.1D15E11-V4 | SYVMH | 1 | YIDPYNEGTKYNEKFKG | 28 | GGVARRSFDY | 178 |
| hu.18B9.3i-V1 | SGYMS | 13 | SIYAGSSGKTYYADWAKG | 29 | DSGGTYYTDYLYA | 43 |
| hu.18B9.3i-V2 | SGYMS | 13 | SIYAGSSGKTYYADWAKG | 29 | DSGGTYYTDYLYL | 44 |
| hu.18B9.3i-V3 | SGYMS | 13 | SIYAGSSGKTYYADWAKG | 29 | DSGGTYYTDYLYL | 44 |

TABLE 2
Anti-CD47 Antibody Light Chain CDR sequences

| | | SEQ | | SEQ | | SEQ |
|---|---|---|---|---|---|---|
| | | ID | | ID | | ID |
| Antibody Name | LC-CDR-1 | NO: | LC-CDR-2 | NO: | LC-CDR-3 | NO: |
| M.1D15E11 | LASQTIGTWLT | 45 | AATTLAD | 58 | QQLYSTPWT | 70 |
| M.20B10E9 | HASQNIHVWLS | 46 | KASNLHT | 59 | QQGQSYPYT | 71 |
| M.69H1H9 | RASQGIRGNLD | 47 | STSNLNS | 60 | LQRNAFPWT | 72 |
| M.30A5C6 | HASQNINVWLS | 48 | KASNLHT | 59 | QQGQNYPYT | 73 |
| M.95D4A11 | RASGNIHNYLA | 49 | NAKTLAD | 61 | QHFWSTPPT | 74 |
| R.1H3 | QASQSISNECS | 50 | RASTLES | 62 | QSTYYGTTG | 75 |
| | | | | | NT | |
| R.9B11 | QASQSINNLLA | 51 | DASDLAS | 63 | QGYFYGSSV | 76 |
| | | | | | DLV | |
| R.13A2 | QASQSISNELS | 52 | RASTLES | 62 | QCTYITYGN | 77 |
| | | | | | A | |
| R.14G4 | QSSESVYRDNC | 53 | KASTLAS | 64 | AGYKSTITD | 78 |
| | LG | | | | GTA | |
| R.15G10 | QASRSITSYLA | 54 | AASNVAS | 65 | QSYYAISTN | 79 |
| | | | | | T | |
| R.18B9 | QASEGISSYLS | 55 | KASTLTS | 66 | QCTYGSSGS | 80 |
| | | | | | SGYGAA | |
| R.19H7 | QSSESVYRDNC | 53 | RASTLAS | 67 | AGYKSTITD | 78 |
| | LG | | | | GTA | |
| R.20H1 | QASQSISNELS | 52 | RASTLES | 62 | QSTYYGTSY | 81 |
| | | | | | GNA | |
| R.1B11 | QASQSITTYLA | 56 | GASNLES | 68 | QSYYAISKS | 82 |
| | | | | | A | |
| hu.1D15E11-V1 | LASQTIGTWLT | 45 | AATTLAE | 69 | QQLYSTPWT | 70 |
| hu.1D15E11-V2 | LASQTIGTWLT | 45 | AATTLAE | 69 | QQLYSTPWT | 70 |
| hu.1D15E11-V3 | LASQTIGTWLT | 45 | AATTLAE | 69 | QQLYSTPWT | 70 |
| hu.1D15E11-V4 | LASQTIGTWLT | 45 | AATTLAE | 69 | QQLYSTPWT | 70 |
| hu.18B9.3i-V1 | RASEGISSYLS | 57 | KASTLTS | 66 | QSTYGSSGS | 83 |
| | | | | | SGYGAA | |
| hu.18B9.3i-V2 | RASEGISSYLS | 57 | KASTLTS | 66 | QSAYGSSGS | 84 |
| | | | | | SGYGAA | |
| hu.18B9.3i-V3 | RASEGISSYLS | 57 | KASTLTS | 66 | QSTYGSSGS | 85 |
| | | | | | SGYAAA | |

In some embodiments, the antibody disclosed herein is a humanized antibody. In some embodiments, the antibody is full-length antibody, comprising a heavy chain (HC) and a light chain (LC). In some embodiments, the heavy chain comprises a heavy chain variable domain. In some embodiments, the light chain comprises a light chain variable domain. In some embodiments, the antibody is in an antibody format selected from Fab, Fab′, scFv, and (Fab′)₂.

In some embodiments, the antibody described herein comprises an IgG framework, an IgA framework, an IgE framework, or an IgM framework. In some instances, the antibody comprises an IgG framework (e.g., IgG1, IgG2, IgG3, or IgG4). In such instances, the antibody comprises an IgG1, IgG2, IgG3, or an IgG4 framework. In some embodiments, the antibody comprises an IgG1 framework. In some embodiments, the antibody comprises an IgG4 framework.

In some embodiments, the heavy chain variable domain is fused to a human IgG1 constant region. In some embodiments, the heavy chain variable domain is fused to a human IgG4 constant region. In some embodiments, the light chain variable domain is fused to a human Kappa constant region.

In some embodiments, the heavy chain variable domain comprises a variable domain of an IgG1, IgG2, IgG3, or IgG4 heavy chain. In some embodiments, the light chain variable domain comprises a variable domain of a Kappa light chain.

In some cases, the antibody further comprises one or more mutations in a framework region, e.g., in the CH1 domain, CH2 domain, CH3 domain, hinge region, or a combination thereof. In some instances, the one or more mutations are to stabilize the antibody and/or to increase half-life. In some instances, the one or more mutations are to modulate Fc receptor interactions, to increase ADCC or complement-dependent cytotoxicity (CDC). In other instances, the one or more mutations are to reduce or eliminate Fc effector functions such as FcγR-binding, ADCC, or CDC. In additional instances, the one or more mutations are to modulate glycosylation, e.g. fucosylation. In some cases, the one or more mutations enhance stability, increase half-life, decrease glycosylation, and/or modulate Fc receptor interactions, e.g., to increase or decrease ADCC and/or CDC.

In some cases, the antibody comprises an IgG1 framework. In some embodiments, the constant region of the antibody is modified at one or more amino acid positions to alter Fc receptor interaction. Exemplary residues that modulate or alter Fc receptor interaction include, but are not limited to, G236, S239, T250, M252, S254, T256, K326, A330, I332, E333A, M428, H433, or N434 (Kabat numbering; EU index of Kabat et al 1991 Sequences of Proteins of Immunological Interest). In some instances, the mutation comprises G236A, S239D, T250Q, M252Y, S254T, T256E, K326W, A330L, I332E, E333A, E333S, M428L, H433K, or N434F.

In some embodiments, the modification at one or more amino acid positions in the IgG1 constant region to alter Fc receptor interaction leads to increased half-life. In some instances, the modification at one or more amino acid positions comprise T250, M252, S254, T256, M428, H433, N434, or a combination thereof, e.g., comprising T250Q/M428L or M252Y/S254T/T256E and H433K/N434F.

In some embodiments, a antibody described above comprises a knobs-into-holes (KIH) format. In some cases, the KIH is located in the Fc region, in which the residues within the CH3 domain are optionally modified based on the disclosure of WO96/027011; Ridgway, et. al., Protein Eng. 9 (1996) 617-621; Merchant, et. al., Nat. Biotechnol. 16 (1998) 677-681; PCT/US19/61884; or Carter, J. Immunol. Protein Engineering 9(7) 617-621, 1996. In some cases, one member of a CH3 domain pair is the “knob” chain while the other is the “hole” chain, and additional disulfide bridges are optionally introduced to further stabilize the antibody and/or to increase yield.

In some instances, the antibody is an IgG1, and the CH3 domain of the “knob” chain comprises a T366W mutation and the CH3 domain of the “hole” chain comprises mutations T366S, L368A, and Y407V. In some cases, the CH3 domain of the “knob” chain further comprises a Y349C mutation which forms an interchain disulfide bridge with either E356C or S354C in the CH3 domain of the “hole” chain.

In some instances, the CH3 domain of the “knob” chain comprises R409D and K370E mutations, and the CH3 domain of the “hole” chain comprises D399K and E357K. In some cases, the CH3 domain of the “knob” chain further comprises a T366W mutation, and the CH3 domain of the “hole” chain further comprises mutations T366S, L368A, and Y407V.

In some embodiments, the modification at one or more amino acid positions in the IgG1 constant region to alter Fc receptor interaction leads to increased ADCC and/or CDC. In some instances, the modification at one or more amino acid positions comprises S239, K326, A330, I332, E333, or a combination thereof. In some instances, the modification at one or more amino acid positions for increased ADCC and/or CDC comprises, e.g., E333A, S239D/A330L/I332E, or K326W/E333S. In some cases, the modification at one or more amino acid positions for increased ADCC comprises S239D/A330L/I332E. In some cases, the modification at one or more amino acid positions for increased CDC comprises K326W/E333S.

In some embodiments, the modification at one or more amino acid positions in the IgG1 constant region to alter Fc receptor interaction leads to increased macrophage phagocytosis. In some instances, the modification at one or more amino acid positions comprises G236, S239, I332, or a combination thereof. In some cases, the modification at one or more amino acid positions for increased macrophage phagocytosis comprises the combination of S239D/I332I/G236A.

In some embodiments, the IgG1 constant region is modified at amino acid N297 (Kabat numbering) in which residue N297 is afucosylated, wherein the oligosaccharides do not contain fucose sugar units.

In some embodiments, the antibody comprises an IgG4 framework. In some instances, one or more amino acid positions in the IgG4 framework are modified to alter Fc receptor interaction, e.g., to increase ADCC and/or CDC. For example, mutations to increase ADCC comprises, in some embodiments, S239D, I332E, and A330L (amino acid numbering is according to the EU index in Kabat et al), such as described in U.S. Pat. No. 8,093,359. In some cases, one or more amino acid positions in the IgG4 framework are modified to stabilize the antibody and/or to increase half-life. In some instances, one or more amino acid positions in the IgG4 framework are modified to modulate glycosylation. In some cases, the constant region is modified at a hinge region to prevent or reduce strand exchange. In some instances, the amino acid that is modified is S228 (e.g., S228P).

In some embodiments, the antibody binds to extracellular domain of human CD47 with an EC50 of between 0.01 nM-5 nM as determined by an ELISA binding assay. In some embodiments, the antibody binds to extracellular domain of human CD47 with an EC50 of between 0.01 nM-0.5 nM as determined by an ELISA binding assay. In some embodiments, the antibody binds to extracellular domain of human CD47 with an EC50 of between 0.01 nM-0.1 nM as determined by an ELISA binding assay. In some embodiments, the antibody binds to extracellular domain of human CD47 with an EC50 of between 0.1 nM-0.5 nM as determined by an ELISA binding assay. In some embodiments, the antibody binds to extracellular domain of human CD47 with an EC50 of about 0.01 nM, about 0.02 nM, about 0.03 nM, about 0.04 nM, about 0.05 nM, about 0.06 nM, about 0.07 nM, about 0.08 nM, about 0.09 nM, about 0.1 nM, about 0.2 nM, about 0.3 nM, about 0.4 nM, about 0.5 nM as determined by an ELISA binding assay. In some embodiments, the extracellular domain of human CD47 comprises an amino acid sequence according to SEQ ID NO: 86.

In some embodiments, the ELISA binding assay comprises the following steps: coating a 96-well plate for at least 12 hours with 1p g/ml of recombinant CD47; washing the plate three times; blocking the plate with 300 μL of 1% bovine serum albumin in phosphate-buffered saline solution with tween (PBST) at 37° C. for 1 hour; washing the plate four times with PBST; incubating a serial dilution of the antibody that binds specifically to CD47 at 37° C. for 1 hour; washing the plate four times with PBST; incubating a 1:5000 dilution of anti-human IgG-peroxidase antibody at 37° C. for 1 hour within the plate; washing the plate 4 times with PBST; incubating 3,3′,5,5′ tetramethylbenzidine substrate for 15 minutes at room temperature within the plate; terminating the reaction within the plate with 1N HCl; and reading the plate at 450 nm to determine the EC50 of the antibody for binding to the extracellular domain of human CD47.

In some embodiments, the antibody binds to CD47+ cell lines with an EC50 of between 0.1 nM-10 nM as determined by flow cytometry. In some embodiments, the antibody binds to CD47+ cell lines with an EC50 of between 0.1 nM-5 nM as determined by flow cytometry. In some embodiments, the antibody binds to CD47+ cell lines with an EC50 of between 0.1 nM-0.5 nM as determined by flow cytometry. In some embodiments, the antibody binds to CD47+ cell lines with an EC50 of between 0.5 nM-5 nM as determined by flow cytometry. In some embodiments, the antibody binds to CD47+ cell lines with an EC50 of about 0.1 nM, about 0.2 nM, about 0.3 nM, about 0.4 nM, about 0.5 nM, about 0.6 nM, about 0.7 nM, about 0.8 nM, about 0.9 nM, about 1.0 nM, about 2.0 nM, about 3.0 nM, about 4.0 nM, about 5.0 nM as determined by flow cytometry.

In some embodiments, the EC50 as determined by flow cytometry comprises the following steps and assay conditions: centrifuging CD47+ cells at 2000 rpm for 5 minutes to obtain centrifuged cells; resuspending the centrifuged cells in 10-15 mL of culture medium; resuspending cells in blocking buffer comprising phosphate buffer saline with 2% fetal bovine serum (PBS plus 2% FBS) at a concentration of 3×10⁶ cells/mL to obtain a cell suspension; dispensing the cell suspension into wells of a 96-well plate; diluting the antibody that binds specifically to CD47 in the blocking buffer to a desired concentration followed by addition of 100 μL of the antibody per well and incubating at 1 hour for 4° C.; washing the cells three times with PBS plus 2% FBS; resuspending the cells in 100 μL 1:500 diluted Alexa Fluor 488 labeled Mouse anti-Human IgG1 Fc secondary antibody and incubating at 4° C. in the dark for one hour; washing the cells three times with 200 μL PBS and centrifuging at 2000 rpm for 5 minutes; resuspending cells in 300 μL cold PBS; and analyzing with a flow cytometer to determine the EC50 of the antibody for the CD47+ cell line.

In some embodiments, the antibody blocks SIRPa activity with a IC50 of between 0.1 nM-10 nM as determined by flow cytometry. In some embodiments, the antibody blocks SIRPa activity with a IC50 of between 0.1 nM-5 nM as determined by flow cytometry. In some embodiments, the antibody blocks SIRPa activity with a IC50 of between 0.1 nM-1 nM as determined by flow cytometry. In some embodiments, the antibody blocks SIRPa activity with a IC50 of between 1 nM-5 nM as determined by flow cytometry. In some embodiments, the antibody blocks SIRPa activity with a IC50 of about 0.1 nM, about 0.2 nM, about 0.3 nM, about 0.4 nM, about 0.5 nM, about 0.6 nM, about 0.7 nM, about 0.8 nM, about 0.9 nM, about 1.0 nM, about 2.0 nM, about 3.0 nM, about 4.0 nM, about 5.0 nM as determined by flow cytometry.

In some embodiments, the IC50 for SIRPa activity as determined by flow cytometry comprises the following steps and assay conditions: harvesting, centrifuging, and resuspending Raji tumor cells or HCT-15 tumor cells in FACS buffer comprising phosphate buffer saline with 2% fetal bovine serum (PBS plus 2% FBS) at a concentration of 2×10⁶ cells/mL; dispensing 100 μL of the cell suspension into wells of a 96-well plate; centrifuging the plate for 5 minutes at 300×g followed by discarding of supernatants; incubating cells from the plate with 50 μL per well of serially diluted antibody and a constant amount of SIRPA-mIgG2a fusion protein (0.2 μg/mL for Raji cells, 1 μg/mL for HCT-15 cells) in FACS buffer for 1 hour at 4° C.; washing the plate with FACS buffer followed by incubating for one hour at 4° C. in the dark with 100 μL of Alexa Fluor 488 donkey anti-Mouse IgG(H+L) secondary antibody; washing twice with FACS buffer; resuspending cells in the plate with 300 μL FACS buffer; and analyzing with a flow cytometer to determine the IC50 of the antibody for blocking SIRPa activity.

In some embodiments, the antibody induces increased antibody-dependent cellular phagocytosis (ADCP) as compared to a control antibody that comprises amino acid sequences according to SEQ ID NOs: 173 and 174 when tested under substantially equivalent assay conditions in an assay for ADCP. In some embodiments, the antibody induces increased antibody-dependent cellular phagocytosis (ADCP) by at least 10%, 20%, 30%, 40%, 50%, 60%, 70%, 80%, 90%, 100%, 150%, 200%, 300%, 400%, or more as compared to a control antibody that comprises amino acid sequences according to SEQ ID NOs: 173 and 174.

In some embodiments, the antibody has less binding on human red blood cells (RBC) as compared to a control antibody that comprises amino acid sequences SEQ ID NOs: 173 and 174 when tested under substantially equivalent assay conditions for assessing RBC binding. In some embodiments, the antibody has less binding on human red blood cells (RBC) by at least 10%, 20%, 30%, 40%, 50%, 60%, 70%, 80%, 90%, 100%, 150%, 200%, 300%, 400%, or more as compared to a control antibody that comprises amino acid sequences according to SEQ ID NOs: 173 and 174. In some embodiments, a concentration of 600 nM of the antibody does not induce hemolysis of red blood cells in a hemagglutination assay.

In some embodiments, the antibody induces increased reduction in tumor volume as compared to a control antibody that comprises amino acid sequences according to SEQ ID NOs: 173 and 174 when tested under substantially equivalent assay conditions in a tumor growth animal model. In some embodiments, the antibody induces increased reduction in tumor volume of at least 1-fold, 2-fold, 3-fold, 4-fold, 5-fold, 6-fold, 7-fold, 8-fold, 9-fold, 10-fold, 15-fold, 20-fold, 30-fold, 40-fold, 50-fold, as compared to a control antibody that comprises amino acid sequences according to SEQ ID NOs: 173 and 174 when tested under substantially equivalent assay conditions in a tumor growth animal model.

In some embodiments, the heavy chain variable domain comprises an amino acid sequence with at least 90%, 95%, 96%, 97%, 98%, 99%, or 100% identity to any one of SEQ ID NOs: 143, 149, 155, 161, or 167.

In some embodiments, the light chain variable domain comprises an amino acid sequence with at least 90%, 95%, 96%, 97%, 98%, 99%, or 100% identity to any one of SEQ ID NOs: 144, 150, 156, 162, or 168.

In some embodiments, the heavy chain variable domain comprises an amino acid sequence with at least 80% sequence identity to SEQ ID NO: 143, and the light chain variable domain comprises an amino acid sequence with at least 80% sequence identity to SEQ ID NO: 144. In some embodiments, the heavy chain variable domain comprises an amino acid sequence with at least 90% sequence identity to SEQ ID NO: 143, and the light chain variable domain comprises an amino acid sequence with at least 90% sequence identity to SEQ ID NO: 144. In some embodiments, the heavy chain variable domain comprises an amino acid sequence with at least 95% sequence identity to SEQ ID NO: 143, and the light chain variable domain comprises an amino acid sequence with at least 95% sequence identity to SEQ ID NO: 144. In some embodiments, the heavy chain variable domain comprises an amino acid sequence with at least 98% sequence identity to SEQ ID NO: 143, and the light chain variable domain comprises an amino acid sequence with at least 98% sequence identity to SEQ ID NO: 144. In some embodiments, the heavy chain variable domain comprises an amino acid sequence with at least 99% sequence identity to SEQ ID NO: 143, and the light chain variable domain comprises an amino acid sequence with at least 99% sequence identity to SEQ ID NO: 144. In some embodiments, the heavy chain variable domain comprises an amino acid sequence according to SEQ ID NO: 143, and the light chain variable domain comprises an amino acid sequence according to SEQ ID NO: 144.

In some embodiments, the heavy chain variable domain comprises an amino acid sequence with at least 110 consecutive amino acid residues of SEQ ID NO: 143. In some embodiments, the heavy chain variable domain comprises an amino acid sequence with at least 110 consecutive amino acid residues of SEQ ID NO: 143 and has at least 90%, 95%, 99% sequence identity to the at least 110 consecutive amino acid residues of SEQ ID NO: 143. In some embodiments, the light chain variable domain comprises an amino acid sequence with at least 100 consecutive amino acid residues of SEQ ID NO: 144. In some embodiments, the light chain variable domain comprises an amino acid sequence with at least 100 consecutive amino acid residues of SEQ ID NO: 144 and has at least 90%, 95%, 99% sequence identity to the at least 100 consecutive amino acid residues of SEQ ID NO: 144.

In some embodiments, the heavy chain variable domain comprises an amino acid sequence with at least 80% sequence identity to SEQ ID NO: 149, and the light chain variable domain comprises an amino acid sequence with at least 80% sequence identity to SEQ ID NO: 150. In some embodiments, the heavy chain variable domain comprises an amino acid sequence with at least 90% sequence identity to SEQ ID NO: 149, and the light chain variable domain comprises an amino acid sequence with at least 90% sequence identity to SEQ ID NO: 150. In some embodiments, the heavy chain variable domain comprises an amino acid sequence with at least 95% sequence identity to SEQ ID NO: 149, and the light chain variable domain comprises an amino acid sequence with at least 95% sequence identity to SEQ ID NO: 150. In some embodiments, the heavy chain variable domain comprises an amino acid sequence with at least 98% sequence identity to SEQ ID NO: 149, and the light chain variable domain comprises an amino acid sequence with at least 98% sequence identity to SEQ ID NO: 150. In some embodiments, the heavy chain variable domain comprises an amino acid sequence with at least 99% sequence identity to SEQ ID NO: 149, and the light chain variable domain comprises an amino acid sequence with at least 99% sequence identity to SEQ ID NO: 150. In some embodiments, the heavy chain variable domain comprises an amino acid sequence according to SEQ ID NO: 149, and the light chain variable domain comprises an amino acid sequence according to SEQ ID NO: 150.

In some embodiments, the heavy chain variable domain comprises an amino acid sequence with at least 110 consecutive amino acid residues of SEQ ID NO: 149. In some embodiments, the heavy chain variable domain comprises an amino acid sequence with at least 110 consecutive amino acid residues of SEQ ID NO: 149 and has at least 90%, 95%, 99% sequence identity to the at least 110 consecutive amino acid residues of SEQ ID NO: 149. In some embodiments, the light chain variable domain comprises an amino acid sequence with at least 100 consecutive amino acid residues of SEQ ID NO: 150. In some embodiments, the light chain variable domain comprises an amino acid sequence with at least 100 consecutive amino acid residues of SEQ ID NO: 150 and has at least 90%, 95%, 99% sequence identity to the at least 100 consecutive amino acid residues of SEQ ID NO: 150.

In some embodiments, the heavy chain variable domain comprises an amino acid sequence with at least 80% sequence identity to SEQ ID NO: 181, and the light chain variable domain comprises an amino acid sequence with at least 80% sequence identity to SEQ ID NO: 144. In some embodiments, the heavy chain variable domain comprises an amino acid sequence with at least 90% sequence identity to SEQ ID NO: 181, and the light chain variable domain comprises an amino acid sequence with at least 90% sequence identity to SEQ ID NO: 144. In some embodiments, the heavy chain variable domain comprises an amino acid sequence with at least 95% sequence identity to SEQ ID NO: 181, and the light chain variable domain comprises an amino acid sequence with at least 95% sequence identity to SEQ ID NO: 144. In some embodiments, the heavy chain variable domain comprises an amino acid sequence with at least 98% sequence identity to SEQ ID NO: 181, and the light chain variable domain comprises an amino acid sequence with at least 98% sequence identity to SEQ ID NO: 144. In some embodiments, the heavy chain variable domain comprises an amino acid sequence with at least 99% sequence identity to SEQ ID NO: 181, and the light chain variable domain comprises an amino acid sequence with at least 99% sequence identity to SEQ ID NO: 144. In some embodiments, the heavy chain variable domain comprises an amino acid sequence according to SEQ ID NO: 181, and the light chain variable domain comprises an amino acid sequence according to SEQ ID NO: 144.

In some embodiments, the heavy chain variable domain comprises an amino acid sequence with at least 110 consecutive amino acid residues of SEQ ID NO: 181. In some embodiments, the heavy chain variable domain comprises an amino acid sequence with at least 110 consecutive amino acid residues of SEQ ID NO: 181 and has at least 90%, 95%, 99% sequence identity to the at least 110 consecutive amino acid residues of SEQ ID NO: 181. In some embodiments, the light chain variable domain comprises an amino acid sequence with at least 100 consecutive amino acid residues of SEQ ID NO: 144. In some embodiments, the light chain variable domain comprises an amino acid sequence with at least 100 consecutive amino acid residues of SEQ ID NO: 144 and has at least 90%, 95%, 99% sequence identity to the at least 100 consecutive amino acid residues of SEQ ID NO: 144.

In some embodiments, the heavy chain variable domain comprises an amino acid sequence with at least 80% sequence identity to SEQ ID NO: 184, and the light chain variable domain comprises an amino acid sequence with at least 80% sequence identity to SEQ ID NO: 144. In some embodiments, the heavy chain variable domain comprises an amino acid sequence with at least 90% sequence identity to SEQ ID NO: 184, and the light chain variable domain comprises an amino acid sequence with at least 90% sequence identity to SEQ ID NO: 144. In some embodiments, the heavy chain variable domain comprises an amino acid sequence with at least 95% sequence identity to SEQ ID NO: 184, and the light chain variable domain comprises an amino acid sequence with at least 95% sequence identity to SEQ ID NO: 144. In some embodiments, the heavy chain variable domain comprises an amino acid sequence with at least 98% sequence identity to SEQ ID NO: 184, and the light chain variable domain comprises an amino acid sequence with at least 98% sequence identity to SEQ ID NO: 144. In some embodiments, the heavy chain variable domain comprises an amino acid sequence with at least 99% sequence identity to SEQ ID NO: 184, and the light chain variable domain comprises an amino acid sequence with at least 99% sequence identity to SEQ ID NO: 144. In some embodiments, the heavy chain variable domain comprises an amino acid sequence according to SEQ ID NO: 184, and the light chain variable domain comprises an amino acid sequence according to SEQ ID NO: 144.

In some embodiments, the heavy chain variable domain comprises an amino acid sequence with at least 110 consecutive amino acid residues of SEQ ID NO: 184. In some embodiments, the heavy chain variable domain comprises an amino acid sequence with at least 110 consecutive amino acid residues of SEQ ID NO: 184 and has at least 90%, 95%, 99% sequence identity to the at least 110 consecutive amino acid residues of SEQ ID NO: 184. In some embodiments, the light chain variable domain comprises an amino acid sequence with at least 100 consecutive amino acid residues of SEQ ID NO: 144. In some embodiments, the light chain variable domain comprises an amino acid sequence with at least 100 consecutive amino acid residues of SEQ ID NO: 144 and has at least 90%, 95%, 99% sequence identity to the at least 100 consecutive amino acid residues of SEQ ID NO: 144.

In some embodiments, the heavy chain variable domain comprises an amino acid sequence with at least 80% sequence identity to SEQ ID NO: 155, and the light chain variable domain comprises an amino acid sequence with at least 80% sequence identity to SEQ ID NO: 156. In some embodiments, the heavy chain variable domain comprises an amino acid sequence with at least 90% sequence identity to SEQ ID NO: 155, and the light chain variable domain comprises an amino acid sequence with at least 90% sequence identity to SEQ ID NO: 156. In some embodiments, the heavy chain variable domain comprises an amino acid sequence with at least 95% sequence identity to SEQ ID NO: 155, and the light chain variable domain comprises an amino acid sequence with at least 95% sequence identity to SEQ ID NO: 156. In some embodiments, the heavy chain variable domain comprises an amino acid sequence with at least 98% sequence identity to SEQ ID NO: 155, and the light chain variable domain comprises an amino acid sequence with at least 98% sequence identity to SEQ ID NO: 156. In some embodiments, the heavy chain variable domain comprises an amino acid sequence with at least 99% sequence identity to SEQ ID NO: 155, and the light chain variable domain comprises an amino acid sequence with at least 99% sequence identity to SEQ ID NO: 156. In some embodiments, the heavy chain variable domain comprises an amino acid sequence according to SEQ ID NO: 155, and the light chain variable domain comprises an amino acid sequence according to SEQ ID NO: 156.

In some embodiments, the heavy chain variable domain comprises an amino acid sequence with at least 110 consecutive amino acid residues of SEQ ID NO: 155. In some embodiments, the heavy chain variable domain comprises an amino acid sequence with at least 110 consecutive amino acid residues of SEQ ID NO: 155 and has at least 90%, 95%, 99% sequence identity to the at least 110 consecutive amino acid residues of SEQ ID NO: 155. In some embodiments, the light chain variable domain comprises an amino acid sequence with at least 100 consecutive amino acid residues of SEQ ID NO: 156. In some embodiments, the light chain variable domain comprises an amino acid sequence with at least 100 consecutive amino acid residues of SEQ ID NO: 156 and has at least 90%, 95%, 99% sequence identity to the at least 100 consecutive amino acid residues of SEQ ID NO: 156.

In some embodiments, the heavy chain variable domain comprises an amino acid sequence with at least 80% sequence identity to SEQ ID NO: 161, and the light chain variable domain comprises an amino acid sequence with at least 80% sequence identity to SEQ ID NO: 162. In some embodiments, the heavy chain variable domain comprises an amino acid sequence with at least 90% sequence identity to SEQ ID NO: 161, and the light chain variable domain comprises an amino acid sequence with at least 90% sequence identity to SEQ ID NO: 162. In some embodiments, the heavy chain variable domain comprises an amino acid sequence with at least 95% sequence identity to SEQ ID NO: 161, and the light chain variable domain comprises an amino acid sequence with at least 95% sequence identity to SEQ ID NO: 162. In some embodiments, the heavy chain variable domain comprises an amino acid sequence with at least 98% sequence identity to SEQ ID NO: 161, and the light chain variable domain comprises an amino acid sequence with at least 98% sequence identity to SEQ ID NO: 162. In some embodiments, the heavy chain variable domain comprises an amino acid sequence with at least 99% sequence identity to SEQ ID NO: 161, and the light chain variable domain comprises an amino acid sequence with at least 99% sequence identity to SEQ ID NO: 162. In some embodiments, the heavy chain variable domain comprises an amino acid sequence according to SEQ ID NO: 161, and the light chain variable domain comprises an amino acid sequence according to SEQ ID NO: 162.

In some embodiments, the heavy chain variable domain comprises an amino acid sequence with at least 110 consecutive amino acid residues of SEQ ID NO: 161. In some embodiments, the heavy chain variable domain comprises an amino acid sequence with at least 110 consecutive amino acid residues of SEQ ID NO: 161 and has at least 90%, 95%, 99% sequence identity to the at least 110 consecutive amino acid residues of SEQ ID NO: 161. In some embodiments, the light chain variable domain comprises an amino acid sequence with at least 100 consecutive amino acid residues of SEQ ID NO: 162. In some embodiments, the light chain variable domain comprises an amino acid sequence with at least 100 consecutive amino acid residues of SEQ ID NO: 162 and has at least 90%, 95%, 99% sequence identity to the at least 100 consecutive amino acid residues of SEQ ID NO: 162.

In some embodiments, the heavy chain variable domain comprises an amino acid sequence with at least 80% sequence identity to SEQ ID NO: 167, and the light chain variable domain comprises an amino acid sequence with at least 80% sequence identity to SEQ ID NO: 168. In some embodiments, the heavy chain variable domain comprises an amino acid sequence with at least 90% sequence identity to SEQ ID NO: 167, and the light chain variable domain comprises an amino acid sequence with at least 90% sequence identity to SEQ ID NO: 168. In some embodiments, the heavy chain variable domain comprises an amino acid sequence with at least 95% sequence identity to SEQ ID NO: 167, and the light chain variable domain comprises an amino acid sequence with at least 95% sequence identity to SEQ ID NO: 168. In some embodiments, the heavy chain variable domain comprises an amino acid sequence with at least 98% sequence identity to SEQ ID NO: 167, and the light chain variable domain comprises an amino acid sequence with at least 98% sequence identity to SEQ ID NO: 168. In some embodiments, the heavy chain variable domain comprises an amino acid sequence with at least 99% sequence identity to SEQ ID NO: 167, and the light chain variable domain comprises an amino acid sequence with at least 99% sequence identity to SEQ ID NO: 168. In some embodiments, the heavy chain variable domain comprises an amino acid sequence according to SEQ ID NO: 167, and the light chain variable domain comprises an amino acid according to identity to SEQ ID NO: 168.

In some embodiments, the heavy chain variable domain comprises an amino acid sequence with at least 110 consecutive amino acid residues of SEQ ID NO: 167. In some embodiments, the heavy chain variable domain comprises an amino acid sequence with at least 110 consecutive amino acid residues of SEQ ID NO: 167 and has at least 90%, 95%, 99% sequence identity to the at least 110 consecutive amino acid residues of SEQ ID NO: 167. In some embodiments, the light chain variable domain comprises an amino acid sequence with at least 100 consecutive amino acid residues of SEQ ID NO: 168. In some embodiments, the light chain variable domain comprises an amino acid sequence with at least 100 consecutive amino acid residues of SEQ ID NO: 168 and has at least 90%, 95%, 99% sequence identity to the at least 100 consecutive amino acid residues of SEQ ID NO: 168.

In some embodiments, the antibody comprises a heavy chain sequence according to any of the heavy chain (HC) sequences of Tables 4, 5, 9, and 10. In some embodiments, the heavy chain sequence comprises an amino acid sequence with at least 80%, 90%, 95%, 96%, 97%, 98%, or 99% sequence identity to any of the heavy chain (HC) sequences of Tables 4, 5, 9, and 10.

In some embodiments, the antibody comprises a light chain sequence according to any of the light chain (LC) sequences of Tables 4, 5, 9, and 10. In some embodiments, the light chain sequence comprises an amino acid sequence with at least 80%, 90%, 95%, 96%, 97%, 98%, or 99% sequence identity to any of the light chain (LC) sequences of Tables 4, 5, 9, and 10.

In some embodiments, the heavy chain sequence comprises an amino acid sequence with at least 80% sequence identity to SEQ ID NO: 145, and the light chain sequence comprises an amino acid with at least 80% sequence identity to SEQ ID NO: 147. In some embodiments, the heavy chain sequence comprises an amino acid sequence with at least 90% sequence identity to SEQ ID NO: 145, and the light chain sequence comprises an amino acid with at least 90% sequence identity to SEQ ID NO: 147. In some embodiments, the heavy chain sequence comprises an amino acid sequence with at least 95% sequence identity to SEQ ID NO: 145, and the light chain sequence comprises an amino acid with at least 95% sequence identity to SEQ ID NO: 147. In some embodiments, the heavy chain sequence comprises an amino acid sequence with at least 98% sequence identity to SEQ ID NO: 145, and the light chain sequence comprises an amino acid with at least 98% sequence identity to SEQ ID NO: 147. In some embodiments, the heavy chain sequence comprises an amino acid sequence with at least 99% sequence identity to SEQ ID NO: 145, and the light chain sequence comprises an amino acid with at least 99% sequence identity to SEQ ID NO: 147. In some embodiments, the heavy chain sequence comprises an amino acid sequence according to SEQ ID NO: 145, and the light chain sequence comprises an amino acid according to SEQ ID NO: 147.

In some embodiments, the heavy chain sequence comprises an amino acid sequence with at least 430 consecutive amino acid residues of SEQ ID NO: 145. In some embodiments, the heavy chain sequence comprises an amino acid sequence with at least 430 consecutive amino acid residues of SEQ ID NO: 145 and has at least 90%, 95%, 99% sequence identity to the at least 430 consecutive amino acid residues of SEQ ID NO: 145. In some embodiments, the light chain sequence comprises an amino acid sequence with at least 200 consecutive amino acid residues of SEQ ID NO: 147. In some embodiments, the light chain sequence comprises an amino acid sequence with at least 200 consecutive amino acid residues of SEQ ID NO: 147 and has at least 90%, 95%, 99% sequence identity to the at least 200 consecutive amino acid residues of SEQ ID NO: 147.

In some embodiments, the heavy chain sequence comprises an amino acid sequence with at least 80% sequence identity to SEQ ID NO: 151, and the light chain sequence comprises an amino acid with at least 80% sequence identity to SEQ ID NO: 153. In some embodiments, the heavy chain sequence comprises an amino acid sequence with at least 90% sequence identity to SEQ ID NO: 151, and the light chain sequence comprises an amino acid with at least 90% sequence identity to SEQ ID NO: 153. In some embodiments, the heavy chain sequence comprises an amino acid sequence with at least 95% sequence identity to SEQ ID NO: 151, and the light chain sequence comprises an amino acid with at least 95% sequence identity to SEQ ID NO: 153. In some embodiments, the heavy chain sequence comprises an amino acid sequence with at least 98% sequence identity to SEQ ID NO: 151, and the light chain sequence comprises an amino acid with at least 98% sequence identity to SEQ ID NO: 153. In some embodiments, the heavy chain sequence comprises an amino acid sequence with at least 99% sequence identity to SEQ ID NO: 151, and the light chain sequence comprises an amino acid with at least 99% sequence identity to SEQ ID NO: 153. In some embodiments, the heavy chain sequence comprises an amino acid sequence according to SEQ ID NO: 151, and the light chain sequence comprises an amino acid according to SEQ ID NO: 153.

In some embodiments, the heavy chain sequence comprises an amino acid sequence with at least 430 consecutive amino acid residues of SEQ ID NO: 151. In some embodiments, the heavy chain sequence comprises an amino acid sequence with at least 430 consecutive amino acid residues of SEQ ID NO: 151 and has at least 90%, 95%, 99% sequence identity to the at least 430 consecutive amino acid residues of SEQ ID NO: 151. In some embodiments, the light chain sequence comprises an amino acid sequence with at least 200 consecutive amino acid residues of SEQ ID NO: 153. In some embodiments, the light chain sequence comprises an amino acid sequence with at least 200 consecutive amino acid residues of SEQ ID NO: 153 and has at least 90%, 95%, 99% sequence identity to the at least 200 consecutive amino acid residues of SEQ ID NO: 153.

In some embodiments, the heavy chain sequence comprises an amino acid sequence with at least 80% sequence identity to SEQ ID NO: 182, and the light chain sequence comprises an amino acid with at least 80% sequence identity to SEQ ID NO: 147. In some embodiments, the heavy chain sequence comprises an amino acid sequence with at least 90% sequence identity to SEQ ID NO: 182, and the light chain sequence comprises an amino acid with at least 90% sequence identity to SEQ ID NO: 147. In some embodiments, the heavy chain sequence comprises an amino acid sequence with at least 95% sequence identity to SEQ ID NO: 182, and the light chain sequence comprises an amino acid with at least 95% sequence identity to SEQ ID NO: 147. In some embodiments, the heavy chain sequence comprises an amino acid sequence with at least 98% sequence identity to SEQ ID NO: 182, and the light chain sequence comprises an amino acid with at least 98% sequence identity to SEQ ID NO: 147. In some embodiments, the heavy chain sequence comprises an amino acid sequence with at least 99% sequence identity to SEQ ID NO: 182, and the light chain sequence comprises an amino acid with at least 99% sequence identity to SEQ ID NO: 147. In some embodiments, the heavy chain sequence comprises an amino acid sequence according to SEQ ID NO: 182, and the light chain sequence comprises an amino acid according to SEQ ID NO: 147.

In some embodiments, the heavy chain sequence comprises an amino acid sequence with at least 430 consecutive amino acid residues of SEQ ID NO: 182. In some embodiments, the heavy chain sequence comprises an amino acid sequence with at least 430 consecutive amino acid residues of SEQ ID NO: 182 and has at least 90%, 95%, 99% sequence identity to the at least 430 consecutive amino acid residues of SEQ ID NO: 182. In some embodiments, the light chain sequence comprises an amino acid sequence with at least 200 consecutive amino acid residues of SEQ ID NO: 147. In some embodiments, the light chain sequence comprises an amino acid sequence with at least 200 consecutive amino acid residues of SEQ ID NO: 147 and has at least 90%, 95%, 99% sequence identity to the at least 200 consecutive amino acid residues of SEQ ID NO: 147.

In some embodiments, the heavy chain sequence comprises an amino acid sequence with at least 80% sequence identity to SEQ ID NO: 185, and the light chain sequence comprises an amino acid with at least 80% sequence identity to SEQ ID NO: 147. In some embodiments, the heavy chain sequence comprises an amino acid sequence with at least 90% sequence identity to SEQ ID NO: 185, and the light chain sequence comprises an amino acid with at least 90% sequence identity to SEQ ID NO: 147. In some embodiments, the heavy chain sequence comprises an amino acid sequence with at least 95% sequence identity to SEQ ID NO: 185, and the light chain sequence comprises an amino acid with at least 95% sequence identity to SEQ ID NO: 147. In some embodiments, the heavy chain sequence comprises an amino acid sequence with at least 98% sequence identity to SEQ ID NO: 185, and the light chain sequence comprises an amino acid with at least 98% sequence identity to SEQ ID NO: 147. In some embodiments, the heavy chain sequence comprises an amino acid sequence with at least 99% sequence identity to SEQ ID NO: 185, and the light chain sequence comprises an amino acid with at least 99% sequence identity to SEQ ID NO: 147. In some embodiments, the heavy chain sequence comprises an amino acid sequence according to SEQ ID NO: 185, and the light chain sequence comprises an amino acid according to SEQ ID NO: 147.

In some embodiments, the heavy chain sequence comprises an amino acid sequence with at least 430 consecutive amino acid residues of SEQ ID NO: 185. In some embodiments, the heavy chain sequence comprises an amino acid sequence with at least 430 consecutive amino acid residues of SEQ ID NO: 185 and has at least 90%, 95%, 99% sequence identity to the at least 430 consecutive amino acid residues of SEQ ID NO: 185. In some embodiments, the light chain sequence comprises an amino acid sequence with at least 200 consecutive amino acid residues of SEQ ID NO: 147. In some embodiments, the light chain sequence comprises an amino acid sequence with at least 200 consecutive amino acid residues of SEQ ID NO: 147 and has at least 90%, 95%, 99% sequence identity to the at least 200 consecutive amino acid residues of SEQ ID NO: 147.

In some embodiments, the heavy chain sequence comprises an amino acid sequence with at least 80% sequence identity to SEQ ID NO: 157, and the light chain sequence comprises an amino acid with at least 80% sequence identity to SEQ ID NO: 159. In some embodiments, the heavy chain sequence comprises an amino acid sequence with at least 90% sequence identity to SEQ ID NO: 157, and the light chain sequence comprises an amino acid with at least 90% sequence identity to SEQ ID NO: 159. In some embodiments, the heavy chain sequence comprises an amino acid sequence with at least 95% sequence identity to SEQ ID NO: 157, and the light chain sequence comprises an amino acid with at least 95% sequence identity to SEQ ID NO: 159. In some embodiments, the heavy chain sequence comprises an amino acid sequence with at least 98% sequence identity to SEQ ID NO: 157, and the light chain sequence comprises an amino acid with at least 98% sequence identity to SEQ ID NO: 159. In some embodiments, the heavy chain sequence comprises an amino acid sequence with at least 99% sequence identity to SEQ ID NO: 157, and the light chain sequence comprises an amino acid with at least 99% sequence identity to SEQ ID NO: 159. In some embodiments, the heavy chain sequence comprises an amino acid sequence according to SEQ ID NO: 157, and the light chain sequence comprises an amino acid according to SEQ ID NO: 159.

In some embodiments, the heavy chain sequence comprises an amino acid sequence with at least 430 consecutive amino acid residues of SEQ ID NO: 157. In some embodiments, the heavy chain sequence comprises an amino acid sequence with at least 430 consecutive amino acid residues of SEQ ID NO: 157 and has at least 90%, 95%, 99% sequence identity to the at least 430 consecutive amino acid residues of SEQ ID NO: 157. In some embodiments, the light chain sequence comprises an amino acid sequence with at least 200 consecutive amino acid residues of SEQ ID NO: 159. In some embodiments, the light chain sequence comprises an amino acid sequence with at least 200 consecutive amino acid residues of SEQ ID NO: 159 and has at least 90%, 95%, 99% sequence identity to the at least 200 consecutive amino acid residues of SEQ ID NO: 159.

In some embodiments, the heavy chain sequence comprises an amino acid sequence with at least 80% sequence identity to SEQ ID NO: 163, and the light chain sequence comprises an amino acid with at least 80% sequence identity to SEQ ID NO: 165. In some embodiments, the heavy chain sequence comprises an amino acid sequence with at least 90% sequence identity to SEQ ID NO: 163, and the light chain sequence comprises an amino acid with at least 90% sequence identity to SEQ ID NO: 165. In some embodiments, the heavy chain sequence comprises an amino acid sequence with at least 95% sequence identity to SEQ ID NO: 163, and the light chain sequence comprises an amino acid with at least 95% sequence identity to SEQ ID NO: 165. In some embodiments, the heavy chain sequence comprises an amino acid sequence with at least 98% sequence identity to SEQ ID NO: 163, and the light chain sequence comprises an amino acid with at least 98% sequence identity to SEQ ID NO: 165. In some embodiments, the heavy chain sequence comprises an amino acid sequence with at least 99% sequence identity to SEQ ID NO: 163, and the light chain sequence comprises an amino acid with at least 99% sequence identity to SEQ ID NO: 165. In some embodiments, the heavy chain sequence comprises an amino acid sequence according to SEQ ID NO: 163, and the light chain sequence comprises an amino acid according to SEQ ID NO: 165.

In some embodiments, the heavy chain sequence comprises an amino acid sequence with at least 430 consecutive amino acid residues of SEQ ID NO: 163. In some embodiments, the heavy chain sequence comprises an amino acid sequence with at least 430 consecutive amino acid residues of SEQ ID NO: 163 and has at least 90%, 95%, 99% sequence identity to the at least 430 consecutive amino acid residues of SEQ ID NO: 163. In some embodiments, the light chain sequence comprises an amino acid sequence with at least 200 consecutive amino acid residues of SEQ ID NO: 165. In some embodiments, the light chain sequence comprises an amino acid sequence with at least 200 consecutive amino acid residues of SEQ ID NO: 165 and has at least 90%, 95%, 99% sequence identity to the at least 200 consecutive amino acid residues of SEQ ID NO: 165.

In some embodiments, the heavy chain sequence comprises an amino acid sequence with at least 80% sequence identity to SEQ ID NO: 169, and the light chain sequence comprises an amino acid with at least 80% sequence identity to SEQ ID NO: 171. In some embodiments, the heavy chain sequence comprises an amino acid sequence with at least 90% sequence identity to SEQ ID NO: 169, and the light chain sequence comprises an amino acid with at least 90% sequence identity to SEQ ID NO: 171. In some embodiments, the heavy chain sequence comprises an amino acid sequence with at least 95% sequence identity to SEQ ID NO: 169, and the light chain sequence comprises an amino acid with at least 95% sequence identity to SEQ ID NO: 171. In some embodiments, the heavy chain sequence comprises an amino acid sequence with at least 98% sequence identity to SEQ ID NO: 169, and the light chain sequence comprises an amino acid with at least 98% sequence identity to SEQ ID NO: 171. In some embodiments, the heavy chain sequence comprises an amino acid sequence with at least 99% sequence identity to SEQ ID NO: 169, and the light chain sequence comprises an amino acid with at least 99% sequence identity to SEQ ID NO: 171. In some embodiments, the heavy chain sequence comprises an amino acid sequence according to SEQ ID NO: 169, and the light chain sequence comprises an amino acid according to SEQ ID NO: 171.

In some embodiments, the heavy chain sequence comprises an amino acid sequence with at least 430 consecutive amino acid residues of SEQ ID NO: 169. In some embodiments, the heavy chain sequence comprises an amino acid sequence with at least 430 consecutive amino acid residues of SEQ ID NO: 169 and has at least 90%, 95%, 99% sequence identity to the at least 430 consecutive amino acid residues of SEQ ID NO: 169. In some embodiments, the light chain sequence comprises an amino acid sequence with at least 200 consecutive amino acid residues of SEQ ID NO: 171. In some embodiments, the light chain sequence comprises an amino acid sequence with at least 200 consecutive amino acid residues of SEQ ID NO: 171 and has at least 90%, 95%, 99% sequence identity to the at least 200 consecutive amino acid residues of SEQ ID NO: 171.

Polynucleotides Encoding Antibodies that Bind Specifically to CD47

Disclosed herein, are isolated recombinant nucleic acid molecules encoding polypeptide sequences of Tables 4, 5, 9, and 10.

Disclosed herein are isolated recombinant nucleic acid molecules encoding polypeptide sequences of Tables 4, 5, 9, and 10. or amino acid sequences that have at least 90%, 95%, 96%, 97%, 98%, or 99% sequence identity to polypeptide sequences of Table 4, 5, 9, and 10.

### Pharmaceutical Compositions

Disclosed herein, in some embodiments, are pharmaceutical compositions comprising: (a) the antibodies that bind specifically as disclosed herein; and (b) a pharmaceutically acceptable excipient.

In some embodiments, the antibody further comprises a detectable label, a therapeutic agent, or a pharmacokinetic modifying moiety. In some embodiments, the detectable label comprises a fluorescent label, a radiolabel, an enzyme, a nucleic acid probe, or a contrast agent.

For administration to a subject, the antibody as disclosed herein, may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc. and a physician will ultimately determine appropriate dosages to be used.

### Production of Antibodies

In some embodiments, polypeptides described herein (e.g., antibodies and its binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or a cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding is optionally generated by immunizing an animal, such as a rabbit, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term and high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in the culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

Expression Vectors

In some embodiments, vectors include any suitable vectors derived from eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA,B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary Algae Vectors Include pChlamy-4 Vector or MCS Vector.

Examples of mammalian vectors include transient expression vectors or stable expression vectors. Mammalian transient expression vectors may include pRK5, p3×FLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3×FLAG-CMV 7.1, pFLAG-CMV 20, p3×FLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3×FLAG-CMV 9, p3×FLAG-CMV 13, pFLAG-Myc-CMV 21, p3×FLAG-Myc-CMV 25, pFLAG-CMV 4, p3×FLAG-CMV 10, p3×FLAG-CMV 14, pFLAG-Myc-CMV 22, p3×FLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

Host Cells

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes, or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes, or Synergistetes. Other bacteria can be Acidobacteria, Chloroflexi, Chrysiogenetes, Cyanobacteria, Deferribacteres, Dictyoglomi, Thermodesulfobacteria, or Thermotogae. A bacterial cell can be Escherichia coli, Clostridium botulinum, or Coli bacilli.

Exemplary prokaryotic host cells include, but are not limited to, BL21, Mach1™, DH10B™, TOP10, DH5α, DH10Bac™, OmniMax™, MegaX™, DH12S™, INV110, TOP10F′, INVαF, TOP10/P3, ccdB Survival, PIR1, PIR2, Stb12™, Stb13™, or Stb14™.

In some instances, an animal cell includes a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVSc1.

In some instances, additional animal cells include cells obtained from a mollusk, arthropod, annelid, or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293 H cells, CHO DG44 cells, CHO-S cells, CHO-K1 cells, FUT8 KO CHO-K1, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some instances, a mammalian host cell is a stable cell line or a cell line that has incorporated a genetic material of interest into its own genome and has the capability to express the product of the genetic material after many generations of cell division. In some cases, a mammalian host cell is a transient cell line” or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells.

In some instances, plant cells include a cell from algae. Exemplary algal cell lines include, but are not limited to, strains from Chlamydomonas reinhardtii 137c, or Synechococcus elongatus PPC 7942.

### Methods of Treatment

In some embodiments are methods of treating a subject having cancer, the method comprising: administering to the subject any of the antibodies that bind specifically to CD47 as disclosed herein. In some embodiments, the cancer comprises cancer cells that express CD47. In some embodiments, the cancer cells that express CD47 are lysed. In some embodiments, the antibody induces antibody-dependent cellular phagocytosis (ADCP) of the cancer cells that express CD47. In some embodiments, the cancer is a hematological malignancy. In some embodiments, the cancer is B cell cancer. In some embodiments, the cancer is leukemia or lymphoma. In some embodiments, the cancer is lymphoma, and wherein the lymphoma is B-cell lymphoma. In some embodiments, the cancer is lymphoma, and wherein the lymphoma is T-cell lymphoma. In some embodiments, the cancer is a solid tumor. In some embodiments, the solid tumor is sarcoma, breast cancer, lung cancer, carcinoma, ovarian cancer, pancreatic cancer, gastric cancer, colorectal cancer, endometrial cancer, esophageal cancer, prostate cancer, cervical cancer, kidney cancer, urothelial cancer, or head and neck cancer. In some embodiments, the solid tumor is lung cancer, and wherein the lung cancer is non-small cell lung cancer. In some embodiments, the solid tumor is lung cancer, and wherein the lung cancer is small cell lung cancer. In some embodiments, the solid tumor is a sarcoma. In some embodiments, the solid tumor is breast cancer. In some embodiments, the solid tumor is a carcinoma. In some embodiments, the solid tumor is ovarian cancer. In some embodiments, the solid tumor is pancreatic cancer. In some embodiments, the solid tumor is gastric cancer. In some embodiments, the solid tumor is colorectal cancer. In some embodiments, the solid tumor is head and neck cancer.

In some embodiments, the method further comprises administering to the subject an anti-cancer agent. In some embodiments, the anti-cancer agent is a chemotherapeutic agent or a biologic agent. In some embodiments, the administering is sufficient to reduce or eliminate the cancer as compared to a comparable method lacking the administering.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is an antibody that specifically binds to CD47.

The label or package insert indicates that the composition is used for treating the condition of choice. Moreover, the article of manufacture may comprise (a) a first container with a composition contained therein, wherein the composition comprises the bispecific antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. The section headings used herein are for organizational purposes only and are not to be construed as limiting the subject matter described. In this application, the use of the singular includes the plural unless specifically stated otherwise. It is noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

“Antibodies” and “immunoglobulins” (Igs) are glycoproteins having the same structural characteristics. The terms are used synonymously. In some instances, the antigen specificity of the immunoglobulin is known.

The term “antibody” is used in the broadest sense and covers fully assembled antibodies, antibody fragments that can bind antigen (e.g., Fab, F(ab′)2, Fv, single chain antibodies, diabodies, antibody chimeras, hybrid antibodies, bispecific antibodies, and the like), and recombinant peptides comprising the forgoing.

The terms “monoclonal antibody” and “mAb” as used herein refer to an antibody obtained from a substantially homogeneous population of antibodies, i.e., the individual antibodies comprising the population are identical except for possible naturally occurring mutations that may be present in minor amounts.

“Native antibodies” and “native immunoglobulins” are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy-chain variable domains.

The term “variable” refers to the fact that certain portions of the variable domains differ extensively in sequence among antibodies. Variable regions confer antigen-binding specificity. However, the variability is not evenly distributed throughout the variable domains of antibodies. It is concentrated in three segments called complementarity determining regions (CDRs) or hypervariable regions, both in the light chain and the heavy-chain variable domains. The more highly conserved portions of variable domains are celled in the framework (FR) regions. The variable domains of native heavy and light chains each comprise four FR regions, largely adopting a β-pleated-sheet configuration, connected by three CDRs, which form loops connecting, and in some cases forming part of, the β-pleated-sheet structure. The CDRs in each chain are held together in close proximity by the FR regions and, with the CDRs from the other chain, contribute to the formation of the antigen-binding site of antibodies (see, Kabat et al. (1991) NIH PubL. No. 91-3242, Vol. I, pages 647-669). The constant domains are not involved directly in binding an antibody to an antigen, but exhibit various effector functions, such as Fc receptor (FcR) binding, participation of the antibody in antibody-dependent cellular cytotoxicity, initiation of complement dependent cytotoxicity, and mast cell degranulation.

The term “hypervariable region,” when used herein, refers to the amino acid residues of an antibody that are responsible for antigen-binding. The hypervariable region comprises amino acid residues from a “complementarily determining region” or “CDR” (i.e., residues 24-34 (L1), 50-56 (L2), and 89-97 (L3) in the light-chain variable domain and 31-35 (H1), 50-65 (H2), and 95-102 (H3) in the heavy-chain variable domain; Kabat et al. (1991) Sequences of Proteins of Immunological Interest, 5th Ed. Public Health Service, National Institute of Health, Bethesda, Md.) and/or those residues from a “hypervariable loop” (i.e., residues 26-32 (L1), 50-52 (L2), and 91-96 (L3) in the light-chain variable domain and (H1), 53-55 (H2), and 96-101 (13) in the heavy chain variable domain; Clothia and Lesk, (1987) J. Mol. Biol., 196:901-917). “Framework” or “FR” residues are those variable domain residues other than the hypervariable region residues, as herein deemed.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of human immunoglobulins: IgA, IgD, IgE, IgG, IgM, and IgY, and several of these may be further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity.

In some instances, the CDRs of an antibody is determined according to (i) the Kabat numbering system (Kabat et al. (197) Ann. NY Acad. Sci. 190:382-391 and, Kabat et al. (1991) Sequences of Proteins of Immunological Interest Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215(1): 175-82; and U.S. Pat. No. 7,709,226); or (iii) the ImMunoGeneTics (IMGT) numbering system, for example, as described in Lefranc, M.-P., 1999, The Immunologist, 7: 132-136 and Lefranc, M.-P. et al, 1999, Nucleic Acids Res., 27:209-212 (“IMGT CDRs”); or (iv) MacCallum et al, 1996, J. Mol. Biol., 262:732-745. See also, e.g., Martin, A., “Protein Sequence and Structure Analysis of Antibody Variable Domains,” in Antibody Engineering, Kontermann and Diibel, eds., Chapter 31, pp. 422-439, Springer-Verlag, Berlin (2001).

With respect to the Kabat numbering system, CDRs within an antibody heavy chain molecule are typically present at amino acid positions 31 to 35, which optionally can include one or two additional amino acids, following 35 (referred to in the Kabat numbering scheme as 35 A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). As is well known to those of skill in the art, using the Kabat numbering system, the actual linear amino acid sequence of the antibody variable domain can contain fewer or additional amino acids due to a shortening or lengthening of a FR and/or CDR and, as such, an amino acid's Kabat number is not necessarily the same as its linear amino acid number.

The term “chimeric” antibody refers to an antibody in which a portion of the heavy and/or light chain is derived from a particular source or species, while the remainder of the heavy and/or light chain is derived from a different source or species.

The term “recombinant human antibody”, as used herein, is intended to include all human antibodies that are prepared, expressed, created or isolated by recombinant means, such as antibodies isolated from a host cell such as a NSO or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. In some cases, the recombinant human antibodies have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

The terms “individual(s)”, “subject(s)” and “patient(s)” are used interchangeably herein and refer to any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “cancer” and “tumor” are used interchangeably herein, encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the invention are used to delay development of a disease or to slow the progression of a disease.

### Embodiments

Embodiment 1 comprises an antibody that binds specifically to CD47 comprising at least one complementarity determining region (CDR) according to SEQ ID NOs: 1-85, 176-178 or an amino acid sequence that has 0-1 amino acid mutations, substitutions, or deletions relative to SEQ ID NOs: 1-13, 58-69, or an amino acid sequence that has 0-3 amino acid mutations, substitutions, or deletions to relative to SEQ ID NOs: 14-57, 70-85, 176-178.

Embodiment 2 comprises an antibody of embodiment 1, wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain comprises a set of sequences selected from the group consisting of the following set of sequences: HC-CDR1: SEQ ID NO: 1, HC-CDR2: SEQ ID NO: 14, HC-CDR3: SEQ ID NO: 30; HC-CDR1: SEQ ID NO: 2, HC-CDR2: SEQ ID NO: 15, HC-CDR3: SEQ ID NO: 31; HC-CDR1: SEQ ID NO: 3, HC-CDR2: SEQ ID NO: 16, HC-CDR3: SEQ ID NO: 32; HC-CDR1: SEQ ID NO: 2, HC-CDR2: SEQ ID NO: 17, HC-CDR3: SEQ ID NO: 33; HC-CDR1: SEQ ID NO: 4, HC-CDR2: SEQ ID NO: 18, HC-CDR3: SEQ ID NO: 34; HC-CDR1: SEQ ID NO: 5, HC-CDR2: SEQ ID NO: 19, HC-CDR3: SEQ ID NO: 35; HC-CDR1: SEQ ID NO: 6, HC-CDR2: SEQ ID NO: 20, HC-CDR3: SEQ ID NO: 176; HC-CDR1: SEQ ID NO: 7, HC-CDR2: SEQ ID NO: 21, HC-CDR3: SEQ ID NO: 36; HC-CDR1: SEQ ID NO: 8, HC-CDR2: SEQ ID NO: 22, HC-CDR3: SEQ ID NO: 37; HC-CDR1: SEQ ID NO: 9, HC-CDR2: SEQ ID NO: 23, HC-CDR3: SEQ ID NO: 38; HC-CDR1: SEQ ID NO: 10, HC-CDR2: SEQ ID NO: 24, HC-CDR3: SEQ ID NO: 39; HC-CDR1: SEQ ID NO: 8, HC-CDR2: SEQ ID NO: 25, HC-CDR3: SEQ ID NO: 40; HC-CDR1: SEQ ID NO: 11, HC-CDR2: SEQ ID NO: 26, HC-CDR3: SEQ ID NO: 41; HC-CDR1: SEQ ID NO: 12, HC-CDR2: SEQ ID NO: 27, HC-CDR3: SEQ ID NO: 42; HC-CDR1: SEQ ID NO: 1, HC-CDR2: SEQ ID NO: 28, HC-CDR3: SEQ ID NO: 30; HC-CDR1: SEQ ID NO: 1, HC-CDR2: SEQ ID NO: 28, HC-CDR3: SEQ ID NO: 177; HC-CDR1: SEQ ID NO: 1, HC-CDR2: SEQ ID NO: 28, HC-CDR3: SEQ ID NO: 178; HC-CDR1: SEQ ID NO: 13, HC-CDR2: SEQ ID NO: 29, HC-CDR3: SEQ ID NO: 43; and HC-CDR1: SEQ ID NO: 13, HC-CDR2: SEQ ID NO: 29, HC-CDR3: SEQ ID NO: 44.

Embodiment 3 comprises an antibody of embodiment 1 or 2, wherein the antibody comprises a light chain variable domain that comprises CDRs: LC-CDR1, LC-CDR2, LC-CDR3, wherein the LC-CDR1, LC-CDR2, LC-CDR3 of the light chain variable domain comprises a set of sequences selected from the group consisting of the following set of sequences: LC-CDR1: SEQ ID NO: 45, LC-CDR2: SEQ ID NO: 58, LC-CDR3: SEQ ID NO: 70; LC-CDR1: SEQ ID NO: 46, LC-CDR2: SEQ ID NO: 59, LC-CDR3: SEQ ID NO: 71; LC-CDR1: SEQ ID NO: 47, LC-CDR2: SEQ ID NO: 60, LC-CDR3: SEQ ID NO: 72; LC-CDR1: SEQ ID NO: 48, LC-CDR2: SEQ ID NO: 59, LC-CDR3: SEQ ID NO: 73; LC-CDR1: SEQ ID NO: 49, LC-CDR2: SEQ ID NO: 61, LC-CDR3: SEQ ID NO: 74; LC-CDR1: SEQ ID NO: 50, LC-CDR2: SEQ ID NO: 62, LC-CDR3: SEQ ID NO: 75; LC-CDR1: SEQ ID NO: 51, LC-CDR2: SEQ ID NO: 63, LC-CDR3: SEQ ID NO: 76; LC-CDR1: SEQ ID NO: 52, LC-CDR2: SEQ ID NO: 62, LC-CDR3: SEQ ID NO: 77; LC-CDR1: SEQ ID NO: 53, LC-CDR2: SEQ ID NO: 64, LC-CDR3: SEQ ID NO: 78; LC-CDR1: SEQ ID NO: 54, LC-CDR2: SEQ ID NO: 65, LC-CDR3: SEQ ID NO: 79; LC-CDR1: SEQ ID NO: 55, LC-CDR2: SEQ ID NO: 66, LC-CDR3: SEQ ID NO: 80; LC-CDR1: SEQ ID NO: 53, LC-CDR2: SEQ ID NO: 67, LC-CDR3: SEQ ID NO: 78; LC-CDR1: SEQ ID NO: 52, LC-CDR2: SEQ ID NO: 62, LC-CDR3: SEQ ID NO: 81; LC-CDR1: SEQ ID NO: 56, LC-CDR2: SEQ ID NO: 68, LC-CDR3: SEQ ID NO: 82; LC-CDR1: SEQ ID NO: 45, LC-CDR2: SEQ ID NO: 69, LC-CDR3: SEQ ID NO: 70; LC-CDR1: SEQ ID NO: 57, LC-CDR2: SEQ ID NO: 66, LC-CDR3: SEQ ID NO: 83; LC-CDR1: SEQ ID NO: 57, LC-CDR2: SEQ ID NO: 66, LC-CDR3: SEQ ID NO: 84; LC-CDR1: SEQ ID NO: 57, LC-CDR2: SEQ ID NO: 66, LC-CDR3: SEQ ID NO: 85.

Embodiment 4 comprises an antibody of embodiment 1, wherein the antibody comprises at least one complementarity determining region (CDR) according to SEQ ID NOs: 1, 13, 66, 69, 28-30, 30, 43, 44, 45, 57, 70, 83-85, 177-178 or an amino acid sequence that has 0-1 amino acid mutations, substitutions, or deletions relative to SEQ ID NOs: 1, 13, 66, 69, or an amino acid sequence that has 0-3 amino acid mutations, substitutions, or deletions to relative to SEQ ID NOs: 28-30, 30, 43, 44, 45, 57, 70, 83-85, 177-178.

Embodiment 5 comprises an antibody of embodiment 4, wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain comprises a set of sequences selected from the group consisting of the following set of sequences: HC-CDR1: SEQ ID NO: 1, HC-CDR2: SEQ ID NO: 28, HC-CDR3: SEQ ID NO: 30; HC-CDR1: SEQ ID NO: 1, HC-CDR2: SEQ ID NO: 28, HC-CDR3: SEQ ID NO: 177; HC-CDR1: SEQ ID NO: 1, HC-CDR2: SEQ ID NO: 28, HC-CDR3: SEQ ID NO: 178; HC-CDR1: SEQ ID NO: 13, HC-CDR2: SEQ ID NO: 29, HC-CDR3: SEQ ID NO: 43; and HC-CDR1: SEQ ID NO: 13, HC-CDR2: SEQ ID NO: 29, HC-CDR3: SEQ ID NO: 44; and wherein the CDRs comprise 0-2 amino acid mutations, substitutions, or deletions in at least one of the HC-CDR1, HC-CDR2, HC-CDR3.

Embodiment 6 comprises an antibody of embodiment 4 or 5, wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain comprises a set of sequences selected from the group consisting of the following set of sequences: HC-CDR1: SEQ ID NO: 1, HC-CDR2: SEQ ID NO: 28, HC-CDR3: SEQ ID NO: 30; HC-CDR1: SEQ ID NO: 1, HC-CDR2: SEQ ID NO: 28, HC-CDR3: SEQ ID NO: 177; HC-CDR1: SEQ ID NO: 1, HC-CDR2: SEQ ID NO: 28, HC-CDR3: SEQ ID NO: 178; HC-CDR1: SEQ ID NO: 13, HC-CDR2: SEQ ID NO: 29, HC-CDR3: SEQ ID NO: 43; and HC-CDR1: SEQ ID NO: 13, HC-CDR2: SEQ ID NO: 29, HC-CDR3: SEQ ID NO: 44.

Embodiment 7 comprises an antibody of any one of embodiments 4-6, wherein the antibody comprises alight chain variable domain that comprises CDRs: LC-CDR1, LC-CDR2, LC-CDR3, wherein the LC-CDR1, LC-CDR2, LC-CDR3 of the light chain variable domain comprises a set of sequences selected from the group consisting of the following set of sequences: LC-CDR1: SEQ ID NO: 45, LC-CDR2: SEQ ID NO: 69, LC-CDR3: SEQ ID NO: 70; LC-CDR1: SEQ ID NO: 57, LC-CDR2: SEQ ID NO: 66, LC-CDR3: SEQ ID NO: 83; LC-CDR1: SEQ ID NO: 57, LC-CDR2: SEQ ID NO: 66, LC-CDR3: SEQ ID NO: 84; LC-CDR1: SEQ ID NO: 57, LC-CDR2: SEQ ID NO: 66, LC-CDR3: SEQ ID NO: 85; and wherein the CDRs comprise 0-2 amino acid mutations, substitutions, or deletions in at least one of the LC-CDR1, LC-CDR2, LC-CDR3.

Embodiment 8 comprises an antibody of any one of embodiments 4-7, wherein the antibody comprises alight chain variable domain that comprises CDRs: LC-CDR1, LC-CDR2, LC-CDR3, wherein the LC-CDR1, LC-CDR2, LC-CDR3 of the light chain variable domain comprises a set of sequences selected from the group consisting of the following set of sequences: LC-CDR1: SEQ ID NO: 45, LC-CDR2: SEQ ID NO: 69, LC-CDR3: SEQ ID NO: 70; LC-CDR1: SEQ ID NO: 57, LC-CDR2: SEQ ID NO: 66, LC-CDR3: SEQ ID NO: 83; LC-CDR1: SEQ ID NO: 57, LC-CDR2: SEQ ID NO: 66, LC-CDR3: SEQ ID NO: 84; and LC-CDR1: SEQ ID NO: 57, LC-CDR2: SEQ ID NO: 66, LC-CDR3: SEQ ID NO: 85.

Embodiment 9 comprises an antibody of embodiment 8, wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain comprises a set of sequences: HC-CDR1: SEQ ID NO: 13, HC-CDR2: SEQ ID NO: 29, HC-CDR3: SEQ ID NO: 44; and wherein the CDRs comprise 0-2 amino acid mutations, substitutions, or deletions in at least one of the HC-CDR1, HC-CDR2, HC-CDR3.

Embodiment 10 comprises an antibody of embodiment 9, wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain comprises a set of sequences: HC-CDR1: SEQ ID NO: 13, HC-CDR2: SEQ ID NO: 29, HC-CDR3: SEQ ID NO: 44.

Embodiment 11 comprises an antibody of embodiment 8, wherein the antibody comprises alight chain variable domain that comprises CDRs: LC-CDR1, LC-CDR2, LC-CDR3, wherein the LC-CDR1, LC-CDR2, LC-CDR3 of the light chain variable domain comprises LC-CDR1: SEQ ID NO: 57, LC-CDR2: SEQ ID NO: 66, LC-CDR3: SEQ ID NO: 84; or SEQ ID NO: 57, LC-CDR2: SEQ ID NO: 66, LC-CDR3: SEQ ID NO: 85; and wherein the CDRs comprise 0-2 amino acid mutations, substitutions, or deletions in at least one of the LC-CDR1, LC-CDR2, LC-CDR3.

Embodiment 12 comprises an antibody of embodiment 11, wherein the antibody comprises a light chain variable domain that comprises CDRs: LC-CDR1, LC-CDR2, LC-CDR3, wherein the LC-CDR1, LC-CDR2, LC-CDR3 of the light chain variable domain comprises SEQ ID NO: 57, LC-CDR2: SEQ ID NO: 66, LC-CDR3: SEQ ID NO: 84; or SEQ ID NO: 57, LC-CDR2: SEQ ID NO: 66, LC-CDR3: SEQ ID NO: 85.

Embodiment 13 comprises an antibody of embodiment 4, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise a set of sequences selected from the group consisting of the following set of sequences: HC-CDR1: SEQ ID NO: 1, HC-CDR2: SEQ ID NO: 28, HC-CDR3: SEQ ID NO: 30, LC-CDR1: SEQ ID NO: 45, LC-CDR2: SEQ ID NO: 69, LC-CDR3: SEQ ID NO: 70; HC-CDR1: SEQ ID NO: 1, HC-CDR2: SEQ ID NO: 28, HC-CDR3: SEQ ID NO: 177, LC-CDR1: SEQ ID NO: 45, LC-CDR2: SEQ ID NO: 69, LC-CDR3: SEQ ID NO: 70; HC-CDR1: SEQ ID NO: 1, HC-CDR2: SEQ ID NO: 28, HC-CDR3: SEQ ID NO: 178, LC-CDR1: SEQ ID NO: 45, LC-CDR2: SEQ ID NO: 69, LC-CDR3: SEQ ID NO: 70; HC-CDR1: SEQ ID NO: 13, HC-CDR2: SEQ ID NO: 29, HC-CDR3: SEQ ID NO: 43, LC-CDR1: SEQ ID NO: 57, LC-CDR2: SEQ ID NO: 66, LC-CDR3: SEQ ID NO: 83; HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 84; and HC-CDR1: SEQ ID NO: 13, HC-CDR2: SEQ ID NO: 29, HC-CDR3: SEQ ID NO: 44; LC-CDR1: SEQ ID NO: 57, LC-CDR2: SEQ ID NO: 66, LC-CDR3: SEQ ID NO: 85; wherein the CDRs comprise 0-2 amino acid mutations, substitutions, or deletions in at least one of the HC-CDR1, HC-CDR2, HC-CDR3, LC-CDR1, LC-CDR2, LC-CDR3.

Embodiment 14 comprises an antibody of embodiment 13, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise a set of sequences selected from the group consisting of the following set of sequences: HC-CDR1: SEQ ID NO: 1, HC-CDR2: SEQ ID NO: 28, HC-CDR3: SEQ ID NO: 30, LC-CDR1: SEQ ID NO: 45, LC-CDR2: SEQ ID NO: 69, LC-CDR3: SEQ ID NO: 70; HC-CDR1: SEQ ID NO: 1, HC-CDR2: SEQ ID NO: 28, HC-CDR3: SEQ ID NO: 177, LC-CDR1: SEQ ID NO: 45, LC-CDR2: SEQ ID NO: 69, LC-CDR3: SEQ ID NO: 70; HC-CDR1: SEQ ID NO: 1, HC-CDR2: SEQ ID NO: 28, HC-CDR3: SEQ ID NO: 178, LC-CDR1: SEQ ID NO: 45, LC-CDR2: SEQ ID NO: 69, LC-CDR3: SEQ ID NO: 70; HC-CDR1: SEQ ID NO: 13, HC-CDR2: SEQ ID NO: 29, HC-CDR3: SEQ ID NO: 43, LC-CDR1: SEQ ID NO: 57, LC-CDR2: SEQ ID NO: 66, LC-CDR3: SEQ ID NO: 83; HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 84; and HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 85.

Embodiment 15 comprises an antibody of embodiment 4, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 30; LC-CDR1: SEQ ID NO: 45, LC-CDR2: SEQ ID NO: 69, LC-CDR3: SEQ ID NO: 70; wherein the CDRs comprise 0-2 amino acid mutations, substitutions, or deletions in at least one of the HC-CDR1, HC-CDR2, HC-CDR3, LC-CDR1, LC-CDR2, LC-CDR3.

Embodiment 16 comprises an antibody of embodiment 15, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 30; LC-CDR1: SEQ ID NO: 45, LC-CDR2: SEQ ID NO: 69, LC-CDR3: SEQ ID NO: 70.

Embodiment 17 comprises an antibody of embodiment 4, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 177; LC-CDR1: SEQ ID NO: 45, LC-CDR2: SEQ ID NO: 69, LC-CDR3: SEQ ID NO: 70; wherein the CDRs comprise 0-2 amino acid mutations, substitutions, or deletions in at least one of the HC-CDR1, HC-CDR2, HC-CDR3, LC-CDR1, LC-CDR2, LC-CDR3.

Embodiment 18 comprises an antibody of embodiment 17, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 177; LC-CDR1: SEQ ID NO: 45, LC-CDR2: SEQ ID NO: 69, LC-CDR3: SEQ ID NO: 70.

Embodiment 19 comprises an antibody of embodiment 4, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 178; LC-CDR1: SEQ ID NO: 45, LC-CDR2: SEQ ID NO: 69, LC-CDR3: SEQ ID NO: 70; wherein the CDRs comprise 0-2 amino acid mutations, substitutions, or deletions in at least one of the HC-CDR1, HC-CDR2, HC-CDR3, LC-CDR1, LC-CDR2, LC-CDR3.

Embodiment 20 comprises an antibody of embodiment 19, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 178; LC-CDR1: SEQ ID NO: 45, LC-CDR2: SEQ ID NO: 69, LC-CDR3: SEQ ID NO: 70.

Embodiment 21 comprises an antibody of embodiment 4, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 43; LC-CDR1: SEQ ID NO: 57, LC-CDR2: SEQ ID NO: 66, LC-CDR3: SEQ ID NO: 83; wherein the CDRs comprise 0-2 amino acid mutations, substitutions, or deletions in at least one of the HC-CDR1, HC-CDR2, HC-CDR3, LC-CDR1, LC-CDR2, LC-CDR3.

Embodiment 22 comprises an antibody of embodiment 21, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 43; LC-CDR1: SEQ ID NO: 57, LC-CDR2: SEQ ID NO: 66, LC-CDR3: SEQ ID NO: 83.

Embodiment 23 comprises an antibody of embodiment 4, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 44; LC-CDR1: SEQ ID NO: 57, LC-CDR2: SEQ ID NO: 66, LC-CDR3: SEQ ID NO: 84; wherein the CDRs comprise 0-2 amino acid mutations, substitutions, or deletions in at least one of the HC-CDR1, HC-CDR2, HC-CDR3, LC-CDR1, LC-CDR2, LC-CDR3.

Embodiment 24 comprises an antibody of embodiment 23, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 44; LC-CDR1: SEQ ID NO: 57, LC-CDR2: SEQ ID NO: 66, LC-CDR3: SEQ ID NO: 84.

Embodiment 25 comprises an antibody of embodiment 4, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 44; LC-CDR1: SEQ ID NO: 57, LC-CDR2: SEQ ID NO: 66, LC-CDR3: SEQ ID NO: 85; wherein the CDRs comprise 0-2 amino acid mutations, substitutions, or deletions in at least one of the HC-CDR1, HC-CDR2, HC-CDR3, LC-CDR1, LC-CDR2, LC-CDR3.

Embodiment 26 comprises an antibody of embodiment 25, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 44; LC-CDR1: SEQ ID NO: 57, LC-CDR2: SEQ ID NO: 66, LC-CDR3: SEQ ID NO: 85.

Embodiment 27 comprises an antibody of any one of embodiments 1-26, wherein the antibody comprises an antibody format selected from Fab, Fab′, scFv, and (Fab′)₂.

Embodiment 28 comprises an antibody of any one of embodiments 1-27, wherein the heavy chain variable domain is fused to a human IgG1 constant region.

Embodiment 29 comprises an antibody of any one of embodiments 1-27, wherein the heavy chain variable domain is fused to a human IgG4 constant region.

Embodiment 30 comprises an antibody of any one of embodiments 1-29, wherein the light chain variable domain is fused to a human Kappa constant region.

Embodiment 31 comprises an antibody of any one of embodiments 1-27, wherein the heavy chain variable domain comprises a variable domain of an IgG1, IgG2, IgG3, or IgG4 heavy chain.

Embodiment 32 comprises an antibody of any one of embodiments 1-27, wherein the light chain variable domain comprises a variable domain of a Kappa light chain.

Embodiment 33 comprises an antibody of any one of embodiments 1-32, wherein the heavy chain variable domain comprises an amino acid sequence with at least 90%, 95%, 96%, 97%, 98%, 99%, or 100% identity to any one of SEQ ID NOs: 143, 149, 181, 184, 155, 161, or 167.

Embodiment 34 comprises an antibody of any one of embodiments 1-33, wherein the light chain variable domain comprises an amino acid sequence with at least 90%, 95%, 96%, 97%, 98%, 99%, or 100% identity to any one of SEQ ID NOs: 144, 150, 156, 162, or 168.

Embodiment 35 comprises an antibody of embodiment 34, wherein the heavy chain variable domain comprises an amino acid sequence with at least 90% sequence identity to SEQ ID NO: 143, and the light chain variable domain comprises an amino acid sequence with at least 90% sequence identity to SEQ ID NO: 144.

Embodiment 36 comprises an antibody of embodiment 34, wherein the heavy chain variable domain comprises an amino acid sequence with at least 90% sequence identity to SEQ ID NO: 149, and the light chain variable domain comprises an amino acid sequence with at least 90% sequence identity to SEQ ID NO: 150.

Embodiment 37 comprises an antibody of embodiment 34, wherein the heavy chain variable domain comprises an amino acid sequence with at least 90% sequence identity to SEQ ID NO: 181, and the light chain variable domain comprises an amino acid sequence with at least 90% sequence identity to SEQ ID NO: 144.

Embodiment 38 comprises an antibody of embodiment 34, wherein the heavy chain variable domain comprises an amino acid sequence with at least 90% sequence identity to SEQ ID NO: 184, and the light chain variable domain comprises an amino acid sequence with at least 90% sequence identity to SEQ ID NO: 144.

Embodiment 39 comprises an antibody of embodiment 34, wherein the heavy chain variable domain comprises an amino acid sequence with at least 90% sequence identity to SEQ ID NO: 155, and the light chain variable domain comprises an amino acid sequence with at least 90% sequence identity to SEQ ID NO: 156.

Embodiment 40 comprises an antibody of embodiment 34, wherein the heavy chain variable domain comprises an amino acid sequence with at least 90% sequence identity to SEQ ID NO: 161, and the light chain variable domain comprises an amino acid sequence with at least 90% sequence identity to SEQ ID NO: 162.

Embodiment 41 comprises an antibody of embodiment 34, wherein the heavy chain variable domain comprises an amino acid sequence with at least 90% sequence identity to SEQ ID NO: 167, and the light chain variable domain comprises an amino acid sequence with at least 90% sequence identity to SEQ ID NO: 168.

Embodiment 42 comprises an antibody of embodiment 34, wherein the antibody comprises amino acid sequences with at least 90% sequence identity to SEQ ID NO: 145, and at least 90% sequence identity to SEQ ID NO: 147.

Embodiment 43 comprises an antibody of embodiment 34, wherein the antibody comprises amino acid sequences with at least 90% sequence identity to SEQ ID NO: 151, and at least 90% sequence identity to SEQ ID NO: 153.

Embodiment 44 comprises an antibody of embodiment 34, wherein the antibody comprises amino acid sequences with at least 90% sequence identity to SEQ ID NO: 182, and at least 90% sequence identity to SEQ ID NO: 147.

Embodiment 45 comprises an antibody of embodiment 34, wherein the antibody comprises amino acid sequences with at least 90% sequence identity to SEQ ID NO: 185, and at least 90% sequence identity to SEQ ID NO: 147.

Embodiment 46 comprises an antibody of embodiment 34, wherein the antibody comprises amino acid sequences with at least 90% sequence identity to SEQ ID NO: 157, and at least 90% sequence identity to SEQ ID NO: 159.

Embodiment 47 comprises an antibody of embodiment 34, wherein the antibody comprises amino acid sequences with at least 90% sequence identity to SEQ ID NO: 163, and at least 90% sequence identity to SEQ ID NO: 165.

Embodiment 48 comprises an antibody of embodiment 34, wherein the antibody comprises amino acid sequences with at least 90% sequence identity to SEQ ID NO: 169, and at least 90% sequence identity to SEQ ID NO: 171.

Embodiment 49 comprises an antibody of any one of embodiments 1-48, wherein the antibody binds to extracellular domain of human CD47 with an EC50 of between 0.01 nM-0.5 nM as determined by an ELISA binding assay.

Embodiment 50 comprises an antibody of embodiment 49, wherein the ELISA binding assay comprises the following steps: coating a 96-well plate for at least 12 hours with 1p g/ml of recombinant CD47; washing the plate three times; blocking the plate with 300 μL of 1% bovine serum albumin in phosphate-buffered saline solution with tween (PBST) at 37° C. for 1 hour; washing the plate four times with PBST; incubating a serial dilution of the antibody that binds specifically to CD47 at 37° C. for 1 hour; washing the plate four times with PBST; incubating a 1:5000 dilution of anti-human IgG-peroxidase antibody at 37° C. for 1 hour within the plate; washing the plate 4 times with PBST; incubating 3,3′,5,5′ tetramethylbenzidine substrate for 15 minutes at room temperature within the plate; terminating the reaction within the plate with 1N HCl; and reading the plate at 450 nm to determine the EC50 of the antibody for binding to the extracellular domain of human CD47.

Embodiment 51 comprises an antibody of embodiment 49 or 50, wherein the extracellular domain of human CD47 comprises an amino acid sequence according to SEQ ID NO: 86.

Embodiment 52 comprises an antibody of any one of embodiments 1-51, wherein the antibody binds to CD47+ cell lines with an EC50 of between 0.1 nM-10 nM as determined by flow cytometry.

Embodiment 53 comprises an antibody of embodiment 52, wherein the flow cytometry comprises the following steps and assay conditions: centrifuging CD47+ cells at 2000 rpm for 5 minutes to obtain centrifuged cells; resuspending the centrifuged cells in 10-15 mL of culture medium; resuspending cells in blocking buffer comprising phosphate buffer saline with 2% fetal bovine serum (PBS plus 2% FBS) at a concentration of 3×10⁶ cells/mL to obtain a cell suspension; dispensing the cell suspension into wells of a 96-well plate; diluting the antibody that binds specifically to CD47 in the blocking buffer to a desired concentration followed by addition of 100 μL of the antibody per well and incubating at 1 hour for 4° C.; washing the cells three times with PBS plus 2% FBS; resuspending the cells in 100 μL 1:500 diluted Alexa Fluor 488 labeled Mouse anti-Human IgG1 Fc secondary antibody and incubating at 4° C. in the dark for one hour; washing the cells three times with 200 μL PBS and centrifuging at 2000 rpm for 5 minutes; resuspending cells in 300 μL cold PBS; and analyzing with a flow cytometer to determine the EC50 of the antibody for the CD47+ cell line.

Embodiment 54 comprises an antibody of any one of embodiments 1-53, wherein the antibody blocks SIRPa activity with an IC50 of between 0.1 nM-10 nM as determined by flow cytometry.

Embodiment 55 comprises an antibody of any one of embodiments 1-54, wherein the IC50 for SIRPa activity as determined by flow cytometry comprises the following steps and assay conditions: harvesting, centrifuging, and resuspending Raji tumor cells or HCT-15 tumor cells in FACS buffer comprising phosphate buffer saline with 2% fetal bovine serum (PBS plus 2% FBS) at a concentration of 2×10⁶ cells/mL; dispensing 100 μL of the cell suspension into wells of a 96-well plate; centrifuging the plate for 5 minutes at 300×g followed by discarding of supernatants; incubating cells from the plate with 50 μL per well of serially diluted antibody and a constant amount of SIRPA-mIgG2a fusion protein (0.2 μg/mL for Raji cells, 1 μg/mL for HCT-15 cells) in FACS buffer for 1 hour at 4° C.; washing the plate with FACS buffer followed by incubating for one hour at 4° C. in the dark with 100 μL of Alexa Fluor 488 donkey anti-Mouse IgG(H+L) secondary antibody; washing twice with FACS buffer; resuspending cells in the plate with 300 μL FACS buffer; and analyzing with a flow cytometer to determine the IC50 of the antibody for blocking SIRPa activity.

Embodiment 56 comprises an antibody of any one of embodiments 1-55, wherein the antibody induces increased antibody-dependent cellular phagocytosis (ADCP) as compared to a control antibody that comprises amino acid sequences according to SEQ ID NOs: 173 and 174 when tested under substantially equivalent assay conditions in an assay for ADCP.

Embodiment 57 comprises an antibody of any one of embodiments 1-56, wherein the antibody has less binding on human red blood cells (RBC) as compared to a control antibody that comprises amino acid sequences according to SEQ ID NOs: 173 and 174 when tested under substantially equivalent assay conditions for assessing RBC binding.

Embodiment 58 comprises an antibody of any one of embodiments 1-57, wherein a concentration of 600 nM of the antibody does not induce hemolysis of red blood cells in a hemagglutination assay.

Embodiment 59 comprises an antibody of any one of embodiments 1-58, wherein the antibody induces increased reduction in tumor volume as compared to a control antibody that comprises amino acid sequences according to SEQ ID NOs: 173 and 174 when tested under substantially equivalent assay conditions in a tumor growth animal model.

Embodiment 60 comprises an antibody that binds specifically to CD47 wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain are selected from the following amino acid sequences HC-CDR1: SEQ ID NOs: 1-13, HC-CDR2: SEQ ID NOs: 14-29, HC-CDR3: SEQ ID NOs: 30-44, 176-178; and a light chain variable domain that comprises CDRs: LC-CDR1, LC-CDR2, LC-CDR3, wherein the LC-CDR1, LC-CDR2, LC-CDR3 of the light chain variable domain are selected from the following amino acid sequences LC-CDR1: SEQ ID NOs: 45-57, LC-CDR2: SEQ ID NOs: 58-69, LC-CDR3: SEQ ID NOs: 70-85 and wherein the CDRs comprise 0-2 amino acid mutations, substitutions, or deletions in at least one of the HC-CDR1, HC-CDR2, HC-CDR3, LC-CDR1, LC-CDR2, LC-CDR3.

Embodiment 61 comprises an antibody of embodiment 60, wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain are selected from the following amino acid sequences HC-CDR1: SEQ ID NOs: 1-13, HC-CDR2: SEQ ID NOs: 14-29, HC-CDR3: SEQ ID NOs: 30-44, 176-178; and a light chain variable domain that comprises CDRs: LC-CDR1, LC-CDR2, LC-CDR3, wherein the LC-CDR1, LC-CDR2, LC-CDR3 of the light chain variable domain are selected from the following amino acid sequences LC-CDR1: SEQ ID NOs: 45-57, LC-CDR2: SEQ ID NOs: 58-69, LC-CDR3: SEQ ID NOs: 70-85 and wherein the CDRs comprise 0-2 amino acid mutations, substitutions, or deletions in at least one of the HC-CDR3 or LC-CDR3.

Embodiment 62 comprises an antibody of embodiment 61, wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain are selected from the following amino acid sequences HC-CDR1: SEQ ID NOs: 1-13, HC-CDR2: SEQ ID NOs: 14-29, HC-CDR3: SEQ ID NOs: 30-44, 176-178; and a light chain variable domain that comprises CDRs: LC-CDR1, LC-CDR2, LC-CDR3, wherein the LC-CDR1, LC-CDR2, LC-CDR3 of the light chain variable domain are selected from the following amino acid sequences LC-CDR1: SEQ ID NOs: 45-57, LC-CDR2: SEQ ID NOs: 58-69, LC-CDR3: SEQ ID NOs: 70-85.

Embodiment 63 comprises an antibody of any one of embodiments 60-62, wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain are selected from the following amino acid sequences HC-CDR1: SEQ ID NOs: 1 or 10, HC-CDR2: SEQ ID NOs: 14 or 24, HC-CDR3: SEQ ID NOs: 30 or 39; and a light chain variable domain that comprises CDRs: LC-CDR1, LC-CDR2, LC-CDR3, wherein the LC-CDR1, LC-CDR2, LC-CDR3 of the light chain variable domain are selected from the following amino acid sequences LC-CDR1: SEQ ID NOs: 45 or 55, LC-CDR2: SEQ ID NOs: 58 or 66, LC-CDR3: SEQ ID NOs: 70 or 80 and wherein the CDRs comprise 0-2 amino acid mutations, substitutions, or deletions in at least one of the HC-CDR1, HC-CDR2, HC-CDR3, LC-CDR1, LC-CDR2, LC-CDR3.

Embodiment 64 comprises an antibody of embodiment 63, wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain are selected from the following amino acid sequences HC-CDR1: SEQ ID NOs: 1 or 10, HC-CDR2: SEQ ID NOs: 14 or 24, HC-CDR3: SEQ ID NOs: 30 or 39; and a light chain variable domain that comprises CDRs: LC-CDR1, LC-CDR2, LC-CDR3, wherein the LC-CDR1, LC-CDR2, LC-CDR3 of the light chain variable domain are selected from the following amino acid sequences LC-CDR1: SEQ ID NOs: 45 or 55, LC-CDR2: SEQ ID NOs: 58 or 66, LC-CDR3: SEQ ID NOs: 70 or 80 and wherein the CDRs comprise 0-2 amino acid mutations, substitutions, or deletions in at least one of the HC-CDR3 or LC-CDR3.

Embodiment 65 comprises an antibody of any one of embodiments 60-62, wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain are selected from the following amino acid sequences HC-CDR1: SEQ ID NOs: 1, HC-CDR2: SEQ ID NOs: 14, HC-CDR3: SEQ ID NOs: 30; and a light chain variable domain that comprises CDRs: LC-CDR1, LC-CDR2, LC-CDR3, wherein the LC-CDR1, LC-CDR2, LC-CDR3 of the light chain variable domain are selected from the following amino acid sequences LC-CDR1: SEQ ID NOs: 45, LC-CDR2: SEQ ID NOs: 58, LC-CDR3: SEQ ID NOs: 70 and wherein the CDRs comprise 0-2 amino acid mutations, substitutions, or deletions in at least one of the HC-CDR1, HC-CDR2, HC-CDR3, LC-CDR1, LC-CDR2, LC-CDR3.

Embodiment 66 comprises an antibody of embodiment 65, wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain are selected from the following amino acid sequences HC-CDR1: SEQ ID NOs: 1, HC-CDR2: SEQ ID NOs: 14, HC-CDR3: SEQ ID NOs: 30; and a light chain variable domain that comprises CDRs: LC-CDR1, LC-CDR2, LC-CDR3, wherein the LC-CDR1, LC-CDR2, LC-CDR3 of the light chain variable domain are selected from the following amino acid sequences LC-CDR1: SEQ ID NOs: 45, LC-CDR2: SEQ ID NOs: 58, LC-CDR3: SEQ ID NOs: 70 and wherein the CDRs comprise 0-2 amino acid mutations, substitutions, or deletions in at least one of the HC-CDR3 or LC-CDR3.

Embodiment 67 comprises an antibody of embodiment 60, wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain are selected from the following amino acid sequences HC-CDR1: SEQ ID NOs: 10, HC-CDR2: SEQ ID NOs: 24, HC-CDR3: SEQ ID NOs: 39; and a light chain variable domain that comprises CDRs: LC-CDR1, LC-CDR2, LC-CDR3, wherein the LC-CDR1, LC-CDR2, LC-CDR3 of the light chain variable domain are selected from the following amino acid sequences LC-CDR1: SEQ ID NOs: 55, LC-CDR2: SEQ ID NOs: 66, LC-CDR3: SEQ ID NOs: 80 and wherein the CDRs comprise 0-2 amino acid mutations, substitutions, or deletions in at least one of the HC-CDR1, HC-CDR2, HC-CDR3, LC-CDR1, LC-CDR2, LC-CDR3.

Embodiment 68 comprises an antibody of embodiment 67, wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain are selected from the following amino acid sequences HC-CDR1: SEQ ID NOs: 10, HC-CDR2: SEQ ID NOs: 24, HC-CDR3: SEQ ID NOs: 39; and a light chain variable domain that comprises CDRs: LC-CDR1, LC-CDR2, LC-CDR3, wherein the LC-CDR1, LC-CDR2, LC-CDR3 of the light chain variable domain are selected from the following amino acid sequences LC-CDR1: SEQ ID NOs: 55, LC-CDR2: SEQ ID NOs: 66, LC-CDR3: SEQ ID NOs: 80 and wherein the CDRs comprise 0-2 amino acid mutations, substitutions, or deletions in at least one of the HC-CDR3 or LC-CDR3.

Embodiment 69 comprises an antibody of embodiment 60, wherein the CDRs of the heavy chain variable domain and the CDRs of the light chain variable domain are selected from the following sets of amino acid sequences: (a) HC-CDR1: SEQ ID NO: 1, HC-CDR2: SEQ ID NO: 14, HC-CDR3: SEQ ID NO: 30, LC-CDR1: SEQ ID NO: 45, LC-CDR2: SEQ ID NO: 58, LC-CDR3: SEQ ID NO: 70; (b) HC-CDR1: SEQ ID NO: 2, HC-CDR2: SEQ ID NO: 15, HC-CDR3: SEQ ID NO: 31, LC-CDR1: SEQ ID NO: 46, LC-CDR2: SEQ ID NO: 59, LC-CDR3: SEQ ID NO: 71; (c) HC-CDR1: SEQ ID NO: 3, HC-CDR2: SEQ ID NO: 16, HC-CDR3: SEQ ID NO: 32, LC-CDR1: SEQ ID NO: 47, LC-CDR2: SEQ ID NO: 60, LC-CDR3: SEQ ID NO: 72; (d) HC-CDR1: SEQ ID NO: 2, HC-CDR2: SEQ ID NO: 17, HC-CDR3: SEQ ID NO: 33, LC-CDR1: SEQ ID NO: 48, LC-CDR2: SEQ ID NO: 59, LC-CDR3: SEQ ID NO: 73; (e) HC-CDR1: SEQ ID NO: 4, HC-CDR2: SEQ ID NO: 18, HC-CDR3: SEQ ID NO: 34, LC-CDR1: SEQ ID NO: 49, LC-CDR2: SEQ ID NO: 61, LC-CDR3: SEQ ID NO: 74; (f) HC-CDR1: SEQ ID NO: 5, HC-CDR2: SEQ ID NO: 19, HC-CDR3: SEQ ID NO: 35, LC-CDR1: SEQ ID NO: 50, LC-CDR2: SEQ ID NO: 62, LC-CDR3: SEQ ID NO: 75; (g) HC-CDR1: SEQ ID NO: 6, HC-CDR2: SEQ ID NO: 20, HC-CDR3: SEQ ID NO: 176, LC-CDR1: SEQ ID NO: 51, LC-CDR2: SEQ ID NO: 63, LC-CDR3: SEQ ID NO: 76; (h) HC-CDR1: SEQ ID NO: 7, HC-CDR2: SEQ ID NO: 21, HC-CDR3: SEQ ID NO: 36, LC-CDR1: SEQ ID NO: 52, LC-CDR2: SEQ ID NO: 62, LC-CDR3: SEQ ID NO: 77; (i) HC-CDR1: SEQ ID NO: 8, HC-CDR2: SEQ ID NO: 22, HC-CDR3: SEQ ID NO: 37, LC-CDR1: SEQ ID NO: 53, LC-CDR2: SEQ ID NO: 64, LC-CDR3: SEQ ID NO: 78; (j) HC-CDR1: SEQ ID NO: 9, HC-CDR2: SEQ ID NO: 23, HC-CDR3: SEQ ID NO: 38, LC-CDR1: SEQ ID NO: 54, LC-CDR2: SEQ ID NO: 65, LC-CDR3: SEQ ID NO: 79; (k) HC-CDR1: SEQ ID NO: 10, HC-CDR2: SEQ ID NO: 24, HC-CDR3: SEQ ID NO: 39, LC-CDR1: SEQ ID NO: 55, LC-CDR2: SEQ ID NO: 66, LC-CDR3: SEQ ID NO: 80; (1) HC-CDR1: SEQ ID NO: 8, HC-CDR2: SEQ ID NO: 25, HC-CDR3: SEQ ID NO: 40, LC-CDR1: SEQ ID NO: 53, LC-CDR2: SEQ ID NO: 67, LC-CDR3: SEQ ID NO: 78; (m) HC-CDR1: SEQ ID NO: 11, HC-CDR2: SEQ ID NO: 26, HC-CDR3: SEQ ID NO: 41, LC-CDR1: SEQ ID NO: 52, LC-CDR2: SEQ ID NO: 62, LC-CDR3: SEQ ID NO: 81; (n) HC-CDR1: SEQ ID NO: 12, HC-CDR2: SEQ ID NO: 27, HC-CDR3: SEQ ID NO: 42, LC-CDR1: SEQ ID NO: 56, LC-CDR2: SEQ ID NO: 68, LC-CDR3: SEQ ID NO: 82; (o) HC-CDR1: SEQ ID NO: 1, HC-CDR2: SEQ ID NO: 28, HC-CDR3: SEQ ID NO: 30, LC-CDR1: SEQ ID NO: 45, LC-CDR2: SEQ ID NO: 69, LC-CDR3: SEQ ID NO: 70; (p) HC-CDR1: SEQ ID NO: 1, HC-CDR2: SEQ ID NO: 28, HC-CDR3: SEQ ID NO: 30, LC-CDR1: SEQ ID NO: 45, LC-CDR2: SEQ ID NO: 69, LC-CDR3: SEQ ID NO: 70; (q) HC-CDR1: SEQ ID NO: 1, HC-CDR2: SEQ ID NO: 28, HC-CDR3: SEQ ID NO: 177, LC-CDR1: SEQ ID NO: 45, LC-CDR2: SEQ ID NO: 69, LC-CDR3: SEQ ID NO: 70; (r) HC-CDR1: SEQ ID NO: 1, HC-CDR2: SEQ ID NO: 28, HC-CDR3: SEQ ID NO: 178, LC-CDR1: SEQ ID NO: 45, LC-CDR2: SEQ ID NO: 69, LC-CDR3: SEQ ID NO: 70; (s) HC-CDR1: SEQ ID NO: 13, HC-CDR2: SEQ ID NO: 29, HC-CDR3: SEQ ID NO: 43, LC-CDR1: SEQ ID NO: 57, LC-CDR2: SEQ ID NO: 66, LC-CDR3: SEQ ID NO: 83; (t) HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 84; and (u) HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 85.

Embodiment 70 comprises an antibody of embodiment 69, wherein the HC-CDR1, HC-CDR2, HC-CDR3, LC-CDR1, LC-CDR2, LC-CDR3 comprise sequences according to (a) HC-CDR1: SEQ ID NO: 1, HC-CDR2: SEQ ID NO: 14, HC-CDR3: SEQ ID NO: 30, LC-CDR1: SEQ ID NO: 45, LC-CDR2: SEQ ID NO: 58, LC-CDR3: SEQ ID NO: 70.

Embodiment 71 comprises an antibody of embodiment 69, wherein the HC-CDR1, HC-CDR2, HC-CDR3, LC-CDR1, LC-CDR2, LC-CDR3 comprise sequences according to (b) HC-CDR1: SEQ ID NO: 2, HC-CDR2: SEQ ID NO: 15, HC-CDR3: SEQ ID NO: 31, LC-CDR1: SEQ ID NO: 46, LC-CDR2: SEQ ID NO: 59, LC-CDR3: SEQ ID NO: 71.

Embodiment 72 comprises an antibody of embodiment 69, wherein the HC-CDR1, HC-CDR2, HC-CDR3, LC-CDR1, LC-CDR2, LC-CDR3 comprise sequences according to (c) HC-CDR1: SEQ ID NO: 3, HC-CDR2: SEQ ID NO: 16, HC-CDR3: SEQ ID NO: 32, LC-CDR1: SEQ ID NO: 47, LC-CDR2: SEQ ID NO: 60, LC-CDR3: SEQ ID NO: 72.

Embodiment 73 comprises an antibody of embodiment 69, wherein the HC-CDR1, HC-CDR2, HC-CDR3, LC-CDR1, LC-CDR2, LC-CDR3 comprise sequences according to (d) HC-CDR1: SEQ ID NO: 2, HC-CDR2: SEQ ID NO: 17, HC-CDR3: SEQ ID NO: 33, LC-CDR1: SEQ ID NO: 48, LC-CDR2: SEQ ID NO: 59, LC-CDR3: SEQ ID NO: 73.

Embodiment 74 comprises an antibody of embodiment 69, wherein the HC-CDR1, HC-CDR2, HC-CDR3, LC-CDR1, LC-CDR2, LC-CDR3 comprise sequences according to (e) HC-CDR1: SEQ ID NO: 4, HC-CDR2: SEQ ID NO: 18, HC-CDR3: SEQ ID NO: 34, LC-CDR1: SEQ ID NO: 49, LC-CDR2: SEQ ID NO: 61, LC-CDR3: SEQ ID NO: 74.

Embodiment 75 comprises an antibody of embodiment 69, wherein the HC-CDR1, HC-CDR2, HC-CDR3, LC-CDR1, LC-CDR2, LC-CDR3 comprise sequences according to (f) HC-CDR1: SEQ ID NO: 5, HC-CDR2: SEQ ID NO: 19, HC-CDR3: SEQ ID NO: 35, LC-CDR1: SEQ ID NO: 50, LC-CDR2: SEQ ID NO: 62, LC-CDR3: SEQ ID NO: 75.

Embodiment 76 comprises an antibody of embodiment 69, wherein the HC-CDR1, HC-CDR2, HC-CDR3, LC-CDR1, LC-CDR2, LC-CDR3 comprise sequences according to (g) HC-CDR1: SEQ ID NO: 6, HC-CDR2: SEQ ID NO: 20, HC-CDR3: SEQ ID NO: 176, LC-CDR1: SEQ ID NO: 51, LC-CDR2: SEQ ID NO: 63, LC-CDR3: SEQ ID NO: 76.

Embodiment 77 comprises an antibody of embodiment 69, wherein the HC-CDR1, HC-CDR2, HC-CDR3, LC-CDR1, LC-CDR2, LC-CDR3 comprise sequences according to (h) HC-CDR1: SEQ ID NO: 7, HC-CDR2: SEQ ID NO: 21, HC-CDR3: SEQ ID NO: 36, LC-CDR1: SEQ ID NO: 52, LC-CDR2: SEQ ID NO: 62, LC-CDR3: SEQ ID NO: 77.

Embodiment 78 comprises an antibody of embodiment 69, wherein the HC-CDR1, HC-CDR2, HC-CDR3, LC-CDR1, LC-CDR2, LC-CDR3 comprise sequences according to (i) HC-CDR1: SEQ ID NO: 8, HC-CDR2: SEQ ID NO: 22, HC-CDR3: SEQ ID NO: 37, LC-CDR1: SEQ ID NO: 53, LC-CDR2: SEQ ID NO: 64, LC-CDR3: SEQ ID NO: 78.

Embodiment 79 comprises an antibody of embodiment 69, wherein the HC-CDR1, HC-CDR2, HC-CDR3, LC-CDR1, LC-CDR2, LC-CDR3 comprise sequences according to (j) HC-CDR1: SEQ ID NO: 9, HC-CDR2: SEQ ID NO: 23, HC-CDR3: SEQ ID NO: 38, LC-CDR1: SEQ ID NO: 54, LC-CDR2: SEQ ID NO: 65, LC-CDR3: SEQ ID NO: 79.

Embodiment 80 comprises an antibody of embodiment 69, wherein the HC-CDR1, HC-CDR2, HC-CDR3, LC-CDR1, LC-CDR2, LC-CDR3 comprise sequences according to (k) HC-CDR1: SEQ ID NO: 10, HC-CDR2: SEQ ID NO: 24, HC-CDR3: SEQ ID NO: 39, LC-CDR1: SEQ ID NO: 55, LC-CDR2: SEQ ID NO: 66, LC-CDR3: SEQ ID NO: 80.

Embodiment 81 comprises an antibody of embodiment 69, wherein the HC-CDR1, HC-CDR2, HC-CDR3, LC-CDR1, LC-CDR2, LC-CDR3 comprise sequences according to (1) HC-CDR1: SEQ ID NO: 8, HC-CDR2: SEQ ID NO: 25, HC-CDR3: SEQ ID NO: 40, LC-CDR1: SEQ ID NO: 53, LC-CDR2: SEQ ID NO: 67, LC-CDR3: SEQ ID NO: 78.

Embodiment 82 comprises an antibody of embodiment 69, wherein the HC-CDR1, HC-CDR2, HC-CDR3, LC-CDR1, LC-CDR2, LC-CDR3 comprise sequences according to (m) HC-CDR1: SEQ ID NO: 11, HC-CDR2: SEQ ID NO: 26, HC-CDR3: SEQ ID NO: 41, LC-CDR1: SEQ ID NO: 52, LC-CDR2: SEQ ID NO: 62, LC-CDR3: SEQ ID NO: 81.

Embodiment 83 comprises an antibody of embodiment 69, wherein the HC-CDR1, HC-CDR2, HC-CDR3, LC-CDR1, LC-CDR2, LC-CDR3 comprise sequences according to (n) HC-CDR1: SEQ ID NO: 12, HC-CDR2: SEQ ID NO: 27, HC-CDR3: SEQ ID NO: 42, LC-CDR1: SEQ ID NO: 56, LC-CDR2: SEQ ID NO: 68, LC-CDR3: SEQ ID NO: 82.

Embodiment 84 comprises an antibody of embodiment 69, wherein the HC-CDR1, HC-CDR2, HC-CDR3, LC-CDR1, LC-CDR2, LC-CDR3 comprise sequences according to (o) HC-CDR1: SEQ ID NO: 1, HC-CDR2: SEQ ID NO: 28, HC-CDR3: SEQ ID NO: 30, LC-CDR1: SEQ ID NO: 45, LC-CDR2: SEQ ID NO: 69, LC-CDR3: SEQ ID NO: 70.

Embodiment 85 comprises an antibody of embodiment 69, wherein the HC-CDR1, HC-CDR2, HC-CDR3, LC-CDR1, LC-CDR2, LC-CDR3 comprise sequences according to (q) HC-CDR1: SEQ ID NO: 1, HC-CDR2: SEQ ID NO: 28, HC-CDR3: SEQ ID NO: 177, LC-CDR1: SEQ ID NO: 45, LC-CDR2: SEQ ID NO: 69, LC-CDR3: SEQ ID NO: 70.

Embodiment 86 comprises an antibody of embodiment 69, wherein the HC-CDR1, HC-CDR2, HC-CDR3, LC-CDR1, LC-CDR2, LC-CDR3 comprise sequences according to (r) HC-CDR1: SEQ ID NO: 1, HC-CDR2: SEQ ID NO: 28, HC-CDR3: SEQ ID NO: 178, LC-CDR1: SEQ ID NO: 45, LC-CDR2: SEQ ID NO: 69, LC-CDR3: SEQ ID NO: 70.

Embodiment 87 comprises an antibody of embodiment 69, wherein the HC-CDR1, HC-CDR2, HC-CDR3, LC-CDR1, LC-CDR2, LC-CDR3 comprise sequences according to (s) HC-CDR1: SEQ ID NO: 13, HC-CDR2: SEQ ID NO: 29, HC-CDR3: SEQ ID NO: 43, LC-CDR1: SEQ ID NO: 57, LC-CDR2: SEQ ID NO: 66, LC-CDR3: SEQ ID NO: 83.

Embodiment 88 comprises an antibody of embodiment 69, wherein the HC-CDR1, HC-CDR2, HC-CDR3, LC-CDR1, LC-CDR2, LC-CDR3 comprise sequences according to (t) HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 84.

Embodiment 89 comprises an antibody of embodiment 69, wherein the HC-CDR1, HC-CDR2, HC-CDR3, LC-CDR1, LC-CDR2, LC-CDR3 comprise sequences according to (u) HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 85.

Embodiment 90 comprises an antibody of embodiment 69, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 30, LC-CDR1: SEQ ID NO: 45, LC-CDR2: SEQ ID NO: 91, LC-CDR3: SEQ ID NO: 70 and the heavy chain variable domain comprises an amino acid sequence with at least 90% sequence identity to SEQ ID NO: 143, and the light chain variable domain comprises an amino acid sequence with at least 90% sequence identity to SEQ ID NO: 144.

Embodiment 91 comprises an antibody of embodiment 69, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 30, LC-CDR1: SEQ ID NO: 45, LC-CDR2: SEQ ID NO: 69, LC-CDR3: SEQ ID NO: 70 and the heavy chain variable domain comprises an amino acid sequence with at least 90% sequence identity to SEQ ID NO: 149, and the light chain variable domain comprises an amino acid sequence with at least 90% sequence identity to SEQ ID NO: 150.

Embodiment 92 comprises an antibody of embodiment 69, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 177, LC-CDR1: SEQ ID NO: 45, LC-CDR2: SEQ ID NO: 69, LC-CDR3: SEQ ID NO: 70 and the heavy chain variable domain comprises an amino acid sequence with at least 90% sequence identity to SEQ ID NO: 181, and the light chain variable domain comprises an amino acid sequence with at least 90% sequence identity to SEQ ID NO: 144.

Embodiment 93 comprises an antibody of embodiment 69, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 178, LC-CDR1: SEQ ID NO: 45, LC-CDR2: SEQ ID NO: 69, LC-CDR3: SEQ ID NO: 70 and the heavy chain variable domain comprises an amino acid sequence with at least 90% sequence identity to SEQ ID NO: 184, and the light chain variable domain comprises an amino acid sequence with at least 90% sequence identity to SEQ ID NO: 144.

Embodiment 94 comprises an antibody of embodiment 69, wherein the HC-CDR1, HC-CDR2, HC-CDR3, LC-CDR1, LC-CDR2, LC-CDR3 comprise sequences according to HC-CDR1 SEQ ID NO: 13, HC-CDR2: SEQ ID NO: 29, HC-CDR3: SEQ ID NO: 43, LC-CDR1: SEQ ID NO: 57, LC-CDR2: SEQ ID NO: 66, LC-CDR3: SEQ ID NO: 83 and the heavy chain variable domain comprises an amino acid sequence with at least 90% sequence identity to SEQ ID NO: 155, and the light chain variable domain comprises an amino acid sequence with at least 90% sequence identity to SEQ ID NO: 156.

Embodiment 95 comprises an antibody of embodiment 69, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 84 and the heavy chain variable domain comprises an amino acid sequence with at least 90% sequence identity to SEQ ID NO: 161, and the light chain variable domain comprises an amino acid sequence with at least 90% sequence identity to SEQ ID NO: 162.

Embodiment 96 comprises an antibody of embodiment 69, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 85 and the heavy chain variable domain comprises an amino acid sequence with at least 90% sequence identity to SEQ ID NO: 167, and the light chain variable domain comprises an amino acid sequence with at least 90% sequence identity to SEQ ID NO: 168.

Embodiment 97 comprises an antibody of embodiment 69, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 30, LC-CDR1: SEQ ID NO: 45, LC-CDR2: SEQ ID NO: 69, LC-CDR3: SEQ ID NO: 70 and the antibody comprises amino acid sequences with at least 90% sequence identity to SEQ ID NO: 145, and at least 90% sequence identity to SEQ ID NO: 147.

Embodiment 98 comprises an antibody of embodiment 69, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 30, LC-CDR1: SEQ ID NO: 45, LC-CDR2: SEQ ID NO: 69, LC-CDR3: SEQ ID NO: 70, and the antibody comprises amino acid sequences with at least 90% sequence identity to SEQ ID NO: 151, and at least 90% sequence identity to SEQ ID NO: 153.

Embodiment 99 comprises an antibody of embodiment 69, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 177, LC-CDR1: SEQ ID NO: 45, LC-CDR2: SEQ ID NO: 69, LC-CDR3: SEQ ID NO: 70 and the antibody comprises amino acid sequences with at least 90% sequence identity to SEQ ID NO: 182, and at least 90% sequence identity to SEQ ID NO: 147.

Embodiment 100 comprises an antibody of embodiment 69, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 178, LC-CDR1: SEQ ID NO: 45, LC-CDR2: SEQ ID NO: 69, LC-CDR3: SEQ ID NO: 70 and the antibody comprises amino acid sequences with at least 90% sequence identity to SEQ ID NO: 185, and at least 90% sequence identity to SEQ ID NO: 147.

Embodiment 101 comprises an antibody of embodiment 69, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 43, LC-CDR1: SEQ ID NO: 57, LC-CDR2: SEQ ID NO: 66, LC-CDR3: SEQ ID NO: 83 and the antibody comprises amino acid sequences with at least 90% sequence identity to SEQ ID NO: 157, and at least 90% sequence identity to SEQ ID NO: 159.

Embodiment 102 comprises an antibody of embodiment 69, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 84 and the antibody comprises amino acid sequences with at least 90% sequence identity to SEQ ID NO: 163, and at least 90% sequence identity to SEQ ID NO: 165.

Embodiment 103 comprises an antibody of embodiment 69, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 85 and the antibody comprises amino acid sequences with at least 90% sequence identity to SEQ ID NO: 169, and at least 90% sequence identity to SEQ ID NO: 171.

Embodiment 104 comprises an antibody of any one of embodiments 60-103, wherein the antibody comprises an antibody format selected from Fab, Fab′, scFv, and (Fab′)₂.

Embodiment 105 comprises an antibody of any one of embodiments 60-104, wherein the heavy chain variable domain is fused to a human IgG1 constant region.

Embodiment 106 comprises an antibody of any one of embodiments 60-104, wherein the heavy chain variable domain is fused to a human IgG4 constant region.

Embodiment 107 comprises an antibody of any one of embodiments 60-106, wherein the light chain variable domain is fused to a human Kappa constant region.

Embodiment 108 comprises an antibody of any one of embodiments 60-104, wherein the heavy chain variable domain comprises a variable domain of an IgG1, IgG2, IgG3, or IgG4 heavy chain.

Embodiment 109 comprises an antibody of any one of embodiments 60-108, wherein the light chain variable domain comprises a variable domain of a Kappa light chain.

Embodiment 110 comprises an antibody of embodiment 60, wherein the antibody comprises amino acid sequences with at least 90% sequence identity to SEQ ID NO: 169, and at least 90% sequence identity to SEQ ID NO: 171.

Embodiment 111 comprises an antibody of any one of embodiments 60-110, wherein the antibody binds to extracellular domain of human CD47 with an EC50 of between 0.01 nM-0.5 nM as determined by an ELISA binding assay.

Embodiment 112 comprises an antibody of embodiment 111, wherein the ELISA binding assay comprises the following steps: coating a 96-well plate for at least 12 hours with 1p g/ml of recombinant CD47; washing the plate three times; blocking the plate with 300 μL of 1% bovine serum albumin in phosphate-buffered saline solution with tween (PBST) at 37° C. for 1 hour; washing the plate four times with PBST; incubating a serial dilution of the antibody that binds specifically to CD47 at 37° C. for 1 hour; washing the plate four times with PBST; incubating a 1:5000 dilution of anti-human IgG-peroxidase antibody at 37° C. for 1 hour within the plate; washing the plate 4 times with PBST; incubating 3,3′,5,5′ tetramethylbenzidine substrate for 15 minutes at room temperature within the plate; terminating the reaction within the plate with 1N HCl; and reading the plate at 450 nm to determine the EC50 of the antibody for binding to the extracellular domain of human CD47.

Embodiment 113 comprises an antibody of embodiment 111, wherein the extracellular domain of human CD47 comprises an amino acid sequence according to SEQ ID NO: 86.

Embodiment 114 comprises an antibody of any one of embodiments 60-113, wherein the antibody binds to CD47+ cell lines with an EC50 of between 0.1 nM-5 nM as determined by flow cytometry.

Embodiment 115 comprises an antibody of embodiment 114, wherein the flow cytometry comprises the following steps and assay conditions: centrifuging CD47+ cells at 2000 rpm for 5 minutes to obtain centrifuged cells; resuspending the centrifuged cells in 10-15 mL of culture medium; resuspending cells in blocking buffer comprising phosphate buffer saline with 2% fetal bovine serum (PBS plus 2% FBS) at a concentration of 3×10⁶ cells/mL to obtain a cell suspension; dispensing the cell suspension into wells of a 96-well plate; diluting the antibody that binds specifically to CD47 in the blocking buffer to a desired concentration followed by addition of 100 μL of the antibody per well and incubating at 1 hour for 4° C.; washing the cells three times with PBS plus 2% FBS; resuspending the cells in 100 μL 1:500 diluted Alexa Fluor 488 labeled Mouse anti-Human IgG1 Fc secondary antibody and incubating at 4° C. in the dark for one hour; washing the cells three times with 200 μL PBS and centrifuging at 2000 rpm for 5 minutes; resuspending cells in 300 μL cold PBS; and analyzing with a flow cytometer to determine the EC50 of the antibody for the CD47+ cell line.

Embodiment 116 comprises an antibody of any one of embodiments 60-115, wherein the antibody blocks SIRPa activity with a IC50 of between 0.1 nM-10 nM as determined by flow cytometry.

Embodiment 117 comprises an antibody of embodiment 116, wherein the IC50 for SIRPa activity as determined by flow cytometry comprises the following steps and assay conditions: harvesting, centrifuging, and resuspending Raji tumor cells or HCT-15 tumor cells in FACS buffer comprising phosphate buffer saline with 2% fetal bovine serum (PBS plus 2% FBS) at a concentration of 2×10⁶ cells/mL; dispensing 100 μL of the cell suspension into wells of a 96-well plate; centrifuging the plate for 5 minutes at 300×g followed by discarding of supernatants; incubating cells from the plate with 50 μL per well of serially diluted antibody and a constant amount of SIRPA-mIgG2a fusion protein (0.2 μg/mL for Raji cells, 1 μg/mL for HCT-15 cells) in FACS buffer for 1 hour at 4° C.; washing the plate with FACS buffer followed by incubating for one hour at 4° C. in the dark with 100 μL of Alexa Fluor 488 donkey anti-Mouse IgG(H+L) secondary antibody; washing twice with FACS buffer; resuspending cells in the plate with 300 μL FACS buffer; and analyzing with a flow cytometer to determine the IC50 of the antibody for blocking SIRPa activity.

Embodiment 118 comprises an antibody of any one of embodiments 60-117, wherein the antibody induces increased antibody-dependent cellular phagocytosis (ADCP) as compared to a control antibody that comprises amino acid sequences according to SEQ ID NOs: 173 and 174 when tested under substantially equivalent assay conditions in an assay for ADCP.

Embodiment 119 comprises an antibody of any one of embodiments 60-118, wherein the antibody has less binding on human red blood cells (RBC) as compared to a control antibody that comprises amino acid sequences according to SEQ ID NOs: 173 and 174 when tested under substantially equivalent assay conditions for assessing RBC binding.

Embodiment 120 comprises an antibody of embodiment 119, wherein a concentration of 600 nM of the antibody does not induce hemolysis of red blood cells in a hemagglutination assay.

Embodiment 121 comprises an antibody of any one of embodiments 60-120, wherein the antibody induces increased reduction in tumor volume as compared to a control antibody that comprises amino acid sequences according to SEQ ID NOs: 173 and 174 when tested under substantially equivalent assay conditions in a tumor growth animal model.

Embodiment 122 1 comprises an antibody that binds specifically to CD47 wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain are selected from the following amino acid sequences HC-CDR1: SEQ ID NOs: 1 and 13, HC-CDR2: SEQ ID NOs: 28 and 29, HC-CDR3: SEQ ID NOs: 30, 177-178, 43-44; and a light chain variable domain that comprises CDRs: LC-CDR1, LC-CDR2, LC-CDR3, wherein the LC-CDR1, LC-CDR2, LC-CDR3 of the light chain variable domain are selected from the following amino acid sequences LC-CDR1: SEQ ID NOs: 45 and 57, LC-CDR2: SEQ ID NOs: 66 and 69, LC-CDR3: SEQ ID NOs: 70, and 83-85, and wherein the CDRs comprise 0-2 amino acid mutations, substitutions, or deletions in at least one of the HC-CDR1, HC-CDR2, HC-CDR3, LC-CDR1, LC-CDR2, LC-CDR3.

Embodiment 123 comprises an antibody of embodiment 122, wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain are selected from the following amino acid sequences HC-CDR1: SEQ ID NOs: 1 and 13, HC-CDR2: SEQ ID NOs: 28 and 29, HC-CDR3: SEQ ID NOs: 30, 177-178, 43-44; and a light chain variable domain that comprises CDRs: LC-CDR1, LC-CDR2, LC-CDR3, wherein the LC-CDR1, LC-CDR2, LC-CDR3 of the light chain variable domain are selected from the following amino acid sequences LC-CDR1: SEQ ID NOs: 45 and 57, LC-CDR2: SEQ ID NOs: 66 and 69, LC-CDR3: SEQ ID NOs: 70, and 83-85, and wherein the CDRs comprise 0-2 amino acid mutations, substitutions, or deletions in at least one of the HC-CDR3 or LC-CDR3.

Embodiment 124 comprises an antibody of embodiment 122 or 123, wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain are selected from the following amino acid sequences HC-CDR1: SEQ ID NOs: 1 and 13, HC-CDR2: SEQ ID NOs: 28 and 29, HC-CDR3: SEQ ID NOs: 30, 177-178, 43-44; and a light chain variable domain that comprises CDRs: LC-CDR1, LC-CDR2, LC-CDR3, wherein the LC-CDR1, LC-CDR2, LC-CDR3 of the light chain variable domain are selected from the following amino acid sequences LC-CDR1: SEQ ID NOs: 45 and 57, LC-CDR2: SEQ ID NOs: 66 and 69, LC-CDR3: SEQ ID NOs: 70, and 83-85.

Embodiment 125 comprises an antibody of any one of embodiments 122-124, wherein the CDRs of the heavy chain variable domain and the CDRs of the light chain variable domain are selected from the following sets of amino acid sequences: (a) HC-CDR1: SEQ ID NO: 1, HC-CDR2: SEQ ID NO: 28, HC-CDR3: SEQ ID NO: 30, LC-CDR1: SEQ ID NO: 45, LC-CDR2: SEQ ID NO: 69, LC-CDR3: SEQ ID NO: 70; (b) HC-CDR1: SEQ ID NO: 1, HC-CDR2: SEQ ID NO: 28, HC-CDR3: SEQ ID NO: 177, LC-CDR1: SEQ ID NO: 45, LC-CDR2: SEQ ID NO: 69, LC-CDR3: SEQ ID NO: 70; (c) HC-CDR1: SEQ ID NO: 1, HC-CDR2: SEQ ID NO: 28, HC-CDR3: SEQ ID NO: 178, LC-CDR1: SEQ ID NO: 45, LC-CDR2: SEQ ID NO: 69, LC-CDR3: SEQ ID NO: 70; (d) HC-CDR1: SEQ ID NO: 13, HC-CDR2: SEQ ID NO: 29, HC-CDR3: SEQ ID NO: 43, LC-CDR1: SEQ ID NO: 57, LC-CDR2: SEQ ID NO: 66, LC-CDR3: SEQ ID NO: 83; (e) HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 84; and (f) HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 85.

Embodiment 126 comprises an antibody of embodiment 125, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 30, LC-CDR1: SEQ ID NO: 45, LC-CDR2: SEQ ID NO: 69, LC-CDR3: SEQ ID NO: 70.

Embodiment 127 comprises an antibody of embodiment 125, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 177, LC-CDR1: SEQ ID NO: 45, LC-CDR2: SEQ ID NO: 69, LC-CDR3: SEQ ID NO: 70.

Embodiment 128 comprises an antibody of embodiment 125, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 178, LC-CDR1: SEQ ID NO: 45, LC-CDR2: SEQ ID NO: 69, LC-CDR3: SEQ ID NO: 70.

Embodiment 129 comprises an antibody of embodiment 125, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 43, LC-CDR1: SEQ ID NO: 57, LC-CDR2: SEQ ID NO: 66, LC-CDR3: SEQ ID NO: 83.

Embodiment 130 comprises an antibody of embodiment 125, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 84.

Embodiment 131 comprises an antibody of embodiment 125, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 85.

Embodiment 132 comprises an antibody of embodiment 125, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 30, LC-CDR1: SEQ ID NO: 45, LC-CDR2: SEQ ID NO: 69, LC-CDR3: SEQ ID NO: 70 and the heavy chain variable domain comprises an amino acid sequence with at least 90% sequence identity to SEQ ID NO: 143, and the light chain variable domain comprises an amino acid sequence with at least 90% sequence identity to SEQ ID NO: 144.

Embodiment 133 comprises an antibody of embodiment 125, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 30, LC-CDR1: SEQ ID NO: 45, LC-CDR2: SEQ ID NO: 69, LC-CDR3: SEQ ID NO: 70 and the heavy chain variable domain comprises an amino acid sequence with at least 90% sequence identity to SEQ ID NO: 149, and the light chain variable domain comprises an amino acid sequence with at least 90% sequence identity to SEQ ID NO: 150.

Embodiment 134 comprises an antibody of embodiment 125, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 177, LC-CDR1: SEQ ID NO: 45, LC-CDR2: SEQ ID NO: 69, LC-CDR3: SEQ ID NO: 70 and the heavy chain variable domain comprises an amino acid sequence with at least 90% sequence identity to SEQ ID NO: 181, and the light chain variable domain comprises an amino acid sequence with at least 90% sequence identity to SEQ ID NO: 144.

Embodiment 135 comprises an antibody of embodiment 125, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 178, LC-CDR1: SEQ ID NO: 45, LC-CDR2: SEQ ID NO: 69, LC-CDR3: SEQ ID NO: 70 and the heavy chain variable domain comprises an amino acid sequence with at least 90% sequence identity to SEQ ID NO: 184, and the light chain variable domain comprises an amino acid sequence with at least 90% sequence identity to SEQ ID NO: 144.

Embodiment 136 comprises an antibody of embodiment 125, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 43, LC-CDR1: SEQ ID NO: 57, LC-CDR2: SEQ ID NO: 66, LC-CDR3: SEQ ID NO: 83 and the heavy chain variable domain comprises an amino acid sequence with at least 90% sequence identity to SEQ ID NO: 155, and the light chain variable domain comprises an amino acid sequence with at least 90% sequence identity to SEQ ID NO: 156.

Embodiment 137 comprises an antibody of embodiment 125, wherein the HC-CDR1, HC-CDR2, HC-CDR3, LC-CDR1, LC-CDR2, LC-CDR3 comprise sequences according to HC-CDR1 SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 84 and the heavy chain variable domain comprises an amino acid sequence with at least 90% sequence identity to SEQ ID NO: 161, and the light chain variable domain comprises an amino acid sequence with at least 90% sequence identity to SEQ ID NO: 162.

Embodiment 138 comprises an antibody of embodiment 125, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 85 and the heavy chain variable domain comprises an amino acid sequence with at least 90% sequence identity to SEQ ID NO: 167, and the light chain variable domain comprises an amino acid sequence with at least 90% sequence identity to SEQ ID NO: 168.

Embodiment 139 comprises an antibody of embodiment 125, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 30, LC-CDR1: SEQ ID NO: 45, LC-CDR2: SEQ ID NO: 69, LC-CDR3: SEQ ID NO: 70 and the antibody comprises amino acid sequences with at least 90% sequence identity to SEQ ID NO: 145, and at least 90% sequence identity to SEQ ID NO: 147.

Embodiment 140 comprises an antibody of embodiment 125, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 30, LC-CDR1: SEQ ID NO: 45, LC-CDR2: SEQ ID NO: 69, LC-CDR3: SEQ ID NO: 70, and the antibody comprises amino acid sequences with at least 90% sequence identity to SEQ ID NO: 151, and at least 90% sequence identity to SEQ ID NO: 153.

Embodiment 141 comprises an antibody of embodiment 125, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 177, LC-CDR1 SEQ ID NO: 45, LC-CDR2: SEQ ID NO: 69, LC-CDR3: SEQ ID NO: 70 and the antibody comprises amino acid sequences with at least 90% sequence identity to SEQ ID NO: 182, and at least 90% sequence identity to SEQ ID NO: 147.

Embodiment 142 comprises an antibody of embodiment 125, wherein the HC-CDR1, HC-CDR2, HC-CDR3, LC-CDR1, LC-CDR2, LC-CDR3 comprise sequences according to HC-CDR1: SEQ ID NO: 1, HC-CDR2: SEQ ID NO: 28, HC-CDR3: SEQ ID NO: 178, LC-CDR1: SEQ ID NO: 45, LC-CDR2: SEQ ID NO: 69, LC-CDR3: SEQ ID NO: 70 and the antibody comprises amino acid sequences with at least 90% sequence identity to SEQ ID NO: 185, and at least 90% sequence identity to SEQ ID NO: 147.

Embodiment 143 comprises an antibody of embodiment 125, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 43, LC-CDR1: SEQ ID NO: 57, LC-CDR2: SEQ ID NO: 66, LC-CDR3: SEQ ID NO: 83 and the antibody comprises amino acid sequences with at least 90% sequence identity to SEQ ID NO: 157, and at least 90% sequence identity to SEQ ID NO: 159.

Embodiment 144 comprises an antibody of embodiment 125, wherein the HC-CDR1, HC-CDR2, HC-CDR3, LC-CDR1, LC-CDR2, LC-CDR3 comprise sequences according to HC-CDR1 SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 84 and the antibody comprises amino acid sequences with at least 90% sequence identity to SEQ ID NO: 163, and at least 90% sequence identity to SEQ ID NO: 165.

Embodiment 145 comprises an antibody of embodiment 125, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 85 and the antibody comprises amino acid sequences with at least 90% sequence identity to SEQ ID NO: 169, and at least 90% sequence identity to SEQ ID NO: 171.

Embodiment 146 comprises an antibody of any one of embodiments 122-145, wherein the antibody comprises an antibody format selected from Fab, Fab′, scFv, and (Fab′)₂.

Embodiment 147 comprises an antibody of any one of embodiments 122-146, wherein the heavy chain variable domain is fused to a human IgG1 constant region.

Embodiment 148 comprises an antibody of any one of embodiments 122-146, wherein the heavy chain variable domain is fused to a human IgG4 constant region.

Embodiment 149 comprises an antibody of any one of embodiments 122-148, wherein the light chain variable domain is fused to a human Kappa constant region.

Embodiment 150 comprises an antibody of any one of embodiments 122-149, wherein the heavy chain variable domain comprises a variable domain of an IgG1, IgG2, IgG3, or IgG4 heavy chain.

Embodiment 151 comprises an antibody of any one of embodiments 122-150, wherein the light chain variable domain comprises a variable domain of a Kappa light chain.

Embodiment 152 comprises an antibody of embodiment 122, wherein the antibody comprises amino acid sequences with at least 90% sequence identity to SEQ ID NO: 169, and at least 90% sequence identity to SEQ ID NO: 171.

Embodiment 153 comprises an antibody of any one of embodiments 122-152, wherein the antibody binds to extracellular domain of human CD47 with an EC50 of between 0.01 nM-0.5 nM as determined by an ELISA binding assay.

Embodiment 154 comprises an antibody of embodiment 153, wherein the ELISA binding assay comprises the following steps: coating a 96-well plate for at least 12 hours with 1p g/ml of recombinant CD47; washing the plate three times; blocking the plate with 300 μL of 1% bovine serum albumin in phosphate-buffered saline solution with tween (PBST) at 37° C. for 1 hour; washing the plate four times with PBST; incubating a serial dilution of the antibody that binds specifically to CD47 at 37° C. for 1 hour; washing the plate four times with PBST; incubating a 1:5000 dilution of anti-human IgG-peroxidase antibody at 37° C. for 1 hour within the plate; washing the plate 4 times with PBST; incubating 3,3′,5,5′ tetramethylbenzidine substrate for 15 minutes at room temperature within the plate; terminating the reaction within the plate with 1N HCl; and reading the plate at 450 nm to determine the EC50 of the antibody for binding to the extracellular domain of human CD47.

Embodiment 155 comprises an antibody of embodiment 153, wherein the extracellular domain of human CD47 comprises an amino acid sequence according to SEQ ID NO: 86.

Embodiment 156 comprises an antibody of any one of embodiments 122-155, wherein the antibody binds to CD47+ cell lines with an EC50 of between 0.01 nM-5 nM as determined by flow cytometry.

Embodiment 157 comprises an antibody of embodiment 156, wherein the flow cytometry comprises the following steps and assay conditions: centrifuging CD47+ cells at 2000 rpm for 5 minutes to obtain centrifuged cells; resuspending the centrifuged cells in 10-15 mL of culture medium; resuspending cells in blocking buffer comprising phosphate buffer saline with 2% fetal bovine serum (PBS plus 2% FBS) at a concentration of 3×10⁶ cells/mL to obtain a cell suspension; dispensing the cell suspension into wells of a 96-well plate; diluting the antibody that binds specifically to CD47 in the blocking buffer to a desired concentration followed by addition of 100 μL of the antibody per well and incubating at 1 hour for 4° C.; washing the cells three times with PBS plus 2% FBS; resuspending the cells in 100 μL 1:500 diluted Alexa Fluor 488 labeled Mouse anti-Human IgG1 Fc secondary antibody and incubating at 4° C. in the dark for one hour; washing the cells three times with 200 μL PBS and centrifuging at 2000 rpm for 5 minutes; resuspending cells in 300 μL cold PBS; and analyzing with a flow cytometer to determine the EC50 of the antibody for the CD47+ cell line.

Embodiment 158 comprises an antibody of any one of embodiments 122-157, wherein the antibody blocks SIRPa activity with a IC50 of between 0.1 nM-10 nM as determined by flow cytometry.

Embodiment 159 comprises an antibody of embodiment 158, wherein the IC50 for SIRPa activity as determined by flow cytometry comprises the following steps and assay conditions: harvesting, centrifuging, and resuspending Raji tumor cells or HCT-15 tumor cells in FACS buffer comprising phosphate buffer saline with 2% fetal bovine serum (PBS plus 2% FBS) at a concentration of 2×10⁶ cells/mL; dispensing 100 μL of the cell suspension into wells of a 96-well plate; centrifuging the plate for 5 minutes at 300×g followed by discarding of supernatants; incubating cells from the plate with 50 μL per well of serially diluted antibody and a constant amount of SIRPA-mIgG2a fusion protein (0.2 μg/mL for Raji cells, 1 μg/mL for HCT-15 cells) in FACS buffer for 1 hour at 4° C.; washing the plate with FACS buffer followed by incubating for one hour at 4° C. in the dark with 100 μL of Alexa Fluor 488 donkey anti-Mouse IgG(H+L) secondary antibody; washing twice with FACS buffer; resuspending cells in the plate with 300 μL FACS buffer; and analyzing with a flow cytometer to determine the IC50 of the antibody for blocking SIRPa activity.

Embodiment 160 comprises an antibody of any one of embodiments 122-159, wherein the antibody induces increased antibody-dependent cellular phagocytosis (ADCP) as compared to a control antibody that comprises amino acid sequences according to SEQ ID NOs: 173 and 174 when tested under substantially equivalent assay conditions in an assay for ADCP.

Embodiment 161 comprises an antibody of any one of embodiments 122-160, wherein the antibody has less binding on human red blood cells (RBC) as compared to a control antibody that comprises amino acid sequences according to SEQ ID NOs: 173 and 174 when tested under substantially equivalent assay conditions for assessing RBC binding.

Embodiment 162 comprises an antibody of embodiment 161, wherein a concentration of 600 nM of the antibody does not induce hemolysis of red blood cells in a hemagglutination assay.

Embodiment 163 comprises an antibody of any one of embodiments 122-162, wherein the antibody induces increased reduction in tumor volume as compared to a control antibody that comprises amino acid sequences according to SEQ ID NOs: 173 and 174 when tested under substantially equivalent assay conditions in a tumor growth animal model.

Embodiment 164 comprises a nucleic acid molecule encoding the antibody of any one of embodiments 1-163.

Embodiment 165 comprises a vector comprising the nucleic acid molecule of embodiment 164.

Embodiment 166 comprises a pharmaceutical composition comprising the antibody of any one of embodiments 1-163.

Embodiment 167 comprises the pharmaceutical composition of embodiment 166, further comprising a pharmaceutically acceptable carrier, an excipient, or any combinations thereof.

Embodiment 168 comprises a method of treating a subject having cancer, the method comprising: administering to the subject the antibody of any one of embodiments 1-163 or the pharmaceutical composition of embodiment 167.

Embodiment 169 comprises the method of embodiment 168, wherein the cancer comprises cancer cells that express CD47.

Embodiment 170 comprises the method of any one of embodiments 168-169, wherein the antibody induces antibody-dependent cellular phagocytosis (ADCP) of the cancer cells that express CD47.

Embodiment 171 comprises the method of any one of embodiments 168-170, wherein the cancer is a hematological malignancy.

Embodiment 172 comprises the method of embodiment 171, wherein the cancer is B cell cancer or T cell cancer.

Embodiment 173 comprises the method of embodiment 171, wherein the cancer is leukemia or lymphoma.

Embodiment 174 comprises the method of embodiment 173, wherein the cancer is lymphoma, and wherein the lymphoma is B-cell lymphoma.

Embodiment 175 comprises the method of embodiment 173, wherein the cancer is lymphoma, and wherein the lymphoma is T-cell lymphoma

Embodiment 176 comprises the method of any one of embodiments 168-170, wherein the cancer is a solid tumor.

Embodiment 177 comprises the method of embodiment 176, wherein the solid tumor is sarcoma, breast cancer, lung cancer, carcinoma, ovarian cancer, pancreatic cancer, gastric cancer, colorectal cancer, endometrial cancer, esophageal cancer, prostate cancer, cervical cancer, kidney cancer, urothelial cancer, or head and neck cancer.

Embodiment 178 comprises the method of embodiment 176, wherein the solid tumor is lung cancer, and wherein the lung cancer is non-small cell lung cancer.

Embodiment 179 comprises the method of embodiment 176, wherein the solid tumor is lung cancer, and wherein the lung cancer is small cell lung cancer.

Embodiment 180 comprises the method of any one of embodiments 168-179, further comprising administering to the subject an anti-cancer agent.

Embodiment 181 comprises the method of embodiment 180, wherein the anti-cancer agent is a chemotherapeutic agent or a biologic agent.

Embodiment 182 comprises the method of any one of embodiments 168-181, wherein the administering is sufficient to reduce or eliminate the cancer as compared to a comparable method lacking the administering.

Embodiment 183 comprises the method of embodiment 182, wherein the reduction is at least about 1 fold, 5 fold, 10 fold, 20 fold, 40 fold, 60 fold, 80 fold, or up to about 100 fold.

Embodiment 184 comprises the method of any one of embodiments 168-183, wherein the cancer is metastatic.

Embodiment 185 comprises a kit that comprises at least one of: the antibody of any one of embodiments 1-163; the vector of embodiment 165; the nucleic acid molecule of embodiment 164; or the pharmaceutical composition of embodiment 167.

### EXAMPLES

### Example 1: Generation of Human/Cyno CD47 Antigens

DNA encoding the extracellular domains (ECD) of human CD47 (19aa-139aa) and cyno CD47 (4aa-126aa) were cloned into pRK5 (ATCC Cat #209784). The resulting constructs with C-terminal 6×His tags (SEQ ID NO: 187) were transfected into Expi293F cells. After 72 hours, CD47-expressing cells were harvested by centrifugation for 5 minutes at 2000 rpm at 4° C. The supernatant was collected. Ni-NTA (Qiagen, Cat #30410) resin was pre-equilibrated with buffer A (137 mM NaCl, 2.7 mM KCl, 10 mM Na2HPO4, 2 mM KH2PO4, pH 7.4) and incubated with the supernatant for 2 hours at 4° C. on a rotator. The resin was filled in the Ni Sepharose excel (GE, Cat #GE17371201) and washed with buffer A until no signal (OD595, about 20-30 column volumes (CV) was observed by Coomassie-Brilliant Blue G-250. The target protein was eluted using buffer B (137 mM NaCl, 2.7 mM KCl, 10 mM Na2HPO4, 2 mM KH2PO4, pH 7.4, 250 mM imidazole) for 3 column volumes (CV). The SuperdexTX 200 increase column (GE, Cat #GE28-9909-44) was pre-equilibrated by buffer A, then the eluate was loaded onto the column. The column was washed with buffer A and fractions were collected. Different fractions were resolved on a 12% SDS-PAGE and desired fractions were combined and neutralized with buffer C (137 mM NaCl, 2.7 mM KCl, 10 mM Na2HPO4, 2 mM KH2PO4, pH 7.4). The target protein was concentrated by using an ultra-filtration tube (Amicon, Cat #42409) with a molecular cutoff of 30 kDa, then aliquoted and snap frozen using liquid N2 and stored at −80° C.

TABLE 3
Antigen Sequences

| | | SEQ |
|---|---|---|
| | | ID |
| Antigen | Sequence | NO: |
| (ECD) of | QLLFNKTKSVEFTFCNDTVVIPCFVTNMEAQ | 86 |
| human CD47 | NTTEVYVKWKFKGRDIYTFDGALNKSTVPTD | |
| (19aa-139aa) | FSSAKIEVSQLLKGDASLKMDKSDAVSHTGN | |
| | YTCEVTELTREGETIIELKYRVVSWFSP | |
| cyno CD47 | QLLFNKTKSVEFTFCNDTVVIPCFVTNMEAQ | 87 |
| (4aa-126aa) | NTTEVYVKWKFKGRDIYTFDGALNKSTAPAN | |
| | FSSAKIEVSQLLKGDASLKMDKSDAVSHTGN | |
| | YTCEVTELTREGETIIELKYRVVSWFSPN | |

### Example 2: Generation of Anti-Hu/Cyno CD47 Antibodies

Generation of Anti-Hu Cyno CD47 Antibodies from Mouse Hybridoma

Female Balb/c & SJL mice were immunized with human CD47 protein mixed with one of the following three types of adjuvant at the ratio of 1:1: Freud's adjuvant (Sigma-Aldrich), Aluminum sulfate, and Ribi. All mice were immunized multiple times before harvesting splenocyte for hybridoma fusion. Serum samples were collected from each immunized mouse, then tested by ELISA & FACS.

Splenocytes were isolated from immunized animals which showed strong ELISA and FACS binding titer on huCD47/cyno CD47 antigen and hu/cyno CD47 over-expressing CHO cell line, followed by electro fusion process (BTX) with SP2/0 myeloma cells. Fused hybridoma cells were cultured in DMEM/10% FBS containing selection reagents including 1% hypoxantin, 1% aminopterin and 1% thymidine. Conditioned media samples after 7 days culture were collected for further ELISA/FACS screening on hu/cyno CD47 antigen and hu/cyno CD47 over-expressing CHO cell lines.

The hybridoma clones which showed ELISA/FACS binding positive on hu/cyno CD47 antigen and hu/cyno CD47 over-expressing CHO cell lines were selected for single hybridoma cell subcloning.

Total RNA were extracted from selected hybridoma cells after subcloning using TRIzol (Ambion, Cat. No.:15596-026), followed by RT-PCR using PrimeScript™ 1stStrand cDNA Synthesis Kit (Takara, Cat. No.: 6110A). Antibody variable region from cDNA samples were then amplified using RACE PCR (GenScript), and then cloned into pMD18-T vector (Takara, CAT #6011) for sequencing (GenScript). DNA sequence of antibody variable region from both heavy chain and light chain were obtained by primers specific to pMD18-T vector.

Chimeric antibodies with mouse variable domains and human constant domains were generated by fusing the VH domain of the anti-CD47 monoclonal antibodies to a human IgG1 constant region and the VL domain of the anti-CD47 monoclonal antibodies to a human Kappa constant region. Table 4 presents sequences of exemplary anti-CD47 antibodies discovered by these methods including their complementarity-determining regions (CDRs), heavy and light chains. CDRs were determined by Kabat numbering system.

TABLE 4
Anti-CD47 Antibody Sequences
from Mouse Hybridoma

| | | SEQ |
|---|---|---|
| Construct | | ID |
| Description | Sequence | NO: |
| M.1D15E11_ | SYVMH | 1 |
| HC: CDR-1 | | |
| M.1D15E11_ | YIDPYNDGTKYNEKFKG | 14 |
| HC: CDR-2 | | |
| M.1D15E11_ | GGMARRSFDY | 30 |
| HC: CDR-3 | | |
| M.1D15E11_ | LASQTIGTWLT | 45 |
| LC: CDR-1 | | |
| M.1D15E11_ | AATTLAD | 58 |
| LC: CDR-2 | | |
| M.1D15E11_ | QQLYSTPWT | 70 |
| LC: CDR-3 | | |
| M.1D15E11_ | EVQLQQSGPELVKPGAPVKMSCKASGYTFTSYV | 88 |
| HC | MHWVKQKPGQGLEWIGYIDPYNDGTKYNEKFKG | |
| | KATLTSDKSSSTAYMELSSLTSEDSAVYYCSRG | |
| | GMARRSFDYWGQGTTLTVSSASTKGPSVFPLAP | |
| | SSKSTSGGTAALGCLVKDYFPEPVTVSWNSGAL | |
| | TSGVHTFPAVLQSSGLYSLSSVVTVPSSSLGTQ | |
| | TYICNVNHKPSNTKVDKKVEPKSCDKTHTCPPC | |
| | PAPELLGGPSVFLFPPKPKDTLMISRTPEVTCV | |
| | VVDVSHEDPEVKFNWYVDGVEVHNAKTKPREEQ | |
| | YNSTYRVVSVLTVLHQDWLNGKEYKCKVSNKAL | |
| | PAPIEKTISKAKGQPREPQVYTLPPSRDELTKN | |
| | QVSLTCLVKGFYPSDIAVEWESNGQPENNYKTT | |
| | PPVLDSDGSFFLYSKLTVDKSRWQQGNVFSCSV | |
| | MHEALHNHYTQKSLSLSPGK | |
| M.1D15E11_ | GAGGTGCAGCTGCAGCAGAGCGGGCCAGAGCTG | 89 |
| HC | GTGAAGCCAGGAGCCCCAGTGAAGATGAGCTGT | |
| (Nucleo- | AAGGCCAGCGGGTACACATTTACAAGCTACGTG | |
| tide) | ATGCACTGGGTGAAGCAGAAGCCCGGACAGGGC | |
| | CTGGAATGGATCGGATATATTGACCCCTATAAT | |
| | GACGGCACAAAGTACAACGAGAAATTCAAGGGC | |
| | AAAGCCACACTGACCAGCGACAAGAGCAGCAGC | |
| | ACCGCCTACATGGAGCTGAGCAGCCTGACCAGC | |
| | GAAGACAGCGCCGTGTACTACTGCAGCAGAGGC | |
| | GGAATGGCCAGGAGATCCTTTGACTACTGGGGC | |
| | CAGGGCACAACACTGACAGTGTCTAGCGCTAGC | |
| | ACCAAAGGTCCCAGCGTGTTCCCACTCGCCCCG | |
| | AGTTCAAAATCAACTTCTGGAGGCACCGCCGCC | |
| | CTGGGTTGCCTGGTAAAGGACTACTTCCCAGAG | |
| | CCCGTGACCGTGAGCTGGAACTCCGGGGCACTG | |
| | ACATCTGGCGTTCATACTTTCCCGGCCGTGCTC | |
| | CAGTCTTCAGGTCTGTATAGTCTCTCCTCTGTG | |
| | GTCACTGTCCCATCTAGCTCTCTGGGCACCCAA | |
| | ACCTACATATGCAACGTTAATCACAAGCCGAGC | |
| | AATACTAAAGTTGACAAAAAGGTGGAACCCAAG | |
| | TCTTGTGACAAGACCCACACGTGTCCCCCCTGC | |
| | CCGGCTCCTGAGCTGCTTGGCGGCCCCAGCGTC | |
| | TTTCTCTTTCCCCCAAAGCCAAAAGATACCTTG | |
| | ATGATCAGCAGAACTCCCGAGGTGACATGCGTC | |
| | GTCGTGGACGTAAGCCATGAAGATCCCGAGGTT | |
| | AAGTTCAACTGGTATGTCGATGGCGTGGAAGTC | |
| | CATAATGCTAAGACTAAACCTCGCGAAGAGCAG | |
| | TACAATTCAACTTACCGGGTCGTTTCCGTTCTG | |
| | ACCGTGCTGCATCAGGACTGGCTGAATGGTAAA | |
| | GAGTACAAGTGCAAAGTGTCTAACAAGGCACTC | |
| | CCCGCCCCAATTGAGAAGACTATCTCCAAAGCT | |
| | AAAGGGCAACCAAGAGAGCCCCAGGTCTACACC | |
| | CTGCCCCCCTCAAGGGATGAGCTTACTAAGAAC | |
| | CAGGTTAGTCTCACCTGCTTGGTTAAAGGATTT | |
| | TATCCAAGCGATATTGCTGTGGAGTGGGAGTCC | |
| | AACGGCCAGCCTGAGAACAATTATAAAACCACC | |
| | CCCCCTGTTCTTGACAGTGACGGTAGTTTCTTC | |
| | CTGTATTCCAAACTGACCGTCGATAAGAGCAGA | |
| | TGGCAACAGGGAAATGTGTTCAGCTGCTCCGTG | |
| | ATGCATGAGGCGCTCCATAATCATTACACACAA | |
| | AAAAGTTTGTCCCTGAGCCCAGGCAAG | |
| M.1D15E11_ | DIQMTQSPASQSASLGESVTITCLASQTIGTWL | 90 |
| LC | TWYQQKPGKSPQLLIYAATTLADGVPSRFSGSG | |
| | SGTKFSFKISSLQAEDFVSYYCQQLYSTPWTFG | |
| | GGTKLEIKRTVAAPSVFIFPPSDEQLKSGTASV | |
| | VCLLNNFYPREAKVQWKVDNALQSGNSQESVTE | |
| | QDSKDSTYSLSSTLTLSKADYEKHKVYACEVTH | |
| | QGLSSPVTKSFNRGEC | |
| M.1D15E11_ | GACATTCAGATGACCCAGAGCCCCGCCAGCCAG | 91 |
| LC | AGCGCCTCACTGGGAGAGAGCGTGACCATCACC | |
| (Nucleo- | TGCCTGGCCAGCCAGACCATCGGAACCTGGCTG | |
| tide) | ACCTGGTACCAGCAGAAACCCGGCAAAAGCCCC | |
| | CAGCTGCTGATTTACGCCGCCACCACCCTGGCC | |
| | GACGGCGTGCCTTCAAGATTTTCCGGCAGCGGC | |
| | AGCGGCACCAAATTCTCCTTCAAGATCAGCAGC | |
| | CTGCAGGCCGAAGACTTCGTGTCCTACTACTGC | |
| | CAGCAGCTGTACTCCACCCCCTGGACCTTCGGC | |
| | GGCGGCACAAAACTGGAGATCAAGCGTACGGTG | |
| | GCAGCTCCATCAGTTTTTATCTTCCCACCAAGC | |
| | GACGAGCAATTGAAGTCCGGCACTGCCTCTGTG | |
| | GTGTGCCTTCTGAACAACTTCTATCCAAGGGAG | |
| | GCCAAGGTCCAGTGGAAGGTCGATAATGCGCTG | |
| | CAGAGCGGGAACAGCCAAGAGTCAGTGACCGAG | |
| | CAGGACTCAAAAGATAGCACATACTCTCTGAGT | |
| | TCCACCCTGACCCTGTCAAAGGCTGACTACGAA | |
| | AAGCATAAGGTATACGCATGCGAAGTGACCCAT | |
| | CAGGGTCTCTCATCTCCCGTAACCAAATCTTTT | |
| | AATAGAGGAGAATGCTAG | |
| M.20B10E9_ | DTYMH | 2 |
| HC: CDR-1 | | |
| M.20B10E9_ | RIDPANGKTKSDPKFQG | 15 |
| HC: CDR-2 | | |
| M.20B10E9_ | GDGYYVGGLDY | 31 |
| HC: CDR-3 | | |
| M.20B10E9_ | HASQNIHVWLS | 46 |
| LC: CDR-1 | | |
| M.20B10E9_ | KASNLHT | 59 |
| LC: CDR-2 | | |
| M.20B10E9_ | QQGQSYPYT | 71 |
| LC: CDR-3 | | |
| M.20B10E9_ | EVQLQQSGAELVRPGASVKLSCTASGFNIEDTY | 92 |
| HC | MHWVKQRPEQGLEWIGRIDPANGKTKSDPKFQG | |
| | KATITTDTSSNTAYLQLSSLTSEDTAVYYCAKG | |
| | DGYYVGGLDYWGQGTTLTVSSASTKGPSVFPLA | |
| | PSSKSTSGGTAALGCLVKDYFPEPVTVSWNSGA | |
| | LTSGVHTFPAVLQSSGLYSLSSVVTVPSSSLGT | |
| | QTYICNVNHKPSNTKVDKKVEPKSCDKTHTCPP | |
| | CPAPELLGGPSVFLFPPKPKDTLMISRTPEVTC | |
| | VVVDVSHEDPEVKFNWYVDGVEVHNAKTKPREE | |
| | QYNSTYRVVSVLTVLHQDWLNGKEYKCKVSNKA | |
| | LPAPIEKTISKAKGQPREPQVYTLPPSRDELTK | |
| | NQVSLTCLVKGFYPSDIAVEWESNGQPENNYKT | |
| | TPPVLDSDGSFFLYSKLTVDKSRWQQGNVFSCS | |
| | VMHEALHNHYTQKSLSLSPGK | |
| M.20B10E9_ | GAGGTGCAGCTGCAGCAGAGCGGGGCCGAGCTG | 93 |
| HC | GTGAGACCAGGAGCCAGCGTGAAGCTGAGCTGT | |
| (Nucleo- | ACAGCCAGCGGCTTCAACATCGAGGACACATAT | |
| tide) | ATGCACTGGGTGAAACAGAGGCCCGAGCAGGGC | |
| | CTGGAGTGGATCGGCAGAATCGACCCCGCTAAC | |
| | GGAAAAACAAAATCAGACCCCAAATTCCAGGGC | |
| | AAAGCCACCATCACCACCGACACCAGCAGCAAC | |
| | ACCGCCTATCTGCAGCTGAGCAGCCTGACCAGC | |
| | GAGGACACCGCCGTGTACTACTGCGCCAAAGGC | |
| | GACGGCTACTACGTGGGCGGACTGGACTACTGG | |
| | GGCCAGGGAACCACCCTGACAGTGAGCAGCGCT | |
| | AGCACCAAAGGTCCCAGCGTGTTCCCACTCGCC | |
| | CCGAGTTCAAAATCAACTTCTGGAGGCACCGCC | |
| | GCCCTGGGTTGCCTGGTAAAGGACTACTTCCCA | |
| | GAGCCCGTGACCGTGAGCTGGAACTCCGGGGCA | |
| | CTGACATCTGGCGTTCATACTTTCCCGGCCGTG | |
| | CTCCAGTCTTCAGGTCTGTATAGTCTCTCCTCT | |
| | GTGGTCACTGTCCCATCTAGCTCTCTGGGCACC | |
| | CAAACCTACATATGCAACGTTAATCACAAGCCG | |
| | AGCAATACTAAAGTTGACAAAAAGGTGGAACCC | |
| | AAGTCTTGTGACAAGACCCACACGTGTCCCCCC | |
| | TGCCCGGCTCCTGAGCTGCTTGGCGGCCCCAGC | |
| | GTCTTTCTCTTTCCCCCAAAGCCAAAAGATACC | |
| | TTGATGATCAGCAGAACTCCCGAGGTGACATGC | |
| | GTCGTCGTGGACGTAAGCCATGAAGATCCCGAG | |
| | GTTAAGTTCAACTGGTATGTCGATGGCGTGGAA | |
| | GTCCATAATGCTAAGACTAAACCTCGCGAAGAG | |
| | CAGTACAATTCAACTTACCGGGTCGTTTCCGTT | |
| | CTGACCGTGCTGCATCAGGACTGGCTGAATGGT | |
| | AAAGAGTACAAGTGCAAAGTGTCTAACAAGGCA | |
| | CTCCCCGCCCCAATTGAGAAGACTATCTCCAAA | |
| | GCTAAAGGGCAACCAAGAGAGCCCCAGGTCTAC | |
| | ACCCTGCCCCCCTCAAGGGATGAGCTTACTAAG | |
| | AACCAGGTTAGTCTCACCTGCTTGGTTAAAGGA | |
| | TTTTATCCAAGCGATATTGCTGTGGAGTGGGAG | |
| | TCCAACGGCCAGCCTGAGAACAATTATAAAACC | |
| | ACCCCCCCTGTTCTTGACAGTGACGGTAGTTTC | |
| | TTCCTGTATTCCAAACTGACCGTCGATAAGAGC | |
| | AGATGGCAACAGGGAAATGTGTTCAGCTGCTCC | |
| | GTGATGCATGAGGCGCTCCATAATCATTACACA | |
| | CAAAAAAGTTTGTCCCTGAGCCCAGGCAAG | |
| M.20B10E9_ | DIQMNQSPSSLSASLGDTITITCHASQNIHVWL | 94 |
| LC | SWYQQKPGNIPKLLIYKASNLHTGVPSRFSGSG | |
| | SGTGFTLTISSLQPEDIATYYCQQGQSYPYTFG | |
| | GGTKLEIKRTVAAPSVFIFPPSDEQLKSGTASV | |
| | VCLLNNFYPREAKVQWKVDNALQSGNSQESVTE | |
| | QDSKDSTYSLSSTLTLSKADYEKHKVYACEVTH | |
| | QGLSSPVTKSFNRGEC | |
| M.20B10E9_ | GACATTCAGATGAACCAGAGCCCCAGCAGCCTG | 95 |
| LC | AGCGCCAGCCTGGGAGACACCATCACCATCACC | |
| (Nucleo- | TGCCACGCCAGCCAGAACATCCACGTGTGGCTG | |
| tide) | AGCTGGTATCAGCAGAAACCCGGCAACATCCCC | |
| | AAACTGCTGATCTACAAAGCCTCCAACCTGCAC | |
| | ACCGGCGTGCCCAGCAGATTCAGCGGAAGCGGC | |
| | AGCGGCACCGGATTCACCCTGACCATCAGCAGC | |
| | CTGCAGCCCGAAGACATCGCCACCTACTACTGC | |
| | CAGCAGGGCCAGAGCTACCCCTACACATTCGGC | |
| | GGCGGCACAAAGCTGGAAATCAAGCGTACGGTG | |
| | GCAGCTCCATCAGTTTTTATCTTCCCACCAAGC | |
| | GACGAGCAATTGAAGTCCGGCACTGCCTCTGTG | |
| | GTGTGCCTTCTGAACAACTTCTATCCAAGGGAG | |
| | GCCAAGGTCCAGTGGAAGGTCGATAATGCGCTG | |
| | CAGAGCGGGAACAGCCAAGAGTCAGTGACCGAG | |
| | CAGGACTCAAAAGATAGCACATACTCTCTGAGT | |
| | TCCACCCTGACCCTGTCAAAGGCTGACTACGAA | |
| | AAGCATAAGGTATACGCATGCGAAGTGACCCAT | |
| | CAGGGTCTCTCATCTCCCGTAACCAAATCTTTT | |
| | AATAGAGGAGAATGCTAG | |
| M.69H1H9_ | EYTIH | 3 |
| HC: CDR-1 | | |
| M.69H1H9_ | SINPNNGLFRYNQKFKG | 16 |
| HC: CDR-2 | | |
| M.69H1H9_ | DYGFGYSDY | 32 |
| HC: CDR-3 | | |
| M.69H1H9_ | RASQGIRGNLD | 47 |
| LC: CDR-1 | | |
| M.69H1H9_ | STSNLNS | 60 |
| LC: CDR-2 | | |
| M.69H1H9_ | LQRNAFPWT | 72 |
| LC: CDR-3 | | |
| M.69H1H9_ | EVQLQQSGPELVKPGASVKMSCKTSGYTFTEYT | 96 |
| HC | IHWVKQSHEKSLEWIGSINPNNGLFRYNQKFKG | |
| | TKALTVDKSSSTAYMELRSLTSEDSAVYYCARD | |
| | YGFGYSDYWGQGTTLTVSSASTKGPSVFPLAPS | |
| | SKSTSGGTAALGCLVKDYFPEPVTVSWNSGALT | |
| | SGVHTFPAVLQSSGLYSLSSVVTVPSSSLGTQT | |
| | YICNVNHKPSNTKVDKKVEPKSCDKTHTCPPCP | |
| | APELLGGPSVFLFPPKPKDTLMISRTPEVTCVV | |
| | VDVSHEDPEVKFNWYVDGVEVHNAKTKPREEQY | |
| | NSTYRVVSVLTVLHQDWLNGKEYKCKVSNKALP | |
| | APIEKTISKAKGQPREPQVYTLPPSRDELTKNQ | |
| | VSLTCLVKGFYPSDIAVEWESNGQPENNYKTTP | |
| | PVLDSDGSFFLYSKLTVDKSRWQQGNVFSCSVM | |
| | HEALHNHYTQKSLSLSPGK | |
| M.69H1H9_ | GAGGTGCAGCTGCAGCAGAGCGGGCCAGAGCTG | 179 |
| HC | GTGAAGCCAGGAGCCAGCGTGAAGATGAGCTGT | |
| (Nucleo- | AAGACAAGCGGGTACACATTTACAGAGTACACA | |
| tide) | ATTCACTGGGTGAAGCAGAGCCATGAGAAAAGC | |
| | CTGGAATGGATCGGGAGCATCAACCCCAACAAT | |
| | GGACTGTTCAGGTACAACCAGAAATTCAAGGGC | |
| | AAAGCCACCCTGACCGTGGACAAAAGCAGTAGC | |
| | ACAGCCTACATGGAACTGAGAAGCCTGACCAGC | |
| | GAGGACAGCGCCGTGTACTATTGTGCCAGAGAC | |
| | TACGGCTTCGGGTACAGCGACTACTGGGGCCAG | |
| | GGAACCACCCTGACCGTGAGCTCCGCTAGCACC | |
| | AAAGGTCCCAGCGTGTTCCCACTCGCCCCGAGT | |
| | TCAAAATCAACTTCTGGAGGCACCGCCGCCCTG | |
| | GGTTGCCTGGTAAAGGACTACTTCCCAGAGCCC | |
| | GTGACCGTGAGCTGGAACTCCGGGGCACTGACA | |
| | TCTGGCGTTCATACTTTCCCGGCCGTGCTCCAG | |
| | TCTTCAGGTCTGTATAGTCTCTCCTCTGTGGTC | |
| | ACTGTCCCATCTAGCTCTCTGGGCACCCAAACC | |
| | TACATATGCAACGTTAATCACAAGCCGAGCAAT | |
| | ACTAAAGTTGACAAAAAGGTGGAACCCAAGTCT | |
| | TGTGACAAGACCCACACGTGTCCCCCCTGCCCG | |
| | GCTCCTGAGCTGCTTGGCGGCCCCAGCGTCTTT | |
| | CTCTTTCCCCCAAAGCCAAAAGATACCTTGATG | |
| | ATCAGCAGAACTCCCGAGGTGACATGCGTCGTC | |
| | GTGGACGTAAGCCATGAAGATCCCGAGGTTAAG | |
| | TTCAACTGGTATGTCGATGGCGTGGAAGTCCAT | |
| | AATGCTAAGACTAAACCTCGCGAAGAGCAGTAC | |
| | AATTCAACTTACCGGGTCGTTTCCGTTCTGACC | |
| | GTGCTGCATCAGGACTGGCTGAATGGTAAAGAG | |
| | TACAAGTGCAAAGTGTCTAACAAGGCACTCCCC | |
| | GCCCCAATTGAGAAGACTATCTCCAAAGCTAAA | |
| | GGGCAACCAAGAGAGCCCCAGGTCTACACCCTG | |
| | CCCCCCTCAAGGGATGAGCTTACTAAGAACCAG | |
| | GTTAGTCTCACCTGCTTGGTTAAAGGATTTTAT | |
| | CCAAGCGATATTGCTGTGGAGTGGGAGTCCAAC | |
| | GGCCAGCCTGAGAACAATTATAAAACCACCCCC | |
| | CCTGTTCTTGACAGTGACGGTAGTTTCTTCCTG | |
| | TATTCCAAACTGACCGTCGATAAGAGCAGATGG | |
| | CAACAGGGAAATGTGTTCAGCTGCTCCGTGATG | |
| | CATGAGGCGCTCCATAATCATTACACACAAAAA | |
| | AGTTTGTCCCTGAGCCCAGGCAAG | |
| M.69H1H9_ | DIQMIQSPSSMFASLGDRVSLSCRASQGIRGNL | 97 |
| LC | DWYQQKPGGTIKLLIYSTSNLNSGVPSRFSGSG | |
| | SGSDYSLTISSLESEDFADYYCLQRNAFPWTFG | |
| | GGTKLEIKRTVAAPSVFIFPPSDEQLKSGTASV | |
| | VCLLNNFYPREAKVQWKVDNALQSGNSQESVTE | |
| | QDSKDSTYSLSSTLTLSKADYEKHKVYACEVTH | |
| | QGLSSPVTKSFNRGEC | |
| M.69H1H9_ | GACATTCAGATGATTCAGAGCCCCAGCAGCATG | 98 |
| LC | TTTGCCAGCCTGGGCGATAGAGTGAGCCTGAGC | |
| (Nucleo- | TGTAGGGCCAGCCAGGGGATCAGGGGCAATCTG | |
| tide) | GATTGGTATCAGCAGAAGCCTGGCGGAACCATC | |
| | AAGCTGCTGATCTACAGCACCAGCAACCTGAAC | |
| | AGCGGAGTGCCAAGCAGATTCAGCGGAAGCGGC | |
| | AGCGGCAGCGATTACAGCCTGACCATCAGCTCA | |
| | CTGGAAAGCGAGGACTTCGCCGACTACTACTGC | |
| | CTGCAGAGAAACGCCTTCCCCTGGACCTTCGGC | |
| | GGCGGCACAAAACTGGAGATCAAGCGTACGGTG | |
| | GCAGCTCCATCAGTTTTTATCTTCCCACCAAGC | |
| | GACGAGCAATTGAAGTCCGGCACTGCCTCTGTG | |
| | GTGTGCCTTCTGAACAACTTCTATCCAAGGGAG | |
| | GCCAAGGTCCAGTGGAAGGTCGATAATGCGCTG | |
| | CAGAGCGGGAACAGCCAAGAGTCAGTGACCGAG | |
| | CAGGACTCAAAAGATAGCACATACTCTCTGAGT | |
| | TCCACCCTGACCCTGTCAAAGGCTGACTACGAA | |
| | AAGCATAAGGTATACGCATGCGAAGTGACCCAT | |
| | CAGGGTCTCTCATCTCCCGTAACCAAATCTTTT | |
| | AATAGAGGAGAATGCTAG | |
| M.30A5C6_ | DTYMH | 2 |
| HC: CDR-1 | | |
| M.30A5C6_ | RIDPANGNTKYDPKFQG | 17 |
| HC: CDR-2 | | |
| M.30A5C6_ | GDGHYVGGLNY | 33 |
| HC: CDR-3 | | |
| VM.30A5C6_ | HASQNINVWLS | 48 |
| LC: CDR-1 | | |
| M.30A5C6_ | KASNLHT | 59 |
| LC: CDR-2 | | |
| M.30A5C6_ | QQGQNYPYT | 73 |
| LC: CDR-3 | | |
| M.30A5C6_ | EVQLQQSGAELVKPGASVKLSCTASGFNIEDTY | 99 |
| HC | MHWVKQRPEQGLEWIGRIDPANGNTKYDPKFQG | |
| | KATVTADTSSNTAYLQLSSLTSEDTAVYCCARG | |
| | DGHYVGGLNYWGQGTSLTVSSASTKGPSVFPLA | |
| | PSSKSTSGGTAALGCLVKDYFPEPVTVSWNSGA | |
| | LTSGVHTFPAVLQSSGLYSLSSVVTVPSSSLGT | |
| | QTYICNVNHKPSNTKVDKKVEPKSCDKTHTCPP | |
| | CPAPELLGGPSVFLFPPKPKDTLMISRTPEVTC | |
| | VVVDVSHEDPEVKFNWYVDGVEVHNAKTKPREE | |
| | QYNSTYRVVSVLTVLHQDWLNGKEYKCKVSNKA | |
| | LPAPIEKTISKAKGQPREPQVYTLPPSRDELTK | |
| | NQVSLTCLVKGFYPSDIAVEWESNGQPENNYKT | |
| | TPPVLDSDGSFFLYSKLTVDKSRWQQGNVFSCS | |
| | VMHEALHNHYTQKSLSLSPGK | |
| M.30A5C6_ | GAGGTGCAGCTGCAGCAGAGCGGGGCCGAGCTG | 100 |
| HC | GTGAAGCCAGGAGCCAGCGTGAAGCTGAGCTGT | |
| (Nucleo- | ACAGCCAGCGGCTTCAACATCGAGGACACATAT | |
| tide) | ATGCACTGGGTGAAACAGAGGCCCGAGCAGGGC | |
| | CTGGAGTGGATCGGCAGAATCGACCCCGCTAAC | |
| | GGAAACACCAAATACGACCCCAAGTTCCAGGGC | |
| | AAAGCCACCGTGACCGCCGACACCTCCAGCAAC | |
| | ACCGCCTATCTGCAGCTGAGCAGCCTGACCAGC | |
| | GAGGACACCGCCGTGTACTGCTGCGCCAGAGGC | |
| | GACGGCCACTACGTGGGAGGACTGAACTACTGG | |
| | GGACAGGGCACCAGCCTGACAGTGTCCAGCGCT | |
| | AGCACCAAAGGTCCCAGCGTGTTCCCACTCGCC | |
| | CCGAGTTCAAAATCAACTTCTGGAGGCACCGCC | |
| | GCCCTGGGTTGCCTGGTAAAGGACTACTTCCCA | |
| | GAGCCCGTGACCGTGAGCTGGAACTCCGGGGCA | |
| | CTGACATCTGGCGTTCATACTTTCCCGGCCGTG | |
| | CTCCAGTCTTCAGGTCTGTATAGTCTCTCCTCT | |
| | GTGGTCACTGTCCCATCTAGCTCTCTGGGCACC | |
| | CAAACCTACATATGCAACGTTAATCACAAGCCG | |
| | AGCAATACTAAAGTTGACAAAAAGGTGGAACCC | |
| | AAGTCTTGTGACAAGACCCACACGTGTCCCCCC | |
| | TGCCCGGCTCCTGAGCTGCTTGGCGGCCCCAGC | |
| | GTCTTTCTCTTTCCCCCAAAGCCAAAAGATACC | |
| | TTGATGATCAGCAGAACTCCCGAGGTGACATGC | |
| | GTCGTCGTGGACGTAAGCCATGAAGATCCCGAG | |
| | GTTAAGTTCAACTGGTATGTCGATGGCGTGGAA | |
| | GTCCATAATGCTAAGACTAAACCTCGCGAAGAG | |
| | CAGTACAATTCAACTTACCGGGTCGTTTCCGTT | |
| | CTGACCGTGCTGCATCAGGACTGGCTGAATGGT | |
| | AAAGAGTACAAGTGCAAAGTGTCTAACAAGGCA | |
| | CTCCCCGCCCCAATTGAGAAGACTATCTCCAAA | |
| | GCTAAAGGGCAACCAAGAGAGCCCCAGGTCTAC | |
| | ACCCTGCCCCCCTCAAGGGATGAGCTTACTAAG | |
| | AACCAGGTTAGTCTCACCTGCTTGGTTAAAGGA | |
| | TTTTATCCAAGCGATATTGCTGTGGAGTGGGAG | |
| | TCCAACGGCCAGCCTGAGAACAATTATAAAACC | |
| | ACCCCCCCTGTTCTTGACAGTGACGGTAGTTTC | |
| | TTCCTGTATTCCAAACTGACCGTCGATAAGAGC | |
| | AGATGGCAACAGGGAAATGTGTTCAGCTGCTCC | |
| | GTGATGCATGAGGCGCTCCATAATCATTACACA | |
| | CAAAAAAGTTTGTCCCTGAGCCCAGGCAAG | |
| M.30A5C6_ | DIQMNQSPSSLSASLGDTITITCHASQNINVWL | 101 |
| LC | SWYQQKPGNIPKLLIYKASNLHTGVPSRFSGSG | |
| | SGTGFTLTISSLQPEDIATYYCQQGQNYPYTFG | |
| | GGTKLEIKRTVAAPSVFIFPPSDEQLKSGTASV | |
| | VCLLNNFYPREAKVQWKVDNALQSGNSQESVTE | |
| | QDSKDSTYSLSSTLTLSKADYEKHKVYACEVTH | |
| | QGLSSPVTKSFNRGEC | |
| M.30A5C6_ | GACATTCAGATGAACCAGAGCCCCAGCAGCCTG | 102 |
| LC | AGCGCCAGCCTGGGAGACACCATCACCATCACC | |
| (Nucleo- | TGCCACGCCAGCCAGAACATCAACGTGTGGCTG | |
| tide) | AGCTGGTATCAGCAGAAACCCGGCAACATCCCC | |
| | AAACTGCTGATCTACAAAGCCTCCAACCTGCAC | |
| | ACCGGCGTGCCCAGCAGATTCAGCGGAAGCGGC | |
| | AGCGGCACCGGATTCACCCTGACCATCAGCAGC | |
| | CTGCAGCCCGAAGACATCGCCACCTACTACTGC | |
| | CAGCAGGGCCAGAACTACCCCTACACCTTCGGC | |
| | GGCGGCACCAAGCTGGAGATCAAGCGTACGGTG | |
| | GCAGCTCCATCAGTTTTTATCTTCCCACCAAGC | |
| | GACGAGCAATTGAAGTCCGGCACTGCCTCTGTG | |
| | GTGTGCCTTCTGAACAACTTCTATCCAAGGGAG | |
| | GCCAAGGTCCAGTGGAAGGTCGATAATGCGCTG | |
| | CAGAGCGGGAACAGCCAAGAGTCAGTGACCGAG | |
| | CAGGACTCAAAAGATAGCACATACTCTCTGAGT | |
| | TCCACCCTGACCCTGTCAAAGGCTGACTACGAA | |
| | AAGCATAAGGTATACGCATGCGAAGTGACCCAT | |
| | CAGGGTCTCTCATCTCCCGTAACCAAATCTTTT | |
| | AATAGAGGAGAATGCTAG | |
| M.95D4A11_ | SYWMN | 4 |
| HC: CDR-1 | | |
| M.95D4A11_ | SIDPYDSETHYNQKFKD | 18 |
| HC: CDR-2 | | |
| M.95D4A11_ | GRGYAMDY | 34 |
| HC: CDR-3 | | |
| M.95D4A11_ | RASGNIHNYLA | 49 |
| LC: CDR-1 | | |
| M.95D4A11_ | NAKTLAD | 61 |
| LC: CDR-2 | | |
| M.95D4A11_ | QHFWSTPPT | 74 |
| LC: CDR-3 | | |
| M.95D4A11_ | QVQLQQPGAELVRPGASVKLSCKASGYTFTSYW | 180 |
| HC | MNWVKQRPEQGLEWIGSIDPYDSETHYNQKFKD | |
| | KAILTVDKSSSTAYMQLSSLTSEDSAVYYCARG | |
| | RGYAMDYWGQGTSVTVSSASTKGPSVFPLAPSS | |
| | KSTSGGTAALGCLVKDYFPEPVTVSWNSGALTS | |
| | GVHTFPAVLQSSGLYSLSSVVTVPSSSLGTQTY | |
| | ICNVNHKPSNTKVDKKVEPKSCDKTHTCPPCPA | |
| | PELLGGPSVFLFPPKPKDTLMISRTPEVTCVVV | |
| | DVSHEDPEVKFNWYVDGVEVHNAKTKPREEQYN | |
| | STYRVVSVLTVLHQDWLNGKEYKCKVSNKALPA | |
| | PIEKTISKAKGQPREPQVYTLPPSRDELTKNQV | |
| | SLTCLVKGFYPSDIAVEWESNGQPENNYKTTPP | |
| | VLDSDGSFFLYSKLTVDKSRWQQGNVFSCSVMH | |
| | EALHNHYTQKSLSLSPGK | |
| M.95D4A11_ | CAGGTGCAGCTGCAGCAGCCAGGCGCCGAGCTG | 103 |
| HC | GTGAGACCCGGAGCTAGCGTGAAACTGAGCTGC | |
| (Nucleo- | AAAGCCAGCGGCTACACCTTCACCTCCTACTGG | |
| tide) | ATGAACTGGGTGAAGCAGAGACCCGAACAGGGA | |
| | CTGGAGTGGATTGGAAGCATTGACCCCTACGAT | |
| | AGCGAGACCCACTACAACCAGAAATTCAAGGAC | |
| | AAAGCCATCCTGACCGTGGACAAAAGCAGCAGC | |
| | ACAGCCTACATGCAGCTGAGCAGCCTGACCAGC | |
| | GAGGACAGCGCCGTGTACTACTGCGCCAGAGGC | |
| | AGAGGCTACGCCATGGACTACTGGGGCCAGGGC | |
| | ACCAGCGTGACCGTGAGCAGCGCTAGCACCAAA | |
| | GGTCCCAGCGTGTTCCCACTCGCCCCGAGTTCA | |
| | AAATCAACTTCTGGAGGCACCGCCGCCCTGGGT | |
| | TGCCTGGTAAAGGACTACTTCCCAGAGCCCGTG | |
| | ACCGTGAGCTGGAACTCCGGGGCACTGACATCT | |
| | GGCGTTCATACTTTCCCGGCCGTGCTCCAGTCT | |
| | TCAGGTCTGTATAGTCTCTCCTCTGTGGTCACT | |
| | GTCCCATCTAGCTCTCTGGGCACCCAAACCTAC | |
| | ATATGCAACGTTAATCACAAGCCGAGCAATACT | |
| | AAAGTTGACAAAAAGGTGGAACCCAAGTCTTGT | |
| | GACAAGACCCACACGTGTCCCCCCTGCCCGGCT | |
| | CCTGAGCTGCTTGGCGGCCCCAGCGTCTTTCTC | |
| | TTTCCCCCAAAGCCAAAAGATACCTTGATGATC | |
| | AGCAGAACTCCCGAGGTGACATGCGTCGTCGTG | |
| | GACGTAAGCCATGAAGATCCCGAGGTTAAGTTC | |
| | AACTGGTATGTCGATGGCGTGGAAGTCCATAAT | |
| | GCTAAGACTAAACCTCGCGAAGAGCAGTACAAT | |
| | TCAACTTACCGGGTCGTTTCCGTTCTGACCGTG | |
| | CTGCATCAGGACTGGCTGAATGGTAAAGAGTAC | |
| | AAGTGCAAAGTGTCTAACAAGGCACTCCCCGCC | |
| | CCAATTGAGAAGACTATCTCCAAAGCTAAAGGG | |
| | CAACCAAGAGAGCCCCAGGTCTACACCCTGCCC | |
| | CCCTCAAGGGATGAGCTTACTAAGAACCAGGTT | |
| | AGTCTCACCTGCTTGGTTAAAGGATTTTATCCA | |
| | AGCGATATTGCTGTGGAGTGGGAGTCCAACGGC | |
| | CAGCCTGAGAACAATTATAAAACCACCCCCCCT | |
| | GTTCTTGACAGTGACGGTAGTTTCTTCCTGTAT | |
| | TCCAAACTGACCGTCGATAAGAGCAGATGGCAA | |
| | CAGGGAAATGTGTTCAGCTGCTCCGTGATGCAT | |
| | GAGGCGCTCCATAATCATTACACACAAAAAAGT | |
| | TTGTCCCTGAGCCCAGGCAAG | |
| M.95D4A11_ | DIQMTQSPASLSASVGETVTITCRASGNIHNYL | 104 |
| LC | AWYQQKQGKSPQLLVYNAKTLADGVPSRFSGSG | |
| | SGTQYSLKINSLQPEDFGSYYCQHFWSTPPTFG | |
| | GGTKLEIKRTVAAPSVFIFPPSDEQLKSGTASV | |
| | VCLLNNFYPREAKVQWKVDNALQSGNSQESVTE | |
| | QDSKDSTYSLSSTLTLSKADYEKHKVYACEVTH | |
| | QGLSSPVTKSFNRGEC | |
| M.95D4A11_ | GACATTCAGATGACCCAGAGCCCCGCCAGCCTG | 105 |
| LC | AGCGCCTCTGTGGGAGAGACCGTGACCATCACC | |
| (Nucleo- | TGCAGAGCCAGCGGCAACATCCACAACTACCTG | |
| tide) | GCCTGGTACCAGCAGAAACAGGGCAAGAGCCCC | |
| | CAGCTGCTGGTGTACAACGCCAAGACCCTGGCC | |
| | GACGGCGTGCCCTCAAGATTCAGCGGAAGCGGA | |
| | AGCGGCACACAGTACAGCCTGAAGATCAACAGC | |
| | CTGCAGCCCGAGGACTTCGGCTCCTACTATTGC | |
| | CAGCACTTCTGGTCTACCCCCCCCACCTTCGGC | |
| | GGCGGAACAAAACTGGAGATCAAGCGTACGGTG | |
| | GCAGCTCCATCAGTTTTTATCTTCCCACCAAGC | |
| | GACGAGCAATTGAAGTCCGGCACTGCCTCTGTG | |
| | GTGTGCCTTCTGAACAACTTCTATCCAAGGGAG | |
| | GCCAAGGTCCAGTGGAAGGTCGATAATGCGCTG | |
| | CAGAGCGGGAACAGCCAAGAGTCAGTGACCGAG | |
| | CAGGACTCAAAAGATAGCACATACTCTCTGAGT | |
| | TCCACCCTGACCCTGTCAAAGGCTGACTACGAA | |
| | AAGCATAAGGTATACGCATGCGAAGTGACCCAT | |
| | CAGGGTCTCTCATCTCCCGTAACCAAATCTTTT | |
| | AATAGAGGAGAATGCTAG | |

Generation of Anti-Hu/Cyno CD47 Antibodies from Rabbit B Cell Cloning

Rabbit monoclonal antibodies specific for CD47 were generated as described in PCT/US2019/061884, which is hereby incorporated by reference in its entirety.

Rabbits were immunized four times with recombinant human CD47 antigen. Serum titers were assayed by enzyme-linked immunosorbent assay (ELISA) using recombinant CD47 proteins. Target recognition was further assayed by flow cytometry. A rabbit with a high ELISA titer and a strong flow cytometry signal was boosted 4 days ahead of splenectomy via IV injection of 400 μg of recombinant huCD47. 1.2E8 freshly isolated splenocytes were cultured overnight in customized B cell medium before sorting. Splenocytes were processed using the SMab™ platform to enrich antigen-recognizing B cells. FACS-sorted B cells were cultured at 1 cell/well in a 96-well plate for 10-14 days.

Positive clones were identified using a direct anti-huCD47 ELISA. To identify monoclonal antibodies suitable for FACS analysis, initial positive clones were screened against Jurkat cells which express high CD47 on its surface. FACS-positive mAb clones were further confirmed using linear expression module (LEM) supernatants from HEK293F cells transiently expressing recombinant IgG genes recovered from the initial positive clones.

Chimeric antibodies with rabbit variable domains and human constant domains were generated by fusing the VH domain of the anti-CD47 monoclonal antibodies to a human IgG1 constant region and the VL domain of the anti-CD47 monoclonal antibodies to a human Kappa constant region. Because the human Kappa constant region does not have a cysteine corresponding to the cysteine in the rabbit Kappa constant region that forms an interdomain disulfide bond with the rabbit VL (Cys80-Cys1171), Cys80 in the VL region was replaced with Ser to eliminate the free cysteine. Table 5 presents sequences of exemplary anti-CD47 antibodies discovered by these methods including their complementarity-determining regions (CDRs), heavy and light chains. CDRs were determined by Kabat numbering system.

TABLE 5
Anti-CD47 Antibody Sequences from
Rabbit B Cell Cloning

| Construct | | SEQ |
|---|---|---|
| Descrip- | | ID |
| tion | Sequence | NO: |
| R.1H3_HC: | SGYYMC | 5 |
| CDR-1 | | |
| R.1H3_HC: | CIYMRSGDTYLASWAKG | 19 |
| CDR-2 | | |
| R.1H3_HC: | GGTSYALNL | 35 |
| CDR-3 | | |
| R.1H3_LC: | QASQSISNECS | 50 |
| CDR-1 | | |
| R.1H3_LC: | RASTLES | 62 |
| CDR-2 | | |
| R.1H3_LC: | QSTYYGTTGNT | 75 |
| CDR-3 | | |
| R.1H3_HC | QSLEESGGDLVKPGASLTLTCTASGFSFSSGYY | 106 |
| | MCWVRQAPGKGLEWIACIYMRSGDTYLASWAKG | |
| | QFTISKTSSTTVTLQMTSLTAADTATYFCGRGG | |
| | TSYALNLWGQGTLVTVSSASTKGPSVFPLAPSS | |
| | KSTSGGTAALGCLVKDYFPEPVTVSWNSGALTS | |
| | GVHTFPAVLQSSGLYSLSSVVTVPSSSLGTQTY | |
| | ICNVNHKPSNTKVDKKVEPKSCDKTHTCPPCPA | |
| | PELLGGPSVFLFPPKPKDTLMISRTPEVTCVVV | |
| | DVSHEDPEVKFNWYVDGVEVHNAKTKPREEQYN | |
| | STYRVVSVLTVLHQDWLNGKEYKCKVSNKALPA | |
| | PIEKTISKAKGQPREPQVYTLPPSRDELTKNQV | |
| | SLTCLVKGFYPSDIAVEWESNGQPENNYKTTPP | |
| | VLDSDGSFFLYSKLTVDKSRWQQGNVFSCSVMH | |
| | EALHNHYTQKSLSLSPGK | |
| R.1H3_HC | CAGTCTCTGGAAGAATCCGGCGGAGACCTGGTG | 107 |
| (Nucleo- | AAGCCTGGAGCTTCTCTGACCCTGACCTGCACA | |
| tide) | GCATCCGGATTTTCCTTTTCCTCCGGCTACTAC | |
| | ATGTGCTGGGTGAGGCAGGCACCCGGAAAAGGA | |
| | CTGGAATGGATTGCCTGTATCTACATGCGTTCC | |
| | GGCGACACCTACCTGGCTTCTTGGGCTAAGGGC | |
| | CAGTTCACCATTTCCAAGACCTCCTCCACCACC | |
| | GTCACCCTGCAGATGACATCCCTGACCGCTGCT | |
| | GACACCGCTACATACTTCTGTGGCCGCGGAGGT | |
| | ACCTCCTACGCTCTGAACCTGTGGGGACAGGGA | |
| | ACCCTGGTGACAGTGTCTTCCGCTAGCACCAAA | |
| | GGTCCCAGCGTGTTCCCACTCGCCCCGAGTTCA | |
| | AAATCAACTTCTGGAGGCACCGCCGCCCTGGGT | |
| | TGCCTGGTAAAGGACTACTTCCCAGAGCCCGTG | |
| | ACCGTGAGCTGGAACTCCGGGGCACTGACATCT | |
| | GGCGTTCATACTTTCCCGGCCGTGCTCCAGTCT | |
| | TCAGGTCTGTATAGTCTCTCCTCTGTGGTCACT | |
| | GTCCCATCTAGCTCTCTGGGCACCCAAACCTAC | |
| | ATATGCAACGTTAATCACAAGCCGAGCAATACT | |
| | AAAGTTGACAAAAAGGTGGAACCCAAGTCTTGT | |
| | GACAAGACCCACACGTGTCCCCCCTGCCCGGCT | |
| | CCTGAGCTGCTTGGCGGCCCCAGCGTCTTTCTC | |
| | TTTCCCCCAAAGCCAAAAGATACCTTGATGATC | |
| | AGCAGAACTCCCGAGGTGACATGCGTCGTCGTG | |
| | GACGTAAGCCATGAAGATCCCGAGGTTAAGTTC | |
| | AACTGGTATGTCGATGGCGTGGAAGTCCATAAT | |
| | GCTAAGACTAAACCTCGCGAAGAGCAGTACAAT | |
| | TCAACTTACCGGGTCGTTTCCGTTCTGACCGTG | |
| | CTGCATCAGGACTGGCTGAATGGTAAAGAGTAC | |
| | AAGTGCAAAGTGTCTAACAAGGCACTCCCCGCC | |
| | CCAATTGAGAAGACTATCTCCAAAGCTAAAGGG | |
| | CAACCAAGAGAGCCCCAGGTCTACACCCTGCCC | |
| | CCCTCAAGGGATGAGCTTACTAAGAACCAGGTT | |
| | AGTCTCACCTGCTTGGTTAAAGGATTTTATCCA | |
| | AGCGATATTGCTGTGGAGTGGGAGTCCAACGGC | |
| | CAGCCTGAGAACAATTATAAAACCACCCCCCCT | |
| | GTTCTTGACAGTGACGGTAGTTTCTTCCTGTAT | |
| | TCCAAACTGACCGTCGATAAGAGCAGATGGCAA | |
| | CAGGGAAATGTGTTCAGCTGCTCCGTGATGCAT | |
| | GAGGCGCTCCATAATCATTACACACAAAAAAGT | |
| | TTGTCCCTGAGCCCAGGCAAG | |
| R.1H3_LC | DVVMTQTPASVSEPVGGTVTINCQASQSISNEC | 108 |
| | SWYQQKPGQPPKLLIYRASTLESGVPSRFSGSG | |
| | SGTEFTLTISDLESADAATYYCQSTYYGTTGNT | |
| | FGGGTEVVVKRTVAAPSVFIFPPSDEQLKSGTA | |
| | SVVCLLNNFYPREAKVQWKVDNALQSGNSQESV | |
| | TEQDSKDSTYSLSSTLTLSKADYEKHKVYACEV | |
| | THQGLSSPVTKSFNRGEC | |
| R.1H3_LC | GATGTGGTGATGACCCAGACCCCAGCAAGCGTG | 109 |
| (Nucleo- | AGTGAGCCTGTGGGAGGAACAGTGACCATTAAT | |
| tide) | TGCCAGGCCTCCCAGTCCATTAGTAACGAGTGT | |
| | AGCTGGTACCAGCAGAAGCCCGGTCAGCCTCCT | |
| | AAGCTGCTGATTTACAGGGCCTCCACCCTGGAG | |
| | AGCGGAGTGCCTTCTAGATTCTCCGGATCCGGC | |
| | TCCGGAACCGAATTTACCCTGACCATCTCCGAC | |
| | CTGGAATCTGCCGACGCTGCAACTTACTACTGC | |
| | CAGTCCACCTACTACGGGACAACCGGAAACACC | |
| | TTCGGCGGAGGAACCGAAGTGGTGGTGAAGcgt | |
| | acgGTGGCAGCTCCATCAGTTTTTATCTTCCCA | |
| | CCAAGCGACGAGCAATTGAAGTCCGGCACTGCC | |
| | TCTGTGGTGTGCCTTCTGAACAACTTCTATCCA | |
| | AGGGAGGCCAAGGTCCAGTGGAAGGTCGATAAT | |
| | GCGCTGCAGAGCGGGAACAGCCAAGAGTCAGTG | |
| | ACCGAGCAGGACTCAAAAGATAGCACATACTCT | |
| | CTGAGTTCCACCCTGACCCTGTCAAAGGCTGAC | |
| | TACGAAAAGCATAAGGTATACGCATGCGAAGTG | |
| | ACCCATCAGGGTCTCTCATCTCCCGTAACCAAA | |
| | TCTTTTAATAGAGGAGAATGC | |
| R.9B11_HC: | SYWMA | 6 |
| CDR-1 | | |
| R.9B11_HC: | CIYIGSGSGTTYYASWAK | 20 |
| CDR-2 | | |
| R.9B11_HC: | WAGSDGHFTL | 176 |
| CDR-3 | | |
| R.9B11_LC: | QASQSINNLLA | 51 |
| CDR-1 | | |
| R.9B11_LC: | DASDLAS | 63 |
| CDR-2 | | |
| R.9B11_LC: | QGYFYGSSVDLV | 76 |
| CDR-3 | | |
| R.9B11_HC | QEQLEESGGDLVKPEGSLTLTCTASGFSFSSYW | 110 |
| | MAWVRQAPGKGLEWIACIYIGSGSGTTYYASWA | |
| | KGRFTISKTSSTTVTLQMTSLTAADTAIYFCAR | |
| | WAGSDGHFTLWGPGTLVTVSLASTKGPSVFPLA | |
| | PSSKSTSGGTAALGCLVKDYFPEPVTVSWNSGA | |
| | LTSGVHTFPAVLQSSGLYSLSSVVTVPSSSLGT | |
| | QTYICNVNHKPSNTKVDKKVEPKSCDKTHTCPP | |
| | CPAPELLGGPSVFLFPPKPKDTLMISRTPEVTC | |
| | VVVDVSHEDPEVKFNWYVDGVEVHNAKTKPREE | |
| | QYNSTYRVVSVLTVLHQDWLNGKEYKCKVSNKA | |
| | LPAPIEKTISKAKGQPREPQVYTLPPSRDELTK | |
| | NQVSLTCLVKGFYPSDIAVEWESNGQPENNYKT | |
| | TPPVLDSDGSFFLYSKLTVDKSRWQQGNVFSCS | |
| | VMHEALHNHYTQKSLSLSPGK | |
| R.9B11_HC | CAGGAACAGCTGGAGGAAAGCGGAGGAGACCTG | 111 |
| (Nucleo- | GTGAAGCCAGAGGGAAGTCTGACACTGACCTGC | |
| tide) | ACCGCTAGTGGCTTCTCCTTTAGCAGCTACTGG | |
| | ATGGCCTGGGTGAGGCAGGCTCCTGGAAAAGGA | |
| | CTGGAGTGGATCGCATGCATTTACATCGGCAGC | |
| | GGCTCTGGCACCACATACTACGCTTCCTGGGCC | |
| | AAGGGCAGGTTCACTATCTCCAAAACCAGTTCA | |
| | ACAACCGTCACCCTGCAGATGACAAGCCTGACC | |
| | GCTGCTGACACCGCTATCTATTTTTGTGCCCGG | |
| | TGGGCCGGTTCCGACGGACATTTCACACTGTGG | |
| | GGCCCTGGAACCCTGGTGACAGTGTCTCTGGCT | |
| | AGCACCAAAGGTCCCAGCGTGTTCCCACTCGCC | |
| | CCGAGTTCAAAATCAACTTCTGGAGGCACCGCC | |
| | GCCCTGGGTTGCCTGGTAAAGGACTACTTCCCA | |
| | GAGCCCGTGACCGTGAGCTGGAACTCCGGGGCA | |
| | CTGACATCTGGCGTTCATACTTTCCCGGCCGTG | |
| | CTCCAGTCTTCAGGTCTGTATAGTCTCTCCTCT | |
| | GTGGTCACTGTCCCATCTAGCTCTCTGGGCACC | |
| | CAAACCTACATATGCAACGTTAATCACAAGCCG | |
| | AGCAATACTAAAGTTGACAAAAAGGTGGAACCC | |
| | AAGTCTTGTGACAAGACCCACACGTGTCCCCCC | |
| | TGCCCGGCTCCTGAGCTGCTTGGCGGCCCCAGC | |
| | GTCTTTCTCTTTCCCCCAAAGCCAAAAGATACC | |
| | TTGATGATCAGCAGAACTCCCGAGGTGACATGC | |
| | GTCGTCGTGGACGTAAGCCATGAAGATCCCGAG | |
| | GTTAAGTTCAACTGGTATGTCGATGGCGTGGAA | |
| | GTCCATAATGCTAAGACTAAACCTCGCGAAGAG | |
| | CAGTACAATTCAACTTACCGGGTCGTTTCCGTT | |
| | CTGACCGTGCTGCATCAGGACTGGCTGAATGGT | |
| | AAAGAGTACAAGTGCAAAGTGTCTAACAAGGCA | |
| | CTCCCCGCCCCAATTGAGAAGACTATCTCCAAA | |
| | GCTAAAGGGCAACCAAGAGAGCCCCAGGTCTAC | |
| | ACCCTGCCCCCCTCAAGGGATGAGCTTACTAAG | |
| | AACCAGGTTAGTCTCACCTGCTTGGTTAAAGGA | |
| | TTTTATCCAAGCGATATTGCTGTGGAGTGGGAG | |
| | TCCAACGGCCAGCCTGAGAACAATTATAAAACC | |
| | ACCCCCCCTGTTCTTGACAGTGACGGTAGTTTC | |
| | TTCCTGTATTCCAAACTGACCGTCGATAAGAGC | |
| | AGATGGCAACAGGGAAATGTGTTCAGCTGCTCC | |
| | GTGATGCATGAGGCGCTCCATAATCATTACACA | |
| | CAAAAAAGTTTGTCCCTGAGCCCAGGCAAG | |
| R.9B11_LC | ALVMTQTPASVEAAVGGTVTINCQASQSINNLL | 112 |
| | AWYQQKPGQRPKLLIYDASDLASGVSSRFKGSG | |
| | SGTEYTLTISGVQSADAATYYCQGYFYGSSVDL | |
| | VFGGGTEVVVKRTVAAPSVFIFPPSDEQLKSGT | |
| | ASVVCLLNNFYPREAKVQWKVDNALQSGNSQES | |
| | VTEQDSKDSTYSLSSTLTLSKADYEKHKVYACE | |
| | VTHQGLSSPVTKSFNRGEC | |
| R.9B11_LC | GCTCTGGTGATGACCCAGACACCTGCTTCCGTG | 113 |
| (Nucleo- | GAGGCTGCTGTGGGAGGAACAGTGACAATCAAC | |
| tide) | TGCCAGGCCAGCCAGTCCATTAACAACCTGCTG | |
| | GCCTGGTACCAGCAGAAACCAGGACAGAGGCCC | |
| | AAGCTGCTGATTTACGATGCATCCGATCTGGCA | |
| | AGCGGCGTGTCTTCTAGGTTCAAGGGCTCCGGA | |
| | TCCGGCACAGAATACACCCTGACCATCTCCGGC | |
| | GTGCAGTCTGCTGATGCTGCAACATACTACTGC | |
| | CAGGGATACTTTTACGGCTCCTCCGTGGACCTG | |
| | GTGTTCGGAGGAGGAACAGAGGTGGTGGTGAAG | |
| | cgtacgGTGGCAGCTCCATCAGTTTTTATCTTC | |
| | CCACCAAGCGACGAGCAATTGAAGTCCGGCACT | |
| | GCCTCTGTGGTGTGCCTTCTGAACAACTTCTAT | |
| | CCAAGGGAGGCCAAGGTCCAGTGGAAGGTCGAT | |
| | AATGCGCTGCAGAGCGGGAACAGCCAAGAGTCA | |
| | GTGACCGAGCAGGACTCAAAAGATAGCACATAC | |
| | TCTCTGAGTTCCACCCTGACCCTGTCAAAGGCT | |
| | GACTACGAAAAGCATAAGGTATACGCATGCGAA | |
| | GTGACCCATCAGGGTCTCTCATCTCCCGTAACC | |
| | AAATCTTTTAATAGAGGAGAATGC | |
| R.13A2_HC: | SYYYVC | 7 |
| CDR-1 | | |
| R.13A2_HC: | CIYAGSSGDTYYASWAKG | 21 |
| CDR-2 | | |
| R.13A2_HC: | GYTGYSYQFNF | 36 |
| CDR-3 | | |
| R.13A2_LC: | QASQSISNELS | 52 |
| CDR-1 | | |
| R.13A2_LC: | RASTLES | 62 |
| CDR-2 | | |
| R.13A2_LC: | QCTYITYGNA | 77 |
| CDR-3 | | |
| R.13A2_HC | QSLEESGGDLVKPGASLTVTCTASGFSFSSYYY | 114 |
| | VCWVRQAPGKGLEWIACIYAGSSGDTYYASWAK | |
| | GRFTISKTSSTTVTLQMTSLTAADTATYFCARG | |
| | YTGYSYQFNFWGPGTLVTVSSASTKGPSVFPLA | |
| | PSSKSTSGGTAALGCLVKDYFPEPVTVSWNSGA | |
| | LTSGVHTFPAVLQSSGLYSLSSVVTVPSSSLGT | |
| | QTYICNVNHKPSNTKVDKKVEPKSCDKTHTCPP | |
| | CPAPELLGGPSVFLFPPKPKDTLMISRTPEVTC | |
| | VVVDVSHEDPEVKFNWYVDGVEVHNAKTKPREE | |
| | QYNSTYRVVSVLTVLHQDWLNGKEYKCKVSNKA | |
| | LPAPIEKTISKAKGQPREPQVYTLPPSRDELTK | |
| | NQVSLTCLVKGFYPSDIAVEWESNGQPENNYKT | |
| | TPPVLDSDGSFFLYSKLTVDKSRWQQGNVFSCS | |
| | VMHEALHNHYTQKSLSLSPGK | |
| R.13A2_HC | CAGTCTCTGGAAGAATCCGGCGGAGACCTGGTG | 115 |
| (Nucleo- | AAGCCTGGAGCTTCTCTGACCGTCACCTGCACA | |
| tide) | GCATCCGGATTTAGCTTTTCCTCCTACTACTAC | |
| | GTCTGCTGGGTCAGGCAGGCCCCTGGAAAGGGA | |
| | CTGGAATGGATTGCCTGCATCTACGCCGGTTCC | |
| | TCCGGAGATACCTACTACGCTTCTTGGGCCAAG | |
| | GGCCGTTTCACCATCTCCAAGACCTCTTCCACC | |
| | ACCGTGACCCTGCAGATGACCTCTCTGACCGCT | |
| | GCTGACACCGCTACATACTTTTGCGCCCGCGGA | |
| | TACACCGGCTACTCTTATCAGTTCAACTTCTGG | |
| | GGCCCCGGCACCCTGGTGACAGTGTCTTCTGCT | |
| | AGCACCAAAGGTCCCAGCGTGTTCCCACTCGCC | |
| | CCGAGTTCAAAATCAACTTCTGGAGGCACCGCC | |
| | GCCCTGGGTTGCCTGGTAAAGGACTACTTCCCA | |
| | GAGCCCGTGACCGTGAGCTGGAACTCCGGGGCA | |
| | CTGACATCTGGCGTTCATACTTTCCCGGCCGTG | |
| | CTCCAGTCTTCAGGTCTGTATAGTCTCTCCTCT | |
| | GTGGTCACTGTCCCATCTAGCTCTCTGGGCACC | |
| | CAAACCTACATATGCAACGTTAATCACAAGCCG | |
| | AGCAATACTAAAGTTGACAAAAAGGTGGAACCC | |
| | AAGTCTTGTGACAAGACCCACACGTGTCCCCCC | |
| | TGCCCGGCTCCTGAGCTGCTTGGCGGCCCCAGC | |
| | GTCTTTCTCTTTCCCCCAAAGCCAAAAGATACC | |
| | TTGATGATCAGCAGAACTCCCGAGGTGACATGC | |
| | GTCGTCGTGGACGTAAGCCATGAAGATCCCGAG | |
| | GTTAAGTTCAACTGGTATGTCGATGGCGTGGAA | |
| | GTCCATAATGCTAAGACTAAACCTCGCGAAGAG | |
| | CAGTACAATTCAACTTACCGGGTCGTTTCCGTT | |
| | CTGACCGTGCTGCATCAGGACTGGCTGAATGGT | |
| | AAAGAGTACAAGTGCAAAGTGTCTAACAAGGCA | |
| | CTCCCCGCCCCAATTGAGAAGACTATCTCCAAA | |
| | GCTAAAGGGCAACCAAGAGAGCCCCAGGTCTAC | |
| | ACCCTGCCCCCCTCAAGGGATGAGCTTACTAAG | |
| | AACCAGGTTAGTCTCACCTGCTTGGTTAAAGGA | |
| | TTTTATCCAAGCGATATTGCTGTGGAGTGGGAG | |
| | TCCAACGGCCAGCCTGAGAACAATTATAAAACC | |
| | ACCCCCCCTGTTCTTGACAGTGACGGTAGTTTC | |
| | TTCCTGTATTCCAAACTGACCGTCGATAAGAGC | |
| | AGATGGCAACAGGGAAATGTGTTCAGCTGCTCC | |
| | GTGATGCATGAGGCGCTCCATAATCATTACACA | |
| | CAAAAAAGTTTGTCCCTGAGCCCAGGCAAG | |
| R.13A2_LC | DVVMTQTPASVEAAVGGTVTIKCQASQSISNEL | 116 |
| | SWYQQKPGQPPKLLIYRASTLESGVPSRFKGSG | |
| | SGTEFTLTISDLESADAATYYCQCTYITYGNAF | |
| | GGGTEVVVKRTVAAPSVFIFPPSDEQLKSGTAS | |
| | VVCLLNNFYPREAKVQWKVDNALQSGNSQESVT | |
| | EQDSKDSTYSLSSTLTLSKADYEKHKVYACEVT | |
| | HQGLSSPVTKSFNRGEC | |
| R.13A2_LC | GATGTGGTGATGACCCAGACCCCAGCAAGCGTG | 117 |
| (Nucleo- | GAAGCAGCTGTGGGAGGAACAGTGACCATTAAG | |
| tide) | TGCCAGGCCTCCCAGTCCATTTCCAACGAGCTG | |
| | AGCTGGTACCAGCAGAAGCCCGGACAGCCTCCT | |
| | AAACTGCTGATCTATAGGGCAAGTACCCTGGAG | |
| | AGCGGAGTGCCTTCTAGGTTCAAAGGATCCGGC | |
| | AGCGGAACTGAATTTACCCTGACTATCTCCGAC | |
| | CTGGAGAGCGCTGACGCTGCTACATACTACTGC | |
| | CAGTGTACCTACATCACATACGGGAATGCCTTT | |
| | GGGGGCGGCACAGAAGTGGTGGTGAAGcgtacg | |
| | GTGGCAGCTCCATCAGTTTTTATCTTCCCACCA | |
| | AGCGACGAGCAATTGAAGTCCGGCACTGCCTCT | |
| | GTGGTGTGCCTTCTGAACAACTTCTATCCAAGG | |
| | GAGGCCAAGGTCCAGTGGAAGGTCGATAATGCG | |
| | CTGCAGAGCGGGAACAGCCAAGAGTCAGTGACC | |
| | GAGCAGGACTCAAAAGATAGCACATACTCTCTG | |
| | AGTTCCACCCTGACCCTGTCAAAGGCTGACTAC | |
| | GAAAAGCATAAGGTATACGCATGCGAAGTGACC | |
| | CATCAGGGTCTCTCATCTCCCGTAACCAAATCT | |
| | TTTAATAGAGGAGAATGC | |
| R.14G4_HC: | SYHMC | 8 |
| CDR-1 | | |
| R.14G4_HC: | CIYPNNGFTWYASWAKG | 22 |
| CDR-2 | | |
| R.14G4_HC: | GSGNSYNQLDL | 37 |
| CDR-3 | | |
| R.14G4_LC: | QSSESVYRDNCLG | 53 |
| CDR-1 | | |
| VR.14G4_ | KASTLAS | 64 |
| LC: CDR-2 | | |
| R.14G4_LC: | AGYKSTITDGTA | 78 |
| CDR-3 | | |
| R.14G4_HC | QEQLKESGGGLVTPGGTLTLTCTASGFTITSYH | 118 |
| | MCWVRQAPGKGLEWIGCIYPNNGFTWYASWAKG | |
| | RFTISKTSSTTVDLKMTSPTTEEMATYFCARgs | |
| | GNSYNQLDLWGQGTLVTVSSASTKGPSVFPLAP | |
| | SSKSTSGGTAALGCLVKDYFPEPVTVSWNSGAL | |
| | TSGVHTFPAVLQSSGLYSLSSVVTVPSSSLGTQ | |
| | TYICNVNHKPSNTKVDKKVEPKSCDKTHTCPPC | |
| | PAPELLGGPSVFLFPPKPKDTLMISRTPEVTCV | |
| | VVDVSHEDPEVKFNWYVDGVEVHNAKTKPREEQ | |
| | YNSTYRVVSVLTVLHQDWLNGKEYKCKVSNKAL | |
| | PAPIEKTISKAKGQPREPQVYTLPPSRDELTKN | |
| | QVSLTCLVKGFYPSDIAVEWESNGQPENNYKTT | |
| | PPVLDSDGSFFLYSKLTVDKSRWQQGNVFSCSV | |
| | MHEALHNHYTQKSLSLSPGK | |
| R.14G4_HC | CAGGAACAGCTGAAGGAGAGCGGAGGAGGACTG | 119 |
| (Nucleo- | GTGACACCAGGAGGAACACTGACACTGACCTGC | |
| tide) | ACCGCAAGCGGATTCACCATCACCAGCTATCAC | |
| | ATGTGCTGGGTGAGACAGGCCCCAGGAAAGGGA | |
| | CTGGAATGGATCGGATGCATTTACCCAAATAAC | |
| | GGCTTTACCTGGTACGCCTCCTGGGCAAAGGGC | |
| | AGATTCACCATCTCCAAAACCTCCTCCACCACA | |
| | GTCGATCTGAAGATGACATCCCCCACCACCGAA | |
| | GAGATGGCCACATACTTCTGCGCCCGCGGATCT | |
| | GGAAACAGCTACAACCAGCTGGACCTGTGGGGC | |
| | CAGGGAACACTGGTGACAGTGAGTAGCGCTAGC | |
| | ACCAAAGGTCCCAGCGTGTTCCCACTCGCCCCG | |
| | AGTTCAAAATCAACTTCTGGAGGCACCGCCGCC | |
| | CTGGGTTGCCTGGTAAAGGACTACTTCCCAGAG | |
| | CCCGTGACCGTGAGCTGGAACTCCGGGGCACTG | |
| | ACATCTGGCGTTCATACTTTCCCGGCCGTGCTC | |
| | CAGTCTTCAGGTCTGTATAGTCTCTCCTCTGTG | |
| | GTCACTGTCCCATCTAGCTCTCTGGGCACCCAA | |
| | ACCTACATATGCAACGTTAATCACAAGCCGAGC | |
| | AATACTAAAGTTGACAAAAAGGTGGAACCCAAG | |
| | TCTTGTGACAAGACCCACACGTGTCCCCCCTGC | |
| | CCGGCTCCTGAGCTGCTTGGCGGCCCCAGCGTC | |
| | TTTCTCTTTCCCCCAAAGCCAAAAGATACCTTG | |
| | ATGATCAGCAGAACTCCCGAGGTGACATGCGTC | |
| | GTCGTGGACGTAAGCCATGAAGATCCCGAGGTT | |
| | AAGTTCAACTGGTATGTCGATGGCGTGGAAGTC | |
| | CATAATGCTAAGACTAAACCTCGCGAAGAGCAG | |
| | TACAATTCAACTTACCGGGTCGTTTCCGTTCTG | |
| | ACCGTGCTGCATCAGGACTGGCTGAATGGTAAA | |
| | GAGTACAAGTGCAAAGTGTCTAACAAGGCACTC | |
| | CCCGCCCCAATTGAGAAGACTATCTCCAAAGCT | |
| | AAAGGGCAACCAAGAGAGCCCCAGGTCTACACC | |
| | CTGCCCCCCTCAAGGGATGAGCTTACTAAGAAC | |
| | CAGGTTAGTCTCACCTGCTTGGTTAAAGGATTT | |
| | TATCCAAGCGATATTGCTGTGGAGTGGGAGTCC | |
| | AACGGCCAGCCTGAGAACAATTATAAAACCACC | |
| | CCCCCTGTTCTTGACAGTGACGGTAGTTTCTTC | |
| | CTGTATTCCAAACTGACCGTCGATAAGAGCAGA | |
| | TGGCAACAGGGAAATGTGTTCAGCTGCTCCGTG | |
| | ATGCATGAGGCGCTCCATAATCATTACACACAA | |
| | AAAAGTTTGTCCCTGAGCCCAGGCAAG | |
| R.14G4_LC | AIVMTQTPASVSATLGGSVTINCQSSESVYRDN | 120 |
| | CLGWFQQKPGQPPKLLIYKASTLASGVPSRFKG | |
| | SGSGTQFTLTISDVVSDDAATYYCAGYKSTITD | |
| | GTAFGGGTEVVVKRTVAAPSVFIFPPSDEQLKS | |
| | GTASVVCLLNNFYPREAKVQWKVDNALQSGNSQ | |
| | ESVTEQDSKDSTYSLSSTLTLSKADYEKHKVYA | |
| | CEVTHQGLSSPVTKSFNRGEC | |
| R.14G4_LC | GCTATTGTGATGACCCAGACCCCCGCATCCGTG | 121 |
| (Nucleo- | TCTGCTACACTGGGAGGAAGTGTGACCATTAAT | |
| tide) | TGTCAGTCCTCCGAGTCCGTGTACCGCGATAAT | |
| | TGTCTGGGATGGTTCCAGCAGAAGCCTGGGCAG | |
| | CCTCCTAAACTGCTGATTTACAAAGCCTCTACA | |
| | CTGGCCTCCGGCGTCCCTTCTAGATTCAAGGGA | |
| | TCCGGGTCCGGCACCCAGTTTACTCTGACTATC | |
| | TCCGACGTGGTGTCTGACGACGCCGCTACATAC | |
| | TACTGCGCAGGTTACAAGTCCACCATCACCGAT | |
| | GGCACCGCCTTCGGAGGAGGAACAGAAGTGGTG | |
| | GTGAAGcgtacgGTGGCAGCTCCATCAGTTTTT | |
| | ATCTTCCCACCAAGCGACGAGCAATTGAAGTCC | |
| | GGCACTGCCTCTGTGGTGTGCCTTCTGAACAAC | |
| | TTCTATCCAAGGGAGGCCAAGGTCCAGTGGAAG | |
| | GTCGATAATGCGCTGCAGAGCGGGAACAGCCAA | |
| | GAGTCAGTGACCGAGCAGGACTCAAAAGATAGC | |
| | ACATACTCTCTGAGTTCCACCCTGACCCTGTCA | |
| | AAGGCTGACTACGAAAAGCATAAGGTATACGCA | |
| | TGCGAAGTGACCCATCAGGGTCTCTCATCTCCC | |
| | GTAACCAAATCTTTTAATAGAGGAGAATGC | |
| R.15G10_HC: | SYHMN | 9 |
| CDR-1 | | |
| R.15G10_HC: | IIYVGSGNADYASWAKG | 23 |
| CDR-2 | | |
| R.15G10_HC: | GPTNTYPTYFNL | 38 |
| CDR-3 | | |
| R.15G10_LC: | QASRSITSYLA | 54 |
| CDR-1 | | |
| R.15G10_LC: | AASNVAS | 65 |
| CDR-2 | | |
| R.15G10_LC: | QSYYAISTNT | 79 |
| CDR-3 | | |
| R.15G10_HC | QSLEESGGRLVTPGTPLTLTCTASGFTISSYHM | 123 |
| | NWVRQAPGEGLEWIGIIYVGSGNADYASWAKGR | |
| | FTISKASTTVDLKITSPTTEDTATYFCARGPTN | |
| | TYPTYFNLWGPGTLVTVSSASTKGPSVFPLAPS | |
| | SKSTSGGTAALGCLVKDYFPEPVTVSWNSGALT | |
| | SGVHTFPAVLQSSGLYSLSSVVTVPSSSLGTQT | |
| | YICNVNHKPSNTKVDKKVEPKSCDKTHTCPPCP | |
| | APELLGGPSVFLFPPKPKDTLMISRTPEVTCVV | |
| | VDVSHEDPEVKFNWYVDGVEVHNAKTKPREEQY | |
| | NSTYRVVSVLTVLHQDWLNGKEYKCKVSNKALP | |
| | APIEKTISKAKGQPREPQVYTLPPSRDELTKNQ | |
| | VSLTCLVKGFYPSDIAVEWESNGQPENNYKTTP | |
| | PVLDSDGSFFLYSKLTVDKSRWQQGNVFSCSVM | |
| | HEALHNHYTQKSLSLSPGK | |
| R.15G10_HC | CAGTCTCTGGAAGAATCCGGCGGAAGGCTGGTG | 124 |
| (Nucleo- | ACACCAGGAACACCTCTGACCCTGACCTGCACA | |
| tide) | GCATCCGGATTCACCATCTCCTCCTACCACATG | |
| | AACTGGGTCCGGCAGGCACCTGGAGAAGGACTG | |
| | GAATGGATCGGAATCATCTACGTCGGCTCCGGC | |
| | AACGCAGACTACGCTTCTTGGGCTAAAGGCCGC | |
| | TTCACTATCTCCAAGGCCTCCACTACCGTCGAC | |
| | CTGAAAATCACAAGCCCCACCACCGAAGACACC | |
| | GCAACATATTTTTGCGCTAGGGGACCAACCAAC | |
| | ACCTACCCTACATACTTCAACCTGTGGGGCCCC | |
| | GGAACCCTGGTGACAGTGTCTTCTGCTAGCACC | |
| | AAAGGTCCCAGCGTGTTCCCACTCGCCCCGAGT | |
| | TCAAAATCAACTTCTGGAGGCACCGCCGCCCTG | |
| | GGTTGCCTGGTAAAGGACTACTTCCCAGAGCCC | |
| | GTGACCGTGAGCTGGAACTCCGGGGCACTGACA | |
| | TCTGGCGTTCATACTTTCCCGGCCGTGCTCCAG | |
| | TCTTCAGGTCTGTATAGTCTCTCCTCTGTGGTC | |
| | ACTGTCCCATCTAGCTCTCTGGGCACCCAAACC | |
| | TACATATGCAACGTTAATCACAAGCCGAGCAAT | |
| | ACTAAAGTTGACAAAAAGGTGGAACCCAAGTCT | |
| | TGTGACAAGACCCACACGTGTCCCCCCTGCCCG | |
| | GCTCCTGAGCTGCTTGGCGGCCCCAGCGTCTTT | |
| | CTCTTTCCCCCAAAGCCAAAAGATACCTTGATG | |
| | ATCAGCAGAACTCCCGAGGTGACATGCGTCGTC | |
| | GTGGACGTAAGCCATGAAGATCCCGAGGTTAAG | |
| | TTCAACTGGTATGTCGATGGCGTGGAAGTCCAT | |
| | AATGCTAAGACTAAACCTCGCGAAGAGCAGTAC | |
| | AATTCAACTTACCGGGTCGTTTCCGTTCTGACC | |
| | GTGCTGCATCAGGACTGGCTGAATGGTAAAGAG | |
| | TACAAGTGCAAAGTGTCTAACAAGGCACTCCCC | |
| | GCCCCAATTGAGAAGACTATCTCCAAAGCTAAA | |
| | GGGCAACCAAGAGAGCCCCAGGTCTACACCCTG | |
| | CCCCCCTCAAGGGATGAGCTTACTAAGAACCAG | |
| | GTTAGTCTCACCTGCTTGGTTAAAGGATTTTAT | |
| | CCAAGCGATATTGCTGTGGAGTGGGAGTCCAAC | |
| | GGCCAGCCTGAGAACAATTATAAAACCACCCCC | |
| | CCTGTTCTTGACAGTGACGGTAGTTTCTTCCTG | |
| | TATTCCAAACTGACCGTCGATAAGAGCAGATGG | |
| | CAACAGGGAAATGTGTTCAGCTGCTCCGTGATG | |
| | CATGAGGCGCTCCATAATCATTACACACAAAAA | |
| | AGTTTGTCCCTGAGCCCAGGCAAG | |
| R.15G10_LC | AFELTQTPSSVEAAVGGTVTIKCQASRSITSYL | 125 |
| | AWYQHKPGQPPTLLIYAASNVASGVSSRFKGSR | |
| | SGTEYTLTISDLESADAATYYCQSYYAISTNTF | |
| | GGGTEVVVKRTVAAPSVFIFPPSDEQLKSGTAS | |
| | VVCLLNNFYPREAKVQWKVDNALQSGNSQESVT | |
| | EQDSKDSTYSLSSTLTLSKADYEKHKVYACEVT | |
| | HQGLSSPVTKSFNRGEC | |
| R.15G10_LC | GCTTTTGAACTGACCCAGACCCCCTCCTCCGTG | 126 |
| (Nucleo- | GAAGCTGCTGTGGGAGGAACAGTGACCATTAAG | |
| tide) | TGTCAGGCCTCTAGGTCTATCACCTCCTACCTG | |
| | GCTTGGTATCAGCACAAGCCCGGTCAGCCTCCA | |
| | ACCCTGCTGATTTACGCTGCCTCTAATGTGGCT | |
| | TCTGGCGTGTCCTCCCGCTTCAAAGGATCCAGA | |
| | TCCGGCACCGAGTACACCCTGACTATCTCCGAC | |
| | CTGGAATCTGCCGACGCCGCTACATACTACTGC | |
| | CAGTCTTACTACGCCATTTCCACCAATACCTTC | |
| | GGCGGCGGAACCGAAGTGGTCGTGAAAcgtacg | |
| | GTGGCAGCTCCATCAGTTTTTATCTTCCCACCA | |
| | AGCGACGAGCAATTGAAGTCCGGCACTGCCTCT | |
| | GTGGTGTGCCTTCTGAACAACTTCTATCCAAGG | |
| | GAGGCCAAGGTCCAGTGGAAGGTCGATAATGCG | |
| | CTGCAGAGCGGGAACAGCCAAGAGTCAGTGACC | |
| | GAGCAGGACTCAAAAGATAGCACATACTCTCTG | |
| | AGTTCCACCCTGACCCTGTCAAAGGCTGACTAC | |
| | GAAAAGCATAAGGTATACGCATGCGAAGTGACC | |
| | CATCAGGGTCTCTCATCTCCCGTAACCAAATCT | |
| | TTTAATAGAGGAGAATGC | |
| R.18B9_HC: | SGYMC | 10 |
| CDR-1 | | |
| R.18B9_HC: | CIYAGSSGKTYYASWATG | 24 |
| CDR-2 | | |
| R.18B9_HC: | DDGGTYYTDYLYL | 39 |
| CDR-3 | | |
| R.18B9_LC: | QASEGISSYLS | 55 |
| CDR-1 | | |
| R.18B9_LC: | KASTLTS | 66 |
| CDR-2 | | |
| R.18B9_LC: | QCTYGSSGSSGYGAA | 80 |
| CDR-3 | | |
| R.18B9_HC | QSLEESGGDLVKPGASLTLTCKASGFSFNSGYM | 127 |
| | CWVRQAPGKGLEWIACIYAGSSGKTYYASWATG | |
| | RFTISKTSSTTVTLQMTSLTVADTATYFCARDD | |
| | GGTYYTDYLYLWGPGTLVTVSSASTKGPSVFPL | |
| | APSSKSTSGGTAALGCLVKDYFPEPVTVSWNSG | |
| | ALTSGVHTFPAVLQSSGLYSLSSVVTVPSSSLG | |
| | TQTYICNVNHKPSNTKVDKKVEPKSCDKTHTCP | |
| | PCPAPELLGGPSVFLFPPKPKDTLMISRTPEVT | |
| | CVVVDVSHEDPEVKFNWYVDGVEVHNAKTKPRE | |
| | EQYNSTYRVVSVLTVLHQDWLNGKEYKCKVSNK | |
| | ALPAPIEKTISKAKGQPREPQVYTLPPSRDELT | |
| | KNQVSLTCLVKGFYPSDIAVEWESNGQPENNYK | |
| | TTPPVLDSDGSFFLYSKLTVDKSRWQQGNVFSC | |
| | SVMHEALHNHYTQKSLSLSPGK | |
| R.18B9_HC | CAGTCTCTGGAAGAATCCGGCGGAGACCTGGTG | 128 |
| (Nucleo- | AAGCCTGGAGCTTCTCTGACCCTGACCTGCAAA | |
| tide) | GCCTCCGGATTTTCCTTTAACTCCGGCTACATG | |
| | TGCTGGGTGCGGCAGGCTCCTGGAAAAGGACTG | |
| | GAATGGATCGCCTGCATTTACGCCGGGTCTTCC | |
| | GGAAAGACCTACTACGCATCCTGGGCTACCGGC | |
| | AGATTCACCATTTCCAAGACTAGCAGTACAACC | |
| | GTGACACTGCAGATGACCTCCCTGACCGTGGCT | |
| | GATACCGCTACATACTTCTGTGCCCGGGACGAC | |
| | GGAGGTACATATTACACCGACTACCTGTACCTG | |
| | TGGGGCCCAGGAACACTGGTGACAGTGTCTTCC | |
| | GCTAGCACCAAAGGTCCCAGCGTGTTCCCACTC | |
| | GCCCCGAGTTCAAAATCAACTTCTGGAGGCACC | |
| | GCCGCCCTGGGTTGCCTGGTAAAGGACTACTTC | |
| | CCAGAGCCCGTGACCGTGAGCTGGAACTCCGGG | |
| | GCACTGACATCTGGCGTTCATACTTTCCCGGCC | |
| | GTGCTCCAGTCTTCAGGTCTGTATAGTCTCTCC | |
| | TCTGTGGTCACTGTCCCATCTAGCTCTCTGGGC | |
| | ACCCAAACCTACATATGCAACGTTAATCACAAG | |
| | CCGAGCAATACTAAAGTTGACAAAAAGGTGGAA | |
| | CCCAAGTCTTGTGACAAGACCCACACGTGTCCC | |
| | CCCTGCCCGGCTCCTGAGCTGCTTGGCGGCCCC | |
| | AGCGTCTTTCTCTTTCCCCCAAAGCCAAAAGAT | |
| | ACCTTGATGATCAGCAGAACTCCCGAGGTGACA | |
| | TGCGTCGTCGTGGACGTAAGCCATGAAGATCCC | |
| | GAGGTTAAGTTCAACTGGTATGTCGATGGCGTG | |
| | GAAGTCCATAATGCTAAGACTAAACCTCGCGAA | |
| | GAGCAGTACAATTCAACTTACCGGGTCGTTTCC | |
| | GTTCTGACCGTGCTGCATCAGGACTGGCTGAAT | |
| | GGTAAAGAGTACAAGTGCAAAGTGTCTAACAAG | |
| | GCACTCCCCGCCCCAATTGAGAAGACTATCTCC | |
| | AAAGCTAAAGGGCAACCAAGAGAGCCCCAGGTC | |
| | TACACCCTGCCCCCCTCAAGGGATGAGCTTACT | |
| | AAGAACCAGGTTAGTCTCACCTGCTTGGTTAAA | |
| | GGATTTTATCCAAGCGATATTGCTGTGGAGTGG | |
| | GAGTCCAACGGCCAGCCTGAGAACAATTATAAA | |
| | ACCACCCCCCCTGTTCTTGACAGTGACGGTAGT | |
| | TTCTTCCTGTATTCCAAACTGACCGTCGATAAG | |
| | AGCAGATGGCAACAGGGAAATGTGTTCAGCTGC | |
| | TCCGTGATGCATGAGGCGCTCCATAATCATTAC | |
| | ACACAAAAAAGTTTGTCCCTGAGCCCAGGCAAG | |
| R.18B9_LC | DVVMTQTPASVEAAVGGTVTIKCQASEGISSYL | 129 |
| | SWYQQKPGQPPKLLIYKASTLTSGVPSRFKGSG | |
| | SGTDFTLTISDLESADAATYYCQCTYGSSGSSG | |
| | YGAAFGGGTEVVVKRTVAAPSVFIFPPSDEQLK | |
| | SGTASVVCLLNNFYPREAKVQWKVDNALQSGNS | |
| | QESVTEQDSKDSTYSLSSTLTLSKADYEKHKVY | |
| | ACEVTHQGLSSPVTKSFNRGEC | |
| R.18B9_LC | GATGTGGTGATGACCCAGACCCCAGCAAGCGTG | 130 |
| (Nucleo- | GAAGCAGCTGTGGGAGGAACAGTGACCATTAAG | |
| tide) | TGCCAGGCCTCCGAAGGAATCAGCAGCTATCTG | |
| | AGCTGGTATCAGCAGAAGCCCGGGCAGCCTCCT | |
| | AAACTGCTGATTTACAAGGCAAGTACCCTGACA | |
| | TCTGGCGTGCCCTCTAGGTTTAAAGGAAGCGGA | |
| | AGCGGCACAGACTTCACCCTGACAATCTCCGAC | |
| | CTGGAAAGCGCCGACGCAGCTACATACTACTGC | |
| | CAGTGCACCTACGGCTCCTCCGGATCTTCTGGT | |
| | TACGGCGCTGCTTTTGGCGGGGGAACAGAAGTG | |
| | GTGGTGAAGcgtacgGTGGCAGCTCCATCAGTT | |
| | TTTATCTTCCCACCAAGCGACGAGCAATTGAAG | |
| | TCCGGCACTGCCTCTGTGGTGTGCCTTCTGAAC | |
| | AACTTCTATCCAAGGGAGGCCAAGGTCCAGTGG | |
| | AAGGTCGATAATGCGCTGCAGAGCGGGAACAGC | |
| | CAAGAGTCAGTGACCGAGCAGGACTCAAAAGAT | |
| | AGCACATACTCTCTGAGTTCCACCCTGACCCTG | |
| | TCAAAGGCTGACTACGAAAAGCATAAGGTATAC | |
| | GCATGCGAAGTGACCCATCAGGGTCTCTCATCT | |
| | CCCGTAACCAAATCTTTTAATAGAGGAGAATGC | |
| R.19H7_HC: | SYHMC | 8 |
| CDR-1 | | |
| R.19H7_HC: | CIYPNNDFTWYATWAKG | 25 |
| CDR-2 | | |
| R.19H7_HC: | ASGNAYNLLDL | 40 |
| CDR-3 | | |
| R.19H7_LC: | QSSESVYRDNCLG | 53 |
| CDR-1 | | |
| R.19H7_LC: | RASTLAS | 67 |
| CDR-2 | | |
| R.19H7_LC: | AGYKSTITDGTA | 78 |
| CDR-3 | | |
| R.19H7_HC | QEQLKESGGGLVTPGGTLTLTCTASGFTISSYH | 131 |
| | MCWVRQAPGKGLEWIGCIYPNNDFTWYATWAKG | |
| | RFTISKTSSTTVDLRMTTPTTEETATYFCARAS | |
| | GNAYNLLDLWGQGTLVTVSSASTKGPSVFPLAP | |
| | SSKSTSGGTAALGCLVKDYFPEPVTVSWNSGAL | |
| | TSGVHTFPAVLQSSGLYSLSSVVTVPSSSLGTQ | |
| | TYICNVNHKPSNTKVDKKVEPKSCDKTHTCPPC | |
| | PAPELLGGPSVFLFPPKPKDTLMISRTPEVTCV | |
| | VVDVSHEDPEVKFNWYVDGVEVHNAKTKPREEQ | |
| | YNSTYRVVSVLTVLHQDWLNGKEYKCKVSNKAL | |
| | PAPIEKTISKAKGQPREPQVYTLPPSRDELTKN | |
| | QVSLTCLVKGFYPSDIAVEWESNGQPENNYKTT | |
| | PPVLDSDGSFFLYSKLTVDKSRWQQGNVFSCSV | |
| | MHEALHNHYTQKSLSLSPGK | |
| R.19H7_HC | CAGGAACAGCTGAAGGAGAGCGGAGGAGGACTG | 132 |
| (Nucleo- | GTGACACCAGGAGGAACACTGACACTGACCTGC | |
| tide) | ACCGCAAGCGGATTCACCATCAGCAGCTACCAC | |
| | ATGTGCTGGGTGAGGCAGGCACCAGGAAAGGGA | |
| | CTGGAATGGATTGGCTGCATTTACCCAAATAAC | |
| | GACTTCACCTGGTACGCTACATGGGCCAAGGGC | |
| | AGATTCACCATCTCCAAAACCTCCTCCACCACC | |
| | GTCGATCTGAGGATGACCACCCCTACTACAGAG | |
| | GAGACCGCCACATACTTCTGCGCCAGAGCTTCC | |
| | GGTAACGCCTACAACCTGCTGGACCTGTGGGGA | |
| | CAGGGAACACTGGTGACAGTCTCCTCCGCTAGC | |
| | ACCAAAGGTCCCAGCGTGTTCCCACTCGCCCCG | |
| | AGTTCAAAATCAACTTCTGGAGGCACCGCCGCC | |
| | CTGGGTTGCCTGGTAAAGGACTACTTCCCAGAG | |
| | CCCGTGACCGTGAGCTGGAACTCCGGGGCACTG | |
| | ACATCTGGCGTTCATACTTTCCCGGCCGTGCTC | |
| | CAGTCTTCAGGTCTGTATAGTCTCTCCTCTGTG | |
| | GTCACTGTCCCATCTAGCTCTCTGGGCACCCAA | |
| | ACCTACATATGCAACGTTAATCACAAGCCGAGC | |
| | AATACTAAAGTTGACAAAAAGGTGGAACCCAAG | |
| | TCTTGTGACAAGACCCACACGTGTCCCCCCTGC | |
| | CCGGCTCCTGAGCTGCTTGGCGGCCCCAGCGTC | |
| | TTTCTCTTTCCCCCAAAGCCAAAAGATACCTTG | |
| | ATGATCAGCAGAACTCCCGAGGTGACATGCGTC | |
| | GTCGTGGACGTAAGCCATGAAGATCCCGAGGTT | |
| | AAGTTCAACTGGTATGTCGATGGCGTGGAAGTC | |
| | CATAATGCTAAGACTAAACCTCGCGAAGAGCAG | |
| | TACAATTCAACTTACCGGGTCGTTTCCGTTCTG | |
| | ACCGTGCTGCATCAGGACTGGCTGAATGGTAAA | |
| | GAGTACAAGTGCAAAGTGTCTAACAAGGCACTC | |
| | CCCGCCCCAATTGAGAAGACTATCTCCAAAGCT | |
| | AAAGGGCAACCAAGAGAGCCCCAGGTCTACACC | |
| | CTGCCCCCCTCAAGGGATGAGCTTACTAAGAAC | |
| | CAGGTTAGTCTCACCTGCTTGGTTAAAGGATTT | |
| | TATCCAAGCGATATTGCTGTGGAGTGGGAGTCC | |
| | AACGGCCAGCCTGAGAACAATTATAAAACCACC | |
| | CCCCCTGTTCTTGACAGTGACGGTAGTTTCTTC | |
| | CTGTATTCCAAACTGACCGTCGATAAGAGCAGA | |
| | TGGCAACAGGGAAATGTGTTCAGCTGCTCCGTG | |
| | ATGCATGAGGCGCTCCATAATCATTACACACAA | |
| | AAAAGTTTGTCCCTGAGCCCAGGCAAG | |
| R.19H7_LC | AIVMTQTPASVSVAVGGSVTINCQSSESVYRDN | 133 |
| | CLGWFQQKPGQPPKLLIYRASTLASGVPSRFKG | |
| | SGSGTQFTLTISDVVSDDAATYYCAGYKSTITD | |
| | GTAFGGGTEVVVKRTVAAPSVFIFPPSDEQLKS | |
| | GTASVVCLLNNFYPREAKVQWKVDNALQSGNSQ | |
| | ESVTEQDSKDSTYSLSSTLTLSKADYEKHKVYA | |
| | CEVTHQGLSSPVTKSFNRGEC | |
| R.19H7_LC | GCTATTGTGATGACCCAGACCCCCGCATCCGTG | 134 |
| (Nucleo- | TCTGTGGCTGTGGGAGGATCTGTGACCATTAAT | |
| tide) | TGTCAGTCCAGTGAATCCGTGTATCGTGACAAC | |
| | TGTCTGGGATGGTTCCAGCAGAAACCCGGGCAG | |
| | CCTCCTAAACTGCTGATTTACAGGGCCTCTACA | |
| | CTGGCTTCCGGCGTGCCTTCTAGGTTCAAGGGA | |
| | TCCGGATCCGGCACACAGTTCACTCTGACTATC | |
| | TCCGACGTGGTGTCTGACGACGCCGCTACATAC | |
| | TACTGCGCAGGATACAAGTCCACCATCACCGAC | |
| | GGCACCGCATTCGGAGGAGGAACAGAAGTGGTG | |
| | GTGAAGcgtacgGTGGCAGCTCCATCAGTTTTT | |
| | ATCTTCCCACCAAGCGACGAGCAATTGAAGTCC | |
| | GGCACTGCCTCTGTGGTGTGCCTTCTGAACAAC | |
| | TTCTATCCAAGGGAGGCCAAGGTCCAGTGGAAG | |
| | GTCGATAATGCGCTGCAGAGCGGGAACAGCCAA | |
| | GAGTCAGTGACCGAGCAGGACTCAAAAGATAGC | |
| | ACATACTCTCTGAGTTCCACCCTGACCCTGTCA | |
| | AAGGCTGACTACGAAAAGCATAAGGTATACGCA | |
| | TGCGAAGTGACCCATCAGGGTCTCTCATCTCCC | |
| | GTAACCAAATCTTTTAATAGAGGAGAATGC | |
| R.20H1_HC: | SGYYMC | 11 |
| CDR-1 | | |
| R.20H1_HC: | CIYVRSDSTYYASWAKG | 26 |
| CDR-2 | | |
| R.20H1_HC: | GVTGDSYRFNF | 41 |
| CDR-3 | | |
| R.20H1_LC: | QASQSISNELS | 52 |
| CDR-1 | | |
| R.20H1_LC: | RASTLES | 62 |
| CDR-2 | | |
| R.20H1_LC: | QSTYYGTSYGNA | 81 |
| CDR-3 | | |
| R.20H1_HC | QEQLVESGGGLVKPGASLTLTCKASGFSFSSGY | 135 |
| | YMCWVRQAPGKGLEWIGCIYVRSDSTYYASWAK | |
| | GRFTISKASSTTVTLQMTSLTAADTATYFCARG | |
| | VTGDSYRFNFWGPGTLVTVSSASTKGPSVFPLA | |
| | PSSKSTSGGTAALGCLVKDYFPEPVTVSWNSGA | |
| | LTSGVHTFPAVLQSSGLYSLSSVVTVPSSSLGT | |
| | QTYICNVNHKPSNTKVDKKVEPKSCDKTHTCPP | |
| | CPAPELLGGPSVFLFPPKPKDTLMISRTPEVTC | |
| | VVVDVSHEDPEVKFNWYVDGVEVHNAKTKPREE | |
| | QYNSTYRVVSVLTVLHQDWLNGKEYKCKVSNKA | |
| | LPAPIEKTISKAKGQPREPQVYTLPPSRDELTK | |
| | NQVSLTCLVKGFYPSDIAVEWESNGQPENNYKT | |
| | TPPVLDSDGSFFLYSKLTVDKSRWQQGNVFSCS | |
| | VMHEALHNHYTQKSLSLSPGK | |
| R.20H1_HC | CAGGAACAGCTGGTGGAAAGTGGCGGAGGACTG | 136 |
| (Nucleo- | GTGAAGCCTGGAGCTTCTCTGACCCTGACATGC | |
| tide) | AAGGCATCCGGCTTCAGCTTCAGTAGCGGCTAC | |
| | TATATGTGCTGGGTGAGACAGGCCCCAGGCAAA | |
| | GGACTGGAATGGATCGGATGTATCTACGTGCGA | |
| | TCCGACTCCACCTACTACGCCTCTTGGGCTAAG | |
| | GGAAGGTTCACCATTTCCAAGGCCTCCTCCACC | |
| | ACCGTCACCCTGCAGATGACATCCCTGACTGCC | |
| | GCTGACACCGCTACATATTTCTGCGCCAGGGGC | |
| | GTGACCGGAGATTCTTATAGGTTCAACTTCTGG | |
| | GGCCCCGGAACCCTGGTGACAGTGTCTTCTGCT | |
| | AGCACCAAAGGTCCCAGCGTGTTCCCACTCGCC | |
| | CCGAGTTCAAAATCAACTTCTGGAGGCACCGCC | |
| | GCCCTGGGTTGCCTGGTAAAGGACTACTTCCCA | |
| | GAGCCCGTGACCGTGAGCTGGAACTCCGGGGCA | |
| | CTGACATCTGGCGTTCATACTTTCCCGGCCGTG | |
| | CTCCAGTCTTCAGGTCTGTATAGTCTCTCCTCT | |
| | GTGGTCACTGTCCCATCTAGCTCTCTGGGCACC | |
| | CAAACCTACATATGCAACGTTAATCACAAGCCG | |
| | AGCAATACTAAAGTTGACAAAAAGGTGGAACCC | |
| | AAGTCTTGTGACAAGACCCACACGTGTCCCCCC | |
| | TGCCCGGCTCCTGAGCTGCTTGGCGGCCCCAGC | |
| | GTCTTTCTCTTTCCCCCAAAGCCAAAAGATACC | |
| | TTGATGATCAGCAGAACTCCCGAGGTGACATGC | |
| | GTCGTCGTGGACGTAAGCCATGAAGATCCCGAG | |
| | GTTAAGTTCAACTGGTATGTCGATGGCGTGGAA | |
| | GTCCATAATGCTAAGACTAAACCTCGCGAAGAG | |
| | CAGTACAATTCAACTTACCGGGTCGTTTCCGTT | |
| | CTGACCGTGCTGCATCAGGACTGGCTGAATGGT | |
| | AAAGAGTACAAGTGCAAAGTGTCTAACAAGGCA | |
| | CTCCCCGCCCCAATTGAGAAGACTATCTCCAAA | |
| | GCTAAAGGGCAACCAAGAGAGCCCCAGGTCTAC | |
| | ACCCTGCCCCCCTCAAGGGATGAGCTTACTAAG | |
| | AACCAGGTTAGTCTCACCTGCTTGGTTAAAGGA | |
| | TTTTATCCAAGCGATATTGCTGTGGAGTGGGAG | |
| | TCCAACGGCCAGCCTGAGAACAATTATAAAACC | |
| | ACCCCCCCTGTTCTTGACAGTGACGGTAGTTTC | |
| | TTCCTGTATTCCAAACTGACCGTCGATAAGAGC | |
| | AGATGGCAACAGGGAAATGTGTTCAGCTGCTCC | |
| | GTGATGCATGAGGCGCTCCATAATCATTACACA | |
| | CAAAAAAGTTTGTCCCTGAGCCCAGGCAAG | |
| R.20H1_LC | DVVMTQTPASVEAAVGGTVTIKCQASQSISNEL | 137 |
| | SWYQQKSGQPPKLLIYRASTLESGVPSRFKGSG | |
| | SGTEFTLTISDLESADAATYYCQSTYYGTSYGN | |
| | AFGGGTEVVVKRTVAAPSVFIFPPSDEQLKSGT | |
| | ASVVCLLNNFYPREAKVQWKVDNALQSGNSQES | |
| | VTEQDSKDSTYSLSSTLTLSKADYEKHKVYACE | |
| | VTHQGLSSPVTKSFNRGEC | |
| R.20H1_LC | GATGTGGTGATGACCCAGACCCCAGCAAGCGTG | 138 |
| (Nucleo- | GAAGCAGCTGTGGGAGGAACAGTGACCATTAAG | |
| tide) | TGCCAGGCCTCCCAGTCCATTTCCAACGAGCTG | |
| | AGCTGGTACCAGCAGAAGAGCGGACAGCCTCCA | |
| | AAACTGCTGATTTACAGGGCAAGTACACTGGAG | |
| | AGCGGAGTGCCATCCAGGTTCAAGGGATCCGGA | |
| | AGCGGAACCGAGTTCACCCTGACTATCTCCGAC | |
| | CTGGAGTCTGCCGACGCTGCTACATACTACTGC | |
| | CAGTCCACCTACTACGGCACCAGTTACGGCAAT | |
| | GCATTCGGCGGAGGCACAGAGGTGGTGGTGAAA | |
| | cgtacgGTGGCAGCTCCATCAGTTTTTATCTTC | |
| | CCACCAAGCGACGAGCAATTGAAGTCCGGCACT | |
| | GCCTCTGTGGTGTGCCTTCTGAACAACTTCTAT | |
| | CCAAGGGAGGCCAAGGTCCAGTGGAAGGTCGAT | |
| | AATGCGCTGCAGAGCGGGAACAGCCAAGAGTCA | |
| | GTGACCGAGCAGGACTCAAAAGATAGCACATAC | |
| | TCTCTGAGTTCCACCCTGACCCTGTCAAAGGCT | |
| | GACTACGAAAAGCATAAGGTATACGCATGCGAA | |
| | GTGACCCATCAGGGTCTCTCATCTCCCGTAACC | |
| | AAATCTTTTAATAGAGGAGAATGC | |
| R.1B11_HC: | SYHMS | 12 |
| CDR-1 | | |
| R.1B11_HC: | IIYADTGNADYASWTKG | 27 |
| CDR-2 | | |
| R.1B11_HC: | GPTNTYPTYFAL | 42 |
| CDR-3 | | |
| R.1B11_LC: | QASQSITTYLA | 56 |
| CDR-1 | | |
| R.1B11_LC: | GASNLES | 68 |
| CDR-2 | | |
| R.1B11_LC: | QSYYAISKSA | 82 |
| CDR-3 | | |
| R.1B11_HC | QSLEESGGRLVTPGTPLTLTCTASGFSLNSYHM | 139 |
| | SWVRQAPGEGLEWIGIIYADTGNADYASWTKGR | |
| | FTISKASTTVELRITSPTSEDTATYFCARGPTN | |
| | TYPTYFALWGPGTLVTVSSASTKGPSVFPLAPS | |
| | SKSTSGGTAALGCLVKDYFPEPVTVSWNSGALT | |
| | SGVHTFPAVLQSSGLYSLSSVVTVPSSSLGTQT | |
| | YICNVNHKPSNTKVDKKVEPKSCDKTHTCPPCP | |
| | APELLGGPSVFLFPPKPKDTLMISRTPEVTCVV | |
| | VDVSHEDPEVKFNWYVDGVEVHNAKTKPREEQY | |
| | NSTYRVVSVLTVLHQDWLNGKEYKCKVSNKALP | |
| | APIEKTISKAKGQPREPQVYTLPPSRDELTKNQ | |
| | VSLTCLVKGFYPSDIAVEWESNGQPENNYKTTP | |
| | PVLDSDGSFFLYSKLTVDKSRWQQGNVFSCSVM | |
| | HEALHNHYTQKSLSLSPGK | |
| R.1B11_HC | CAGTCTCTGGAAGAATCCGGCGGAAGGCTGGTG | 140 |
| (Nucleo- | ACACCAGGAACACCTCTGACCCTGACCTGCACA | |
| tide) | GCATCCGGATTCAGCCTGAACTCCTACCACATG | |
| | TCCTGGGTCCGCCAGGCTCCTGGAGAAGGACTG | |
| | GAATGGATCGGAATCATTTACGCCGACACCGGC | |
| | AATGCCGACTACGCTTCTTGGACCAAGGGCAGA | |
| | TTTACCATCTCCAAAGCCTCCACCACCGTGGAA | |
| | CTGAGGATTACCTCTCCCACCTCCGAAGACACC | |
| | GCCACATACTTCTGCGCTAGGGGACCTACCAAC | |
| | ACCTACCCTACCTACTTCGCCCTGTGGGGACCA | |
| | GGAACACTGGTGACAGTGAGCAGCGCTAGCACC | |
| | AAAGGTCCCAGCGTGTTCCCACTCGCCCCGAGT | |
| | TCAAAATCAACTTCTGGAGGCACCGCCGCCCTG | |
| | GGTTGCCTGGTAAAGGACTACTTCCCAGAGCCC | |
| | GTGACCGTGAGCTGGAACTCCGGGGCACTGACA | |
| | TCTGGCGTTCATACTTTCCCGGCCGTGCTCCAG | |
| | TCTTCAGGTCTGTATAGTCTCTCCTCTGTGGTC | |
| | ACTGTCCCATCTAGCTCTCTGGGCACCCAAACC | |
| | TACATATGCAACGTTAATCACAAGCCGAGCAAT | |
| | ACTAAAGTTGACAAAAAGGTGGAACCCAAGTCT | |
| | TGTGACAAGACCCACACGTGTCCCCCCTGCCCG | |
| | GCTCCTGAGCTGCTTGGCGGCCCCAGCGTCTTT | |
| | CTCTTTCCCCCAAAGCCAAAAGATACCTTGATG | |
| | ATCAGCAGAACTCCCGAGGTGACATGCGTCGTC | |
| | GTGGACGTAAGCCATGAAGATCCCGAGGTTAAG | |
| | TTCAACTGGTATGTCGATGGCGTGGAAGTCCAT | |
| | AATGCTAAGACTAAACCTCGCGAAGAGCAGTAC | |
| | AATTCAACTTACCGGGTCGTTTCCGTTCTGACC | |
| | GTGCTGCATCAGGACTGGCTGAATGGTAAAGAG | |
| | TACAAGTGCAAAGTGTCTAACAAGGCACTCCCC | |
| | GCCCCAATTGAGAAGACTATCTCCAAAGCTAAA | |
| | GGGCAACCAAGAGAGCCCCAGGTCTACACCCTG | |
| | CCCCCCTCAAGGGATGAGCTTACTAAGAACCAG | |
| | GTTAGTCTCACCTGCTTGGTTAAAGGATTTTAT | |
| | CCAAGCGATATTGCTGTGGAGTGGGAGTCCAAC | |
| | GGCCAGCCTGAGAACAATTATAAAACCACCCCC | |
| | CCTGTTCTTGACAGTGACGGTAGTTTCTTCCTG | |
| | TATTCCAAACTGACCGTCGATAAGAGCAGATGG | |
| | CAACAGGGAAATGTGTTCAGCTGCTCCGTGATG | |
| | CATGAGGCGCTCCATAATCATTACACACAAAAA | |
| | AGTTTGTCCCTGAGCCCAGGCAAGTAG | |
| R.1B11_LC | AFELTQTPSSVEAAVGGTVTIKCQASQSITTYL | 141 |
| | AWYQQKPGQPPKLLIYGASNLESGVPSRFKGSR | |
| | SGTEYTLTISDLESADAATYYCQSYYAISKSAF | |
| | GGGTEVVVKRTVAAPSVFIFPPSDEQLKSGTAS | |
| | VVCLLNNFYPREAKVQWKVDNALQSGNSQESVT | |
| | EQDSKDSTYSLSSTLTLSKADYEKHKVYACEVT | |
| | HQGLSSPVTKSFNRGEC | |
| R.1B11_LC | GCTTTTGAACTGACCCAGACCCCCTCCTCCGTG | 142 |
| (Nucleo- | GAAGCTGCTGTGGGAGGAACAGTGACCATTAAG | |
| tide) | TGTCAGGCCTCTCAGTCCATTACCACCTATCTG | |
| | GCCTGGTACCAGCAGAAGCCCGGTCAGCCTCCT | |
| | AAGCTGCTGATCTACGGCGCAAGTAACCTGGAG | |
| | AGCGGAGTGCCTTCCAGGTTCAAAGGATCCAGG | |
| | TCCGGCACAGAGTACACCCTGACAATTAGCGAC | |
| | CTGGAAAGCGCCGACGCCGCTACATATTACTGC | |
| | CAGTCCTACTACGCCATTTCCAAGTCCGCCTTC | |
| | GGCGGTGGAACAGAAGTGGTGGTGAAAcgtacg | |
| | GTGGCAGCTCCATCAGTTTTTATCTTCCCACCA | |
| | AGCGACGAGCAATTGAAGTCCGGCACTGCCTCT | |
| | GTGGTGTGCCTTCTGAACAACTTCTATCCAAGG | |
| | GAGGCCAAGGTCCAGTGGAAGGTCGATAATGCG | |
| | CTGCAGAGCGGGAACAGCCAAGAGTCAGTGACC | |
| | GAGCAGGACTCAAAAGATAGCACATACTCTCTG | |
| | AGTTCCACCCTGACCCTGTCAAAGGCTGACTAC | |
| | GAAAAGCATAAGGTATACGCATGCGAAGTGACC | |
| | CATCAGGGTCTCTCATCTCCCGTAACCAAATCT | |
| | TTTAATAGAGGAGAATGCTGA | |

### Example 3: Binding and Functional Evaluation of Chimeric Anti-CD47 Antibodies

### ELISA Binding Assay

A 96-well plate was coated overnight at 4° C. with 1 μg/ml recombinant CD47. After washing 3 times, the plate was blocked with 300 μL 1% BSA in PBST at 37° C. for 1 hour. Serially diluted antibodies were added and incubated at 37° C. for 1 hour. The plate was then washed 4 times with PBST and incubated with 1:5000 diluted 2nd antibody (Sigma, Cat #A0293) at 37° C. for 1 hour. The plate was washed again 4 times with PBST, incubated with TMB substrate for 15 min at room temperature, terminated with 1N HCl, and then read at 450 nM. Chimeric antibodies bound to CD47 with an EC50 range from 0.01 nM-0.5 nM as shown in Table 6.

TABLE 6
Binding of chimeric anti-CD47 antibodies to
recombinant CD47 as determined by ELISA.

| Anti-CD47 | |
|---|---|
| Antibody | EC50 (nM) |
| R.1H3 | C |
| R.9B11 | A |
| R.13A2 | C |
| R.14G4 | C |
| R.15G10 | A |
| R.18B9 | A |
| R.19H7 | B |
| R.20H1 | B |
| R.1B11 | A |
| M.1D15E11 | A |
| M.20B10E9 | A |
| M.69H1H9 | A |
| M.30A5C6 | A |
| M.95D4A11 | A |
| VIR.CD47.V8 | C |
| 5F9.G4 | A |
| huIgG1 control | NB |

EC50 ranges for CD47- A: 0.01 nM-0.1 nM; B: 0.1 nM-0.5 nM; C: >0.5 nM; NB: not bound.

### Flow Cytometry Binding Assay

Antibody binding to CD47+ cell lines was quantified by flow cytometry. Harvested cells were centrifuged at 2000 rpm for 5 min, resuspended in 10-15 mL ice-cold culture medium, and then counted. Cells were resuspended in blocking buffer (PBS plus 2% FBS) at a concentration of 3×10⁶ cells/mL. 100 μL of the cell suspension was dispensed into each well of a 96-well plate. Purified antibodies were diluted to the desired concentrations with blocking buffer and 100 μL of diluted antibodies were added to the well and incubated for 1 hour at 4° C. The cells were then washed 3 times with PBS plus 2% FBS. After the third wash, the cells were resuspended in 100 μL 1:500 diluted Alexa Fluor 488 labeled Mouse anti-Human IgG1 Fc secondary antibody (Invitrogen, Cat #: A10631) and incubated for 1 hour at 4° C. in the dark. The cells were then washed 3 times with 200 μL PBS by centrifuging at 2000 rpm for 5 min. After the last wash, the cells were resuspended in 300 μL cold PBS and analyzed on a FACSVerse™ (BD Biosciences) flow cytometer. Chimeric antibodies bound to CD47+ cell lines with an EC50 range from 0.1 nM-10 nM.

TABLE 7
Binding of chimeric anti-CD47 antibodies to CD47+
cell lines as quantified by flow cytometry.

| Anti-CD47 | |
|---|---|
| Antibody | EC50 (nM) |
| R.1H3 | A |
| R.9B11 | B |
| R.13A2 | B |
| R.14G4 | C |
| R.15G10 | B |
| R.18B9 | B |
| R.19H7 | A |
| R.20H1 | A |
| R.1B11 | A |
| M.1D15E11 | A |
| M.20B10E9 | A |
| M.69H1H9 | A |
| M.30A5C6 | A |
| M.95D4A11 | A |
| VIR.CD47.V8 | B |
| 5F9.G4 | A |
| huIgG1 control | NB |

EC50 ranges for CD47- A: 0.1 nM-0.5 nM; B: 0.5 nM-5 nM; C: >5 nM; NB: not bound.

### Surface Plasmon Resonance Binding Assay

Surface plasmon resonance (SPR) is a more accurate and sensitive assay for binding kinetics and affinities than an ELISA. Furthermore, binding kinetics measurements of the anti-CD47 antibodies were not affected by antibody valency in the SPR assays because antibodies were immobilized and challenged with monomeric antigens. SPR was performed on a Biacore 8K instrument (GE Healthcare). Antibodies were captured on Biacore Series S CM5 sensor chips coated with a monoclonal mouse anti-human IgG (Fc) antibody (GE Healthcare human antibody capture kit). Serial 3-fold dilutions of CD47 ECD antigen were injected at a flow rate of 30 μL/min. Each sample was analyzed with 1 min association and 10 min dissociation at room temperature (25° C.). After each injection, the chip was regenerated using 3M MgCl₂. A 1:1 Langmuir model of simultaneous fitting of kon and koff was used for kinetics analysis. Chimeric antibody 18B9 from rabbit B cell cloning bounds to human CD47 with a KD of 10 to 100 nM. Chimeric antibody 1D15E11 from mouse hybridoma bounds to human CD47 with a KD of 1 to 10 nM.

### Inhibition of SIPRA Binding to CD47+ Cells

Tumor cells were harvested, centrifuged, and then resuspended in FACS buffer (PBS plus 2% FBS) at a concentration of 2×10⁶ cells/mL. 100 μL of the cell suspension was dispensed into each well of a 96-well plate. The plate was centrifuged for 5 min and 300×g, and the supernatants were discarded. The cells were incubated with 50 μL per well of serially diluted anti-CD47 antibodies and a constant amount of SIRPA-mIgG2a fusion protein (0.2 μg/mL for Raji cells, 1 μg/mL for HCT-15 cells) in FACS buffer for 1 hour at 4° C. Then, the plates were washed twice with FACS buffer and incubated for 1 hour at 4° C. in the dark with 100 μL of Alexa Fluor 488 donkey anti-Mouse IgG(H+L) secondary antibody (Invitrogen, Cat #A21202, 1:1000). After washing twice with FACS buffer, the plates were resuspended with 300 μL FACS buffer and analyzed by flow cytometry. Chimeric antibodies showed SIRPa blocking activity with a IC50 of 0.1 to 10 nM.

TABLE 8
SIRPa blocking activity by chimeric anti-CD47 antibodies
to CD47+ cell lines as analyzed by flow cytometry.

| Anti-CD47 | |
|---|---|
| Antibody | EC50 (nM) |
| R.1H3 | A |
| R.9B11 | B |
| R.13A2 | A |
| R.14G4 | B |
| R.15G10 | A |
| R.18B9 | A |
| R.19H7 | B |
| R.20H1 | A |
| R.1B11 | A |
| M.1D15E11 | A |
| M.20B10E9 | A |
| M.69H1H9 | A |
| M.30A5C6 | A |
| M.95D4A11 | A |
| VIR.CD47.V8 | C |
| 5F9.G4 | A |
| huIgG1 control | NB |

IC50 ranges for CD47- A: 0.1 nM-1 nM; B: 1 nM-5 nM; C: >5 nM; NB: not bound.

### Example 4: Humanization of Anti-CD47 Antibodies 1D15E11 from Mouse Hybridoma and 18B9 from Rabbit B Cell Cloning

The anti-CD47 antibodies 1D15E11 from mouse hybridoma and 18B9 from rabbit B cell cloning were humanized by grafting their CDR sequences into a human framework. The CDR graft variants had reduced affinity for their antigens compared to the chimeric antibodies, and they contained sequences that might reduce their stability. Thus, they were further engineered by replacing human framework residues with the corresponding rabbit/mouse framework residues at positions that contribute to binding and replacing potential chemical hot spots with residues that do not have chemical liabilities.

### Humanization of the Anti-CD47 Antibody, ID15E11

The sequences of 1D15E11 variable domains were aligned to sequences from the international ImMunoGeneTics information System® (IMGT®) library to identify human germline VH and VL-kappa sequences with homology to the HV and VL chains of 1D15E11. IGKV1-39*01 was chosen as the VL human germline acceptor sequence. IGHV1-3*01 was chosen as the VH human germline acceptor sequence.

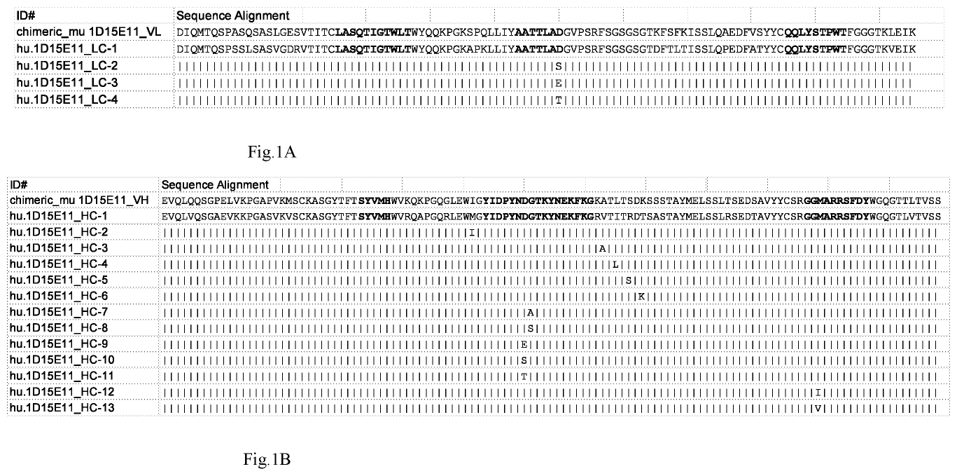

The simple graft variant hu.1D15E11-1 had substantially reduced affinity for CD47 compared to the chimeric1D15E11 chimeric antibody. To restore its affinity, selected amino acids in the framework regions were changed back to the mouse sequence. FIG. 1A showed light chain CDR graft, FIG. 1B showed five heavy chain Vernier positions 148, A67, L69, S71, K73 which were evaluated.

Additional variants were constructed to reduce the potential chemical instability of the final clone. A potential deamidation site (DG) in LC CDR2 was mutated to SG, EG, or TG (FIG. 1A), A potential deamidation site (DG) in HC CDR2 was mutated to SG, EG, TG, DA or DS, A potential methionine oxidation site (M) in HC CDR3 was mutated to I or V (FIG. 1B).

TABLE 9
Humanized variants of anti-CD47 antibody, 1D15E11

| Construct | | SEQ ID |
|---|---|---|
| Description | Sequence | NO: |
| hu.1D15E11-V1 | SYVMH | 1 |
| HC: CDR-1 | | |
| hu.1D15E11-V1 | YIDPYNEGTKYNEKFKG | 28 |
| HC: CDR-2 | | |
| hu.1D15E11-V1 | GGMARRSFDY | 30 |
| HC: CDR-3 | | |
| hu.1D15E11-V1 | LASQTIGTWLT | 45 |
| LC: CDR-1 | | |
| hu.1D15E11-V1 | AATTLAE | 69 |
| LC: CDR-2 | | |
| hu.1D15E11-V1 | QQLYSTPWT | 70 |
| LC: CDR-3 | | |
| hu.1D15E11-V1 | EVQLVQSGAEVKKPGASVKVSCKASGYTFTSYVM | 143 |
| VH | HWVRQAPGQRLEWMGYIDPYNEGTKYNEKFKGRV | |
| | TITRDTSASTAYMELSSLRSEDTAVYYCSRGGM | |
| | ARRSFDYWGQGTLVTVSS | |
| hu.1D15E11-V1 | DIQMTQSPSSLSASVGDRVTITCLASQTIGTWLTW | 144 |
| VL | YQQKPGKAPKLLIYAATTLAEGVPSRFSGSGSGTD | |
| | FTLTISSLQPEDFATYYCQQLYSTPWTFGGGTKVE | |
| | IK | |
| hu.1D15E11-V1 | EVQLVQSGAEVKKPGASVKVSCKASGYTFTSYVM | 145 |
| HC | HWVRQAPGQRLEWMGYIDPYNEGTKYNEKFKGRV | |
| | TITRDTSASTAYMELSSLRSEDTAVYYCSRGGMA | |
| | RRSFDYWGQGTLVTVSSASTKGPSVFPLAPSSKS | |
| | TSGGTAALGCLVKDYFPEPVTVSWNSGALTSGVH | |
| | TFPAVLQSSGLYSLSSVVTVPSSSLGTQTYICNV | |
| | NHKPSNTKVDKKVEPKSCDKTHTCPPCPAPELLG | |
| | GPSVFLFPPKPKDTLMISRTPEVTCVVVDVSHED | |
| | PEVKFNWYVDGVEVHNAKTKPREEQYNSTYRVVS | |
| | VLTVLHQDWLNGKEYKCKVSNKALPAPIEKTISK | |
| | AKGQPREPQVYTLPPSRDELTKNQVSLTCLVKGF | |
| | YPSDIAVEWESNGQPENNYKTTPPVLDSDGSFFL | |
| | YSKLTVDKSRWQQGNVFSCSVMHEALHNHYTQKS | |
| | LSLSPGK | |
| hu.1D15E11-V1 | GAGGTGCAGCTGGTGCAGAGCGGAGCCGAGGTG | 146 |
| HC (Nucleotide) | AAGAAGCCAGGAGCCAGCGTGAAGGTGAGCTGT | |
| | AAGGCTAGTGGATACACATTCACAAGCTACGTG | |
| | ATGCACTGGGTGAGACAGGCCCCTGGACAGAGA | |
| | CTGGAGTGGATGGGATATATCGACCCCTATAAT | |
| | GAGGGCACCAAGTACAACGAAAAATTCAAGGGC | |
| | AGAGTGACCATCACCAGAGACACCAGCGCCAGC | |
| | ACCGCCTACATGGAGCTGAGCAGCCTGAGAAGC | |
| | GAGGACACCGCCGTGTACTACTGCAGCAGAGGC | |
| | GGCATGGCCAGGAGATCCTTTGACTACTGGGGC | |
| | CAGGGAACACTGGTGACCGTGAGCAGCGCTAGC | |
| | ACCAAAGGTCCCAGCGTGTTCCCACTCGCCCCGA | |
| | GTTCAAAATCAACTTCTGGAGGCACCGCCGCCCT | |
| | GGGTTGCCTGGTAAAGGACTACTTCCCAGAGCC | |
| | CGTGACCGTGAGCTGGAACTCCGGGGCACTGAC | |
| | ATCTGGCGTTCATACTTTCCCGGCCGTGCTCCAG | |
| | TCTTCAGGTCTGTATAGTCTCTCCTCTGTGGTCAC | |
| | TGTCCCATCTAGCTCTCTGGGCACCCAAACCTAC | |
| | ATATGCAACGTTAATCACAAGCCGAGCAATACT | |
| | AAAGTTGACAAAAAGGTGGAACCCAAGTCTTGT | |
| | GACAAGACCCACACGTGTCCCCCCTGCCCGGCTC | |
| | CTGAGCTGCTTGGCGGCCCCAGCGTCTTTCTCTT | |
| | TCCCCCAAAGCCAAAAGATACCTTGATGATCAG | |
| | CAGAACTCCCGAGGTGACATGCGTCGTCGTGGA | |
| | CGTAAGCCATGAAGATCCCGAGGTTAAGTTCAA | |
| | CTGGTATGTCGATGGCGTGGAAGTCCATAATGCT | |
| | AAGACTAAACCTCGCGAAGAGCAGTACAATTCA | |
| | ACTTACCGGGTCGTTTCCGTTCTGACCGTGCTGC | |
| | ATCAGGACTGGCTGAATGGTAAAGAGTACAAGT | |
| | GCAAAGTGTCTAACAAGGCACTCCCCGCCCCAA | |
| | TTGAGAAGACTATCTCCAAAGCTAAAGGGCAAC | |
| | CAAGAGAGCCCCAGGTCTACACCCTGCCCCCCTC | |
| | AAGGGATGAGCTTACTAAGAACCAGGTTAGTCT | |
| | CACCTGCTTGGTTAAAGGATTTTATCCAAGCGAT | |
| | ATTGCTGTGGAGTGGGAGTCCAACGGCCAGCCT | |
| | GAGAACAATTATAAAACCACCCCCCCTGTTCTTG | |
| | ACAGTGACGGTAGTTTCTTCCTGTATTCCAAACT | |
| | GACCGTCGATAAGAGCAGATGGCAACAGGGAAA | |
| | TGTGTTCAGCTGCTCCGTGATGCATGAGGCGCTC | |
| | CATAATCATTACACACAAAAAAGTTTGTCCCTGA | |
| | GCCCAGGCAAG | |
| hu.1D15E11-V1 | DIQMTQSPSSLSASVGDRVTITCLASQTIGTWLTW | 147 |
| LC | YQQKPGKAPKLLIYAATTLAEGVPSRFSGSGSGTD | |
| | FTLTISSLQPEDFATYYCQQLYSTPWTFGGGTKVE | |
| | IKRTVAAPSVFIFPPSDEQLKSGTASVVCLLNNFYP | |
| | REAKVQWKVDNALQSGNSQESVTEQDSKDSTYSL | |
| | SSTLTLSKADYEKHKVYACEVTHQGLSSPVTKSFN | |
| | RGEC | |
| hu.1D15E11-V1 | GACATTCAGATGACCCAGAGCCCCAGCAGCCTG | 148 |
| LC (Nucleotide) | AGCGCCAGCGTGGGAGACAGAGTGACCATCACC | |
| | TGCCTGGCCAGCCAGACCATCGGAACCTGGCTG | |
| | ACCTGGTACCAGCAGAAACCCGGCAAAGCCCCC | |
| | AAACTGCTGATCTACGCCGCCACCACACTGGCC | |
| | GAGGGCGTGCCTTCCAGATTCAGCGGGAGCGGC | |
| | AGCGGCACAGACTTCACACTGACTATCAGCAGC | |
| | CTGCAGCCTGAGGATTTCGCCACCTACTACTGCC | |
| | AGCAGCTGTACTCCACCCCCTGGACATTCGGCGG | |
| | CGGCACCAAGGTGGAGATCAAGCGTACGGTGGC | |
| | AGCTCCATCAGTTTTTATCTTCCCACCAAGCGAC | |
| | GAGCAATTGAAGTCCGGCACTGCCTCTGTGGTGT | |
| | GCCTTCTGAACAACTTCTATCCAAGGGAGGCCA | |
| | AGGTCCAGTGGAAGGTCGATAATGCGCTGCAGA | |
| | GCGGGAACAGCCAAGAGTCAGTGACCGAGCAGG | |
| | ACTCAAAAGATAGCACATACTCTCTGAGTTCCAC | |
| | CCTGACCCTGTCAAAGGCTGACTACGAAAAGCA | |
| | TAAGGTATACGCATGCGAAGTGACCCATCAGGG | |
| | TCTCTCATCTCCCGTAACCAAATCTTTTAATAGA | |
| | GGAGAATGC | |
| hu.1D15E11-V2 | SYVMH | 1 |
| HC: CDR-1 | | |
| hu.1D15E11-V2 | YIDPYNEGTKYNEKFKG | 28 |
| HC: CDR-2 | | |
| hu.1D15E11-V2 | GGMARRSFDY | 30 |
| HC: CDR-3 | | |
| hu.1D15E11-V2 | LASQTIGTWLT | 45 |
| LC: CDR-1 | | |
| hu.1D15E11-V2 | AATTLAE | 69 |
| LC: CDR-2 | | |
| hu.1D15E11-V2 | QQLYSTPWT | 70 |
| LC: CDR-3 | | |
| hu.1D15E11-V2 | EVQLVQSGAEVKKPGASVKVSCKASGYTFTSYVM | 149 |
| VH | HWVRQAPGQRLEWMGYIDPYNEGTKYNEKFKGRV | |
| | TITSDTSASTAYMELSSLRSEDTAVYYCSRGGMA | |
| | RRSFDYWGQGTLVTVSS | |
| hu.1D15E11-V2 | DIQMTQSPSSLSASVGDRVTITCLASQTIGTWLTW | 150 |
| VL | YQQKPGKAPKLLIYAATTLAEGVPSRFSGSGSGTD | |
| | FTLTISSLQPEDFATYYCQQLYSTPWTFGGGTKVE | |
| | IK | |
| hu.1D15E11-V2 | EVQLVQSGAEVKKPGASVKVSCKASGYTFTSYVM | 151 |
| HC | HWVRQAPGQRLEWMGYIDPYNEGTKYNEKFKGRV | |
| | TITSDTSASTAYMELSSLRSEDTAVYYCSRGGMA | |
| | RRSFDYWGQGTLVTVSSASTKGPSVFPLAPSSKS | |
| | TSGGTAALGCLVKDYFPEPVTVSWNSGALTSGVHT | |
| | FPAVLQSSGLYSLSSVVTVPSSSLGTQTYICNVNH | |
| | KPSNTKVDKKVEPKSCDKTHTCPPCPAPELLGGPS | |
| | VFLFPPKPKDTLMISRTPEVTCVVVDVSHEDPEVK | |
| | FNWYVDGVEVHNAKTKPREEQYNSTYRVVSVLTVL | |
| | HQDWLNGKEYKCKVSNKALPAPIEKTISKAKGQPR | |
| | EPQVYTLPPSRDELTKNQVSLTCLVKGFYPSDIAV | |
| | EWESNGQPENNYKTTPPVLDSDGSFFLYSKLTVDK | |
| | SRWQQGNVFSCSVMHEALHNHYTQKSLSLSPGK | |
| hu.1D15E11-V2 | GAGGTGCAGCTGGTGCAGAGCGGAGCCGAGGTG | 152 |
| HC (Nucleotide) | AAGAAGCCAGGAGCCAGCGTGAAGGTGAGCTGT | |
| | AAGGCTAGTGGATACACATTCACAAGCTACGTG | |
| | ATGCACTGGGTGAGACAGGCCCCTGGACAGAGA | |
| | CTGGAGTGGATGGGATATATCGACCCCTATAAT | |
| | GAGGGCACCAAGTACAACGAAAAATTCAAGGGC | |
| | AGAGTGACCATCACCAGCGACACCTCTGCCAGC | |
| | ACCGCCTACATGGAGCTGAGCTCACTGAGAAGC | |
| | GAGGACACCGCCGTGTACTACTGCAGCAGAGGC | |
| | GGCATGGCCAGGAGATCCTTTGACTACTGGGGC | |
| | CAGGGAACACTGGTGACCGTGAGCAGCGCTAGC | |
| | ACCAAAGGTCCCAGCGTGTTCCCACTCGCCCCGA | |
| | GTTCAAAATCAACTTCTGGAGGCACCGCCGCCCT | |
| | GGGTTGCCTGGTAAAGGACTACTTCCCAGAGCC | |
| | CGTGACCGTGAGCTGGAACTCCGGGGCACTGAC | |
| | ATCTGGCGTTCATACTTTCCCGGCCGTGCTCCAG | |
| | TCTTCAGGTCTGTATAGTCTCTCCTCTGTGGTCAC | |
| | TGTCCCATCTAGCTCTCTGGGCACCCAAACCTAC | |
| | ATATGCAACGTTAATCACAAGCCGAGCAATACT | |
| | AAAGTTGACAAAAAGGTGGAACCCAAGTCTTGT | |
| | GACAAGACCCACACGTGTCCCCCCTGCCCGGCTC | |
| | CTGAGCTGCTTGGCGGCCCCAGCGTCTTTCTCTT | |
| | TCCCCCAAAGCCAAAAGATACCTTGATGATCAG | |
| | CAGAACTCCCGAGGTGACATGCGTCGTCGTGGA | |
| | CGTAAGCCATGAAGATCCCGAGGTTAAGTTCAA | |
| | CTGGTATGTCGATGGCGTGGAAGTCCATAATGCT | |
| | AAGACTAAACCTCGCGAAGAGCAGTACAATTCA | |
| | ACTTACCGGGTCGTTTCCGTTCTGACCGTGCTGC | |
| | ATCAGGACTGGCTGAATGGTAAAGAGTACAAGT | |
| | GCAAAGTGTCTAACAAGGCACTCCCCGCCCCAA | |
| | TTGAGAAGACTATCTCCAAAGCTAAAGGGCAAC | |
| | CAAGAGAGCCCCAGGTCTACACCCTGCCCCCCTC | |
| | AAGGGATGAGCTTACTAAGAACCAGGTTAGTCT | |
| | CACCTGCTTGGTTAAAGGATTTTATCCAAGCGAT | |
| | ATTGCTGTGGAGTGGGAGTCCAACGGCCAGCCT | |
| | GAGAACAATTATAAAACCACCCCCCCTGTTCTTG | |
| | ACAGTGACGGTAGTTTCTTCCTGTATTCCAAACT | |
| | GACCGTCGATAAGAGCAGATGGCAACAGGGAAA | |
| | TGTGTTCAGCTGCTCCGTGATGCATGAGGCGCTC | |
| | CATAATCATTACACACAAAAAAGTTTGTCCCTGA | |
| | GCCCAGGCAAG | |
| hu.1D15E11-V2 | DIQMTQSPSSLSASVGDRVTITCLASQTIGTWLTW | 153 |
| LC | YQQKPGKAPKLLIYAATTLAEGVPSRFSGSGSGTD | |
| | FTLTISSLQPEDFATYYCQQLYSTPWTFGGGTKVE | |
| | IKRTVAAPSVFIFPPSDEQLKSGTASVVCLLNNFY | |
| | PREAKVQWKVDNALQSGNSQESVTEQDSKDSTYSL | |
| | SSTLTLSKADYEKHKVYACEVTHQGLSSPVTKSFN | |
| | RGEC | |
| hu.1D15E11-V2 | GACATTCAGATGACCCAGAGCCCCAGCAGCCTG | 154 |
| LC (Nucleotide) | AGCGCCAGCGTGGGAGACAGAGTGACCATCACC | |
| | TGCCTGGCCAGCCAGACCATCGGAACCTGGCTG | |
| | ACCTGGTACCAGCAGAAACCCGGCAAAGCCCCC | |
| | AAACTGCTGATCTACGCCGCCACCACACTGGCC | |
| | GAGGGCGTGCCTTCCAGATTCAGCGGGAGCGGC | |
| | AGCGGCACAGACTTCACACTGACTATCAGCAGC | |
| | CTGCAGCCTGAGGATTTCGCCACCTACTACTGCC | |
| | AGCAGCTGTACTCCACCCCCTGGACATTCGGCGG | |
| | CGGCACCAAGGTGGAGATCAAGCGTACGGTGGC | |
| | AGCTCCATCAGTTTTTATCTTCCCACCAAGCGAC | |
| | GAGCAATTGAAGTCCGGCACTGCCTCTGTGGTGT | |
| | GCCTTCTGAACAACTTCTATCCAAGGGAGGCCA | |
| | AGGTCCAGTGGAAGGTCGATAATGCGCTGCAGA | |
| | GCGGGAACAGCCAAGAGTCAGTGACCGAGCAGG | |
| | ACTCAAAAGATAGCACATACTCTCTGAGTTCCAC | |
| | CCTGACCCTGTCAAAGGCTGACTACGAAAAGCA | |
| | TAAGGTATACGCATGCGAAGTGACCCATCAGGG | |
| | TCTCTCATCTCCCGTAACCAAATCTTTTAATAGA | |
| | GGAGAATGC | |
| hu.1D15E11-V3 | SYVMH | 1 |
| HC: CDR-1 | | |
| hu.1D15E11-V3 | YIDPYNEGTKYNEKFKG | 28 |
| HC: CDR-2 | | |
| hu.1D15E11-V3 | GGIARRSFDY | 177 |
| HC: CDR-3 | | |
| hu.1D15E11-V3 | LASQTIGTWLT | 45 |
| LC: CDR-1 | | |
| hu.1D15E11-V3 | AATTLAE | 69 |
| LC: CDR-2 | | |
| hu.1D15E11-V3 | QQLYSTPWT | 70 |
| LC: CDR-3 | | |
| hu.1D15E11-V3 | EVQLVQSGAEVKKPGASVKVSCKASGYTFTSYVM | 181 |
| VH | HWVRQAPGQRLEWMGYIDPYNEGTKYNEKFKGR | |
| | VTITRDTSASTAYMELSSLRSEDTAVYYCSRGGI | |
| | ARRSFDYWGQGTLVTVSS | |
| hu.1D15E11-V3 | DIQMTQSPSSLSASVGDRVTITCLASQTIGTWLTWY | 144 |
| VL | QQKPGKAPKLLIYAATTLAEGVPSRFSGSGSGTDFT | |
| | LTISSLQPEDFATYYCQQLYSTPWTFGGGTKVEIK | |
| hu.1D15E11-V3 | EVQLVQSGAEVKKPGASVKVSCKASGYTFTSYVM | 182 |
| HC | HWVRQAPGQRLEWMGYIDPYNEGTKYNEKFKGR | |
| | VTITRDTSASTAYMELSSLRSEDTAVYYCSRGGIAR | |
| | RSFDYWGQGTLVTVSSASTKGPSVFPLAPSSKSTSG | |
| | GTAALGCLVKDYFPEPVTVSWNSGALTSGVHTFP | |
| | AVLQSSGLYSLSSVVTVPSSSLGTQTYICNVNHKPS | |
| | NTKVDKKVEPKSCDKTHTCPPCPAPELLGGPSVFL | |
| | FPPKPKDTLMISRTPEVTCVVVDVSHEDPEVKFNW | |
| | YVDGVEVHNAKTKPREEQYNSTYRVVSVLTVLHQ | |
| | DWLNGKEYKCKVSNKALPAPIEKTISKAKGQPREP | |
| | QVYTLPPSRDELTKNQVSLTCLVKGFYPSDIAVEW | |
| | ESNGQPENNYKTTPPVLDSDGSFFLYSKLTVDKSR | |
| | WQQGNVFSCSVMHEALHNHYTQKSLSLSPGK | |
| hu.1D15E11-V3 | GAGGTGCAGCTGGTGCAGAGCGGAGCCGAGGTG | 183 |
| HC (Nucleotide) | AAGAAGCCAGGAGCCAGCGTGAAGGTGAGCTGT | |
| | AAGGCTAGTGGATACACATTCACAAGCTACGTG | |
| | ATGCACTGGGTGAGACAGGCCCCTGGACAGAGA | |
| | CTGGAGTGGATGGGATATATCGACCCCTATAAT | |
| | GAGGGCACCAAGTACAACGAAAAATTCAAGGGC | |
| | AGAGTGACCATCACCAGAGACACCAGCGCCAGC | |
| | ACCGCCTACATGGAGCTGAGCAGCCTGAGAAGC | |
| | GAGGACACCGCCGTGTACTACTGCAGCAGAGGC | |
| | GGCATCGCCAGGAGATCCTTTGACTACTGGGGC | |
| | CAGGGAACACTGGTGACCGTGAGCAGCGCTAGC | |
| | ACCAAAGGTCCCAGCGTGTTCCCACTCGCCCCGA | |
| | GTTCAAAATCAACTTCTGGAGGCACCGCCGCCCT | |
| | GGGTTGCCTGGTAAAGGACTACTTCCCAGAGCC | |
| | CGTGACCGTGAGCTGGAACTCCGGGGCACTGAC | |
| | ATCTGGCGTTCATACTTTCCCGGCCGTGCTCCAG | |
| | TCTTCAGGTCTGTATAGTCTCTCCTCTGTGGTCAC | |
| | TGTCCCATCTAGCTCTCTGGGCACCCAAACCTAC | |
| | ATATGCAACGTTAATCACAAGCCGAGCAATACT | |
| | AAAGTTGACAAAAAGGTGGAACCCAAGTCTTGT | |
| | GACAAGACCCACACGTGTCCCCCCTGCCCGGCTC | |
| | CTGAGCTGCTTGGCGGCCCCAGCGTCTTTCTCTT | |
| | TCCCCCAAAGCCAAAAGATACCTTGATGATCAG | |
| | CAGAACTCCCGAGGTGACATGCGTCGTCGTGGA | |
| | CGTAAGCCATGAAGATCCCGAGGTTAAGTTCAA | |
| | CTGGTATGTCGATGGCGTGGAAGTCCATAATGCT | |
| | AAGACTAAACCTCGCGAAGAGCAGTACAATTCA | |
| | ACTTACCGGGTCGTTTCCGTTCTGACCGTGCTGC | |
| | ATCAGGACTGGCTGAATGGTAAAGAGTACAAGT | |
| | GCAAAGTGTCTAACAAGGCACTCCCCGCCCCAA | |
| | TTGAGAAGACTATCTCCAAAGCTAAAGGGCAAC | |
| | CAAGAGAGCCCCAGGTCTACACCCTGCCCCCCTC | |
| | AAGGGATGAGCTTACTAAGAACCAGGTTAGTCT | |
| | CACCTGCTTGGTTAAAGGATTTTATCCAAGCGAT | |
| | ATTGCTGTGGAGTGGGAGTCCAACGGCCAGCCT | |
| | GAGAACAATTATAAAACCACCCCCCCTGTTCTTG | |
| | ACAGTGACGGTAGTTTCTTCCTGTATTCCAAACT | |
| | GACCGTCGATAAGAGCAGATGGCAACAGGGAAA | |
| | TGTGTTCAGCTGCTCCGTGATGCATGAGGCGCTC | |
| | CATAATCATTACACACAAAAAAGTTTGTCCCTGA | |
| | GCCCAGGCAAG | |
| hu.1D15E11-V3 | DIQMTQSPSSLSASVGDRVTITCLASQTIGTWLTWY | 147 |
| LC | QQKPGKAPKLLIYAATTLAEGVPSRFSGSGSGTDFT | |
| | LTISSLQPEDFATYYCQQLYSTPWTFGGGTKVEIKR | |
| | TVAAPSVFIFPPSDEQLKSGTASVVCLLNNFYPREA | |
| | KVQWKVDNALQSGNSQESVTEQDSKDSTYSLSST | |
| | LTLSKADYEKHKVYACEVTHQGLSSPVTKSFNRGE | |
| | C | |
| hu.1D15E11-V3 | GACATTCAGATGACCCAGAGCCCCAGCAGCCTG | 148 |
| LC (Nucleotide) | AGCGCCAGCGTGGGAGACAGAGTGACCATCACC | |
| | TGCCTGGCCAGCCAGACCATCGGAACCTGGCTG | |
| | ACCTGGTACCAGCAGAAACCCGGCAAAGCCCCC | |
| | AAACTGCTGATCTACGCCGCCACCACACTGGCC | |
| | GAGGGCGTGCCTTCCAGATTCAGCGGGAGCGGC | |
| | AGCGGCACAGACTTCACACTGACTATCAGCAGC | |
| | CTGCAGCCTGAGGATTTCGCCACCTACTACTGCC | |
| | AGCAGCTGTACTCCACCCCCTGGACATTCGGCGG | |
| | CGGCACCAAGGTGGAGATCAAGCGTACGGTGGC | |
| | AGCTCCATCAGTTTTTATCTTCCCACCAAGCGAC | |
| | GAGCAATTGAAGTCCGGCACTGCCTCTGTGGTGT | |
| | GCCTTCTGAACAACTTCTATCCAAGGGAGGCCA | |
| | AGGTCCAGTGGAAGGTCGATAATGCGCTGCAGA | |
| | GCGGGAACAGCCAAGAGTCAGTGACCGAGCAGG | |
| | ACTCAAAAGATAGCACATACTCTCTGAGTTCCAC | |
| | CCTGACCCTGTCAAAGGCTGACTACGAAAAGCA | |
| | TAAGGTATACGCATGCGAAGTGACCCATCAGGG | |
| | TCTCTCATCTCCCGTAACCAAATCTTTTAATAGA | |
| | GGAGAATGC | |
| hu.1D15E11-V4 | SYVMH | 1 |
| HC: CDR-1 | | |
| hu.1D15E11-V4 | YIDPYNEGTKYNEKFKG | 28 |
| HC: CDR-2 | | |
| hu.1D15E11-V4 | GGVARRSFDY | 178 |
| HC: CDR-3 | | |
| hu.1D15E11-V4 | LASQTIGTWLT | 45 |
| LC: CDR-1 | | |
| hu.1D15E11-V4 | AATTLAE | 69 |
| LC: CDR-2 | | |
| hu.1D15E11-V4 | QQLYSTPWT | 70 |
| LC: CDR-3 | | |
| hu.1D15E11-V4 | EVQLVQSGAEVKKPGASVKVSCKASGYTFTSYVM | 184 |
| VH | HWVRQAPGQRLEWMGYIDPYNEGTKYNEKFKGR | |
| | VTITRDTSASTAYMELSSLRSEDTAVYYCSRGGVA | |
| | RRSFDYWGQGTLVTVSS | |
| hu.1D15E11-V4 | DIQMTQSPSSLSASVGDRVTITCLASQTIGTWLTWY | 144 |
| VL | QQKPGKAPKLLIYAATTLAEGVPSRFSGSGSGTDFT | |
| | LTISSLQPEDFATYYCQQLYSTPWTFGGGTKVEIK | |
| hu.1D15E11-V4 | EVQLVQSGAEVKKPGASVKVSCKASGYTFTSYVM | 185 |
| HC | HWVRQAPGQRLEWMGYIDPYNEGTKYNEKFKGR | |
| | VTITRDTSASTAYMELSSLRSEDTAVYYCSRGGVA | |
| | RRSFDYWGQGTLVTVSSASTKGPSVFPLAPSSKSTS | |
| | GGTAALGCLVKDYFPEPVTVSWNSGALTSGVHTF | |
| | PAVLQSSGLYSLSSVVTVPSSSLGTQTYICNVNHKP | |
| | SNTKVDKKVEPKSCDKTHTCPPCPAPELLGGPSVF | |
| | LFPPKPKDTLMISRTPEVTCVVVDVSHEDPEVKFN | |
| | WYVDGVEVHNAKTKPREEQYNSTYRVVSVLTVL | |
| | HQDWLNGKEYKCKVSNKALPAPIEKTISKAKGQP | |
| | REPQVYTLPPSRDELTKNQVSLTCLVKGFYPSDIAV | |
| | EWESNGQPENNYKTTPPVLDSDGSFFLYSKLTVDK | |
| | SRWQQGNVFSCSVMHEALHNHYTQKSLSLSPGK | |
| hu.1D15E11-V4 | GAGGTGCAGCTGGTGCAGAGCGGAGCCGAGGTG | 186 |
| HC (Nucleotide) | AAGAAGCCAGGAGCCAGCGTGAAGGTGAGCTGT | |
| | AAGGCTAGTGGATACACATTCACAAGCTACGTG | |
| | ATGCACTGGGTGAGACAGGCCCCTGGACAGAGA | |
| | CTGGAGTGGATGGGATATATCGACCCCTATAAT | |
| | GAGGGCACCAAGTACAACGAAAAATTCAAGGGC | |
| | AGAGTGACCATCACCAGAGACACCAGCGCCAGC | |
| | ACCGCCTACATGGAGCTGAGCAGCCTGAGAAGC | |
| | GAGGACACCGCCGTGTACTACTGCAGCAGAGGC | |
| | GGCGTGGCCAGGAGATCCTTTGACTACTGGGGC | |
| | CAGGGAACACTGGTGACCGTGAGCAGCGCTAGC | |
| | ACCAAAGGTCCCAGCGTGTTCCCACTCGCCCCGA | |
| | GTTCAAAATCAACTTCTGGAGGCACCGCCGCCCT | |
| | GGGTTGCCTGGTAAAGGACTACTTCCCAGAGCC | |
| | CGTGACCGTGAGCTGGAACTCCGGGGCACTGAC | |
| | ATCTGGCGTTCATACTTTCCCGGCCGTGCTCCAG | |
| | TCTTCAGGTCTGTATAGTCTCTCCTCTGTGGTCAC | |
| | TGTCCCATCTAGCTCTCTGGGCACCCAAACCTAC | |
| | ATATGCAACGTTAATCACAAGCCGAGCAATACT | |
| | AAAGTTGACAAAAAGGTGGAACCCAAGTCTTGT | |
| | GACAAGACCCACACGTGTCCCCCCTGCCCGGCTC | |
| | CTGAGCTGCTTGGCGGCCCCAGCGTCTTTCTCTT | |
| | TCCCCCAAAGCCAAAAGATACCTTGATGATCAG | |
| | CAGAACTCCCGAGGTGACATGCGTCGTCGTGGA | |
| | CGTAAGCCATGAAGATCCCGAGGTTAAGTTCAA | |
| | CTGGTATGTCGATGGCGTGGAAGTCCATAATGCT | |
| | AAGACTAAACCTCGCGAAGAGCAGTACAATTCA | |
| | ACTTACCGGGTCGTTTCCGTTCTGACCGTGCTGC | |
| | ATCAGGACTGGCTGAATGGTAAAGAGTACAAGT | |
| | GCAAAGTGTCTAACAAGGCACTCCCCGCCCCAA | |
| | TTGAGAAGACTATCTCCAAAGCTAAAGGGCAAC | |
| | CAAGAGAGCCCCAGGTCTACACCCTGCCCCCCTC | |
| | AAGGGATGAGCTTACTAAGAACCAGGTTAGTCT | |
| | CACCTGCTTGGTTAAAGGATTTTATCCAAGCGAT | |
| | ATTGCTGTGGAGTGGGAGTCCAACGGCCAGCCT | |
| | GAGAACAATTATAAAACCACCCCCCCTGTTCTTG | |
| | ACAGTGACGGTAGTTTCTTCCTGTATTCCAAACT | |
| | GACCGTCGATAAGAGCAGATGGCAACAGGGAAA | |
| | TGTGTTCAGCTGCTCCGTGATGCATGAGGCGCTC | |
| | CATAATCATTACACACAAAAAAGTTTGTCCCTGA | |
| | GCCCAGGCAAG | |
| hu.1D15E11-V4 | DIQMTQSPSSLSASVGDRVTITCLASQTIGTWLTWY | 147 |
| LC | QQKPGKAPKLLIYAATTLAEGVPSRFSGSGSGTDFT | |
| | LTISSLQPEDFATYYCQQLYSTPWTFGGGTKVEIKR | |
| | TVAAPSVFIFPPSDEQLKSGTASVVCLLNNFYPREA | |
| | KVQWKVDNALQSGNSQESVTEQDSKDSTYSLSST | |
| | LTLSKADYEKHKVYACEVTHQGLSSPVTKSFNRGE | |
| | C | |
| hu.1D15E11-V4 | GACATTCAGATGACCCAGAGCCCCAGCAGCCTG | 148 |
| LC (Nucleotide) | AGCGCCAGCGTGGGAGACAGAGTGACCATCACC | |
| | TGCCTGGCCAGCCAGACCATCGGAACCTGGCTG | |
| | ACCTGGTACCAGCAGAAACCCGGCAAAGCCCCC | |
| | AAACTGCTGATCTACGCCGCCACCACACTGGCC | |
| | GAGGGCGTGCCTTCCAGATTCAGCGGGAGCGGC | |
| | AGCGGCACAGACTTCACACTGACTATCAGCAGC | |
| | CTGCAGCCTGAGGATTTCGCCACCTACTACTGCC | |
| | AGCAGCTGTACTCCACCCCCTGGACATTCGGCGG | |
| | CGGCACCAAGGTGGAGATCAAGCGTACGGTGGC | |
| | AGCTCCATCAGTTTTTATCTTCCCACCAAGCGAC | |
| | GAGCAATTGAAGTCCGGCACTGCCTCTGTGGTGT | |
| | GCCTTCTGAACAACTTCTATCCAAGGGAGGCCA | |
| | AGGTCCAGTGGAAGGTCGATAATGCGCTGCAGA | |
| | GCGGGAACAGCCAAGAGTCAGTGACCGAGCAGG | |
| | ACTCAAAAGATAGCACATACTCTCTGAGTTCCAC | |
| | CCTGACCCTGTCAAAGGCTGACTACGAAAAGCA | |
| | TAAGGTATACGCATGCGAAGTGACCCATCAGGG | |
| | TCTCTCATCTCCCGTAACCAAATCTTTTAATAGA | |
| | GGAGAATGC | |

### Humanization of the Anti-CD47 Antibody, 8B9

The sequences of 181B9 variable domains were aligned to sequences from the international ImMunoGeneTics information system R (IMGT®) library to identify human germline VH and VL-kappa sequences with homology to the H and VL chains of 189. IGLV1-39 was chosen as the VL human germline acceptor sequence. IGHV3-66 was chosen as the VH human germline acceptor sequence.

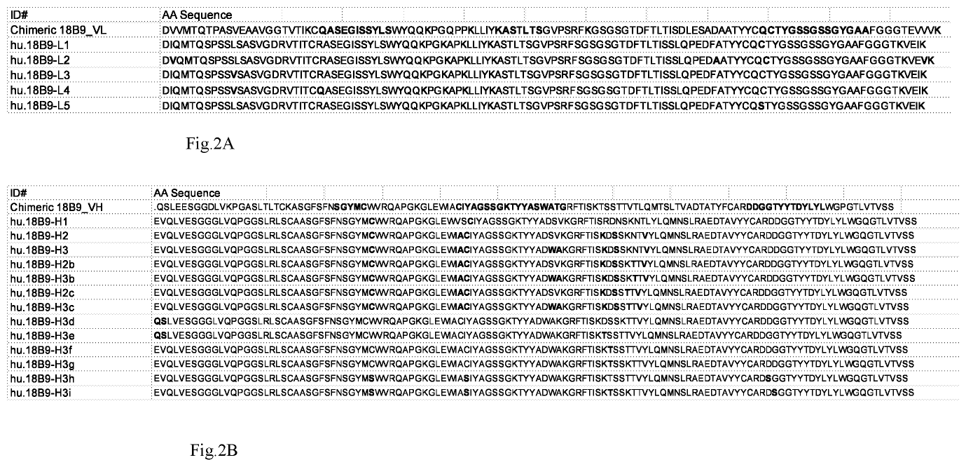

The simple graft variant hu.189-1 had substantially reduced affinity for CD47 compared to the chimeric 189 chimeric antibody. To restore its affinity, selected amino acids in the framework regions were changed back to the rabbit sequence. FIG. 2A-2B show light chain and heavy chain mutants including vernier positions mutants, engineering of potential deamidation site and cysteine residues to seine or alanine, all these variants were evaluated.

TABLE 10
Humanized variants of anti-CD47 antibody, 18B9

| Construct | | SEQ ID |
|---|---|---|
| Description | Sequence | NO: |
| hu.18B9.3i-V1 | SGYMS | 13 |
| HC: CDR-1 | | |
| hu.18B9.3i-V1 | SIYAGSSGKTYYADWAKG | 29 |
| HC: CDR-2 | | |
| hu.18B9.3i-V1 | DSGGTYYTDYLYA | 43 |
| HC: CDR-3 | | |
| hu.18B9.3i-V1 | RASEGISSYLS | 57 |
| LC: CDR-1 | | |
| hu.18B9.3i-V1 | KASTLTS | 66 |
| LC: CDR-2 | | |
| hu.18B9.3i-V1 | QSTYGSSGSSGYGAA | 83 |
| LC: CDR-3 | | |
| hu.18B9.3i-V1 | EVQLVESGGGLVQPGGSLRLSCAASGFSFNSGYMS | 155 |
| VH | WVRQAPGKGLEWIASIYAGSSGKTYYADWAKGR | |
| | FTISKTSSKTTVYLQMNSLRAEDTAVYYCARDSGG | |
| | TYYTDYLYAWGQGTLVTVSS | |
| hu.18B9.3i-V1 | DIQMTQSPSSLSASVGDRVTITCRASEGISSYLSWY | 156 |
| VL | QQKPGKAPKLLIYKASTLTSGVPSRFSGSGSGTDFT | |
| | LTISSLQPEDFATYYCQSTYGSSGSSGYGAAFGGG | |
| | TKVEIK | |
| hu.18B9.3i-V1 | EVQLVESGGGLVQPGGSLRLSCAASGFSFNSGYMS | 157 |
| HC | WVRQAPGKGLEWIASIYAGSSGKTYYADWAKGRF | |
| | TISKTSSKTTVYLQMNSLRAEDTAVYYCARDSGG | |
| | TYYTDYLYAWGQGTLVTVSSASTKGPSVFPLAPS | |
| | SKSTSGGTAALGCLVKDYFPEPVTVSWNSGALTS | |
| | GVHTFPAVLQSSGLYSLSSVVTVPSSSLGTQTYI | |
| | CNVNHKPSNTKVDKKVEPKSCDKTHTCPPCPAPE | |
| | LLGGPSVFLFPPKPKDTLMISRTPEVTCVVVDVS | |
| | HEDPEVKFNWYVDGVEVHNAKTKPREEQYNSTYR | |
| | VVSVLTVLHQDWLNGKEYKCKVSNKALPAPIEKT | |
| | ISKAKGQPREPQVYTLPPSRDELTKNQVSLTCLV | |
| | KGFYPSDIAVEWESNGQPENNYKTTPPVLDSDGS | |
| | FFLYSKLTVDKSRWQQGNVFSCSVMHEALHNHYT | |
| | QKSLSLSPGK | |
| hu.18B9.3i-V1 | GAGGTGCAGCTGGTGGAGAGCGGGGGGGGACTG | 158 |
| HC (Nucleotide) | GTGCAGCCAGGAGGAAGCCTGAGACTGAGCTGT | |
| | GCCGCCAGCGGGTTTAGCTTTAATAGCGGCTATA | |
| | TGAGCTGGGTGAGACAGGCCCCCGGAAAAGGAC | |
| | TGGAGTGGATCGCCTCCATTTACGCCGGAAGTA | |
| | GCGGCAAGACCTACTACGCCGATTGGGCTAAGG | |
| | GAAGATTCACCATTAGCAAGACCAGCAGTAAGA | |
| | CAACCGTGTACCTGCAGATGAACAGCCTGAGAG | |
| | CCGAGGACACCGCCGTGTACTATTGCGCCCGCG | |
| | ACAGCGGAGGAACCTACTACACCGACTACCTGT | |
| | ACGCCTGGGGACAGGGAACCCTGGTGACCGTGA | |
| | GCAGCGCTAGCACCAAAGGTCCCAGCGTGTTCC | |
| | CACTCGCCCCGAGTTCAAAATCAACTTCTGGAGG | |
| | CACCGCCGCCCTGGGTTGCCTGGTAAAGGACTA | |
| | CTTCCCAGAGCCCGTGACCGTGAGCTGGAACTCC | |
| | GGGGCACTGACATCTGGCGTTCATACTTTCCCGG | |
| | CCGTGCTCCAGTCTTCAGGTCTGTATAGTCTCTC | |
| | CTCTGTGGTCACTGTCCCATCTAGCTCTCTGGGC | |
| | ACCCAAACCTACATATGCAACGTTAATCACAAG | |
| | CCGAGCAATACTAAAGTTGACAAAAAGGTGGAA | |
| | CCCAAGTCTTGTGACAAGACCCACACGTGTCCCC | |
| | CCTGCCCGGCTCCTGAGCTGCTTGGCGGCCCCAG | |
| | CGTCTTTCTCTTTCCCCCAAAGCCAAAAGATACC | |
| | TTGATGATCAGCAGAACTCCCGAGGTGACATGC | |
| | GTCGTCGTGGACGTAAGCCATGAAGATCCCGAG | |
| | GTTAAGTTCAACTGGTATGTCGATGGCGTGGAAG | |
| | TCCATAATGCTAAGACTAAACCTCGCGAAGAGC | |
| | AGTACAATTCAACTTACCGGGTCGTTTCCGTTCT | |
| | GACCGTGCTGCATCAGGACTGGCTGAATGGTAA | |
| | AGAGTACAAGTGCAAAGTGTCTAACAAGGCACT | |
| | CCCCGCCCCAATTGAGAAGACTATCTCCAAAGCT | |
| | AAAGGGCAACCAAGAGAGCCCCAGGTCTACACC | |
| | CTGCCCCCCTCAAGGGATGAGCTTACTAAGAAC | |
| | CAGGTTAGTCTCACCTGCTTGGTTAAAGGATTTT | |
| | ATCCAAGCGATATTGCTGTGGAGTGGGAGTCCA | |
| | ACGGCCAGCCTGAGAACAATTATAAAACCACCC | |
| | CCCCTGTTCTTGACAGTGACGGTAGTTTCTTCCT | |
| | GTATTCCAAACTGACCGTCGATAAGAGCAGATG | |
| | GCAACAGGGAAATGTGTTCAGCTGCTCCGTGAT | |
| | GCATGAGGCGCTCCATAATCATTACACACAAAA | |
| | AAGTTTGTCCCTGAGCCCAGGCAAG | |
| hu.18B9.3i-V1 | DIQMTQSPSSLSASVGDRVTITCRASEGISSYLSWY | 159 |
| LC | QQKPGKAPKLLIYKASTLTSGVPSRFSGSGSGTDFT | |
| | LTISSLQPEDFATYYCQSTYGSSGSSGYGAAFGGG | |
| | TKVEIKRTVAAPSVFIFPPSDEQLKSGTASVVCLLN | |
| | NFYPREAKVQWKVDNALQSGNSQESVTEQDSKDS | |
| | TYSLSSTLTLSKADYEKHKVYACEVTHQGLSSPVT | |
| | KSFNRGEC | |
| hu.18B9.3i-V1 | GACATTCAGATGACCCAGAGCCCCAGCAGCCTG | 160 |
| (Nucleotide) | AGCGCCAGCGTGGGAGACAGAGTGACCATCACC | |
| | TGCAGAGCCAGCGAGGGAATCAGCAGCTACCTG | |
| | AGCTGGTATCAGCAGAAACCCGGCAAAGCCCCC | |
| | AAACTGCTGATCTACAAAGCCTCCACACTGACA | |
| | AGCGGAGTGCCCAGCAGATTCAGCGGAAGCGGC | |
| | AGCGGCACAGACTTCACCCTGACCATCAGCAGC | |
| | CTGCAGCCCGAAGATTTCGCCACCTACTACTGCC | |
| | AGAGCACCTACGGCAGCAGCGGCAGTAGCGGAT | |
| | ACGGCGCCGCCTTCGGAGGCGGAACCAAGGTGG | |
| | AGATCAAACGTACGGTGGCAGCTCCATCAGTTTT | |
| | TATCTTCCCACCAAGCGACGAGCAATTGAAGTCC | |
| | GGCACTGCCTCTGTGGTGTGCCTTCTGAACAACT | |
| | TCTATCCAAGGGAGGCCAAGGTCCAGTGGAAGG | |
| | TCGATAATGCGCTGCAGAGCGGGAACAGCCAAG | |
| | AGTCAGTGACCGAGCAGGACTCAAAAGATAGCA | |
| | CATACTCTCTGAGTTCCACCCTGACCCTGTCAAA | |
| | GGCTGACTACGAAAAGCATAAGGTATACGCATG | |
| | CGAAGTGACCCATCAGGGTCTCTCATCTCCCGTA | |
| | ACCAAATCTTTTAATAGAGGAGAATGC | |
| hu.18B9.3i-V2 | SGYMS | 13 |
| HC: CDR-1 | | |
| hu.18B9.3i-V2 | SIYAGSSGKTYYADWAKG | 29 |
| HC: CDR-2 | | |
| hu.18B9.3i-V2 | DSGGTYYTDYLYL | 44 |
| HC: CDR-3 | | |
| hu.18B9.3i-V2 | RASEGISSYLS | 57 |
| LC: CDR-1 | | |
| hu.18B9.3i-V2 | KASTLTS | 66 |
| LC: CDR-2 | | |
| hu.18B9.3i-V2 | QSAYGSSGSSGYGAA | 84 |
| LC: CDR-3 | | |
| hu.18B9.3i-V2 | EVQLVESGGGLVQPGGSLRLSCAASGFSFNSGYMS | 161 |
| VH | WVRQAPGKGLEWIASIYAGSSGKTYYADWAKGR | |
| | FTISKTSSKTTVYLQMNSLRAEDTAVYYCARDSGG | |
| | TYYTDYLYLWGQGTL VTVSS | |
| hu.18B9.3i-V2 | DIQMTQSPSSLSASVGDRVTITCRASEGISSYLSWY | 162 |
| VL | QQKPGKAPKLLIYKASTLTSGVPSRFSGSGSGTDFT | |
| | LTISSLQPEDFATYYCQSAYGSSGSSGYGAAFGGG | |
| | TKVEIK | |
| hu.18B9.3i-V2 | EVQLVESGGGLVQPGGSLRLSCAASGFSFNSGYMS | 163 |
| HC | WVRQAPGKGLEWIASIYAGSSGKTYYADWAKGR | |
| | FTISKTSSKTTVYLQMNSLRAEDTAVYYCARDSGG | |
| | TYYTDYLYLWGQGTLVTVSSASTKGPSVFPLAPSS | |
| | KSTSGGTAALGCLVKDYFPEPVTVSWNSGALTSG | |
| | VHTFPAVLQSSGLYSLSSVVTVPSSSLGTQTYICNV | |
| | NHKPSNTKVDKKVEPKSCDKTHTCPPCPAPELLGG | |
| | PSVFLFPPKPKDTLMISRTPEVTCVVVDVSHEDPEV | |
| | KFNWYVDGVEVHNAKTKPREEQYNSTYRVVSVL | |
| | TVLHQDWLNGKEYKCKVSNKALPAPIEKTISKAK | |
| | GQPREPQVYTLPPSRDELTKNQVSLTCL VKGFYPS | |
| | DIAVEWESNGQPENNYKTTPPVLDSDGSFFLYSKL | |
| | TVDKSRWQQGNVFSCSVMHEALHNHYTQKSLSLS | |
| | PGK | |
| hu.18B9.3i-V2 | GAGGTGCAGCTGGTGGAGAGCGGGGGGGGACTG | 164 |
| HC (Nucleotide) | GTGCAGCCAGGAGGAAGCCTGAGACTGAGCTGT | |
| | GCCGCCAGCGGGTTTAGCTTTAATAGCGGCTATA | |
| | TGAGCTGGGTGAGACAGGCCCCCGGAAAAGGAC | |
| | TGGAGTGGATCGCCTCCATTTACGCCGGAAGTA | |
| | GCGGCAAGACCTACTACGCCGATTGGGCTAAGG | |
| | GAAGATTCACCATTAGCAAGACCAGCAGTAAGA | |
| | CAACCGTGTACCTGCAGATGAACAGCCTGAGAG | |
| | CCGAGGACACCGCCGTGTACTATTGCGCCCGCG | |
| | ACAGCGGAGGAACCTACTACACCGACTACCTGT | |
| | ACCTGTGGGGACAGGGCACCCTGGTGACCGTGA | |
| | GCAGCGCTAGCACCAAAGGTCCCAGCGTGTTCC | |
| | CACTCGCCCCGAGTTCAAAATCAACTTCTGGAGG | |
| | CACCGCCGCCCTGGGTTGCCTGGTAAAGGACTA | |
| | CTTCCCAGAGCCCGTGACCGTGAGCTGGAACTCC | |
| | GGGGCACTGACATCTGGCGTTCATACTTTCCCGG | |
| | CCGTGCTCCAGTCTTCAGGTCTGTATAGTCTCTC | |
| | CTCTGTGGTCACTGTCCCATCTAGCTCTCTGGGC | |
| | ACCCAAACCTACATATGCAACGTTAATCACAAG | |
| | CCGAGCAATACTAAAGTTGACAAAAAGGTGGAA | |
| | CCCAAGTCTTGTGACAAGACCCACACGTGTCCCC | |
| | CCTGCCCGGCTCCTGAGCTGCTTGGCGGCCCCAG | |
| | CGTCTTTCTCTTTCCCCCAAAGCCAAAAGATACC | |
| | TTGATGATCAGCAGAACTCCCGAGGTGACATGC | |
| | GTCGTCGTGGACGTAAGCCATGAAGATCCCGAG | |
| | GTTAAGTTCAACTGGTATGTCGATGGCGTGGAAG | |
| | TCCATAATGCTAAGACTAAACCTCGCGAAGAGC | |
| | AGTACAATTCAACTTACCGGGTCGTTTCCGTTCT | |
| | GACCGTGCTGCATCAGGACTGGCTGAATGGTAA | |
| | AGAGTACAAGTGCAAAGTGTCTAACAAGGCACT | |
| | CCCCGCCCCAATTGAGAAGACTATCTCCAAAGCT | |
| | AAAGGGCAACCAAGAGAGCCCCAGGTCTACACC | |
| | CTGCCCCCCTCAAGGGATGAGCTTACTAAGAAC | |
| | CAGGTTAGTCTCACCTGCTTGGTTAAAGGATTTT | |
| | ATCCAAGCGATATTGCTGTGGAGTGGGAGTCCA | |
| | ACGGCCAGCCTGAGAACAATTATAAAACCACCC | |
| | CCCCTGTTCTTGACAGTGACGGTAGTTTCTTCCT | |
| | GTATTCCAAACTGACCGTCGATAAGAGCAGATG | |
| | GCAACAGGGAAATGTGTTCAGCTGCTCCGTGAT | |
| | GCATGAGGCGCTCCATAATCATTACACACAAAA | |
| | AAGTTTGTCCCTGAGCCCAGGCAAG | |
| hu.18B9.3i-V2 | DIQMTQSPSSLSASVGDRVTITCRASEGISSYLSWY | 165 |
| LC | QQKPGKAPKLLIYKASTLTSGVPSRFSGSGSGTDFT | |
| | LTISSLQPEDFATYYCQSAYGSSGSSGYGAAFGGG | |
| | TKVEIKRTVAAPSVFIFPPSDEQLKSGTASVVCLLN | |
| | NFYPREAKVQWKVDNALQSGNSQESVTEQDSKDS | |
| | TYSLSSTLTLSKADYEKHKVYACEVTHQGLSSPVT | |
| | KSFNRGEC | |
| hu.18B9.3i-V2 | GACATTCAGATGACCCAGAGCCCCAGCAGCCTG | 166 |
| LC (Nucleotide) | AGCGCCAGCGTGGGAGACAGAGTGACCATCACC | |
| | TGCAGAGCCAGCGAGGGAATCAGCAGCTACCTG | |
| | AGCTGGTATCAGCAGAAACCCGGCAAAGCCCCC | |
| | AAACTGCTGATCTACAAAGCCTCCACACTGACA | |
| | AGCGGAGTGCCCAGCAGATTCAGCGGAAGCGGC | |
| | AGCGGCACAGACTTCACCCTGACCATCAGCAGC | |
| | CTGCAGCCCGAAGATTTCGCCACCTACTACTGCC | |
| | AGAGCGCTTACGGCAGCAGCGGAAGCAGCGGAT | |
| | ACGGCGCCGCCTTCGGGGGAGGAACCAAAGTGG | |
| | AAATCAAGCGTACGGTGGCAGCTCCATCAGTTTT | |
| | TATCTTCCCACCAAGCGACGAGCAATTGAAGTCC | |
| | GGCACTGCCTCTGTGGTGTGCCTTCTGAACAACT | |
| | TCTATCCAAGGGAGGCCAAGGTCCAGTGGAAGG | |
| | TCGATAATGCGCTGCAGAGCGGGAACAGCCAAG | |
| | AGTCAGTGACCGAGCAGGACTCAAAAGATAGCA | |
| | CATACTCTCTGAGTTCCACCCTGACCCTGTCAAA | |
| | GGCTGACTACGAAAAGCATAAGGTATACGCATG | |
| | CGAAGTGACCCATCAGGGTCTCTCATCTCCCGTA | |
| | ACCAAATCTTTTAATAGAGGAGAATGC | |
| hu.18B9.3i-V3 | SGYMS | 13 |
| HC: CDR-1 | | |
| hu.18B9.3i-V3 | SIYAGSSGKTYYADWAKG | 29 |
| HC: CDR-2 | | |
| hu.18B9.3i-V3 | DSGGTYYTDYLYL | 44 |
| HC: CDR-3 | | |
| hu.18B9.3i-V3 | RASEGISSYLS | 57 |
| LC: CDR-1 | | |
| hu.18B9.3i-V3 | KASTLTS | 66 |
| LC: CDR-2 | | |
| hu.18B9.3i-V3 | QSTYGSSGSSGYAAA | 85 |
| LC: CDR-3 | | |
| hu.18B9.3i-V3 | EVQLVESGGGLVQPGGSLRLSCAASGFSFNSGYMS | 167 |
| VH | WVRQAPGKGLEWIASIYAGSSGKTYYADWAKGR | |
| | FTISKTSSKTTVYLQMNSLRAEDTAVYYCARDSGG | |
| | TYYTDYLYLWGQGTLVTVSS | |
| hu.18B9.3i-V3 | DIQMTQSPSSLSASVGDRVTITCRASEGISSYLSWY | 168 |
| VL | QQKPGKAPKLLIYKASTLTSGVPSRFSGSGSGTDFT | |
| | LTISSLQPEDFATYYCQSTYGSSGSSGYAAAFGGG | |
| | TKVEIK | |
| hu.18B9.3i-V3 | EVQLVESGGGLVQPGGSLRLSCAASGFSFNSGYMS | 169 |
| HC | WVRQAPGKGLEWIASIYAGSSGKTYYADWAKGR | |
| | FTISKTSSKTTVYLQMNSLRAEDTAVYYCARDSGG | |
| | TYYTDYLYLWGQGTL VTVSSASTKGPSVFPLAPSS | |
| | KSTSGGTAALGCLVKDYFPEPVTVSWNSGALTSG | |
| | VHTFPAVLQSSGLYSLSSVVTVPSSSLGTQTYICNV | |
| | NHKPSNTKVDKKVEPKSCDKTHTCPPCPAPELLGG | |
| | PSVFLFPPKPKDTLMISRTPEVTCVVVDVSHEDPEV | |
| | KFNWYVDGVEVHNAKTKPREEQYNSTYRVVSVL | |
| | TVLHQDWLNGKEYKCKVSNKALPAPIEKTISKAK | |
| | GQPREPQVYTLPPSRDELTKNQVSLTCLVKGFYPS | |
| | DIAVEWESNGQPENNYKTTPPVLDSDGSFFLYSKL | |
| | TVDKSRWQQGNVFSCSVMHEALHNHYTQKSLSLS | |
| | PGK | |
| hu.18B9.3i-V3 | GAGGTGCAGCTGGTGGAGAGCGGGGGGGGACTG | 170 |
| HC (Nucleotide) | GTGCAGCCAGGAGGAAGCCTGAGACTGAGCTGT | |
| | GCCGCCAGCGGGTTTAGCTTTAATAGCGGCTATA | |
| | TGAGCTGGGTGAGACAGGCCCCCGGAAAAGGAC | |
| | TGGAGTGGATCGCCTCCATTTACGCCGGAAGTA | |
| | GCGGCAAGACCTACTACGCCGATTGGGCTAAGG | |
| | GAAGATTCACCATTAGCAAGACCAGCAGTAAGA | |
| | CAACCGTGTACCTGCAGATGAACAGCCTGAGAG | |
| | CCGAGGACACCGCCGTGTACTATTGCGCCCGCG | |
| | ACAGCGGAGGAACCTACTACACCGACTACCTGT | |
| | ACCTGTGGGGACAGGGCACCCTGGTGACCGTGA | |
| | GCAGCGCTAGCACCAAAGGTCCCAGCGTGTTCC | |
| | CACTCGCCCCGAGTTCAAAATCAACTTCTGGAGG | |
| | CACCGCCGCCCTGGGTTGCCTGGTAAAGGACTA | |
| | CTTCCCAGAGCCCGTGACCGTGAGCTGGAACTCC | |
| | GGGGCACTGACATCTGGCGTTCATACTTTCCCGG | |
| | CCGTGCTCCAGTCTTCAGGTCTGTATAGTCTCTC | |
| | CTCTGTGGTCACTGTCCCATCTAGCTCTCTGGGC | |
| | ACCCAAACCTACATATGCAACGTTAATCACAAG | |
| | CCGAGCAATACTAAAGTTGACAAAAAGGTGGAA | |
| | CCCAAGTCTTGTGACAAGACCCACACGTGTCCCC | |
| | CCTGCCCGGCTCCTGAGCTGCTTGGCGGCCCCAG | |
| | CGTCTTTCTCTTTCCCCCAAAGCCAAAAGATACC | |
| | TTGATGATCAGCAGAACTCCCGAGGTGACATGC | |
| | GTCGTCGTGGACGTAAGCCATGAAGATCCCGAG | |
| | GTTAAGTTCAACTGGTATGTCGATGGCGTGGAAG | |
| | TCCATAATGCTAAGACTAAACCTCGCGAAGAGC | |
| | AGTACAATTCAACTTACCGGGTCGTTTCCGTTCT | |
| | GACCGTGCTGCATCAGGACTGGCTGAATGGTAA | |
| | AGAGTACAAGTGCAAAGTGTCTAACAAGGCACT | |
| | CCCCGCCCCAATTGAGAAGACTATCTCCAAAGCT | |
| | AAAGGGCAACCAAGAGAGCCCCAGGTCTACACC | |
| | CTGCCCCCCTCAAGGGATGAGCTTACTAAGAAC | |
| | CAGGTTAGTCTCACCTGCTTGGTTAAAGGATTTT | |
| | ATCCAAGCGATATTGCTGTGGAGTGGGAGTCCA | |
| | ACGGCCAGCCTGAGAACAATTATAAAACCACCC | |
| | CCCCTGTTCTTGACAGTGACGGTAGTTTCTTCCT | |
| | GTATTCCAAACTGACCGTCGATAAGAGCAGATG | |
| | GCAACAGGGAAATGTGTTCAGCTGCTCCGTGAT | |
| | GCATGAGGCGCTCCATAATCATTACACACAAAA | |
| | AAGTTTGTCCCTGAGCCCAGGCAAG | |
| hu.18B9.3i-V3 | DIQMTQSPSSLSASVGDRVTITCRASEGISSYLSWY | 171 |
| LC | QQKPGKAPKLLIYKASTLTSGVPSRFSGSGSGTDFT | |
| | LTISSLQPEDFATYYCQSTYGSSGSSGYAAAFGGG | |
| | TKVEIKRTVAAPSVFIFPPSDEQLKSGTASVVCLLN | |
| | NFYPREAKVQWKVDNALQSGNSQESVTEQDSKDS | |
| | TYSLSSTLTLSKADYEKHK VYACEVTHQGLSSPVT | |
| | KSFNRGEC | |
| hu.18B9.3i-V3 | GACATTCAGATGACCCAGAGCCCCAGCAGCCTG | 172 |
| LC (Nucleotide) | AGCGCCAGCGTGGGAGACAGAGTGACCATCACC | |
| | TGCAGAGCCAGCGAGGGAATCAGCAGCTACCTG | |
| | AGCTGGTATCAGCAGAAACCCGGCAAAGCCCCC | |
| | AAACTGCTGATCTACAAAGCCTCCACACTGACA | |
| | AGCGGAGTGCCCAGCAGATTCAGCGGAAGCGGC | |
| | AGCGGCACAGACTTCACCCTGACCATCAGCAGC | |
| | CTGCAGCCCGAAGATTTCGCCACCTACTACTGCC | |
| | AGAGCACCTACGGCAGCAGCGGCAGTAGCGGAT | |
| | ACGCCGCCGCCTTCGGAGGCGGAACCAAGGTGG | |
| | AAATCAAGCGTACGGTGGCAGCTCCATCAGTTTT | |
| | TATCTTCCCACCAAGCGACGAGCAATTGAAGTCC | |
| | GGCACTGCCTCTGTGGTGTGCCTTCTGAACAACT | |
| | TCTATCCAAGGGAGGCCAAGGTCCAGTGGAAGG | |
| | TCGATAATGCGCTGCAGAGCGGGAACAGCCAAG | |
| | AGTCAGTGACCGAGCAGGACTCAAAAGATAGCA | |
| | CATACTCTCTGAGTTCCACCCTGACCCTGTCAAA | |
| | GGCTGACTACGAAAAGCATAAGGTATACGCATG | |
| | CGAAGTGACCCATCAGGGTCTCTCATCTCCCGTA | |
| | ACCAAATCTTTTAATAGAGGAGAATGC | |

### Alanine Scanning

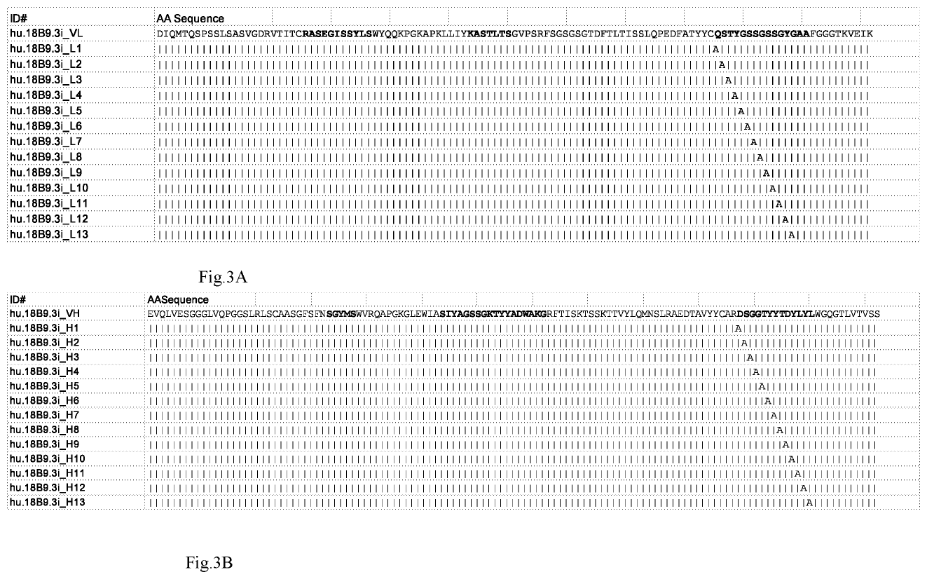

Alanine scanning variants of the hu.18B9.3i antibody were generated to identify CDR3 residues important for binding to human CD47. Alanine mutants were systematically generated by mutating each residue of the heavy and light chain CDR3 regions to alanine, as shown in FIGS. 3A-3B. Each mutant variant was tested for binding to CD47 by ELISA.

TABLE 11
Binding of hu.18B9.3i antibody alanine variants
to recombinant human CD47 as determined by ELISA.

| Anti-CD47 | |
|---|---|
| Antibody | EC50 (nM) |
| hu.18B9.3i | A |
| HC_D95A | NB |
| HC_S96A | B |
| HC_G97A | NB |
| HC_G98A | NB |
| HC_T99A | A |
| HC_Y100A | A |
| HC_Y100a_A | A |
| HC_T100b_A | A |
| HC_D100c_A | NB |
| HC_Y100d_A | NB |
| HC_L100e_A | C |
| HC_Y101A | NB |
| HC_L102A | A |
| LC_Q89A | B |
| LC_S90A | A |
| LC_T91A | A |
| LC_Y92A | A |
| LC_G93A | A |
| LC_S94A | A |
| LC_S95A | A |
| LC_G95a_A | A |
| LC_S95b_A | A |
| LC_S95c_A | A |
| LC_G95d_A | A |
| LC_Y95e_A | A |
| LC_G95f_A | A |
| 5F9.G4 | A |
| huIgG1 control | NB |

EC50 ranges for CD47- A: 0.01 nM-0.5 nM; B: 0.5 nM-5 nM; C: >5 nM; NB: not bound.
Amino acid numbering based on Kabat numbering system.

### Example 5: Binding and Functional Evaluation of Humanized/Engineered Anti-CD47 Antibody Variants

After humanization and alanine scan of two antibodies, 1D15E11 from mouse hybridoma and 18B9 from rabbit B cell cloning, the anti-CD47 antibodies variants hu.1D15E11-Vi, hu.1D15E11-V2, hu.1D15E11-V3, hu.1D15E11-V4, hu.18B9.3i-V1, hu.18B9.3i-V2 and hu.18B9.3i-V3 were selected for further evaluation in vitro and in vivo as compared with BMK-1 and BMK-2.

The sequences of the anti-CD47 benchmark antibody BMK-1 and the anti-CD47 benchmark BMK-2 are provided in Table 12.

TABLE 12
Control amino acid sequences

| Construct | | SEQ ID |
|---|---|---|
| Description | Sequence | NO: |
| BMK-1 HC | QVQLVQSGAEVKKPGASVKVSCKASGYTFTNYN | 173 |
| (5F9.G4) | MHWVRQAPGQRLEWMGTIYPGNDDTSYNQKFK | |
| | DRVTITADTSASTAYMELSSLRSEDTAVYYCARGG | |
| | YRAMDYWGQGTLVTVSSASTKGPSVFPLAPCSRS | |
| | TSESTAALGCLVKDYFPEPVTVSWNSGALTSGVHT | |
| | FPAVLQSSGLYSLSSVVTVPSSSLGTKTYTCNVDH | |
| | KPSNTKVDKRVESKYGPPCPPCPAPEFLGGPSVFL | |
| | FPPKPKDTLMISRTPEVTCVVVDVSQEDPEVQFNW | |
| | YVDGVEVHNAKTKPREEQFNSTYRVVSVLTVLHQD | |
| | WLNGKEYKCKVSNKGLPSSIEKTISKAKGQPREPQ | |
| | VYTLPPSQEEMTKNQVSLTCLVKGFYPSDIAVEWE | |
| | SNGQPENNYKTTPPVLDSDGSFFLYSRLTVDKSRW | |
| | QEGNVFSCSVMHEALHNHYTQKSLSLSLGK | |
| BMK-1 LC | DIVMTQSPLSLPVTPGEPASISCRSSQSIVYSNGNT | 174 |
| (5F9.G4) | YLGWYLQKPGQSPQLLIYKVSNRFSGVPDRFSGSG | |
| | SGTDFTLKISRVEAEDVGVYYCFQGSHVPYTFGQG | |
| | TKLEIKRTVAAPSVFIFPPSDEQLKSGTASVVCLL | |
| | NNFYPREAKVQWKVDNALQSGNSQESVTEQDSKD | |
| | STYSLSSTLTLSKADYEKHKVYACEVTHQGLSSPV | |
| | TKSFNRGEC | |
| BMK-2 | EEELQVIQPDKSVSVAAGESAILHCTVTSLIPVGPI | 175 |
| (TTI-621) | QWFRGAGPARELIYNQKEGHFPRVTTVSESTKRENM | |
| | DFSISISNITPADAGTYYCVKFRKGSPDTEFKSGAG | |
| | TELSVRAKPSDKTHTCPPCPAPELLGGPSVFLFPPK | |
| | PKDTLMISRTPEVTCVVVDVSHEDPEVKFNWYVD | |
| | GVEVHNAKTKPREEQYNSTYRVVSVLTVLHQDWL | |
| | NGKEYKCKVSNKALPAPIEKTISKAKGQPREPQVY | |
| | TLPPSRDELTKNQVSLTCLVKGFYPSDIAVEWESN | |
| | GQPENNYKTTPPVLDSDGSFFLYSKLTVDKSRWQQ | |
| | GNVFSCSVMHEALHNHYTQKSLSLSPGK | |

Antibodies binding to huCD47 and cyno CD47 by ELISA

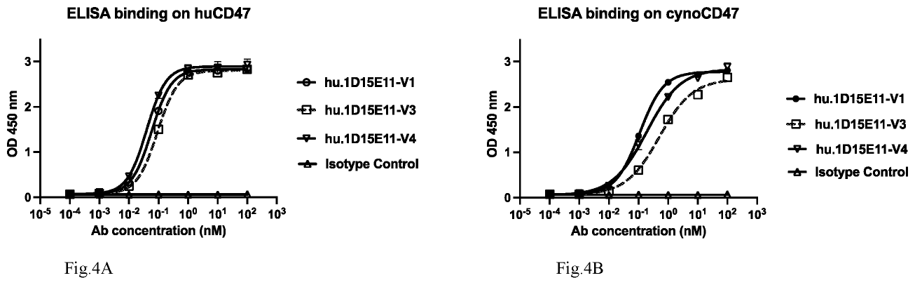

A 96-well plate was coated overnight at 4° C. with 1 μg/mL recombinant CD47. After washing 3 times, the plate was blocked with 300 μL 100 BSA in PBST at 37° C. for 1 hour. Serially diluted antibodies were added and incubated at 37° C. for 1 hour. The plate was then washed 4 times with PBST and incubated with 1:5000 diluted peroxidase labeled goat anti-human IgG (Fab specific) secondary antibody (Sigma, Cat #A0293) for 1 hour at 37° C. The plate was washed again 4 times with PBST, incubated with 3,3′,5,5′-tetramethylbenzidine (TMB) substrate for 15 min at room temperature, terminated with 1N HCl, and then read at 450 nm. FIG. 4s. A-4B showed hu.1D15E11 variants showed robust binding to human and cyno CD47 by ELISA.

### Inhibition of SIPRA Binding to CD47+ Cells

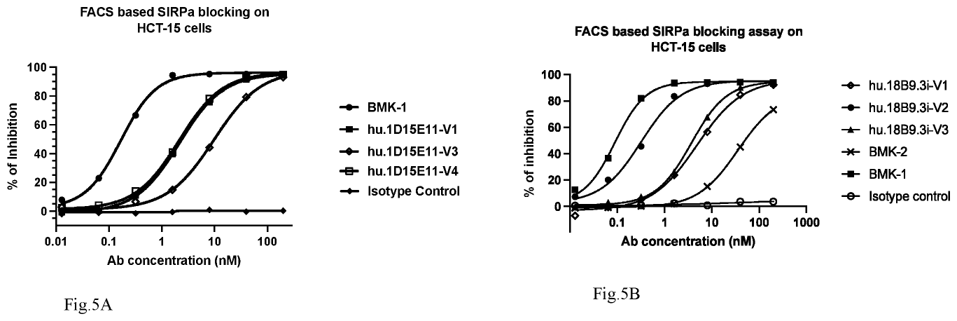

SIRPA blocking assays were performed as described above. As shown in FIG. 5A, hu. 1D15E11 variants showed potent SIRPa blocking activity. In FIG. 5B, hu.18B9.3i-V1, hu.18B9.3i-V2 and hu.18B9.3i-V3 showed more potent SIRPa blocking activity than BMK-2 but less than BMK-1.

### Antibody-Dependent Cellular Phagocytosis

The antibodies were tested in an antibody-dependent cellular phagocytosis (ADCP) assay. Peripheral blood mononuclear cells (PBMCs) were isolated from human donors. Monocytes were enriched using a Human Monocyte Enrichment Kit without CD16 depletion (STEMCELL, Cat #19058). Isolated monocytes were differentiated into macrophages by culturing monocytes in complete culture media (RPMI 1640+10% FBS) with 20 ng/mL of human Macrophage Colony-Stimulating Factor (M-CSF, Peprotech, Cat #: 3-25-10). The media was changed every three days. After 7 days of culturing in M-CSF containing culture media, macrophages were collected and counted. Target tumor cells were collected and washed with D-PBS two times to remove remaining FBS. The washed tumor cells were resuspended in PBS at a cell concentration of 5-10×10⁶/mL. Cancer cells were stained with CFSE (ebiosciences, Cat #: 65-0580-84) at a final concentration of 3 μM and mixed immediately. The cells were stained in the dark at room temperature for 10 minutes. The staining was terminated by adding 4-5 volumes of cold complete media and incubating on ice for 5 minutes. Stained cells were washed three times with RPMI 1640+10% FBS. Cells were resuspended in 1 mL RPMI 1640+10% FBS and counted, then adjusted the cell numbers to 3×10⁵ cells/mL. 50 μL cells were seeded into a 96 well deep U-plate (Axygen, Cat #: P-DW-20-C) wherein each well contained 1.5×10⁴ cells. 50 μL of diluted antibodies were added to each well. 100 μL of macrophages (1.5×10⁴ cells) were added to each well and incubated at 37° C., 5% CO₂ for 1.5 hours. After incubation, cells were washed with 2 mL of 2% FBS in D-PBS once. 100 μL of diluted Fc blocker (Human TruStain FcX (Fc Receptor Blocking Solution), Biolegend Cat #: 422302)) was added and the cells were incubated at room temperature for 10 minutes. 20 μL of diluted anti-human CD11b antibody was added to each well and incubated for 30 minutes at 4° C. in the dark. Cells were washed with 2% FBS-D-PBS once. Phagocytosis was detected in a flow cytometer by the appearance of CFSE/CD11b double positive cells indicative of macrophages that engulfed the tumor cells.

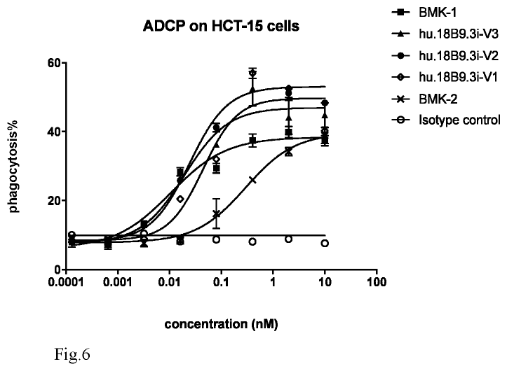

In FIG. 6, hu.18B9.3i-V1, hu.18B9.3i-V2 and hu.18B9.3i-V3 showed more potent ADCP activity compared with BMK-2, but not BMK-1.

### Antibody Binding to Red Blood Cells (RBCs) Platelets and Hemagglutination

The RBC binding assay was performed by spinning down fresh human whole blood at 200 g for 10 minutes. Collected RBCs were washed twice with PBS and counted using flow cytometry. 1×10⁶ cells were dispensed into each well of a 96 well culture plate. Serially diluted anti-CD47 antibodies were added and incubated for 1 hour at 4° C. Cells were washed with FACS buffer (PBS+2% FBS) twice. Secondary antibody (Alexa Fluor® 488 Goat Anti-Human IgG (H+L)) was added and incubated for 1 hour at 4° C. Cells were washed twice and resuspended in 200 μL of FACS buffer and analyzed by flow cytometry.

The hemagglutination assay was performed by diluting human RBCs and incubating RBCs at 37° C. for 2 hours with a titration of CD47 antibodies (from 600 nM) in a round bottom 96 well plate. Hemagglutination is demonstrated by the presence of crosslinked RBCs, which appear as a haze because they do not settle to the bottom of the well, in contrast to non-hemagglutinated RBCs.

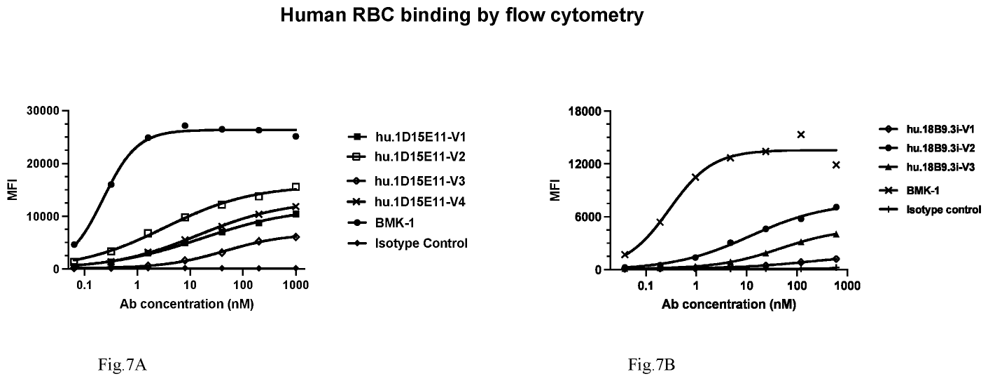

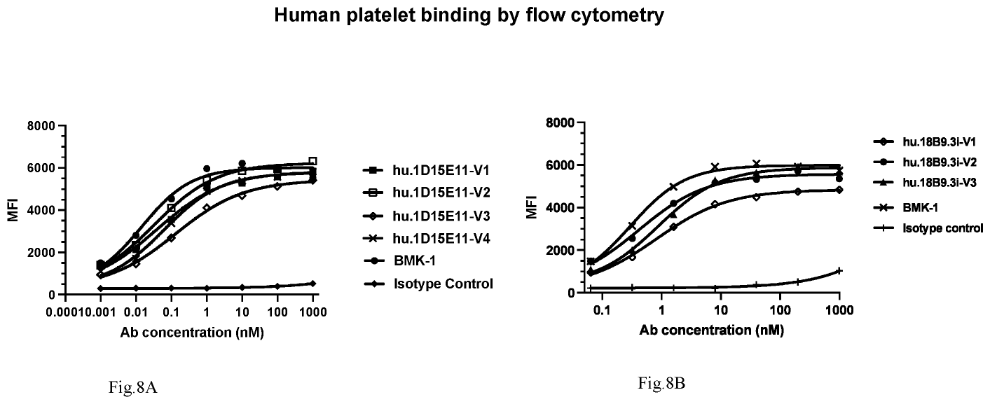

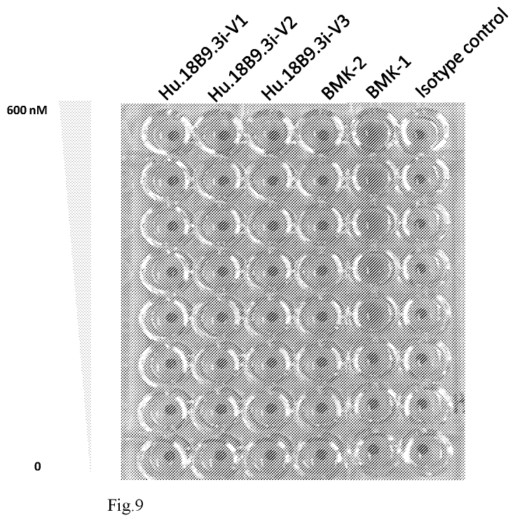

In FIG. 7, hu.1D15E11 variants showed less binding on human RBCs as compared with BMK-1 (FIG. 7A). hu.18B9.3i-V1, hu.18B9.3i-V2 and hu.18B9.3i-V3 showed less binding on human RBCs as compared with BMK-1 (FIG. 7B). FIG. 7, shows binding of anti-CD47 antibodies hu.1D15E11 variants (FIG. 8A), hu.18B9.3i-V1, hu.18B9.3i-V2 and hu.18B9.3i-V3 (FIG. 8B) on human platelets as compared with BMK-1, all antibodies showed less binding on human platelets as compared with BMK-1. In FIG. 9, hu.18B9.3i-V1, hu.18B9.3i-V2 and hu.18B9.3i-V3 did not induce hemagglutination at 600 nM concentration. Hu.1D15E11 variants didn't induce hemagglutination as well, data not shown.

### Example 6: In Vivo Efficacy Study

Ten million Raji cells with Matrigel were injected subcutaneously (sc) into the flank of 6-8 weeks old female CB17/SCID mice. Tumor volume was measured using a digital caliper and volumes were calculated using the formula: tumor volume=(length×width²)/2. When the mean tumor volume reached approximately 100 mm³, animals were randomized by tumor volumes into 6 animals per group and treatment initiated. Test antibodies were administered through bolus tail vein injection at 0.3 mg/kg twice per week for 3 weeks. Tumor volumes were measured twice weekly. Animals were humanely euthanized if their tumor volumes exceeded 2500 mm³ or if they experienced over 20% of body weight loss.

Ten million 786-O cells with Matrigel were injected subcutaneously (sc) into the flank of 6-8-week-old female Balb/C nude mice. Tumor volume was measured as described above. When the mean tumor volume reached approximately 150 mm³, animals were randomized by tumor volumes into 6 animals per group and treatment initiated. Test antibodies were administered through bolus tail vein injection at 10 mg/kg once per week for 6 weeks. Tumor volumes were measured twice weekly. Animals were humanely euthanized if their tumor volumes exceeded 2500 mm³ or if they experienced over 20% of body weight loss.

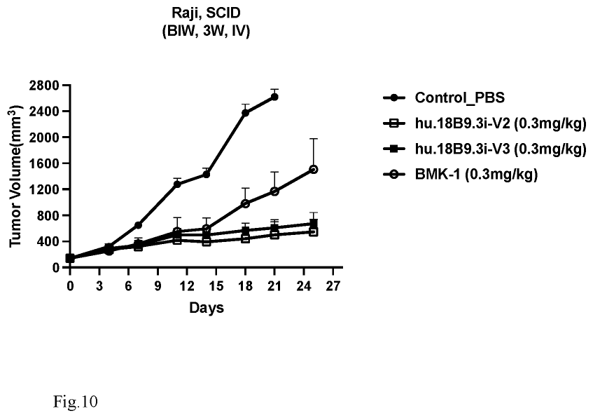

In FIG. 10, hu.18B9.3i-V2 and hu.18B9.3i-V3 showed tumor growth inhibition significantly superior to benchmark antibody BMK-1 at 0.3 mg/kg treatment.

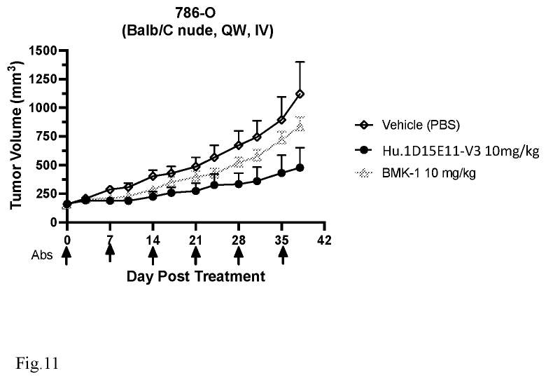

In FIG. 11, hu.1D15E11-V3 showed tumor growth inhibition significantly superior to benchmark antibody BMK-1 at 10 mg/kg treatment.

What is claimed is:

1. An antibody that binds specifically to CD47 comprising at least one complementarity determining region (CDR) according to SEQ ID NOs: 1-85, 176-178 or an amino acid sequence that has 0-1 amino acid mutations, substitutions, or deletions relative to SEQ ID NOs: 1-13, 58-69, or an amino acid sequence that has 0-3 amino acid mutations, substitutions, or deletions to relative to SEQ ID NOs: 14-57, 70-85, 176-178.

2. The antibody according to claim 1, wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain comprises a set of sequences selected from the group consisting of the following set of sequences:
HC-CDR1: SEQ ID NO: 1, HC-CDR2: SEQ ID NO: 14, HC-CDR3: SEQ ID NO: 30;
HC-CDR1: SEQ ID NO: 2, HC-CDR2: SEQ ID NO: 15, HC-CDR3: SEQ ID NO: 31;
HC-CDR1: SEQ ID NO: 3, HC-CDR2: SEQ ID NO: 16, HC-CDR3: SEQ ID NO: 32;
HC-CDR1: SEQ ID NO: 2, HC-CDR2: SEQ ID NO: 17, HC-CDR3: SEQ ID NO: 33;
HC-CDR1: SEQ ID NO: 4, HC-CDR2: SEQ ID NO: 18, HC-CDR3: SEQ ID NO: 34;
HC-CDR1: SEQ ID NO: 5, HC-CDR2: SEQ ID NO: 19, HC-CDR3: SEQ ID NO: 35;
HC-CDR1: SEQ ID NO: 6, HC-CDR2: SEQ ID NO: 20, HC-CDR3: SEQ ID NO: 176;
HC-CDR1: SEQ ID NO: 7, HC-CDR2: SEQ ID NO: 21, HC-CDR3: SEQ ID NO: 36;
HC-CDR1: SEQ ID NO: 8, HC-CDR2: SEQ ID NO: 22, HC-CDR3: SEQ ID NO: 37;
HC-CDR1: SEQ ID NO: 9, HC-CDR2: SEQ ID NO: 23, HC-CDR3: SEQ ID NO: 38;
HC-CDR1: SEQ ID NO: 10, HC-CDR2: SEQ ID NO: 24, HC-CDR3: SEQ ID NO: 39;
HC-CDR1: SEQ ID NO: 8, HC-CDR2: SEQ ID NO: 25, HC-CDR3: SEQ ID NO: 40;
HC-CDR1: SEQ ID NO: 11, HC-CDR2: SEQ ID NO: 26, HC-CDR3: SEQ ID NO: 41;
HC-CDR1: SEQ ID NO: 12, HC-CDR2: SEQ ID NO: 27, HC-CDR3: SEQ ID NO: 42;
HC-CDR1: SEQ ID NO: 1, HC-CDR2: SEQ ID NO: 28, HC-CDR3: SEQ ID NO: 30;
HC-CDR1: SEQ ID NO: 1, HC-CDR2: SEQ ID NO: 28, HC-CDR3: SEQ ID NO: 177;
HC-CDR1: SEQ ID NO: 1, HC-CDR2: SEQ ID NO: 28, HC-CDR3: SEQ ID NO: 178;
HC-CDR1: SEQ ID NO: 13, HC-CDR2: SEQ ID NO: 29, HC-CDR3: SEQ ID NO: 43; and
HC-CDR1: SEQ ID NO: 13, HC-CDR2: SEQ ID NO: 29, HC-CDR3: SEQ ID NO: 44.

3. The antibody according to claim 2, wherein the antibody comprises a light chain variable domain that comprises CDRs: LC-CDR1, LC-CDR2, LC-CDR3, wherein the LC-CDR1, LC-CDR2, LC-CDR3 of the light chain variable domain comprises a set of sequences selected from the group consisting of the following set of sequences:
LC-CDR1: SEQ ID NO: 45, LC-CDR2: SEQ ID NO: 58, LC-CDR3: SEQ ID NO: 70;
LC-CDR1: SEQ ID NO: 46, LC-CDR2: SEQ ID NO: 59, LC-CDR3: SEQ ID NO: 71;
LC-CDR1: SEQ ID NO: 47, LC-CDR2: SEQ ID NO: 60, LC-CDR3: SEQ ID NO: 72;
LC-CDR1: SEQ ID NO: 48, LC-CDR2: SEQ ID NO: 59, LC-CDR3: SEQ ID NO: 73;
LC-CDR1: SEQ ID NO: 49, LC-CDR2: SEQ ID NO: 61, LC-CDR3: SEQ ID NO: 74;
LC-CDR1: SEQ ID NO: 50, LC-CDR2: SEQ ID NO: 62, LC-CDR3: SEQ ID NO: 75;
LC-CDR1: SEQ ID NO: 51, LC-CDR2: SEQ ID NO: 63, LC-CDR3: SEQ ID NO: 76;
LC-CDR1: SEQ ID NO: 52, LC-CDR2: SEQ ID NO: 62, LC-CDR3: SEQ ID NO: 77;
LC-CDR1: SEQ ID NO: 53, LC-CDR2: SEQ ID NO: 64, LC-CDR3: SEQ ID NO: 78;
LC-CDR1: SEQ ID NO: 54, LC-CDR2: SEQ ID NO: 65, LC-CDR3: SEQ ID NO: 79;
LC-CDR1: SEQ ID NO: 55, LC-CDR2: SEQ ID NO: 66, LC-CDR3: SEQ ID NO: 80;
LC-CDR1: SEQ ID NO: 53, LC-CDR2: SEQ ID NO: 67, LC-CDR3: SEQ ID NO: 78;
LC-CDR1: SEQ ID NO: 52, LC-CDR2: SEQ ID NO: 62, LC-CDR3: SEQ ID NO: 81;
LC-CDR1: SEQ ID NO: 56, LC-CDR2: SEQ ID NO: 68, LC-CDR3: SEQ ID NO: 82;
LC-CDR1: SEQ ID NO: 45, LC-CDR2: SEQ ID NO: 69, LC-CDR3: SEQ ID NO: 70;
LC-CDR1: SEQ ID NO: 57, LC-CDR2: SEQ ID NO: 66, LC-CDR3: SEQ ID NO: 83;
LC-CDR1: SEQ ID NO: 57, LC-CDR2: SEQ ID NO: 66, LC-CDR3: SEQ ID NO: 84;
LC-CDR1: SEQ ID NO: 57, LC-CDR2: SEQ ID NO: 66, LC-CDR3: SEQ ID NO: 85.

4. The antibody according to claim 1, wherein the antibody comprises at least one complementarity determining region (CDR) according to SEQ ID NOs: 1, 13, 66, 69, 28-30, 30, 43, 44, 45, 57, 70, 83-85, 177-178 or an amino acid sequence that has 0-1 amino acid mutations, substitutions, or deletions relative to SEQ ID NOs: 1, 13, 66, 69, or an amino acid sequence that has 0-3 amino acid mutations, substitutions, or deletions to relative to SEQ ID NOs:28-30, 30, 43, 44, 45, 57, 70, 83-85, 177-178.

5. The antibody according to claim 4, wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain comprises a set of sequences selected from the group consisting of the following set of sequences:
HC-CDR1: SEQ ID NO: 1, HC-CDR2: SEQ ID NO: 28, HC-CDR3: SEQ ID NO: 30;
HC-CDR1: SEQ ID NO: 1, HC-CDR2: SEQ ID NO: 28, HC-CDR3: SEQ ID NO: 177;
HC-CDR1: SEQ ID NO: 1, HC-CDR2: SEQ ID NO: 28, HC-CDR3: SEQ ID NO: 178;
HC-CDR1: SEQ ID NO: 13, HC-CDR2: SEQ ID NO: 29, HC-CDR3: SEQ ID NO: 43; and
HC-CDR1: SEQ ID NO: 13, HC-CDR2: SEQ ID NO: 29, HC-CDR3: SEQ ID NO: 44; and wherein the CDRs comprise 0-2 amino acid mutations, substitutions, or deletions in at least one of the HC-CDR1, HC-CDR2, HC-CDR3.

6. The antibody according to claim 4, wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain comprises a set of sequences selected from the group consisting of the following set of sequences:
HC-CDR1: SEQ ID NO: 1, HC-CDR2: SEQ ID NO: 28, HC-CDR3: SEQ ID NO: 30;
HC-CDR1: SEQ ID NO: 1, HC-CDR2: SEQ ID NO: 28, HC-CDR3: SEQ ID NO: 177;
HC-CDR1: SEQ ID NO: 1, HC-CDR2: SEQ ID NO: 28, HC-CDR3: SEQ ID NO: 178;
HC-CDR1: SEQ ID NO: 13, HC-CDR2: SEQ ID NO: 29, HC-CDR3: SEQ ID NO: 43; and
HC-CDR1: SEQ ID NO: 13, HC-CDR2: SEQ ID NO: 29, HC-CDR3: SEQ ID NO: 44.

7. The antibody according to claim 4, wherein the antibody comprises a light chain variable domain that comprises CDRs: LC-CDR1, LC-CDR2, LC-CDR3, wherein the LC-CDR1, LC-CDR2, LC-CDR3 of the light chain variable domain comprises a set of sequences selected from the group consisting of the following set of sequences:
LC-CDR1: SEQ ID NO: 45, LC-CDR2: SEQ ID NO: 69, LC-CDR3: SEQ ID NO: 70;
LC-CDR1: SEQ ID NO: 57, LC-CDR2: SEQ ID NO: 66, LC-CDR3: SEQ ID NO: 83;
LC-CDR1: SEQ ID NO: 57, LC-CDR2: SEQ ID NO: 66, LC-CDR3: SEQ ID NO: 84;
LC-CDR1: SEQ ID NO: 57, LC-CDR2: SEQ ID NO: 66, LC-CDR3: SEQ ID NO: 85; and
wherein the CDRs comprise 0-2 amino acid mutations, substitutions, or deletions in at least one of the LC-CDR1, LC-CDR2, LC-CDR3.

8. The antibody according to claim 4, wherein the antibody comprises a light chain variable domain that comprises CDRs: LC-CDR1, LC-CDR2, LC-CDR3, wherein the LC-CDR1, LC-CDR2, LC-CDR3 of the light chain variable domain comprises a set of sequences selected from the group consisting of the following set of sequences:
LC-CDR1: SEQ ID NO: 45, LC-CDR2: SEQ ID NO: 69, LC-CDR3: SEQ ID NO: 70;
LC-CDR1: SEQ ID NO: 57, LC-CDR2: SEQ ID NO: 66, LC-CDR3: SEQ ID NO: 83;
LC-CDR1: SEQ ID NO: 57, LC-CDR2: SEQ ID NO: 66, LC-CDR3: SEQ ID NO: 84; and
LC-CDR1: SEQ ID NO: 57, LC-CDR2: SEQ ID NO: 66, LC-CDR3: SEQ ID NO: 85.

9. The antibody according to claim 4, wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain comprises a set of sequences:
HC-CDR1: SEQ ID NO: 13, HC-CDR2: SEQ ID NO: 29, HC-CDR3: SEQ ID NO: 44;
and wherein the CDRs comprise 0-2 amino acid mutations, substitutions, or deletions in at least one of the HC-CDR1, HC-CDR2, HC-CDR3.

10. The antibody according to claim 4, wherein the antibody comprises a heavy chain variable domain that comprises CDRs: HC-CDR1, HC-CDR2, HC-CDR3, wherein the HC-CDR1, HC-CDR2, HC-CDR3 of the heavy chain variable domain comprises a set of sequences:
HC-CDR1: SEQ ID NO: 13, HC-CDR2: SEQ ID NO: 29, HC-CDR3: SEQ ID NO: 44.

11. The antibody according to claim 4, wherein the antibody comprises a light chain variable domain that comprises CDRs: LC-CDR1, LC-CDR2, LC-CDR3, wherein the LC-CDR1, LC-CDR2, LC-CDR3 of the light chain variable domain comprises:
LC-CDR1; SEQ ID NO: 57, LC-CDR2: SEQ ID NO: 66, LC-CDR3: SEQ ID NO: 84; or
LC-CDR1: SEQ ID NO: 57, LC-CDR2: SEQ ID NO: 66, LC-CDR3: SEQ ID NO: 85;
and wherein the CDRs comprise 0-2 amino acid mutations, substitutions, or deletions in at least one of the LC-CDR1, LC-CDR2, LC-CDR3.

12. The antibody according to claim 4, wherein the antibody comprises a light chain variable domain that comprises CDRs: LC-CDR1, LC-CDR2, LC-CDR3, wherein the LC-CDR1, LC-CDR2, LC-CDR3 of the light chain variable domain comprises
LC-CDR1, SEQ ID NO: 57, LC-CDR2: SEQ ID NO: 66, LC-CDR3: SEQ ID NO: 84; or
LC-CDR1, SEQ ID NO: 57, LC-CDR2: SEQ ID NO: 66, LC-CDR3: SEQ ID NO: 85.

13. The antibody according to claim 4, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise a set of sequences selected from the group consisting of the following set of sequences:
HC-CDR1: SEQ ID NO: 1, HC-CDR2: SEQ ID NO: 28, HC-CDR3: SEQ ID NO: 30, LC-CDR1: SEQ ID NO: 45, LC-CDR2: SEQ ID NO: 69, LC-CDR3: SEQ ID NO: 70;
HC-CDR1: SEQ ID NO: 1, HC-CDR2: SEQ ID NO: 28, HC-CDR3: SEQ ID NO: 177, LC-CDR1: SEQ ID NO: 45, LC-CDR2: SEQ ID NO: 69, LC-CDR3: SEQ ID NO: 70;
HC-CDR1 SEQ ID NO: 1, HC-CDR2: SEQ ID NO: 28, HC-CDR3: SEQ ID NO: 178, LC-CDR1 SEQ ID NO: 45, LC-CDR2: SEQ ID NO: 69, LC-CDR3: SEQ ID NO: 70;
HC-CDR1 SEQ ID NO: 13, HC-CDR2: SEQ ID NO: 29, HC-CDR3: SEQ ID NO: 43, LC-CDR1: SEQ ID NO: 57, LC-CDR2: SEQ ID NO: 66, LC-CDR3: SEQ ID NO: 83;
HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 84; and
HC-CDR1: SEQ ID NO: 13, HC-CDR2: SEQ ID NO: 29, HC-CDR3: SEQ ID NO: 44; LC-CDR1: SEQ ID NO: 57, LC-CDR2: SEQ ID NO: 66, LC-CDR3: SEQ ID NO: 85;
wherein the CDRs comprise 0-2 amino acid mutations, substitutions, or deletions in at least one of the HC-CDR1, HC-CDR2, HC-CDR3, LC-CDR1, LC-CDR2, LC-CDR3.

14. The antibody according to claim 4, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise a set of sequences selected from the group consisting of the following set of sequences:
HC-CDR1: SEQ ID NO: 1, HC-CDR2: SEQ ID NO: 28, HC-CDR3: SEQ ID NO: 30, LC-CDR1: SEQ ID NO: 45, LC-CDR2: SEQ ID NO: 69, LC-CDR3: SEQ ID NO: 70;
HC-CDR1: SEQ ID NO: 1, HC-CDR2: SEQ ID NO: 28, HC-CDR3: SEQ ID NO: 177, LC-CDR1: SEQ ID NO: 45, LC-CDR2: SEQ ID NO: 69, LC-CDR3: SEQ ID NO: 70;
HC-CDR1 SEQ ID NO: 1, HC-CDR2: SEQ ID NO: 28, HC-CDR3: SEQ ID NO: 178, LC-CDR1 SEQ ID NO: 45, LC-CDR2: SEQ ID NO: 69, LC-CDR3: SEQ ID NO: 70;
HC-CDR1 SEQ ID NO: 13, HC-CDR2: SEQ ID NO: 29, HC-CDR3: SEQ ID NO: 43, LC-CDR1: SEQ ID NO: 57, LC-CDR2: SEQ ID NO: 66, LC-CDR3: SEQ ID NO: 83;
HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 84; and
HC-CDR1: SEQ ID NO: 13, HC-CDR2: SEQ ID NO: 29, HC-CDR3: SEQ ID NO: 44, LC-CDR1: SEQ ID NO: 57, LC-CDR2: SEQ ID NO: 66, LC-CDR3: SEQ ID NO: 85.

15. The antibody according to claim 4, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to
HC-CDR1: SEQ ID NO: 1, HC-CDR2: SEQ ID NO: 28, HC-CDR3: SEQ ID NO: 30; LC-CDR1: SEQ ID NO: 45, LC-CDR2: SEQ ID NO: 69, LC-CDR3: SEQ ID NO: 70;
wherein the CDRs comprise 0-2 amino acid mutations, substitutions, or deletions in at least one of the HC-CDR1, HC-CDR2, HC-CDR3, LC-CDR1, LC-CDR2, LC-CDR3.

16. The antibody according to claim 4, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to
HC-CDR1: SEQ ID NO: 1, HC-CDR2: SEQ ID NO: 28, HC-CDR3: SEQ ID NO: 30; LC-CDR1: SEQ ID NO: 45, LC-CDR2: SEQ ID NO: 69, LC-CDR3: SEQ ID NO: 70.

17. The antibody according to claim 4, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to
HC-CDR1: SEQ ID NO: 1, HC-CDR2: SEQ ID NO: 28, HC-CDR3: SEQ ID NO: 177; LC-CDR1: SEQ ID NO: 45, LC-CDR2: SEQ ID NO: 69, LC-CDR3: SEQ ID NO: 70;
wherein the CDRs comprise 0-2 amino acid mutations, substitutions, or deletions in at least one of the HC-CDR1, HC-CDR2, HC-CDR3, LC-CDR1, LC-CDR2, LC-CDR3.

18. The antibody according to claim 4, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to
HC-CDR1: SEQ ID NO: 1, HC-CDR2: SEQ ID NO: 28, HC-CDR3: SEQ ID NO: 177; LC-CDR1: SEQ ID NO: 45, LC-CDR2: SEQ ID NO: 69, LC-CDR3: SEQ ID NO: 70.

19. The antibody according to claim 4, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to
HC-CDR1: SEQ ID NO: 1, HC-CDR2: SEQ ID NO: 28, HC-CDR3: SEQ ID NO: 178; LC-CDR1: SEQ ID NO: 45, LC-CDR2: SEQ ID NO: 69, LC-CDR3: SEQ ID NO: 70;
wherein the CDRs comprise 0-2 amino acid mutations, substitutions, or deletions in at least one of the HC-CDR1, HC-CDR2, HC-CDR3, LC-CDR1, LC-CDR2, LC-CDR3.

20. The antibody according to claim 4, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to
HC-CDR1: SEQ ID NO: 1, HC-CDR2: SEQ ID NO: 28, HC-CDR3: SEQ ID NO: 178; LC-CDR1: SEQ ID NO: 45, LC-CDR2: SEQ ID NO: 69, LC-CDR3: SEQ ID NO: 70.

21. The antibody according to claim 4, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to
HC-CDR1: SEQ ID NO: 13, HC-CDR2: SEQ ID NO: 29, HC-CDR3: SEQ ID NO: 43; LC-CDR1: SEQ ID NO: 57, LC-CDR2: SEQ ID NO: 66, LC-CDR3: SEQ ID NO: 83;
wherein the CDRs comprise 0-2 amino acid mutations, substitutions, or deletions in at least one of the HC-CDR1, HC-CDR2, HC-CDR3, LC-CDR1, LC-CDR2, LC-CDR3.

22. The antibody according to claim 4, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to HC-CDR1: SEQ ID NO: 13, HC-CDR2: SEQ ID NO: 29, HC-CDR3: SEQ ID NO: 43; LC-CDR1: SEQ ID NO: 57, LC-CDR2: SEQ ID NO: 66, LC-CDR3: SEQ ID NO: 83.

23. The antibody according to claim 4, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to
HC-CDR1: SEQ ID NO: 13, HC-CDR2: SEQ ID NO: 29, HC-CDR3: SEQ ID NO: 44; LC-CDR1: SEQ ID NO: 57, LC-CDR2: SEQ ID NO: 66, LC-CDR3: SEQ ID NO: 84;
wherein the CDRs comprise 0-2 amino acid mutations, substitutions, or deletions in at least one of the HC-CDR1, HC-CDR2, HC-CDR3, LC-CDR1, LC-CDR2, LC-CDR3.

24. The antibody according to claim 4, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to
HC-CDR1: SEQ ID NO: 13, HC-CDR2: SEQ ID NO: 29, HC-CDR3: SEQ ID NO: 44; LC-CDR1: SEQ ID NO: 57, LC-CDR2: SEQ ID NO: 66, LC-CDR3: SEQ ID NO: 84.

25. The antibody according to claim 4, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to
HC-CDR1: SEQ ID NO: 13, HC-CDR2: SEQ ID NO: 29, HC-CDR3: SEQ ID NO: 44; LC-CDR1: SEQ ID NO: 57, LC-CDR2: SEQ ID NO: 66, LC-CDR3: SEQ ID NO: 85;
wherein the CDRs comprise 0-2 amino acid mutations, substitutions, or deletions in at least one of the HC-CDR1, HC-CDR2, HC-CDR3, LC-CDR1, LC-CDR2, LC-CDR3.

26. The antibody according to claim 4, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise sequences according to
HC-CDR1: SEQ ID NO: 13, HC-CDR2: SEQ ID NO: 29, HC-CDR3: SEQ ID NO: 44; LC-CDR1: SEQ ID NO: 57, LC-CDR2: SEQ ID NO: 66, LC-CDR3: SEQ ID NO: 85.

27. The antibody according to claim 1, wherein the antibody comprises an antibody format selected from Fab, Fab′, scFv, and (Fab′)₂.

28. The antibody according to claim 1, wherein the heavy chain variable domain is fused to a human IgG1 constant region.

29. The antibody according to claim 1, wherein the heavy chain variable domain is fused to a human IgG4 constant region.

30. The antibody according to claim 1, wherein the light chain variable domain is fused to a human Kappa constant region.

31. The antibody according to claim 1, wherein the heavy chain variable domain comprises a variable domain of an IgG1, IgG2, IgG3, or IgG4 heavy chain.

32. The antibody according to claim 1, wherein the light chain variable domain comprises a variable domain of a Kappa light chain.

33. The antibody according to claim 1, wherein the heavy chain variable domain comprises an amino acid sequence with at least 90%, 95%, 96%, 97%, 98%, 99%, or 100% identity to any one of SEQ ID NOs: 143, 149, 181, 184, 155, 161, or 167.

34. The antibody according to claim 1, wherein the light chain variable domain comprises an amino acid sequence with at least 90%, 95%, 96%, 97%, 98%, 99%, or 100% identity to any one of SEQ ID NOs: 144, 150, 156, 162, or 168.

35. The antibody according to claim 1, wherein the heavy chain variable domain comprises an amino acid sequence with at least 90% sequence identity to SEQ ID NO: 143, and the light chain variable domain comprises an amino acid sequence with at least 90% sequence identity to SEQ ID NO: 144.

36. The antibody according to claim 1, wherein the heavy chain variable domain comprises an amino acid sequence with at least 90% sequence identity to SEQ ID NO: 149, and the light chain variable domain comprises an amino acid sequence with at least 90% sequence identity to SEQ ID NO: 150.

37. The antibody according to claim 1, wherein the heavy chain variable domain comprises an amino acid sequence with at least 90% sequence identity to SEQ ID NO: 181, and the light chain variable domain comprises an amino acid sequence with at least 90% sequence identity to SEQ ID NO: 144.

38. The antibody according to claim 1, wherein the heavy chain variable domain comprises an amino acid sequence with at least 90% sequence identity to SEQ ID NO: 184, and the light chain variable domain comprises an amino acid sequence with at least 90% sequence identity to SEQ ID NO: 144.

39. The antibody according to claim 1, wherein the heavy chain variable domain comprises an amino acid sequence with at least 90% sequence identity to SEQ ID NO: 155, and the light chain variable domain comprises an amino acid sequence with at least 90% sequence identity to SEQ ID NO: 156.

40. The antibody according to claim 1, wherein the heavy chain variable domain comprises an amino acid sequence with at least 90% sequence identity to SEQ ID NO: 161, and the light chain variable domain comprises an amino acid sequence with at least 90% sequence identity to SEQ ID NO: 162.

41. The antibody according to claim 1, wherein the heavy chain variable domain comprises an amino acid sequence with at least 90% sequence identity to SEQ ID NO: 167, and the light chain variable domain comprises an amino acid sequence with at least 90% sequence identity to SEQ ID NO: 168.

42. The antibody according to claim 1, wherein the antibody comprises amino acid sequences with at least 90% sequence identity to SEQ ID NO: 145, and at least 90% sequence identity to SEQ ID NO: 147.

43. The antibody according to claim 1, wherein the antibody comprises amino acid sequences with at least 90% sequence identity to SEQ ID NO: 151, and at least 90% sequence identity to SEQ ID NO: 153.

44. The antibody according to claim 1, wherein the antibody comprises amino acid sequences with at least 90% sequence identity to SEQ ID NO: 182, and at least 90% sequence identity to SEQ ID NO: 147.

45. The antibody according to claim 1, wherein the antibody comprises amino acid sequences with at least 90% sequence identity to SEQ ID NO: 185, and at least 90% sequence identity to SEQ ID NO: 147.

46. The antibody according to claim 1, wherein the antibody comprises amino acid sequences with at least 90% sequence identity to SEQ ID NO: 157, and at least 90% sequence identity to SEQ ID NO: 159.

47. The antibody according to claim 1, wherein the antibody comprises amino acid sequences with at least 90% sequence identity to SEQ ID NO: 163, and at least 90% sequence identity to SEQ ID NO: 165.

48. The antibody according to claim 1, wherein the antibody comprises amino acid sequences with at least 90% sequence identity to SEQ ID NO: 169, and at least 90% sequence identity to SEQ ID NO: 171.

49. The antibody according to claim 1, wherein the antibody binds to extracellular domain of human CD47 with an EC50 of between 0.01 nM-0.5 nM as determined by an ELISA binding assay.

50. The antibody according to claim 49, wherein the ELISA binding assay comprises the following steps:
(a) coating a 96-well plate for at least 12 hours with 1 μg/ml of recombinant CD47;
(b) washing the plate three times;
(c) blocking the plate with 300 μL of 1% bovine serum albumin in phosphate-buffered saline solution with tween (PBST) at 37° C. for 1 hour;
(d) washing the plate four times with PBST;
(e) incubating a serial dilution of the antibody that binds specifically to CD47 at 37° C. for 1 hour;
(f) washing the plate 3 times with PBST;
(g) incubating a 1:5000 dilution of anti-human IgG-peroxidase antibody at 37° C. for 1 hour within the plate;
(h) washing the plate 4 times with PBST;
(i) incubating 3,3′,5,5′ tetramethylbenzidine substrate for 15 minutes at room temperature within the plate;
(j) terminating the reaction within the plate with 1N HCl; and
(k) reading the plate at 450 nM to determine the EC50 of the antibody for binding to the extracellular domain of human CD47.

51. The antibody according to claim 49, wherein the extracellular domain of human CD47 comprises an amino acid sequence according to SEQ ID NO: 86.

52. The antibody according to claim 1, wherein the antibody binds to CD47+ cell lines with an EC50 of between 0.01 nM-5 nM as determined by flow cytometry.

53. The antibody according to claim 52, wherein the EC50 as determined by flow cytometry comprises the following steps and assay conditions:
(a) centrifuging CD47+ cells at 2000 rpm for 5 minutes to obtain centrifuged cells;
(b) resuspending the centrifuged cells in 10-15 mL of culture medium;
(c) resuspending cells in blocking buffer comprising phosphate buffer saline with 2% fetal bovine serum (PBS plus 2% FBS) at a concentration of 3×10⁶ cells/mL to obtain a cell suspension;
(d) dispensing the cell suspension into wells of a 96-well plate;
(e) diluting the antibody that binds specifically to CD47 in the blocking buffer to a desired concentration followed by addition of 100 μL of the antibody per well and incubating at 1 hour for 4° C.;
(f) washing the cells three times with PBS plus 2% FBS;
(g) resuspending the cells in 100 μL 1:500 diluted Alexa Fluor 488 labeled Mouse anti-Human IgG1 Fc secondary antibody and incubating at 4° C. in the dark for one hour;
(h) washing the cells three times with 200 μL PBS and centrifuging at 2000 rpm for 5 minutes;
(i) resuspending cells in 300 μL cold PBS; and
(j) analyzing with a flow cytometer to determine the EC50 of the antibody for the CD47+ cell line.

54. The antibody according to claim 1, wherein the antibody blocks SIRPa activity with a IC50 of between 0.1 nM-5 nM as determined by flow cytometry.

55. The antibody according to claim 54, wherein the IC50 as determined by flow cytometry comprises the following steps and assay conditions:
(a) harvesting, centrifuging, and resuspending Raji tumor cells or HCT-15 tumor cells in FACS buffer comprising phosphate buffer saline with 2% fetal bovine serum (PBS plus 2% FBS) at a concentration of 2×10⁶ cells/mL;
(b) dispensing 100 μL of the cell suspension into wells of a 96-well plate;
(c) centrifuging the plate for 5 minutes at 300×g followed by discarding of supernatants;
(d) incubating cells from the plate with 50 μL per well of serially diluted antibody and a constant amount of SIRPA-mIgG2a fusion protein (0.2 μg/mL for Raji cells, 1 μg/mL for HCT-15 cells) in FACS buffer for 1 hour at 4° C.;
(e) washing the plate with FACS buffer followed by incubating for one hour at 4° C. in the dark with 100 μL of Alexa Fluor 488 donkey anti-Mouse IgG(H+L) secondary antibody;
(f) washing twice with FACS buffer,
(g) resuspending cells in the plate with 300 μL FACS buffer; and
(h) analyzing with a flow cytometer to determine the IC50 of the antibody for blocking SIRPa activity.

56. The antibody according to claim 1, wherein the antibody induces increased antibody-dependent cellular phagocytosis (ADCP) as compared to a control antibody that comprises amino acid sequences according to SEQ ID NOs: 173 and 174 when tested under substantially equivalent assay conditions in an assay for ADCP.

57. The antibody according to claim 1, wherein the antibody has less binding on human red blood cells (RBC) as compared to a control antibody that comprises amino acid sequences according to SEQ ID NOs: 173 and 174 when tested under substantially equivalent assay conditions for assessing RBC binding.

58. The antibody according to claim 1, wherein a concentration of 600 nM of the antibody does not induce hemolysis of red blood cells in a hemagglutination assay.

59. The antibody according to claim 1, wherein the antibody induces increased reduction in tumor volume as compared to a control antibody that comprises amino acid sequences according to SEQ ID NOs: 173 and 174 when tested under substantially equivalent assay conditions in a tumor growth animal model.

60. A nucleic acid molecule encoding the antibody according to claim 1.

61. A vector comprising the nucleic acid molecule of claim 60.

62. A pharmaceutical composition comprising the antibody according to claim 1.

63. The pharmaceutical composition of claim 62, further comprising a pharmaceutically acceptable carrier, an excipient, or any combinations thereof.

64. A method of treating a subject having cancer, the method comprising: administering to the subject the antibody of claim 1, or the pharmaceutical composition of claim 62.

65. The method of claim 64, wherein the cancer comprises cancer cells that express CD47.

66. The method of claim 64, wherein the antibody induces antibody-dependent cellular phagocytosis (ADCP) of the cancer cells that express CD47.

67. The method of claim 64, wherein the cancer is a hematological malignancy.

68. The method of claim 64, wherein the cancer is B cell cancer or T cell cancer.

69. The method of claim 64, wherein the cancer is leukemia or lymphoma.

70. The method of claim 64, wherein the cancer is lymphoma, and wherein the lymphoma is B-cell lymphoma.

71. The method of claim 64, wherein the cancer is lymphoma, and wherein the lymphoma is T-cell lymphoma.

72. The method of claim 64, wherein the cancer is a solid tumor.

73. The method of claim 72, wherein the solid tumor is sarcoma, breast cancer, lung cancer, carcinoma, ovarian cancer, pancreatic cancer, gastric cancer, colorectal cancer, endometrial cancer, esophageal cancer, prostate cancer, cervical cancer, kidney cancer, urothelial cancer, or head and neck cancer.

74. The method of claim 73, wherein the solid tumor is colorectal cancer.

75. The method of claim 73, wherein the solid tumor is gastric cancer.

76. The method of claim 73, wherein the solid tumor is lung cancer, and wherein the lung cancer is non-small cell lung cancer.

77. The method of claim 73, wherein the solid tumor is lung cancer, and wherein the lung cancer is small cell lung cancer.

78. The method of claim 64, further comprising administering to the subject an anti-cancer agent.

79. The method of claim 78, wherein the anti-cancer agent is a chemotherapeutic agent or a biologic agent.

80. The method of claim 64, wherein the administering is sufficient to reduce or eliminate the cancer as compared to a comparable method lacking the administering.

81. The method of claim 80, wherein the reduction is at least about 1 fold, 5 fold, 10 fold, 20 fold, 40 fold, 60 fold, 80 fold, or up to about 100 fold.

82. The method of claim 64, wherein the cancer is metastatic.

83. A kit that comprises at least one of:
(a) the antibody of claim 1;
(b) the vector of claim 61;
(c) the nucleic acid molecule of claim 60; or
(d) the pharmaceutical composition of claim 62.
